(19) United States
(12) Patent Application Publication
(10) Pub. No.: US 2023/0383277 A1
(43) Pub. Date: Nov. 30, 2023

(54) COMPOSITIONS AND METHODS FOR TREATING GLYCOGEN STORAGE DISEASE TYPE 1A

(71) Applicant: Beam Therapeutics Inc., Cambridge, MA (US)

(72) Inventors: Brian CAFFERTY, Cambridge, MA (US); Tanggis BOHNUUD, Cambridge, MA (US); Lo-I CHENG, Cambridge, MA (US); Michael PACKER, Cambridge, MA (US); Yvonne ARATYN-SCHAUS, Cambridge, MA (US)

(73) Assignee: Beam Therapeutics Inc., Cambridge, MA (US)

(21) Appl. No.: 18/031,547

(22) PCT Filed: Oct. 14, 2021
(86) PCT No.: PCT/US2021/055057

§ 371 Date: Apr. 12, 2023

Related U.S. Application Data
(60) Provisional application No. 63/091,891, filed on Oct. 14, 2020.
(60) Provisional application No. 63/248,081, filed on Sep. 24, 2021.

(51) Int. Cl. C12N 9/78; A61K 48/00; C12N 9/22; C12N 15/11; C12N 15/113

(52) U.S. Cl. CPC C12N 9/78; A61K 48/0066; C12Y 305/04004; C12N 9/22; C12N 15/11; C12N 15/1137; C12Y 301/03009; C07K 2319/80; C12N 2310/20; C12N 2310/321; C12N 2310/315

(57) ABSTRACT

Described and provided are adenosine base editors and compositions comprising adenosine base editors that have increased efficiency. Also described and provided are methods of using base editors comprising adenosine deaminase variants for altering mutations associated with Glycogen Storage Disease Type 1a (GSD1a).

### CROSS REFERENCE TO RELATED APPLICATIONS

This application claims priority to and benefit of Provisional Patent Application Nos. 63/091,891, filed on Oct. 14, 2020 and 63/248,081, filed on Sep. 24, 2021, the contents of all of which are hereby incorporated by reference in their entireties.

### SEQUENCE LISTING

This application contains a Sequence Listing which has been submitted electronically in ASCII format and is hereby incorporated by reference in its entirety. The ASCII copy, created on Oct. 14, 2021, is named 180802-047002PCT_SL and is 2,088,767 bytes in size.

### BACKGROUND OF THE DISCLOSURE

For most known genetic diseases, correction of a point mutation in the target locus, rather than stochastic disruption of the gene, is needed to study or address the underlying cause of the disease. Current genome editing technologies utilizing the clustered regularly interspaced short palindromic repeat (CRISPR) system introduce double-stranded DNA breaks at a target locus as the first step to gene correction. In response to double-stranded DNA breaks, cellular DNA repair processes mostly result in random insertions or deletions (indels) at the site of DNA cleavage through non-homologous end joining. Although most genetic diseases arise from point mutations, current approaches to point mutation correction are inefficient and typically induce an abundance of random insertions and deletions (indels) at the target locus resulting from the cellular response to dsDNA breaks. Therefore, there is a need for an improved form of genome editing that is more efficient and with far fewer undesired products such as stochastic insertions or deletions (indels) or translocations.

Glycogen Storage Disease Type 1 (also known as GSD1 or Von Gierke Disease) is an inherited disorder that results in a deficiency in glycogenolysis and gluconeogenesis, with accumulation of glycogen and lipids in tissues, causing life-threatening hypoglycemia and lactic acidosis and leading to potential CNS damage and long-term liver and renal complications, such as steatosis, hepatic adenomas and hepatocellular carcinomas.

There are two types of GSD1, Type 1a (GSD1a or GSD-Ia) and Type 1b (GSD1b), which are caused by different genetic mutations. GSD1a is caused by a mutation in the glucose-6-phosphatase (G6PC) gene and affects about 80% of patients with GSD1. About one in 100,000 newborns in the US have GSD1a with about 22% of patients carrying the recessive mutation Q347* and 37% of patients carrying the recessive mutation R83C.

There are no drug therapies approved for GSD1a. Although liver transplants are curative, there are no approved therapies and the current treatment regimen involves nearly continuous cornstarch feeding. If chronically untreated, patients develop severe lactic acidosis, can progress to renal failure, and die in infancy or childhood. GSD1a is an area of significant unmet medical need. Therefore, there is a need for novel compositions and methods for treating patients with GSD1a.

### SUMMARY OF THE DISCLOSURE AND EMBODIMENTS

Featured, provided and described herein are compositions and methods for the precise correction of pathogenic amino acids using a programmable nucleobase editor. In particular, the compositions and methods disclosed and described herein are useful for the treatment of Glycogen Storage Disease Type 1a (GSD1a). Thus, compositions and methods are provided for treating GSD1a using an adenosine (A) base editor (ABE) to precisely correct a single nucleotide polymorphism in the endogenous G6PC gene to correct a deleterious mutation (e.g., Q347X, R83C).

In an aspect, an adenosine deaminase variant including a glycine (G) at amino acid position 82, a threonine (T) or an aspartic acid (D) at amino acid position 147, a serine (S) at amino acid position 154, and one or more of a histidine (H) at amino acid position 36, a tyrosine at amino acid position 76, a tyrosine at amino acid position 149, a lysine (K) at amino acid position 157, and an asparagine (N) at amino acid position 167 of the following amino acid sequence is provided, wherein the adenosine deaminase has at least about 85% identity to said amino acid sequence: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1), or corresponding alterations in another adenosine deaminase.

Another aspect provides an adenosine deaminase variant, including any of the following combinations of alterations: a) I76Y+V82G+Y147T+Q154S; b) L36H+V82G+Y147T+Q154S+N157K; c) V82G+Y147D+F149Y+Q154S+D167N; d) L36H+V82G+Y147D+F149Y+Q154S+N157K+D167N; e) L36H+I76Y+V82G+Y147T+Q154S+N157K; f) I76Y+V82G+Y147D+F149Y+Q154S+D167N; g) Y147D+F149Y+D167N; h) L36H; I76Y; V82G; Q154S; and N157K; i) I76Y; V82G; Q154S; or j) L36H+I76Y+V82G+Y147D+F149Y+Q154S+N157K+D167N with reference to SEQ ID NO: 1: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1), or corresponding combinations of alterations in another adenosine deaminase.

In an embodiment of the above-delineated adenosine deaminase variants, the adenosine deaminase variant includes the following combination of alterations I76Y+V82G+Y147D+F149Y+Q154S+D167N of SEQ ID NO: 1, or corresponding alterations in another adenosine deaminase. In another embodiment, the adenosine deaminase has at least about 90% identity to SEQ ID NO: 1. In another embodiment, the adenosine deaminase has at least about 95% identity to SEQ ID NO: 1. In another embodiment, the adenosine deaminase comprises or consists essentially of SEQ ID NO: 1.

In another aspect, a fusion protein or complex including a polynucleotide programmable DNA binding domain and at least one adenosine deaminase variant domain is provided, wherein the adenosine deaminase variant domain comprises a glycine (G) at amino acid position 82, a threonine (T) or an aspartic acid (D) at amino acid position 147, a serine (S) at amino acid position 154, and one or more of a histidine (H) at amino acid position 36, a tyrosine at amino acid position 76, a tyrosine at amino acid position 149, a lysine (K) at amino acid position 157, and an asparagine (N) at amino acid position 167 of the following amino acid sequence, wherein the adenosine deaminase has at least about 85% identity to said amino acid sequence MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1), or corresponding alterations in another adenosine deaminase. In an embodiment of the fusion protein or complex, the adenosine deaminase variant domain has at least about 90% identity to SEQ ID NO: 1. In another embodiment of the fusion protein or complex, the adenosine deaminase variant domain at least about 95% identity to SEQ ID NO: 1. In another embodiment of the fusion protein or complex, the adenosine deaminase variant domain comprises or consists essentially of SEQ ID NO: 1.

Yet another aspect provides a fusion protein or complex including a polynucleotide programmable DNA binding domain and at least one adenosine deaminase variant domain, wherein the adenosine deaminase variant domain comprises any of the following combinations of alterations: a) I76Y+V82G+Y147T+Q154S; b) L36H+V82G+Y147T+Q154S+N157K; c) V82G+Y147D+F149Y+Q154S+D167N; d) L36H+V82G+Y147D+F149Y+Q154S+N157K+D167N; e) L36H+I76Y+V82G+Y147T+Q154S+N157K; f) I76Y+V82G+Y147D+F149Y+Q154S+D167N; g) Y147D+F149Y+D167N; h) L36H; I76Y; V82G; Q154S; and N157K; i) I76Y; V82G; Q154S; or j) L36H+I76Y+V82G+Y147D+F149Y+Q154S+N157K+D167N with reference to SEQ ID NO: 1: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1), or corresponding combinations of alterations in another adenosine deaminase.

In some embodiments of the above-delineated fusion protein or complex, the adenosine deaminase variant includes the following combination of alterations I76Y+V82G+Y147D+F149Y+Q154S+D167N of SEQ ID NO: 1, or corresponding alterations in another adenosine deaminase. In some embodiments, the fusion protein or complex includes one adenosine deaminase variant domain. In some embodiments, the fusion protein or complex includes a wild-type adenosine deaminase domain and an adenosine deaminase variant domain. In some embodiments, the fusion protein or complex includes a TadA*7.10 adenosine deaminase domain and an adenosine deaminase variant domain. In some embodiments, the polynucleotide programmable DNA binding domain is a Cas9 domain. In some embodiments, the Cas9 domain comprises a nuclease dead Cas9 (dCas9), a Cas9 nickase (nCas9), or a nuclease active Cas9. In some embodiments, the polynucleotide programmable DNA binding domain is a Staphylococcus aureus Cas9 (SaCas9), Streptococcus thermophilus 1 Cas9 (St1Cas9), a Streptococcus pyogenes Cas9 (SpCas9), or variants thereof. In some embodiments, the polynucleotide programmable DNA binding domain comprises a modified SaCas9 having an altered protospacer-adjacent motif (PAM) specificity. In some embodiments, the SaCas9 has protospacer-adjacent motif (PAM) specificity for the nucleic acid sequence 5′-NNGRRT-3′. In some embodiments, the SaCas9 has specificity for the nucleic acid sequence 5′-GAGAAT-3′. In some embodiments, the SaCas9 is a nuclease active SaCas9, a nuclease inactive SaCas9 (SaCas9d), or a SaCas9 nickase (SaCas9n). In some embodiments, the SaCas9 is a nickase comprising an amino acid substitution N579A or a corresponding amino acid substitution thereof. In some embodiments, the SaCas9 is a Streptococcus pyogenes Cas9 (SpCas9) or a variant thereof. In some embodiments of the above-delineated fusion protein or complex, the adenosine deaminase variant is capable of deaminating adenine in deoxyribonucleic acid (DNA). In some embodiments, the fusion protein or complex further includes a linker between the polynucleotide programmable DNA binding domain and the adenosine deaminase variant domain. In some embodiments, the linker comprises the amino acid sequence: SGGSSGGSSGSETPGTSESATPES (SEQ ID NO: 359). In some embodiments, the fusion protein or complex includes one or more nuclear localization signal. In some embodiments, the nuclear localization signal is a bipartite nuclear localization signal. In some embodiments, the polynucleotide programmable DNA binding domain non-covalently associates with the deaminase.

Another aspect provides a base editor system including any of the fusion proteins or complexes as provided herein and one or more guide polynucleotides. In some embodiments, the one or more guide polynucleotides target the fusion protein to effect an A·T to G·C alteration of a single nucleotide polymorphism (SNP) associated with a genetic disease. In some embodiments, the genetic disease is Glycogen Storage Disease Type 1a (GSD1a). In some embodiments, the guide polynucleotide comprises ribonucleic acid (RNA), or deoxyribonucleic acid (DNA). In some embodiments, the guide polynucleotide comprises a nucleic acid sequence: 5′-CAGUAUGGACACUGUCCAAA-3′ (SEQ ID NO: 370). In some embodiments, the guide comprises or consists of one of the following nucleic acid sequences: CACCAGUAUGGACACUGUCCAAAGUUUUAGUACUCUGUAAUGAAAAUUACAGAAUCUACUAA AACAAGGCAAAAUGCCGUGUUUAUCUCGUCAACUUGUUGGCGAGAUUUU (SEQ ID NO: 409), or CCACCAGUAUGGACACUGUCCAAAGUUUUAGUACUCUGUAAUGAAAAUUACAGAAUCUACUA AAACAAGGCAAAAUGCCGUGUUUAUCUCGUCAACUUGUUGGCGAGAUUUU (SEQ ID NO: 410). In some embodiments, the guide polynucleotide comprises one or more modified nucleosides at the 5′ end and/or the 3′ end of the guide. In some embodiments, the guide polynucleotide comprises two, three, four or more modified nucleosides at the 5′ end and/or the 3′ end of the guide. In some embodiments, the guide polynucleotide comprises two, three, four or more modified nucleosides at the 5′ end and/or the 3′ end of the guide. In some embodiments, the guide polynucleotide comprises four modified nucleosides at the 5′ end and four modified nucleosides at the 3′ end of the guide. In some embodiments, the modified nucleoside comprises a 2′ O-methyl or a phosphorothioate. In some embodiments, the guide polynucleotide comprises or consists essentially of one of the following sequences: mCsmAsmCsCAGUAUGGACACUGUCCAAAGUUUUAGUACUCUGUAAUGAAAAUUACAGAAUC UACUAAAACAAGGCAAAAUGCCGUGUUUAUCUCGUCAACUUGUUGGCGAGAmUsmUsmUsU (SEQ ID NO: 409) or mCsmCsmAsCCAGUAUGGACACUGUCCAAAGUUUUAGUACUCUGUAAUGAAAAUUACAGAAU CUACUAAAACAAGGCAAAAUGCCGUGUUUAUCUCGUCAACUUGUUGGCGAGAmUsmUsmUsU (SEQ ID NO: 410), wherein “m” denotes a 2′ O-methyl and the “s” denotes a phosphorothioate. In some embodiments, the guide polynucleotide comprises a nucleic acid sequence: 5′-CAGUAUGGACACUGUCCAAA-3′ (SEQ ID NO: 370). In some embodiments, the guide polynucleotide comprises a nucleic acid sequence, from 5′ to 3′, as follows: CCACCAGUAUGGACACUGUC (SEQ ID NO: 371); CACCAGUAUGGACACUGUCC (SEQ ID NO: 372); ACCAGUAUGGACACUGUCCA (SEQ ID NO: 373); CCAGUAUGGACACUGUCCAA (SEQ ID NO: 374); CAGUAUGGACACUGUCCAAA (SEQ ID NO: 370); AGUAUGGACACUGUCCAAAG (SEQ ID NO: 375); GUAUGGACACUGUCCAAAGA (SEQ ID NO: 376); or UAUGGACACUGUCCAAAGAG (SEQ ID NO: 377). In some embodiments, the adenosine deaminase variant domain is internal to the Cas protein.

Another aspect provides a polynucleotide encoding any of the adenosine deaminase variants as provided herein, any of the fusion proteins or complexes as provided herein, or any of the base editor systems as provided herein. In an embodiment, the polynucleotide comprises one or more modified nucleosides or nucleotides. In an embodiment, the polynucleotide is DNA or RNA. In embodiments, the polynucleotide comprises a modification selected from the group consisting of 2′O-methyl (2′-OMe), phosphorothioate (PS), 2′O-methyl thioPACE (MSP), 2′O-methyl-PACE (MP), 2′-fluoro RNA (2′-F-RNA), and constrained ethyl (S-cEt).

Another aspect provides a cell including any of the polynucleotides as provided herein.

Yet another aspect provides a cell including any of the adenosine deaminase variants as provided herein, any of the fusion proteins or complexes as provided herein and one or more guide polynucleotides, any of the base editor systems as provided herein, or any of the polynucleotides as provided herein.

In some embodiments of the above-delineated aspects, the cell is a hepatocyte, a hepatocyte precursor, or an iPSc-derived hepatocyte. In some embodiments, the cell expresses a G6PC polypeptide. In some embodiments, the cell is from a subject having Glycogen Storage Disease Type 1a (GSD1a). In some embodiments, the cell is a mammalian cell in vivo, ex vivo, or in vitro. In some embodiments, the cell is a human cell. In some embodiments, the fusion protein and the one or more guide polynucleotides form a complex in the cell.

In another aspect, a method of treating a genetic disease in a subject in need thereof is provided, in which the method involves administering to a cell of the subject any of the base editor systems as provided herein or a polynucleotide encoding the base editor system.

In another aspect, a method of treating a genetic disease in a subject in need thereof is provided, in which the method involves administering to the subject any of the cells as provided herein.

In some embodiments of the above-delineated methods, after treatment the cell expresses a G6PC polypeptide capable of catalyzing the hydrolysis of D-glucose 6-phosphate to D-glucose and orthophosphate. In some embodiments, the cell is autologous, allogeneic, or xenogeneic to the subject. In some embodiments, the genetic disease is Glycogen Storage Disease Type 1a (GSD1a) and/or symptoms thereof. In an embodiment of the above-denoted treatment methods, the subject sustains at least a 24 hour fasting period after treatment.

In another aspect, a method for correcting a single nucleotide polymorphism (SNP) in a polynucleotide is provided, in which the method involves contacting a target nucleotide sequence, at least a portion of which is located in the polynucleotide or its reverse complement, with any of the base editor systems as provided herein; and editing the SNP by deaminating the SNP or its complement nucleobase upon targeting of the base editor to the target nucleotide sequence, wherein deaminating the SNP or its complement nucleobase corrects the SNP. In some embodiments, the SNP is associated with Glycogen Storage Disease Type 1a (GSD1a). In some embodiments, the SNP is in the G6PC gene.

In another aspect, a method of editing a glucose-6-phosphatase (G6PC) polynucleotide comprising a single nucleotide polymorphism (SNP) associated with Glycogen Storage Disease Type 1a (GSD1a) is provided, in which the method includes contacting the G6PC polynucleotide with any of the fusion proteins or complexes as provided herein in a complex with one or more guide polynucleotides, and wherein one or more of said guide polynucleotides target said base editor to effect an A·T to G·C alteration of the SNP associated with GSD1a.

In some embodiments of the above-delineated methods, the contacting is in a cell, a eukaryotic cell, a mammalian cell, or human cell. In some embodiments, the SNP changes a glutamine (Q) to a non-glutamine (X) amino acid or changes an arginine (R) to a non-arginine (X) in a G6PC polypeptide. In some embodiments, the SNP results in expression of an G6PC polypeptide having a non-glutamine (X) amino acid at position 347 or a non-arginine (X) amino acid at position 83. In some embodiments, the base editor correction replaces the non-glutamine amino acid (X) at position 347 with a glutamine or the non-arginine amino acid (X) at position 83 with an arginine (R). In some embodiments, the SNP results in expression of a G6PC polypeptide that prematurely terminates at amino acid position 347 or at a cysteine at position 83. In some embodiments, the SNP encodes one or more of Q347X and/or R83C. In some embodiments, the SNP encodes R83C. In some embodiments, the editing results in less than 0.5% indel formation. In some embodiments, the editing rescues G6PC catalytic activity. In some embodiments, the guide polynucleotide comprises a nucleic acid sequence: 5′-CAGUAUGGACACUGUCCAAA-3′ (SEQ ID NO: 370). In some embodiments, the guide polynucleotide comprises a nucleic acid sequence, from 5′ to 3′, as follows: CCACCAGUAUGGACACUGUC (SEQ ID NO: 371); CACCAGUAUGGACACUGUCC (SEQ ID NO: 372); ACCAGUAUGGACACUGUCCA (SEQ ID NO: 373); CCAGUAUGGACACUGUCCAA (SEQ ID NO: 374); CAGUAUGGACACUGUCCAAA (SEQ ID NO: 370); AGUAUGGACACUGUCCAAAG (SEQ ID NO: 375); GUAUGGACACUGUCCAAAGA (SEQ ID NO: 376); or UAUGGACACUGUCCAAAGAG (SEQ ID NO: 377). In some embodiments of the methods, the subject sustains at least a 24 hour fasting period after treatment.

Another aspect provides a vector comprising any of the polynucleotides as provided and described herein. In some embodiments, the vector is a viral vector. In some embodiments, the viral vector is a retroviral vector, adenoviral vector, lentiviral vector, herpesvirus vector, or adeno-associated viral vector (AAV).

Another aspect provides a composition including any of the fusion proteins or complexes as provided herein, any of the base editor systems as provided herein, any of the polynucleotides as provided herein, any of the cells as provided herein, or any of the vectors as provided herein. In some embodiments, the composition further includes a pharmaceutically acceptable excipient, carrier, or vehicle. In some embodiments, the one or more guide polynucleotides and the fusion protein are formulated together or separately. In some embodiments, the composition further includes a ribonucleoparticle suitable for expression in a mammalian cell. In some embodiments, the composition further includes a lipid. In some embodiments, the composition comprises a lipid nanoparticle (LNP).

Yet another aspect provides a kit including any of the fusion proteins or complexes as provided herein, any of the base editor systems as provided herein, any of the polynucleotides as provided herein, any of the cells as provided herein, any of the vectors as provided herein, or any of the compositions as provided herein. In some embodiments, the kit further includes written instructions for the use of the kit in the treatment of Glycogen Storage Disease Type 1a (GSD1a).

Another aspect provides any of the fusion proteins or complexes as provided and described herein, any of the base editor systems as provided and described herein, any of the polynucleotides as provided and described herein, any of the cells as provided and described herein, any of the vectors as provided and described herein, or any of the compositions as provided and described herein, wherein the base editor comprises an mRNA sequence as set forth in SEQ ID NO: 396.

Another aspect provides any of the fusion proteins or complexes as provided and described herein, any of the base editor systems as provided and described herein, any of the polynucleotides as provided and described herein, any of the cells as provided and described herein, any of the vectors as provided and described herein, or any of the compositions as provided and described herein, wherein the base editor comprises a DNA sequence as set forth in SEQ ID NO: 397.

Another aspect provides any of the fusion proteins or complexes as provided and described herein, any of the base editor systems as provided and described herein, any of the polynucleotides as provided and described herein, any of the cells as provided and described herein, any of the vectors as provided and described herein, or any of the compositions as provided and described herein, wherein the base editor comprises an amino acid sequence as set forth in SEQ ID NO: 398.

In another aspect, a modified guide RNA (gRNA) comprising modified nucleotides is provided, wherein the gRNA comprises from 5′ to 3′ a polynucleotide sequence selected from the group consisting of:

(SEQ ID NO: 404)
CAGUAUGGACACUGUCCAAAGUUUUAGUACUCUGUAAUGAAAAUUACA
GAAUCUACUAAAACAAGGCAAAAUGCCGUGUUUAUCUCGUCAACUUGU
UGGCGAGAUUUU;
(SEQ ID NO: 405)
UUUCAGUAUGGACACUGUCCAAAGUUUUAGUACUCUGUAAUGAAAAUU
ACAGAAUCUACUAAAACAAGGCAAAAUGCCGUGUUUAUCUCGUCAACU
UGUUGGCGAGAUUUU;
(SEQ ID NO: 406)
CAGUAUGGACACUGUCCAAAGUUUUAGUACUCUGGAAACAGAAUCUAC
UAAAACAAGGCAAAAUGCCGUGUUUAUCUCGUCAACUUGUUGGCGAGA
UUUU;
(SEQ ID NO: 407)
CCAGUAUGGACACUGUCCAAAGUUUUAGUACUCUGUAAUGAAAAUUAC
AGAAUCUACUAAAACAAGGCAAAAUGCCGUGUUUAUCUCGUCAACUUG
UUGGCGAGAUUUU;
(SEQ ID NO: 408)
ACCAGUAUGGACACUGUCCAAAGUUUUAGUACUCUGUAAUGAAAAUUA
CAGAAUCUACUAAAACAAGGCAAAAUGCCGUGUUUAUCUCGUCAACUU
GUUGGCGAGAUUUU;
(SEQ ID NO: 409)
CACCAGUAUGGACACUGUCCAAAGUUUUAGUACUCUGUAAUGAAAAUU
ACAGAAUCUACUAAAACAAGGCAAAAUGCCGUGUUUAUCUCGUCAACU
UGUUGGCGAGAUUUU;
(SEQ ID NO: 410)
CCACCAGUAUGGACACUGUCCAAAGUUUUAGUACUCUGUAAUGAAAAU
UACAGAAUCUACUAAAACAAGGCAAAAUGCCGUGUUUAUCUCGUCAAC
UUGUUGGCGAGAUUUU;
(SEQ ID NO: 411)
CAGUAUGGACACUGUCCAAAGUUUUAGUACUCUGUAGAAAUACAGAAU
CUACUAAAACAAGGCAAAAUGCCGUGUUUAUCUCGUCAACUUGUUGGC
GAGAUUUU;
(SEQ ID NO: 412)
CAGUAUGGACACUGUCCAAAGUUUUAGUACUCUGCGGAAACGCAGAAU
CUACUAAAACAAGGCAAAAUGCCGUGUUUAUCUCGUCAACUUGUUGGC
GAGAUUUU;
(SEQ ID NO: 413)
CAGUAUGGACACUGUCCAAAGUUUUAGUACUCGAAAGAAUCUACUAAA
ACAAGGCAAAAUGCCGUGUUUAUCUCGUCAACUUGUUGGCGAGAUUUU;
(SEQ ID NO: 414)
CAGUAUGGACACUGUCCAAAGUUUUAGUACCCGAAAGCAUCUACUAAA
ACAAGGCAAAAUGCCGUGUUUAUCUCGUCAACUUGUUGGCGAGAUUUU;
and
(SEQ ID NO: 415)
CAGUAUGGACACUGUCCAAAGUUUUAGUACUCUGUAAUGAAAAUUACA
GAAUCUACUAAAACAAGGCAAAAUGCCGUGUUUAUCUCGUCAACUUGU
UGGCGAGAUUUU.

In some embodiments, the guide comprises at least about 50%-75% modified nucleotides. In some embodiments, the guide comprises at least about 85% or more modified nucleotides. In some embodiments, at least about 1-5 nucleotides at the 5′ end of the gRNA are modified and at least about 1-5 nucleotides at the 3′ end of the gRNA are modified. In some embodiments, at least about 3-5 contiguous nucleotides at each of the 5′ and 3′ termini of the gRNA are modified. In some embodiments, at least about 20% of the nucleotides present in a direct repeat or anti-direct repeat are modified. In some embodiments, at least about 50% of the nucleotides present in a direct repeat or anti-direct repeat are modified. In some embodiments, at least about 50-75% of the nucleotides present in a direct repeat or anti-direct repeat are modified. In some embodiments, at least about 100 of the nucleotides present in a direct repeat or anti-direct repeat are modified. In some embodiments, at least about 20% or more of the nucleotides present in a hairpin present in the gRNA scaffold are modified. In some embodiments, at least about 50% or more of the nucleotides present in a hairpin present in the gRNA scaffold are modified. In some embodiments, the guide comprises a variable length protospacer. In some embodiments, the guide comprises a 20-40 nucleotide protospacer. In some embodiments, the guide comprises a protospacer comprising at least about 20-25 nucleotides or at least about 30-35 nucleotides. In some embodiments, the protospacer comprises modified nucleotides. In some embodiments, the guide comprises two or more of the following:
at least about 1-5 nucleotides at the 5′ end of the gRNA are modified and at least about 1-5 nucleotides at the 3′ end of the gRNA are modified;
at least about 20% of the nucleotides present in a direct repeat or anti-direct repeat are modified;
at least about 50-75% of the nucleotides present in a direct repeat or anti-direct repeat are modified;
at least about 20% or more of the nucleotides present in a hairpin present in the gRNA scaffold are modified;
a variable length protospacer; and
a protospacer comprising modified nucleotides.

In some embodiments of the modified gRNA, the gRNA comprises one or more modifications selected from the group consisting of 2′-O-methyl (2′-OMe), phosphorothioate (PS), 2′-O-methyl thioPACE (MSP), 2′-O-methyl-PACE (MP), 2′-fluoro RNA (2′-F-RNA), and constrained ethyl (S-cEt). In embodiments, the gRNA comprises 2′O-methyl or phosphorothioate modifications. In an embodiment, the gRNA comprises 2′-O-methyl and phosphorothioate modifications. In an embodiment, the modifications increase base editing by at least about 2 fold.

In another aspect, a modified guide RNA (gRNA) is provided, wherein the gRNA comprises a nucleic acid sequence, from 5′ to 3′, selected from

(SEQ ID NO: 404)
mCsmAsmGsmUAUmGmGmACAmCUGUCCAAAmGUUUUmAmGmUACUCm
UGmUmAmAmUGmAAAmAmUmUmACmAGAAUCUACmUmAAAACAAGGCA
AmAAUGmCCmGUGUmUmUmAmUmCmUmCmGmUmCmAmAmCmUmUmGmU
mUmGmGmCmGmAmGmAmUsmUsmUsmU;
(SEQ ID NO: 405)
mUsmUsmUsCAGmUAUmGmGmACAmCUGUCCAAAmGUUUUmAmGmUAC
UCmUGmUmAmAmUGmAAAmAmUmUmACmAGAAUCUACmUmAAAACAAG
GCAAmAAUGmCCmGUGUmUmUmAmUmCmUmCmGmUmCmAmAmCmUmUm
GmUmUmGmGmCmGmAmGmAmUsmUsmUsmU;
(SEQ ID NO: 404)
mCsmAsGsmUAUmGmGmAmCAmCmUGUCCAAmAmGUmUUmUmAmGmUA
CUmCmUmGmUmAmAmUGmAmAmAmAmUmUmACmAmGAAmUCUACmUmA
mAAACAAGmGCAAmAAUGmCmCmGUGmUmUmUmAmUmCmUmCmGmUmC
mAmAmCmUmUmGmUmUmGmGmCmGmAmGmAmUsmUsmUsmU;
(SEQ ID NO: 404)
mCsmAsGsmUmAUmGmGmAmCAmCUGUCCAAmAmGUUUmUAmGmUACU
mCmUmGmUmAmAmUmGmAmAmAmAmUmUmAmCmAmGmAAmUCUACUmA
mAAACAAmGmGmCmAmAmAAUmGmCmCGUGmUmUmUmAmUmCmUmCmG
mUmCmAmAmCmUmUmGmUmUmGmGmCmGmAmGmAmUsmUsmUsmU;
or
(SEQ ID NO: 404)
mCsmAsGsmUmAUmGmGmAmCAmCmUGUCmCmAAmAmGmUmUmUmUmA
mGmUAmCmUmCmUmGmUmAmAmUmGmAmAmAmAmUmUmAmCmAmGmAm
AmUCUACmUmAmAmAAmCAmAmGmGmCmAmAmAAUmGmCmCmGmUGmU
mUmUmAmUmCmUmCmGmUmCmAmAmCmUmUmGmUmUmGmGmCmGmAmG
mAmUsmUsmUsmU.

In another aspect, a modified guide RNA (gRNA) is provided, which comprises a nucleic acid sequence, from 5′ to 3′, selected from

(SEQ ID NO: 404)
mCsmAsmGsUAUGGACACUGUCCAAAGUUUUAGUACmUmCmUmGmUmA
mAmUmGmAmAmAmAmUmUmAmCmAmGmAAUCUACUAAAACAAGGCAAA
AUGCCGUGUUUAUCUCGUCAACUUGUUGGCGAGAUsmUsmUsmU;
(SEQ ID NO: 404)
mCsmAsmGsUAUGGACACUGUCCAAAGUUUUAGUACmUmCmUmGmUmA
mAmUmGmAmAmAmAmUmUmAmCmAmGmAAUCUACUAAAACAAGGCAAA
AUGCCGUGUmUmUmAmUmCmUmCmGmUmCmAmAmCmUmUmGmUmUmGm
GmCmGmAmGmAmUsmUsmUsmU;
(SEQ ID NO: 404)
mCsmAsmGsmUAUmGmGmACAmCUGUCCAAAmGUUUUAGUACmUmCmU
mGmUmAmAmUmGmAmAmAmAmUmUmAmCmAmGmAAUCUACUAAAACAA
GGCAAAAUGCCmGUGUmUmUAmUmCmUmCmGmUmCmAmAmCmUmUmGm
UmUmGmGmCmGmAmGmAUsmUsmUsmU;
(SEQ ID NO: 406)
mCsmAsmGsUAUGGACACUGUCCAAAGUUUUAGUACmUmCmUmGGmAm
AmAmCmAmGmAAUCUACUAAAACAAGGCAAAAUGCCGUGUmUmUmAmU
mCmUmCmGmUmCmAmAmCmUmUmGmUmUmGmGmCmGmAmGmAmUsmUs
mUsmU;
or
(SEQ ID NO: 406)
mCsmAsmGsmUAUmGmGmACAmCUGUCCAAAmGUUUUAGUACmUmCmU
mGmGmAmAmAmCmAmGmAAUCUACUAAAACAAGGCAAAAUGCCmGUGU
mUmUAmUmCmUmCmGmUmCmAmAmCmUmUmGmUmUmGmGmCmGmAmGm
AUsmUsmUsmU.

In another aspect, a modified guide RNA (gRNA) is provided, which comprises a nucleic acid sequence, from 5′ to 3′, selected from

(SEQ ID NO: 407)
mCsmCsmAsGUAUGGACACUGUCCAAAGUUUUAGUACUCUGUAAUGAAA
AUUACAGAAUCUACUAAAACAAGGCAAAAUGCCGUGUUUAUCUCGUCAA
CUUGUUGGCGAGAmUsmUsmUsU;
(SEQ ID NO: 408)
mAsmCsmCsAGUAUGGACACUGUCCAAAGUUUUAGUACUCUGUAAUGAA
AAUUACAGAAUCUACUAAAACAAGGCAAAAUGCCGUGUUUAUCUCGUCA
ACUUGUUGGCGAGAmUsmUsmUsU;
(SEQ ID NO: 409)
mCsmAsmCsCAGUAUGGACACUGUCCAAAGUUUUAGUACUCUGUAAUGA
AAAUUACAGAAUCUACUAAAACAAGGCAAAAUGCCGUGUUUAUCUCGUC
AACUUGUUGGCGAGAmUsmUsmUsU;
or
(SEQ ID NO: 410)
mCsmCsmAsCCAGUAUGGACACUGUCCAAAGUUUUAGUACUCUGUAAUG
AAAAUUACAGAAUCUACUAAAACAAGGCAAAAUGCCGUGUUUAUCUCGU
CAACUUGUUGGCGAGAmUsmUsmUsU.

In another aspect, a modified guide RNA (gRNA) is provided, which comprises a nucleic acid sequence, from 5′ to 3′, selected from

(SEQ ID NO: 411)
mCsmAsmGsUAUGGACACUGUCCAAAGUUUUAGUACUCUGUAGAAAUAC
AGAAUCUACUAAAACAAGGCAAAAUGCCGUGUUUAUCUCGUCAACUUGU
UGGCGAGAUsmUsmUsmU;
(SEQ ID NO: 412)
mCsmAsmGsUAUGGACACUGUCCAAAGUUUUAGUACUCUGCGGAAACGC
AGAAUCUACUAAAACAAGGCAAAAUGCCGUGUUUAUCUCGUCAACUUGU
UGGCGAGAUsmUsmUsmU;
(SEQ ID NO: 406)
mCsmAsmGsUAUGGACACUGUCCAAAGUUUUAGUACUCUGGAAACAGAA
UCUACUAAAACAAGGCAAAAUGCCGUGUUUAUCUCGUCAACUUGUUGGC
GAGAUsmUsmUsmU;
(SEQ ID NO: 413)
mCsmAsmGsUAUGGACACUGUCCAAAGUUUUAGUACUCGAAAGAAUCUA
CUAAAACAAGGCAAAAUGCCGUGUUUAUCUCGUCAACUUGUUGGCGAGA
UsmUsmUsmU;
or
(SEQ ID NO: 414)
mCsmAsmGsUAUGGACACUGUCCAAAGUUUUAGUACCCGAAAGCAUCUA
CUAAAACAAGGCAAAAUGCCGUGUUUAUCUCGUCAACUUGUUGGCGAGA
UsmUsmUsmU.

In another aspect, a modified guide RNA (gRNA) is provided, wherein the gRNA comprises a nucleic acid sequence, from 5′ to 3′, selected from

(SEQ ID NO: 415)
mCsmAsmGsUAUGGACACUGUCCAAAGUUUUAGUACUCUGUAAUGAAAA
UUACAGAAUCUACUAAAACAAGGCAAAAUGCCGUGUUUAUCUCGUCAAC
UUGUUGGCGAGAUsmUsmUsmU;
or
(SEQ ID NO: 415)
mCsmAsmGsUAUGGACACUGUCCAAAGUUUUAGUACUCUGUAAUGAAAA
UUACAGAAUCUACUAAAACAAGGCAAAAUGCCGUGUUUAUCUCGUCAAC
UUGUUGGCGAGAmUsmUsmUsU.

In the above-delineated modified gRNAs, the “m” denotes a 2′-O-methyl and “s” denotes a phosphorothioate.

In another aspect, a formulation is provided which comprises a lipid nanoparticle comprising an mRNA expressing a base editor and a gRNA, wherein the base editor comprises a Cas9 domain and at least one adenosine deaminase variant comprising V82G, Y147T/D, Q154S, and one or more of L36H, I76Y, F149Y, N157K, and D167N with reference to SEQ ID NO: 1: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1), or corresponding alterations in another adenosine deaminase; and the gRNA comprises CAGUAUGGACACUGUCCAAA (SEQ ID NO: 370).

In another aspect, a formulation is provided which comprises a lipid nanoparticle comprising an mRNA expressing a base editor, wherein the base editor comprises a Cas9 domain and at least one adenosine deaminase variant comprising V82G, Y147T/D, Q154S, and one or more of L36H, I76Y, F149Y, N157K, and D167N with reference to SEQ ID NO: 1: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1), or corresponding alterations in another adenosine deaminase; and a gRNA comprising CAGUAUGGACACUGUCCAAA (SEQ ID NO: 370).

In an embodiment of the above-delineated formulations, the adenosine deaminase variant domain comprises the following combination of alterations I76Y+V82G+Y147D+F149Y+Q154S+D167N of SEQ ID NO: 1, or corresponding alterations in another adenosine deaminase. In some embodiments, the gRNA comprises 2′O-methyl and/or phosphorothioate modifications. In some embodiments, the gRNA comprises 2′-O-methyl and phosphorothioate modifications. In some embodiments, the mRNA comprises one or more pseudouridines. In some embodiments, the mRNA comprises an N1-methylpseudouridine (m1Ψ).

Other aspects as described herein provide modified guide RNA sequences (gRNAs), (e.g., heavily modified gRNA sequences or “heavy mods”), such as described in Example 5 and as set forth in SEQ ID NOS: 404-415.

### Definitions

Unless defined otherwise, all technical and scientific terms used herein have the meaning commonly understood by a person skilled in the pertinent art. The following references provide one of skill with a general definition of many of the terms used in this disclosure and the embodiments described herein: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

In this application, the use of the singular includes the plural unless specifically stated otherwise. It must be noted that, as used in the specification, the singular forms “a,” “an,” and “the” include plural references unless the context clearly dictates otherwise. In this application, the use of “or” means “and/or,” unless stated otherwise, and is understood to be inclusive. Furthermore, use of the term “including” as well as other forms, such as “include,” “includes,” and “included,” is not limiting.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Any embodiments specified as “comprising” a particular component(s) or element(s) are also contemplated as “consisting of” or “consisting essentially of” the particular component(s) or element(s) in some embodiments. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art. Such acceptable range will depend in part on how the value is measured or determined, i.e., the limitations of the measurement system.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

By “adenine” or “9H-Purin-6-amine” is meant a purine nucleobase with the molecular formula C₅H₅N₅, having the structure

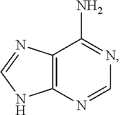

and corresponding to CAS No. 73-24-5.

By “adenosine” or “4-Amino-1-[(2R,3R,4S,5R)-3,4-dihydroxy-5-(hydroxymethyl)oxolan-2-yl]pyrimidin-2(1H)-one” is meant an adenine molecule attached to a ribose sugar via a glycosidic bond, having the structure

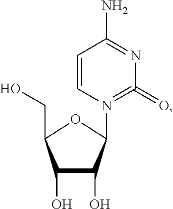

and corresponding to CAS No. 65-46-3. Its molecular formula is C₁₀H₁₃N₅O₄.

By “adenosine deaminase” or “adenine deaminase” is meant a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). The adenosine deaminases (e.g. engineered adenosine deaminases, evolved adenosine deaminases) provided herein may be from any organism (e.g., eukaryotic, prokaryotic), including but not limited to algae, bacteria, fungi, plants, invertebrates (e.g., insects), and vertebrates (e.g., amphibians, mammals). In some embodiments, the adenosine deaminase is an adenosine deaminase variant with one or more alterations and is capable of deaminating both adenine and cytosine in a target polynucleotide (e.g., DNA, RNA). In some embodiments, the target polynucleotide is single or double stranded. In some embodiments, the adenosine deaminase variant is capable of deaminating both adenine and cytosine in DNA. In some embodiments, the adenosine deaminase variant is capable of deaminating both adenine and cytosine in single-stranded DNA. In some embodiments, the adenosine deaminase variant is capable of deaminating both adenine and cytosine in RNA.

By “adenosine deaminase activity” is meant catalyzing the deamination of adenine or adenosine to guanine in a polynucleotide. In some embodiments, an adenosine deaminase variant as provided herein maintains adenosine deaminase activity (e.g., at least about 30%, 40%, 50%, 60%, 70%, 80%, 90% or more of the activity of a reference adenosine deaminase (e.g., TadA*8.20 or TadA*8.19)).

By “Adenosine Base Editor (ABE)” is meant a base editor comprising an adenosine deaminase.

By “Adenosine Base Editor 8 (ABE8) polypeptide” or “ABE8” is meant a base editor as defined herein comprising an adenosine deaminase variant comprising an alteration at amino acid position 82 and/or 166 of the following reference sequence: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1). In some embodiments, ABE8 comprises further alterations, as described herein, relative to the reference sequence.

By “Adenosine Base Editor 8 (ABE8) polynucleotide” is meant a polynucleotide encoding an ABE8 polypeptide.

By “Adenosine Deaminase polynucleotide” is meant a polynucleotide encoding an adenosine deaminase polypeptide. In particular embodiments, the adenosine deaminase polynucleotide encodes an adenosine deaminase variant comprising V82G, Y147T/D, Q154S, and one or more of L36H, I76Y, F149Y, N157K, and D167N. In some embodiments, the adenosine deaminase polynucleotide encodes an adenosine deaminase variant comprising one of the following combinations of alterations: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; or L36H+I76Y+V82G+Y147D+F149Y+Q154S+N157K+D167N.

In some embodiments, the deaminase or deaminase domain is a variant of a naturally occurring deaminase from an organism, such as a human, chimpanzee, gorilla, monkey, cow, dog, rat, or mouse. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to a naturally occurring deaminase. In some embodiments, the adenosine deaminase is from a bacterium, such as, E. coli, S. aureus, B. subtilis, S. typhi, S. putrefaciens, H. influenzae, C. crescentus, or G. sulfurreducens.

In some embodiments, the adenosine deaminase is a TadA deaminase. In some embodiments, the TadA deaminase is an E. coli TadA (ecTadA) deaminase or a fragment thereof. In some embodiments, the ecTadA deaminase is truncated ecTadA. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine. In some embodiments, the TadA deaminase is an N-terminal truncated TadA. In particular embodiments, the TadA is any one of the TadAs described in PCT/US2017/045381, which is incorporated herein by reference in its entirety.

In some embodiments, the TadA deaminase is TadA variant. In some embodiments, the TadA variant is TadA*7.10 comprising V82G, Y147T/D, Q154S, and one or more of L36H, I76Y, F149Y, N157K, and D167N. In some embodiments, the TadA variant is TadA*7.10 comprising a combination of alterations selected from among the following: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; or L36H+I76Y+V82G+Y147D+F149Y+Q154S+N157K+D167N. In some embodiments, the TadA variant is MSP605, MSP680, MSP823, MSP824, MSP825, MSP827, MSP828, or MSP829.

“Administering” is referred to herein as providing one or more compositions described herein to a patient or a subject.

By “agent” is meant any small molecule chemical compound, antibody, nucleic acid molecule, or polypeptide, or fragments thereof.

By “alteration” is meant a change (e.g. increase or decrease) in the structure, expression levels or activity of a gene or polypeptide as detected by standard art known methods such as those described herein. As used herein, an alteration includes a change in a polynucleotide or polypeptide sequence or a change in expression levels, such as a 10% change, a 25% change, a 40% change, a 50% change, or greater.

By “ameliorate” is meant decrease, suppress, attenuate, diminish, arrest, or stabilize the development or progression of a disease.

By “analog” is meant a molecule that is not identical, but has analogous functional or structural features. For example, a polynucleotide or polypeptide analog retains the biological activity of a corresponding naturally-occurring polynucleotide or polypeptide, while having certain modifications that enhance the analog's function relative to a naturally occurring polynucleotide or polypeptide. Such modifications could increase the analog's affinity for DNA, efficiency, specificity, protease or nuclease resistance, membrane permeability, and/or half-life, without altering, for example, ligand binding. An analog may include an unnatural nucleotide or amino acid.

By “base editor (BE),” or “nucleobase editor polypeptide (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain (e.g., Cas9 or Cpf1) in conjunction with a guide polynucleotide (e.g., guide RNA (gRNA)). Representative nucleic acid and protein sequences of base editors are provided in the Sequence Listing as SEQ ID NOs: 2-11 and 378.

By “base editing activity” is meant acting to chemically alter a base within a polynucleotide. In one embodiment, a first base is converted to a second base. In one embodiment, the base editing activity is adenosine or adenine deaminase activity, e.g., converting A·T to G·C.

In some embodiments, base editing activity is assessed by efficiency of editing. Base editing efficiency may be measured by any suitable means, for example, by sanger sequencing or next generation sequencing. In some embodiments, base editing efficiency is measured by percentage of total sequencing reads with nucleobase conversion effected by the base editor, for example, percentage of total sequencing reads with target A·T base pair converted to a G·C base pair or target C·G base pair to a T·A base pair. In some embodiments, base editing efficiency is measured by percentage of total cells with nucleobase conversion effected by the base editor, when base editing is performed in a population of cells.

The term “base editor system” refers to an intermolecular complex for editing a nucleobase of a target nucleotide sequence. In various embodiments, the base editor (BE) system comprises (1) a polynucleotide programmable nucleotide binding domain, a deaminase domain (e.g., cytidine deaminase or adenosine deaminase) for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine or cytosine base editor (CBE).

The term “Cas9” or “Cas9 domain” refers to an RNA guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casn1 nuclease or a CRISPR (clustered regularly interspaced short palindromic repeat) associated nuclease.

The term “Cas9” or “Cas9 domain” refers to an RNA guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a Casn1 nuclease or a CRISPR (clustered regularly interspaced short palindromic repeat) associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA,” or simply “gRNA”) can be engineered so as to incorporate aspects of both the crRNA and tracrRNA into a single RNA species. See, e.g., Jinek M., et al. Science 337:816-821(2012), the entire contents of which is hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., Proc. Natl. Acad. Sci. U.S.A. 98:4658-4663(2001); “CRISPR RNA maturation by trans-encoded small RNA and host factor RNase III.” Deltcheva E., et al., Nature 471:602-607(2011); and “A programmable dual-RNA-guided DNA endonuclease in adaptive bacterial immunity.” Jinek M., et al., Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

A nuclease-inactivated Cas9 protein may interchangeably be referred to as a “dCas9” protein (for nuclease-“dead” Cas9) or catalytically inactive Cas9. Methods for generating a Cas9 protein (or a fragment thereof) having an inactive DNA cleavage domain are known (See, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)). In some embodiments, dCas9 corresponds to, or comprises in part or in whole, a Cas9 amino acid sequence having one or more mutations that inactivate the Cas9 nuclease activity. In some embodiments, a dCas9 domain comprises D10A and an H840A mutation or corresponding mutations in another Cas9. In some embodiments, a Cas9 nuclease has an inactive (e.g., an inactivated) DNA cleavage domain, that is, the Cas9 is a nickase, referred to as an “nCas9” protein (for “nickase” Cas9). It should be appreciated that additional Cas9 proteins (e.g., a nuclease dead Cas9 (dCas9), a Cas9 nickase (nCas9), or a nuclease active Cas9), including variants and homologs thereof, are within the scope of this disclosure. Exemplary Cas9 proteins include, without limitation, those provided herein. In some embodiments, the Cas9 protein is a nuclease dead Cas9 (dCas9). In some embodiments, the Cas9 protein is a Cas9 nickase (nCas9). In some embodiments, the Cas9 protein is a nuclease active Cas9.

In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to wild-type Cas9. In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to wild-type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild-type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild-type Cas9.

In some embodiments, the fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

In some embodiments, Cas9 refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisI (NCBI Ref: NC_018721.1); Streptococcus thermophilus (NCBI Ref: YP_820832.1), Listeria innocua (NCBI Ref: NP_472073.1), Campylobacter jejuni (NCBI Ref: YP_002344900.1) or Neisseria meningitidis (NCBI Ref: YP_002342100.1) or to a Cas9 from any other organism.

In some embodiments, the Cas9 is from Neisseria meningitidis (Nme). In some embodiments, the Cas9 is Nme1, Nme2 or Nme3. In some embodiments, the PAM-interacting domains for Nme1, Nme2 or Nme3 are N₄GAT, N₄CC, and N₄CAAA, respectively (see e.g., Edraki, A., et al., A Compact, High-Accuracy Cas9 with a Dinucleotide PAM for In Vivo Genome Editing, Molecular Cell (2018)).

In some embodiments, Cas9 fusion proteins as provided herein comprise the full-length amino acid sequence of a Cas9 protein, e.g., one of the Cas9 sequences provided herein. In other embodiments, however, fusion proteins as provided herein do not comprise a full-length Cas9 sequence, but only one or more fragments thereof. For example, in some embodiments, a Cas9 fusion protein provided herein comprises a Cas9 fragment, wherein the fragment binds crRNA and tracrRNA or sgRNA, but does not comprise a functional nuclease domain, e.g., in that it comprises only a truncated version of a nuclease domain or no nuclease domain at all. Exemplary amino acid sequences of suitable Cas9 domains and Cas9 fragments are provided herein, and additional suitable sequences of Cas9 domains and fragments will be apparent to those of skill in the art.

In some embodiments, Cas9 refers to a Cas9 from archaea (e.g. nanoarchaea), which constitute a domain and kingdom of single-celled prokaryotic microbes. In some embodiments, Cas9 refers to CasX or CasY, which have been described in, for example, Burstein et al., “New CRISPR-Cas systems from uncultivated microbes.” Cell Res. 2017 Feb. 21. doi: 10.1038/cr.2017.21, the entire contents of which is hereby incorporated by reference. Using genome-resolved metagenomics, a number of CRISPR-Cas systems were identified, including the first reported Cas9 in the archaeal domain of life. This divergent Cas9 protein was found in little-studied nanoarchaea as part of an active CRISPR-Cas system. In bacteria, two previously unknown systems were discovered, CRISPR-CasX and CRISPR-CasY, which are among the most compact systems yet discovered. In some embodiments, Cas9 refers to CasX, or a variant of CasX. In some embodiments, Cas9 refers to a CasY, or a variant of CasY. It should be appreciated that other RNA-guided DNA binding proteins may be used as a nucleic acid programmable DNA binding protein (napDNAbp), and are within the scope of this disclosure.

In particular embodiments, napDNAbps useful in the methods described herein include circular permutants, which are known in the art and described, for example, by Oakes et al., Cell 176, 254-267, 2019.

“Co-administration” or “co-administered” refers to administering two or more therapeutic agents or pharmaceutical compositions during a course of treatment. Such co-administration can be simultaneous administration or sequential administration. Sequential administration of a later-administered therapeutic agent or pharmaceutical composition can occur at any time during the course of treatment after administration of the first pharmaceutical composition or therapeutic agent.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Non-limiting examples of conservative mutations include amino acid substitutions of amino acids, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained.

The term “coding sequence” or “protein coding sequence” as used interchangeably herein refers to a segment of a polynucleotide that codes for a protein. Coding sequences can also be referred to as open reading frames. The region or sequence is bounded nearer the 5′ end by a start codon and nearer the 3′ end with a stop codon. Stop codons useful with the base editors described herein include the following:
Glutamine CAG→TAG Stop codon
   CAA→TAA
Arginine CGA→TGA
Tryptophan TGG→TGA
   TGG→TAG
   TGG→TAA

By “codon optimization” is meant a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/(visited Jul. 9, 2002), and these tables can be adapted in a number of ways. See, Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding an engineered nuclease correspond to the most frequently used codon for a particular amino acid.

By “complex” is meant a combination of two or more molecules whose interaction relies on inter-molecular forces. Non-limiting examples of inter-molecular forces include covalent and non-covalent interactions. Non-limiting examples of non-covalent interactions include hydrogen bonding, ionic bonding, halogen bonding, hydrophobic bonding, van der Waals interactions (e.g., dipole-dipole interactions, dipole-induced dipole interactions, and London dispersion forces), and π-effects. In an embodiment, a complex comprises polypeptides, polynucleotides, or a combination of one or more polypeptides and one or more polynucleotides. In one embodiment, a complex comprises one or more polypeptides that associate to form a base editor (e.g., base editor comprising a nucleic acid programmable DNA binding protein, such as Cas9, and a deaminase) and a polynucleotide (e.g., a guide RNA). In an embodiment, the complex is held together by hydrogen bonds. It should be appreciated that one or more components of a base editor (e.g., a deaminase, or a nucleic acid programmable DNA binding protein) may associate covalently or non covalently. As one example, a base editor may include a deaminase covalently linked to a nucleic acid programmable DNA binding protein (e.g., by a peptide bond). Alternatively, a base editor may include a deaminase and a nucleic acid programmable DNA binding protein that associate noncovalently (e.g., where one or more components of the base editor are supplied in trans and associate directly or via another molecule such as a protein or nucleic acid). In an embodiment, one or more components of the complex are held together by hydrogen bonds.

By “cytosine” or “4-Aminopyrimidin-2(1H)-one” is meant a purine nucleobase with the molecular formula C₄H₅N₃O, having the structure

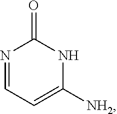

and corresponding to CAS No. 71-30-7.

By “cytidine” is meant a cytosine molecule attached to a ribose sugar via a glycosidic bond, having the structure

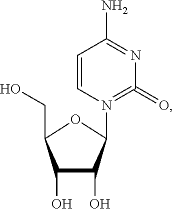

and corresponding to CAS No. 65-46-3.
Its molecular formula is C₉H₁₃N₃O₅.

By “Cytidine Base Editor (CBE)” is meant a base editor comprising a cytidine deaminase.

By “Cytidine Base Editor (CBE) polynucleotide” is meant a polynucleotide comprising a CBE.

By “cytidine deaminase” or “cytosine deaminase” is meant a polypeptide or fragment thereof capable of deaminating cytidine or cytosine. In one embodiment, the cytidine deaminase converts cytosine to uracil or 5-methylcytosine to thymine. The terms “cytidine deaminase” and “cytosine deaminase” are used interchangeably throughout the application. Petromyzon marinus cytosine deaminase 1 (PmCDA1) (SEQ ID NO: 13-14), Activation-induced cytidine deaminase (AICDA) (SEQ ID NOs: 15-21), and APOBEC (SEQ ID NOs: 12-61) are exemplary cytidine deaminases. Further exemplary cytidine deaminase (CDA) sequences are provided in the Sequence Listing as SEQ ID NOs: 62-66 and SEQ ID NOs: 67-189.

By “cytosine” is meant a pyrimidine nucleobase with the molecular formula C4H5N3O.

By “cytosine deaminase activity” is meant catalyzing the deamination of cytosine or cytidine. In one embodiment, a polypeptide having cytosine deaminase activity converts an amino group to a carbonyl group. In an embodiment, a cytosine deaminase converts cytosine to uracil (i.e., C to U) or 5-methylcytosine to thymine (i.e., 5 mC to T). In some embodiments, a cytosine deaminase as provided herein has increased cytosine deaminase activity (e.g., at least 10-fold, 20-fold, 30-fold, 40-fold, 50-fold, 60-fold, 70-fold, 80-fold, 90-fold, 100-fold or more) relative to a reference cytosine deaminase.

The term “deaminase” or “deaminase domain,” as used herein, refers to a protein or fragment thereof that catalyzes a deamination reaction. Exemplary deaminases include cytidine and adenosine deaminases.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an enzyme-linked immunosorbent assay (ELISA)), biotin, digoxigenin, or haptens.

By “disease” is meant any condition or disorder that damages or interferes with the normal function of a cell, tissue, or organ. An example of a disease includes Glycogen Storage Disease Type 1 (also known as GSD1 or Von Gierke Disease). In some embodiments, the GSD1 is Type 1a (GSD1a).

The term “effective amount,” as used herein, refers to an amount of a biologically active agent that is sufficient to elicit a desired biological response. In some embodiments, an effect amount is an amount required to ameliorate the symptoms of a disease relative to an untreated patient. The effective amount of an active agent(s) used to practice therapeutic methods and treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount of a base editor described herein (e.g., a fusion protein comprising a programmable DNA binding protein, a nucleobase editor and gRNA) sufficient to introduce an alteration in a gene of interest (e.g., G6PC) in a cell (e.g., a cell in vitro, in vivo, or ex vivo). In one embodiment, an effective amount is the amount of a base editor required to achieve a therapeutic effect (e.g., to reduce or control GSD1a or a symptom or condition thereof). Such therapeutic effect need not be sufficient to alter G6PC in all cells of a subject, tissue or organ, but only to alter G6PC in about 1%, 5%, 10%, 25%, 50%, 75% or more of the cells present in a subject, tissue or organ. In one embodiment, an effective amount is sufficient to ameliorate one or more symptoms of GSD1a.

In some embodiments, an effective amount of a fusion protein provided herein, e.g., of a nucleobase editor comprising a nCas9 domain and a deaminase domain (e.g., adenosine deaminase) refers to the amount of the fusion protein that is sufficient to induce editing of a target site specifically bound and edited by the nucleobase editors described herein. As will be appreciated by the skilled artisan, the effective amount of an agent, e.g., a fusion protein, a nuclease, a hybrid protein, a protein dimer, a complex of a protein (or protein dimer) and a polynucleotide, or a polynucleotide, may vary depending on various factors as, for example, on the desired biological response, e.g., on the specific allele, genome, or target site to be edited, on the cell or tissue being targeted, and/or on the agent being used.

By “fragment” is meant a portion of a polypeptide or nucleic acid molecule. This portion contains, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “glucose-6-phosphatase (G6PC) polypeptide” is meant a polypeptide or fragment thereof having at least about 95% amino acid sequence identity to NCBI Accession No. AAA16222.1. A wild-type G6PC polypeptide is capable of catalyzing the hydrolysis of D-glucose 6-phosphate to D-glucose and orthophosphate, while a G6PC polypeptide comprising a deleterious mutation lacks or has reduced catalytic activity. In particular embodiments, a method of editing a G6PC polynucleotide is provided, in which the method comprises a single nucleotide polymorphism (SNP) associated with Glycogen Storage Disease Type 1a (GSD1a). In one embodiment, the A·T to G·C alteration at the SNP associated with GSD1a changes a glutamine (Q) to a non-glutamine (X) amino acid in the G6PC polypeptide. In another embodiment, the A·T to G·C alteration at the SNP associated with GSD1a changes an arginine (R) to a non-arginine (X) in the G6PC polypeptide. In one embodiment, the SNP associated with GSD1a results in expression of an G6PC polypeptide having a non-glutamine (X) amino acid at position 347 or a non-arginine (X) amino acid at position 83. In one embodiment, the base editor correction replaces the glutamine at position 347 with a non-glutamine amino acid (X). In another embodiment, the base editor correction replaces the arginine at position 83 with a non-arginine amino acid (X). Mutations associated with GSD1a are known in the art and described, for example, in Chou et al., Hum. Mutat. 29:921-930, 2008, which is incorporated herein by reference. Methods for detecting glucose-6-phosphatase activity are known in the art and described, for example by Varga et al., Int. J.

Mol. Sci. 2019, 20, 5039, which is incorporated herein by reference.

In particular embodiments, G6PC comprises one or more alterations relative to the following reference sequence. In particular embodiments, G6PC associated with GSD1a comprises one or more mutations selected from Q347X and R83C. An exemplary G6PC amino acid sequence from Homo Sapiens is provided below:

| (SEQ ID NO: 379) | |
|---|---|
| 1 | MEEGMNVLHD FGIQSTHYLQ VNYQDSQDWF ILVSVIADLR NAFYVLFPIW FHLQEAVGIK |
| 61 | LLWVAVIGDW LNLVFKWILF GQRPYWWVLD TDYYSNTSVP LIKQFPVTCE TGPGSPSGHA |
| 121 | MGTAGVYYVM VTSTLSIFQG KIKPTYRFRC LNVILWLGFW AVQLNVCLSR IYLAAHFPHQ |
| 181 | VVAGVLSGIA VAETFSHIHS IYNASLKKYF LITFFLFSFA IGFYLLLKGL GVDLLWTLEK |
| 241 | AQRWCEQPEW VHIDTTPFAS LLKNLGTLFG LGLALNSSMY RESCKGKLSK WLPFRLSSIV |
| 301 | ASLVLLHVFD SLKPPSQVEL VFYVLSFCKS AVVPLASVSV IPYCLAQVLG QPHKKSL |

By “glucose-6-phosphatase (G6PC) polynucleotide” is meant a nucleic acid molecule encoding an G6PC polypeptide, as well as the introns, exons, and regulatory sequences associated with its expression, or fragments thereof. In embodiments, an G6PC polynucleotide is the genomic sequence, mRNA, or gene associated with and/or required for G6PC expression. An exemplary G6PC nucleotide sequence from Homo Sapiens is provided below (GenBank: U01120.1):

| (SEQ ID NO: 380) | |
|---|---|
| 1 | ATAGCAGAGC AATCACCACC AAGCCTGGAA TAACTGCAAG GGCTCTGCTG ACATCTTCCT |
| 61 | GAGGTGCCAA GGAAATGAGG ATGGAGGAAG GAATGAATGT TCTCCATGAC TTTGGGATCC |
| 121 | AGTCAACACA TTACCTCCAG GTGAATTACC AAGACTCCCA GGACTGGTTC ATCTTGGTGT |
| 181 | CCGTGATCGC AGACCTCAGG AATGCCTTCT ACGTCCTCTT CCCCATCTGG TTCCATCTTC |
| 241 | AGGAAGCTGT GGGCATTAAA CTCCTTTGGG TAGCTGTGAT TGGAGACTGG CTCAACCTCG |
| 301 | TCTTTAAGTG GTAAGAACCA TATAGAGAGG AGATCAGCAA GAAAAGAGGC TGGCATTCGC |
| 361 | TCTCGCAATG TCTGTCCATC AGAAGTTGCT TTCCCCAGGC TATTCAGGAA GCCACGGGCT |
| 421 | ACTCATGCTT CCAACCCCTC TCTCTGACTT TGGATCATCT ACATAAAGGG GGAAGACAGA |
| 481 | AAAAATCCTA CCAGTGAGTT GAAAATACAG GAAAGCCTAT TTCATATGGG TTAAAGGGTA |
| 541 | GGACAGTTGA ATTTCGTGAA AAGTCTGAGT TATATAGGCT TTGAGCAAAG AGTTTTATTA |
| 601 | GTATGAAGCA GAAGAGGTAA CATAAAGAAA GATGTATGGG GCCAGGCATG GTGGCTCACA |
| 661 | CCTGTAATCC CAGCACTTTG GGAGGCCGAG GTGGGCGAAT CACTCCTGGG TGAACTCAGG |
| 721 | AGTTCAAGAC CAGCCTGGGC AACATGGCGA AACTCCATCT CTACAAAAAC ATTACGAAAA |
| 781 | TTAGCTGGGC GTGTTGGTGC TGTAGTCCCA GCTACTCAGG AGGCTGAGGT GAGAGGCGGA |
| 841 | GGAGGTTGCA GTGAGTCAAG ATCATGCCAC TGCACTCCAG CCTGGGCAAC AGAGTAAGAC |
| 901 | CCTGTCTCAA AAAAAAAAAA AAGATAGATG ATGTATGCTG TATGAAAAAA GGAAACACAC |
| 961 | AGATGATTCA ACAGCCTGTT TTGTGGGGTA ATGAAAAGTC ACCCTGGGAA CTGGGCTCCA |
| 1021 | GCCCTCGTTC TGCCACCCAC CAACTACATG TCCTTGGCAA GTCATATCAA TTATCTGAGT |
| 1081 | TTCTGTTTTA TAATCTACAA ATAGGTTATC TCTGGCAGCT TAATAATAAT CAGGGTTAAC |
| 1141 | ATTTATTAAA CAGTGTGTGC CAGTCCATGT GCTATGTGCT TTTCTGTGAG GTAGTTACTG |
| 1201 | CTATTTACAG AAACAGTAGA TGCAGAGACC AAGGTGCTGA GTTAAATGAT TAGGCCAACA |
| 1261 | AGGTTAGTAC ATGCCGAGCC AGGATGGAAG CCCAGGTAGG CAGGCTGGCT TCCGCGGCAA |
| 1321 | TGCTCTTATG AACTATGTTA CGTCCAGTGC TGATAAACTG ACTCTCTGGG GAGCAGGGGA |
| 1381 | AAGCCCTGAG TTTAGCATTT GCCAATTTCT ATCACGTAAA CATTCCCATT CTGGCCACTT |
| 1441 | TCTTTCTTTC TTTCTTTTGT TTGTTTGTTT GAGATGGAGT CTCGCACTGT TGCCTGGCTG |
| 1501 | GAGTGCAATG GTGCAATCTC AGCTCACTGC AACCTCTGCC TCTCCGGTTC AAGTGATTCT |
| 1561 | CCTGCCTCAG CCTCCCAAGT AGCTGGGATT ACAGGTGCCC GCCACCATGC CCAGCTAATT |
| 1621 | TTTTTTGTAT TTTTAGTAGA GACATGGTTT CACTATGTTG ACTAGGCTGG TCTCGAACTC |
| 1681 | CTGACCTCAT GATCTGCCTG CCTTGGCCTC CCTAAGTGCT AGGATTACAG GCGTGAGCCA |
| 1741 | CTACACCCAG CCGCATGATT CTAAAAAATA AAAAGATGAA GTGTTATTCC AAACATCTGA |
| 1801 | TCTCCATTGA AGAACCATGC AATCTCTCTG GGTTGATAGA GGCCAGAGTT AGTGGCTCTC |
| 1861 | CCTGATTTCG GTGAGAAATC ACTATTCCAC CATCACGGGA TAAAAGGCAT CCTGACTGGC |
| 1921 | GGTTGACACC TATTTCCACA GTGAAAGATA TATCTAGTAC TTTTAAAGGG GAAGTGGTTT |
| 1981 | GTCTGAGATA CTCTGTTTCA AAGTAGAGAG GATACAGAAC AAGCATCTGA AGCTATATAC |
| 2041 | ATCCTTACAG AGAGCAATTC TGATGGAAAT GCAGGCCATG TTTCCCTGGG GGGGGCTCGT |
| 2101 | CCTAGGGGCT GGAGTGCATT CTCTGATGTC AGAGGAAATG CAAGATTCCC TGAGGCCTGA |
| 2161 | GGGAACCCAT GGTATATGCA AGTCCAAGTT TCAAACTGTA GTTCCATATG CATTCTTCCA |
| 2221 | GGACAAATAC TTCTTGAGGT TAAAAAAAAA AAGTCACATA GCTGCCATTT TATGGATTTC |
| 2281 | AGGATTTTTT TTTTTTTTTT TTTGAGATGG AGTCTTGCTC TGTCACCCAG CCTGTAGTGC |
| 2341 | AGTGGCATAA TCTCGGCTCA CGGCAACCTC CGCCTCCCAG GTTCAAGCGA TTCTCTTGCC |
| 2401 | TTAGCCTCCC GAGTAGCTGG GATTACAGTC ACGCACCACC ACATCTGGCT AATTCTTTAT |
| 2461 | ATTTTTTGGT AGAAACGGTG TTTCACCATG TTGGCCAGGC TGGTCTCAAA CTCCTGACCT |
| 2521 | CATGTGATCT GCCTGCCTTG GCCTCCCAAA GTGCTGAGAT TACAGGTGTG AGCCACCGCG |
| 2581 | CCTGCCTGGA GTTCAGAATC TTGGGCTTCA TTATTTGTGT TTAAATAGAT CATACAGTCA |
| 2641 | GGCACGGTGG CTCATGCCTG TAATCCCAGC ACTTTGGGAG GCTGAGGTGG GAGGATTGCC |
| 2701 | TGAGTTCAGG AGATGGAGAC CAGCCTGGGC AACATGGTGA AACCCCGTCT CTACTAAAAA |
| 2761 | TACAAAAACT AGCTGGATGT GGTGGCACAC ACCTGTAGTC CCAGCTATTC AGGAGGCTGA |
| 2821 | GGTGGGAGGA TCCCAGGAGG TAGAGGTCAC AATGAGCCGA GATTGCGCCA CTGCACTCCA |
| 2881 | GGCTGGGTTA CTGAGCCAGA TCCTGTCTCA AAAAAAAAAA AGATAATACA TTCAAACAGT |
| 2941 | TCAAAATGCA AAAGTTACAT ACATAAGGAA GTGTCATGAA ATATCTCCCT CTCACACTTC |
| 3001 | TCCCCAGCCA CCCAGTTCTC CCTTCTAGAG GCAACATGTG AAATCCTTCT CAGGCTACAC |
| 3061 | TCTTCTTGAA GGTGTAGGCT TTGGGCAAAA GCATTCATTC AGTAACCCCA GAAACTTGTT |
| 3121 | CTGTTTTTCC ATAGGATTCT CTTTGGACAG CGTCCATACT GGTGGGTTTT GGATACTGAC |
| 3181 | TACTACAGCA ACACTTCCGT GCCCCTGATA AAGCAGTTCC CTGTAACCTG TGAGACTGGA |
| 3241 | CCAGGTAAGC GTCCCAGCCC CTGCAGACAG AAGCTGAGTG GACCTCGTTT ACCTGTTATG |
| 3301 | GATGAAACTG ACCTTGAGGG GACATGAGGA GAGCCATTCC TTTGTACTTT TGTCATGCTC |
| 3361 | TTCAATTGGC ACAAATTAAT TCACTTCTGC AATACTTTCC TGAATAGCAC AGTAGTATTG |
| 3421 | GAAATCTGCC TATTACAGAA CCTGGATGGA GTCCAGAGAG GCACGGGCAT CCATGGGCAA |
| 3481 | AGGGCTCGTG AGAGTCACCG CCCTGCAGCG CTGTGTCCTG AGAAAGGAGG GGGCAGAAGC |
| 3541 | CTGAGCTTCT GGGGGTCCTT CCCAATGGCC TGGCCCACTG GATGTGCCCT CCTGAGCTGA |
| 3601 | CCGTCCAATC CCTTGCCCTC TCTGTGCCTA CGTTTTATTA GTTACAGCCA GATGGTTACT |
| 3661 | GTCAAATCAA ATGATAGATT TCATTTTCAG TATGTAATAG GAAGCCCCTC CCTCACCCTA |
| 3721 | AAGTCTCAGC TGCCCTCTAA GACTAGTACT CTCTAAGGTA CTAGTATCCC TTCCTCAGAG |
| 3781 | ACCCTTTCCC TGACCCCAAA ACTAGGGAAG GTCCCTTAGT TATTTGCTCT CACAGACCAC |
| 3841 | GCATTTACCT CAGAGCATAT TCACTCATTC AGCTGTTACT TACCAAGCAC CTACTGGGAG |
| 3901 | CTATACACTG TTCTATGTGC TAGGGATACC TCTGTCAGTG AACAACACAG ACACAAAGAT |
| 3961 | CCCTGCCCTT GTGGAGCTGA AATCTGAATA GAGGAGGTGA AATATACAAA AATTATAATA |
| 4021 | AATAAGTAAA CTAGGCCAGT TGTGGTTGCT CATGCCTGTA ATCCCAGCAC TTTGGGAAGC |
| 4081 | CAAGGTAGGT AGATCACCTG AGGTCAGGAG TTCAAAACCA GCCTGGCCAA CATTGCAAAA |
| 4141 | TCCTGTCTTT ACTAAAAATG GAAAAATTGG TCAGGCGTGA TGGCACACGC CTGTAGTCTC |
| 4201 | AGCTACCTGG GAGGCTGAGG CAGGAGAATC GCTTGAACCT GGGAGGCAGA GGTTGCAGTG |
| 4261 | AACCGAGATC GGACCACTGC ACTCCAGCCT GAATGACAGA ACGAGACTCT GTCTCAAAAA |
| 4321 | AAAAGTAAAC TATTAATATG TAGGATAGGC CAGGCACGGT GGCTCACCCT GTAATCCCAG |
| 4381 | CACTTTGGGA GGCTGAGGCG GGTGGATCAC CTGAGGTGAG GAGTTCAAGA CCAGCCTGGC |
| 4441 | CAACATGGCA AAACCCTGTC TCTACTAAAA ATACAAAAAT TAGCTGGGTG TCCTGGTGCA |
| 4501 | TGCCTGTAAT CTGAGCTACT CAGGAGGCTA AGGCAGGAGA ATCGCTTGAA CCTGGGAGGT |
| 4561 | GGTGAGCCAA GATTGCGCCA TTGCACTCCA GCCTGGGCGA CAAAATGAGA CACCATCTGA |
| 4621 | AAAAAAAAAA AAAATATATA TATATATACA CACACACACA CACACACACA CACACACACA |
| 4681 | TATAATACTA GAAAATGATT GTTTATAGGC AAAAAAAAAA AAAAAGAAGA AGAAGAAGAA |
| 4741 | AAGGAAAGGA GAAGGAAAGA AGGACCAAAC ATCTTTTGTA GAAATATGTT TGCTTTCATC |
| 4801 | ATAACAGCTT GTTATCAAGG ATGAATTTCT CCCTGAAATT AATGGAGGCA CAGACTGGAA |
| 4861 | AGTTTAAAGT GGCTTTAAGA GGTTATTTTA TTTAGTCCTC TGTCTTAATA GAAGCAAATT |
| 4921 | ATTATCTCTG CTCCTTAGGT AGAGTAGCTA AGGCTCAGAA AGTAGGCCGG GCGCGGTGGC |
| 4981 | TCACGCCTGT AATCCTAGCA CTTTGGGAGG CCAACGCAGG TGGATCACCT GAGGTCAGGA |
| 5041 | GTTTGAGACC AGCCTGGCCA ACATGGTGAA ACCTCGTCAC TAATAAAAAA ATACAAAAAC |
| 5101 | TTAGCCAGGC ATGGTGGCGG GCGCCTGTAA TCCCAGCTAC CCAGGAGGCT GCGGCAGGAG |
| 5161 | AATCACTTCA ACCCGGGAGG CAGAGGTTGC AGTGAGCTGA AATCACACCA CTGCACTCCA |
| 5221 | GCCTTGGTGA CAGAGAAAGA TTCTGTCAGG AAAAAAAAAA AAAAGTTTAA ATGAATTACC |
| 5281 | CAAGGTATAT AATTGTTAGT GTTAGAAGGA AGAAGAAGGG AGGGAGGAAG GAAGGGAGAA |
| 5341 | AGAAAGGGAA GGAGGAAGGG AGGGAGGGAA GAAAGCCTTT ATTTATCTAT GGGGTTCCCT |
| 5401 | GGAAAGCAGG CTGAAATGGA GATTCACGTG CAGGAGTTTA GATACTCTGG GGAACTATAC |
| 5461 | TTGTAGAAGG GAAGGAACAG GAACAGGGCA GAAGGAGAGG TCCGGTTGTG ATTCTGCCTC |
| 5521 | ATCCAACCCC ACAGCGAGCT CTGAAGCTGG GGATGGCTCC TCAGAGTTGG TCCAAGTTGG |
| 5581 | GACAAGGGAA TCAGACCCTG GGGAGAGCGT AACCTTGATC AAGGCGACTC TCTTTAGCCC |
| 5641 | AGGGCAATGC CAGGAGAAGG CTGAGAGCAG AAAGCCATCT ACCATCACAC TCTCAACAGC |
| 5701 | TACGAAATAA GTCCTGCAGT TCAGGAGGGA GGTCTGGGCG GCACATCTCA GGACCCTCTA |
| 5761 | TCTCTCAGGG TAGAGGAATT AAGAATGGGA TGGGAACCAG ACGGGCCATG GTGGCTCACA |
| 5821 | CCTATAATCC CAACACTTTG GGAGGCCAAG GGTAGGAGGA TTGCTTGAGC CCAAGAGTTC |
| 5881 | AAAACCAGCC TGGGCAAAAA CAATCAAACA AACAAACAAA ACACATTTAA AAAATTTGCT |
| 5941 | GTGTGTGGTG GTGTGCACCT GTGGTCCCAG CTACTCAGGG GGCTGAGGTG GGAGGATTGC |
| 6001 | TTGAGTCCAG GAGGTCGAGG CTGCAGTGAG CTATGATCAT GGCACTGCAT TGCAGCCTAG |
| 6061 | GAGACAAAGC AAGACACTGT CTCTAAAAAA ACAAAAAACA AACAAATAAA AAAACGGAAC |
| 6121 | CGGTTGCAAG CAGGGTTAAA TAGCGTGGTC AGAGTAGGAC TCACTGAGAA TATGAGATCT |
| 6181 | GAGTCAAGTC TTCAAGGATG TGAGGAAGTA AGTTTCTGGC AGAAGAGCTG TGAAGGGCTG |
| 6241 | TCTGGCCAGA GAAGATTGCA ATGCAAAAGC CCTGAGGTGG GAACGTGTTT GGTGTGTTTA |
| 6301 | AAGGAAAGCA ATGAGGCCAG TGTAGCCAGA ACAGAGTGTG CAAGGAGAGA AGGAACAGAA |
| 6361 | GATGTGGAGG GCAGATCAGT TTGTAATTGT ACGCCCAGTA TGCTGATTCT TTGTGTAATC |
| 6421 | TCCAGACTGT ATTAAACTGC AAGAGCAGGG CCCCTCTCTG GCTTTGCTCA TCATTGTATT |
| 6481 | CCCAGAGCCT TGCACAATGC TTGGTGCATA GGAGATGGAA ATTTGTTAAA TAAATGAATT |
| 6541 | ATGGATAACG AATGGATGGT AAGATGGGTG GATGGATGGG GGGTGAACGG ATGGATGGGG |
| 6601 | GGTGAATGGA TGGATGAATG GGTAGATGGG TGGATAGGGG GATGGCTGGG TGGCTGGGTA |
| 6661 | GATGATGCAC TGTCTCCCAG ATGAGGACCT TTTCACCTTT ACTCCATTCT CTTTCCTGCC |
| 6721 | CTTTAGGGAG CCCCTCTGGC CATGCCATGG GCACAGCAGG TGTATACTAC GTGATGGTCA |
| 6781 | CATCTACTCT TTCCATCTTT CAGGGAAAGA TAAAGCCGAC CTACAGATTT CGGTAAGAAC |
| 6841 | TCACCACTGG GGTGTAGGTG GTGGAGGGCA GGAGGCAGCT CTCTCTGTAG CTGACACACC |
| 6901 | ACGTATTCTT CCTCACATCC CCCTAGCCCG CTCCCACACC TGGGCAGCCG CTGATTAAGA |
| 6961 | GTTGTGGCAC TTTGGATAGG GATAAACCTC AGAGTCAGGG AATGTTTGGG CTGAAAGGGA |
| 7021 | TCCAGTAGTG CAATCCGTTG TTTTACAGAT AAGGAAACAA AGCCCAACAC CATGAAGGGA |
| 7081 | CTTATAAAAA TAAGGTAGTG AAGTAGCAGC AGGGCTTAAA TAAAAACCCA TGTCTGTACC |
| 7141 | AACCACAGAG TCACCCATCC AGGTTAAAAT AACCAGAGAA ACAGAAGATA TTCCTACTAC |
| 7201 | AGAGAATTCC GGGTGTGCAG CCACAGTGCA AATCCTTTTT ATTTTTATTT TTGAGATGCA |
| 7261 | GTCTCGCTCT GTCATCCAGG CTGAAGTGCA GTGGCACGAT CATGTCTCGC TGCAACCTCT |
| 7321 | GCCTCCCAGG CTCAAGCGAT CCTCCCACCT CAGCCATCTG AGTAGCTGGG ACCACAGGCC |
| 7381 | ACACACCACA CCCAGCTAAT TTCTCGTATC TTTTTGTAGA GACAGAGTTC TGCTATGTTG |
| 7441 | CCCAGGCTCA GGCTGGTCTT GATCTCAAGC AATTGGCTTG CCTCAGCCTC CTAAAATATT |
| 7501 | GGGATTACAG GCATGAGCCA CCGCGCCAGC CATGCAAATC CTTAATTATC AAACAGATAA |
| 7561 | AATAGGGAAG TTAAAATTCA TATACACAAG GGTTAACCAC TTGCCACAGG CATTTTTTTT |
| 7621 | TTTTTTTTGA GACGGAATCT CGCTCTGTTG CCCAGGCTGG AGTGCAGTGG CGCCATCTCG |
| 7681 | CCTCACTGCA ACCTCCGCTT CCTGGGTTCA AGCTATTCTT CTGCCTCAGC CTACCGAGTA |
| 7741 | GCTGGGACTA CAGGCACGTG CCACCACACC TGGCTAATTT TTTTATTTTT AGTAGAGATG |
| 7801 | GGGTTTCACC ATATTGGCCA GGCTGGTCTT GAACTCCTGA CCTAGTGATC CATCCGCCTC |
| 7861 | AGCCTCCCAA AGTGCTGGGA TTGCAGGCAT GAGCCACCGC GCCTGGCCTT TTTTTTTTTT |
| 7921 | TTTTGAGACG GAGTTTTGCT CTTGTTGCCC AGGCTAGAGT GCAGTGGCGC AGTCTCGGCT |
| 7981 | CACTGTAACC TCCACCTCCT GAGTTCAAGC AATTCTCCTG CCTCAGCCTC TCAAATAGCT |
| 8041 | GGGATTACAG GCGTGAGCCA CCCCACCTGG CTAATTTTGT AATTTTTTTT TTAGTAGAGA |
| 8101 | TGGGGTTTCA CCTGTTGATC AGGCTGGTCT CAAACTCCTG ACCTCAAGTG ATCCACCCAC |
| 8161 | CTCGGCCTCC CAAAGTGCTG GGATTACAAG CATAAGCCAC CGTGCCTGGT CAATTTTGAT |
| 8221 | CTTTTTTAAA GAGACAGGGG TCTTGCTATG TTGCCCAGAC TAGTCTTGAA CTCCTGGCCT |
| 8281 | CAAGTGATCC TCTCACCTCG GCCTCCCAAA GTATTGGGAT TACAGGTCTG AGCCGCTGCA |
| 8341 | CCCAGCCCCC AACAGGCATC TTTGGACTTT TGAGTACTGG CTTTAATTTA CAAAAATTCC |
| 8401 | ACTGAGAGCA CCTAAGTTTG CCAGGCTCCA ACATTTCTGC AGGGGCTGTT TTCTTTGCTG |
| 8461 | AAGGATCTGC ACCTGTGTTC TGTTATGGTT GCCTCTTCTG TTGCAGGTGC TTGAATGTCA |
| 8521 | TTTTGTGGTT GGGATTCTGG GCTGTGCAGC TGAATGTCTG TCTGTCACGA ATCTACCTTG |
| 8581 | CTGCTCATTT TCCTCATCAA GTTGTTGCTG GAGTCCTGTC AGGTATGGGC TGATCTGACT |
| 8641 | CCCTTCCTTC TCCCCCAAAC CCCATTCCGT TTCTCTCCCT AATCAGGACA AAATCCCAGC |
| 8701 | ATTCCAGCCA CATCCTGTGT GTAATCAGTA CTGTTAGCAT TTCTGTGGGT TGAAAGTCAA |
| 8761 | GAATGAGCAA CTTGAAATGA TTAATTTCTA TAAGAGTGCC CAGATCTATA GAATGAATTG |
| 8821 | TGTAGAAGTT ACCATACATC AAATTAACGC ACCAAATTGA ATTAGCTTGA AATCTCAGAG |
| 8881 | CTTTTTACAA TCTTTATTTC TTACTGGTCT TCAACAGGCC CTAATTTACT TTTCAGGGAA |
| 8941 | TCTGCCAAAT TTAACAAATT AACACGATGT CCTAGGAAAG CTGTTCATTT AAATACATTC |
| 9001 | ATTTGCAAAC CTAATAGATA ACTGCAGTTG ATCTCTTTTA TAGGTTCAGA GTTTTGAATA |
| 9061 | TGTTTTTTTT TGTTTTTTTT TTTTGAGATG GAGTCTCGCT CTGTGACCCA GGCTAGAGTG |
| 9121 | CAGTGGTGCG ATCTCGGCTC ACTGCAAGCT CCACCTCCTG GGTTCACGCC ATTCTCCTGC |
| 9181 | CTCAGCCTCT CCGAGTAGCT GGGACTACAG GCGCCCGCCA CCATGCCCGG CTAATTTTTT |
| 9241 | GTATTTTTAG CAGAGACGGG GTTTCACCGT GGTCTTGATC TCCTGACCTC GTGATCCGCC |
| 9301 | CGCCTCGGCC TCCCAAAGCG CTGGGATTAC AAGGGTGAGC CACCGCACCC TGCCTGAATA |
| 9361 | TGTGTTTTCT TAGATCCAAT TAACAAGGGT AAGACAAGAT TTAAGTTAAG CATAAGAAAG |
| 9421 | ATTTTGTGGG AGGCACTGGA ATATAAGACC TTAACAAAAC TGTGGAATTT CTCCCCTGGA |
| 9481 | GATTTGTAAG AACGGAACAT AGCAGCATTC AAAGAAGAAT GTTGAGAACA AGGGAGATAA |
| 9541 | TGGTTTCATG GTAATCACAA AAGTAACACA GCATTTAGTA CTGGGTTCCA TGTTTGAGGA |
| 9601 | AGAACCTGGA AGCCATATCA CATGAAAAAC CTGGGAATGT TTAGGTTAGA GAGAATAACT |
| 9661 | GTGTTCAAAT GTGTGACAGA GGGACTAGAT TCATCACTTA CTAACTCCTG CAGAAAGAAC |
| 9721 | TGAGAAAAAT AGACAGTATT AGAGGGGGAC CAGTTTCACA CAGACAAGGA AGAACTATTC |
| 9781 | AGCAATCAAT TCCGTTCAAA GATAAAATGG ACTGTTATAG TGGGGGTGAG CTCCCTACCT |
| 9841 | CTGAGGGTAT TTCAAGTAGA GATAGGAGGA CCTCCTGGTA GGAAATTTGC ATACGGTGGG |
| 9901 | AGATTGTACG TGATATGGCA CCTCCATCTG AAAGAGTCTA TATTGAGGGC AGGCTGGAGT |
| 9961 | CACACATGGG AATAAGCCAG GCGACCCTCC CATCTGCCAT CTGTGATTTA ATTCCACAGT |
| 10021 | CGCAGAACGG ATGGCATGTC ACCCACTCCT CCAAACCCAC CTCTAGCAAA GGTCCCAAAT |
| 10081 | CCTTCCTATC TCTCACAGTC ATGCTTTCTT CCACTCAGGC ATTGCTGTTA CAGAAACTTT |
| 10141 | CAGCCACATC CACAGCATCT ATAATGCCAG CCTCAAGAAA TATTTTCTCA TTACCTTCTT |
| 10201 | CCTGTTCAGC TTCGCCATCG GATTTTATCT GCTGCTCAAG GGACTGGGTG TAGACCTCCT |
| 10261 | GTGGACTCTG GAGAAAGCCC AGAGGTGGTG CGAGCAGCCA GAATGGGTCC ACATTGACAC |
| 10321 | CACACCCTTT GCCAGCCTCC TCAAGAACCT GGGCACGCTC TTTGGCCTGG GGCTGGCTCT |
| 10381 | CAACTCCAGC ATGTACAGGG AGAGCTGCAA GGGGAAACTC AGCAAGTGGC TCCCATTCCG |
| 10441 | CCTCAGCTCT ATTGTAGCCT CCCTCGTCCT CCTGCACGTC TTTGACTCCT TGAAACCCCC |
| 10501 | ATCCCAAGTC GAGCTGGTCT TCTACGTCTT GTCCTTCTGC AAGAGTGCGG TAGTGCCCCT |
| 10561 | GGCATCCGTC AGTGTCATCC CCTACTGCCT CGCCCAGGTC CTGGGCCAGC CGCACAAGAA |
| 10621 | GTCGTTGTAA GAGATGTGGA GTCTTCGGTG TTTAAAGTCA ACAACCATGC CAGGGATTGA |
| 10681 | GGAGGACTAC TATTTGAAGC AATGGGCACT GGTATTTGGA GCAAGTGACA TGCCATCCAT |
| 10741 | TCTGCCGTCG TGGAATTAAA TCACGGATGG CAGATTGGAG GGTCGCCTGG CTTATTCCCA |
| 10801 | TGTGTGACTC CAGCCTGCCC TCAGCACAGA CTCTTTCAGA TGGAGGTGCC ATATCACGTA |
| 10861 | CACCATATGC AAGTTTCCCG CCAGGAGGTC CTCCTCTCTC TACTTGAATA CTCTCACAAG |
| 10921 | TAGGGAGCTC ACTCCCACTG GAACAGCCCA TTTTATCTTT GAATGGTCTT CTGCCAGCCC |
| 10981 | ATTTTGAGGC CAGAGGTGCT GTCAGCTCAG GTGGTCCTCT TTTACAATCC TAATCATATT |
| 11041 | GGGTAATGTT TTTGAAAAGC TAATGAAGCT ATTGAGAAAG ACCTGTTGCT AGAAGTTGGG |
| 11101 | TTGTTCTGGA TTTTCCCCTG AAGACTTACT TATTCTTCCG TCACATATAC AAAAGCAAGA |
| 11161 | CTTCCAGGTA GGGCCAGCTC ACAAGCCCAG GCTGGAGATC CTAACTGAGA ATTTTCTACC |
| 11221 | TGTGTTCATT CTTACCGAGA AAAGGAGAAA GGAGCTCTGA ATCTGATAGG AAAAGAAGGC |
| 11281 | TGCCTAAGGA GGAGTTTTTA GTATGTGGCG TATCATGCAA GTGCTATGCC AAGCCATGTC |
| 11341 | TAAATGGCTT TAATTATATA GTAATGCACT CTCAGTAATG GGGGACCAGC TTAAGTATAA |
| 11401 | TTAATAGATG GTTAGTGGGG TAATTCTGCT TCTAGTATTT TTTTTACTGT GCATACATGT |
| 11461 | TCATCGTATT TCCTTGGATT TCTGAATGGC TGCAGTGACC CAGATATTGC ACTAGGTCAA |
| 11521 | AACATTCAGG TATAGCTGAC ATCTCCTCTA TCACATTACA TCATCCTCCT TATAAGCCCA |
| 11581 | GCTCTGCTTT TTCCAGATTC TTCCACTGGC TCCACATCCA CCCCACTGGA TCTTCAGAAG |
| 11641 | GCTAGAGGGC GACTCTGGTG GTGCTTTTGT ATGTTTCAAT TAGGCTCTGA AATCTTGGGC |
| 11701 | AAAATGACAA GGGGAGGGCC AGGATTCCTC TCTCAGGTCA CTCCAGTGTT ACTTTTAATT |
| 11761 | CCTAGAGGGT AAATATGACT CCTTTCTCTA TCCCAAGCCA ACCAAGAGCA CATTCTTAAA |
| 11821 | GGAAAAGTCA ACATCTTCTC TCTTTTTTTT TTTTTTTGAG ACAGGGTCTC ACTATGTTGC |
| 11881 | CCAGGCTGCT CTTGAATTCC TGGGCTCAAG CAGTCCTCCC ACCCTACCAC AGCGTCCCGC |
| 11941 | GTAGCTGGGA CTACAGGTGC AAGCCACTAT GTCCAGCTAG CCAACTCCTC CTTGCCTGCT |
| 12001 | TTTCTTTTTT TTTCTTTTTT TGAGACGGCG CACCTATCAC CCAGGCTGGA GTGGAGTGGC |
| 12061 | ACGATCTTGG CTCACTGCAA CCTCTTCCTC CTGGTTCAAG CGATTCTCAT GTCTCAGCCT |
| 12121 | CCTCAGTAGC TAGGACTACC GGCGTGCACC ACCATGCCAG GCTAATTTTT ATATTTTTAG |
| 12181 | AATTTTAGAA GAGATGGGAT TTCATCATGT TGGCCAGGCT GGTCTCGAAC TCCTGACCTC |
| 12241 | AAGTGATCCA CCTGCCTTGG CCTCCCAAGG TGCTAGGATT ACAGGCATGA GCCACCGCAC |
| 12301 | CGGGCCCTCC TTGCCTGTTT TTCAATCTCA TCTGATATGC AGAGTATTTC TGCCCCACCC |
| 12361 | ACCTACCCCC CAAAAAAAGC TGAAGCCTAT TTATTTGAAA GTCCTTGTTT TTGCTACTAA |
| 12421 | TTATATAGTA TACCATACAT TATCATTCAA AACAACCATC CTGCTCATAA CATCTTTGAA |
| 12481 | AAGAAAAATA TATATGTGCA GTATTTTATT AAAGCAACAT TTTATTTAAG AATAAAGTCT |
| 12541 | TGTTAATTAC TATATTTTAG ATGCAATGTG ATCTGAAGTT TCTAATTCTG GCCCAACTAA |
| 12601 | ATTTCTAGCT CTGTTTCCCT AAACAAATAA TTTGGTTTCT CTGTGCCTGC ATTTTCCCTT |
| 12661 | TGGAGAAGAA AAGTGCTCTC TCTTGAGTTG ACCGAGAGTC CCATTAGGGA TAGGGAGACT |
| 12721 | TAAATGCATC CACAGGGGCA CAGGCAGAGT TGAGCACATA AACGGAGGCC CAAAATCAGC |
| 12781 | ATAGAACCAG AAAGATTCAG AGTTGGCCAA GAATGAACAT TGGCTACCAG ACCACAAGTC |
| 12841 | AGCATGAGTT GCTCTATGGC ATCAAATTGC AACTTGAGAG TAGATGGGCA GGGTCACTAT |
| 12901 | CAAATTAAGC AATCAGGGCA CACAAGTTGC AGTAACACAA CAAGACTAGG CCAGCTCTGG |
| 12961 | AATCCAGTAA CTCAGTGTCA GCAAGGTTTT GGGTTATAGT TCAAGAAAGT CTAAACAGAG |
| 13021 | CCAGTCACAG CACCAAGGAA TGCTCAAGGG AGCTATTGCA GGTTTCTCTG CTAAGAGATT |
| 13081 | TATTTCATCC TGGGTGCAGG GTTCGACCTC CAAAGGCCTC AAATCATCAC CGTATCAATG |
| 13141 | GATTTCCTGA GGGTAAGCTC CGCTATTTCA CACCTGAACT CCGGAGTCTG TATATTCAGG |
| 13201 | GAAGATTGCA TTCTCCTACT GGATTTGGGC TCTCAGAGGG CGTTGTGGGA ACCAGGCCCC |
| 13261 | TCACAGAATC AAATGGTCCC AACCAGGGAG AAAGAAAATA GTCTTTTTTT TTTTTTTAAT |
| 13321 | AGAGATGGGG GTCTCACTAT GCTGCCCAGG CTGGTCTTGA ACTCCTGGGT TCAAGTGATC |
| 13381 | CTCCTGCCTC AGCCTCCCAA AGTGCTGGGA TTACAGTGTG AGCCACTGCG CTTGGCCAGA |
| 13441 | AATGGTTTTG ATCTGTCTGA ACTGAACCCT ACTGCTTAGG CATAGCCCCA TCCTTGATAA |
| 13501 | TCTATTTGCT CCCAAGGACC AAGTCCAAGA TCCTTACAAG AAAGGTCTGC CAGAAAGTAA |
| 13561 | ATACTGCCCC CACTCCCTGA AGTTTATGAG GTTGATAAGA AAACATAACA GATAAAGTTT |
| 13621 | ATTGAGTGCT AACTTTA. |

By “guide polynucleotide” is meant a polynucleotide or polynucleotide complex which is specific for a target sequence and can form a complex with a polynucleotide programmable nucleotide binding domain protein (e.g., Cas9 or Cpf1). In an embodiment, the guide polynucleotide is a guide RNA (gRNA). gRNAs can exist as a complex of two or more RNAs, or as a single RNA molecule.

By “heterodimer” is meant a fusion protein comprising two domains, such as a wild type TadA domain and a variant of TadA domain or two variant TadA domains.

By “heterologous,” or “exogenous” is meant a polynucleotide or polypeptide that 1) has been experimentally incorporated to a polynucleotide or polypeptide sequence to which the polynucleotide or polypeptide is not normally found in nature; or 2) has been experimentally placed into a cell that does not normally comprise the polynucleotide or polypeptide. In some embodiments, “heterologous” means that a polynucleotide or polypeptide has been experimentally placed into a non-native context. In some embodiments, a heterologous polynucleotide or polypeptide is derived from a first species or host organism, and is incorporated into a polynucleotide or polypeptide derived from a second species or host organism. In some embodiments, the first species or host organism is different from the second species or host organism. In some embodiments the heterologous polynucleotide is DNA. In some embodiments the heterologous polynucleotide is RNA.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “increases” is meant a positive alteration of at least 10%, 25%, 50%, 75%, or 100%.

The term “inhibitor of base repair” or “IBR” refers to a protein that is capable in inhibiting the activity of a nucleic acid repair enzyme, for example a base excision repair (BER) enzyme. In some embodiments, the IBR is an inhibitor of inosine base excision repair. Exemplary inhibitors of base repair include inhibitors of APE1, Endo III, Endo IV, Endo V, Endo VIII, Fpg, hOGG1, hNEIL1, T7 Endol, T4PDG, UDG, hSMUGI, and hAAG. In some embodiments, the IBR is an inhibitor of Endo V or hAAG. In some embodiments, the IBR is a catalytically inactive EndoV or a catalytically inactive hAAG. In some embodiments, the base repair inhibitor is an inhibitor of Endo V or hAAG. In some embodiments, the base repair inhibitor is a catalytically inactive EndoV or a catalytically inactive hAAG.

In some embodiments, the base repair inhibitor is uracil glycosylase inhibitor (UGI). UGI refers to a protein that is capable of inhibiting a uracil-DNA glycosylase base-excision repair enzyme. In some embodiments, a UGI domain comprises a wild-type UGI or a fragment of a wild-type UGI. In some embodiments, the UGI proteins provided herein include fragments of UGI and proteins homologous to a UGI or a UGI fragment. In some embodiments, the base repair inhibitor is an inhibitor of inosine base excision repair. In some embodiments, the base repair inhibitor is a “catalytically inactive inosine specific nuclease” or “dead inosine specific nuclease. Without wishing to be bound by any particular theory, catalytically inactive inosine glycosylases (e.g., alkyl adenine glycosylase (AAG)) can bind inosine, but cannot create an abasic site or remove the inosine, thereby sterically blocking the newly formed inosine moiety from DNA damage/repair mechanisms. In some embodiments, the catalytically inactive inosine specific nuclease can be capable of binding an inosine in a nucleic acid but does not cleave the nucleic acid. Non-limiting exemplary catalytically inactive inosine specific nucleases include catalytically inactive alkyl adenosine glycosylase (AAG nuclease), for example, from a human, and catalytically inactive endonuclease V (EndoV nuclease), for example, from E. coli. In some embodiments, the catalytically inactive AAG nuclease comprises an E125Q mutation or a corresponding mutation in another AAG nuclease.

An “intein” is a fragment of a protein that is able to excise itself and join the remaining fragments (the exteins) with a peptide bond in a process known as protein splicing.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide as described herein is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule as described is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide as described that has been separated from components that naturally accompany it. Typically, the polypeptide is isolated when it is at least 60%, by weight, free from the proteins and naturally-occurring organic molecules with which it is naturally associated. Preferably, the preparation comprises at least 75%, more preferably at least 90%, and most preferably at least 99%, by weight, a polypeptide as described herein. An isolated polypeptide as described herein may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “linker”, as used herein, refers to a molecule that links two moieties. In one embodiment, the term “linker” refers to a covalent linker (e.g., covalent bond) or a non-covalent linker.

By “marker” is meant any protein or polynucleotide having an alteration in expression level or activity that is associated with a disease or disorder, such as, GSD1a and/or symptoms thereof.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The term “non-conservative mutations” involve amino acid substitutions between different groups, for example, lysine for tryptophan, or phenylalanine for serine, etc. In this case, it is preferable for the non-conservative amino acid substitution to not interfere with, or inhibit the biological activity of, the functional variant. The non-conservative amino acid substitution can enhance the biological activity of the functional variant, such that the biological activity of the functional variant is increased as compared to the wild-type protein.

The term “nuclear localization sequence,” “nuclear localization signal,” or “NLS” refers to an amino acid sequence that promotes import of a protein into the cell nucleus. Nuclear localization sequences are known in the art and described, for example, in Plank et al., International PCT application, PCT/EP2000/011690, filed Nov. 23, 2000, published as WO/2001/038547 on May 31, 2001, the contents of which are incorporated herein by reference for their disclosure of exemplary nuclear localization sequences. In other embodiments, the NLS is an optimized NLS described, for example, by Koblan et al., Nature Biotech. 2018 doi:10.1038/nbt.4172. In some embodiments, an NLS comprises the amino acid sequence In some embodiments, an NLS comprises the amino acid sequence

(SEQ ID NO: 190)
KRTADGSEFESPKKKRKV,
(SEQ ID NO: 191)
KRPAATKKAGQAKKKK,
(SEQ ID NO: 192)
KKTELQTTNAENKTKKL,
(SEQ ID NO: 193)
KRGINDRNFWRGENGRKTR,
(SEQ ID NO: 194)
RKSGKIAAIVVKRPRK,
(SEQ ID NO: 195)
PKKKRKV,
or
(SEQ ID NO: 196)
MDSLLMNRRKFLYQFKNVRWAKGRRETYLC.

The term “nucleobase,” “nitrogenous base,” or “base,” used interchangeably herein, refers to a nitrogen-containing biological compound that forms a nucleoside, which in turn is a component of a nucleotide. The ability of nucleobases to form base pairs and to stack one upon another leads directly to long-chain helical structures such as ribonucleic acid (RNA) and deoxyribonucleic acid (DNA). Five nucleobases—adenine (A), cytosine (C), guanine (G), thymine (T), and uracil (U)—are called primary or canonical. Adenine and guanine are derived from purine, and cytosine, uracil, and thymine are derived from pyrimidine. DNA and RNA can also contain other (non-primary) bases that are modified. Non-limiting exemplary modified nucleobases can include hypoxanthine, xanthine, 7-methylguanine, 5,6-dihydrouracil, 5-methylcytosine (m5C), and 5-hydromethylcytosine. Hypoxanthine and xanthine can be created through mutagen presence, both of them through deamination (replacement of the amine group with a carbonyl group). Hypoxanthine can be modified from adenine. Xanthine can be modified from guanine. Uracil can result from deamination of cytosine. A “nucleoside” consists of a nucleobase and a five carbon sugar (either ribose or deoxyribose). Examples of a nucleoside include adenosine, guanosine, uridine, cytidine, 5-methyluridine (m5U), deoxyadenosine, deoxyguanosine, thymidine, deoxyuridine, and deoxycytidine. Examples of a nucleoside with a modified nucleobase includes inosine (I), xanthosine (X), 7-methylguanosine (m7G), dihydrouridine (D), 5-methylcytidine (m5C), and pseudouridine (Ψ). A “nucleotide” consists of a nucleobase, a five carbon sugar (either ribose or deoxyribose), and at least one phosphate group.

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, a polynucleotide described herein comprises one or more of the following modifications: 2′-O-methyl (2′-OMe), phosphorothioate (PS), 2′-O-methyl thioPACE (MSP), 2′-O-methyl-PACE (MP), 2′-fluoro RNA (2′-F-RNA), and constrained ethyl (S-cEt).

The term “nucleic acid programmable DNA binding protein” or “napDNAbp” may be used interchangeably with “polynucleotide programmable nucleotide binding domain” to refer to a protein that associates with a nucleic acid (e.g., DNA or RNA), such as a guide nucleic acid or guide polynucleotide (e.g., gRNA), that guides the napDNAbp to a specific nucleic acid sequence. In some embodiments, the polynucleotide programmable nucleotide binding domain is a polynucleotide programmable DNA binding domain. In some embodiments, the polynucleotide programmable nucleotide binding domain is a polynucleotide programmable RNA binding domain. In some embodiments, the polynucleotide programmable nucleotide binding domain is a Cas9 protein. A Cas9 protein can associate with a guide RNA that guides the Cas9 protein to a specific DNA sequence that is complementary to the guide RNA. In some embodiments, the napDNAbp is a Cas9 domain, for example a nuclease active Cas9, a Cas9 nickase (nCas9), or a nuclease inactive Cas9 (dCas9). Non-limiting examples of nucleic acid programmable DNA binding proteins include, Cas9 (e.g., dCas9 and nCas9), Cas12a/Cpf1, Cas12b/C2c1, Cas12c/C2c3, Cas12d/CasY, Cas12e/CasX, Cas12g, Cas12h, Cas12i, and Cas12j/CasΦ(Cas12j/CasΦ). Non-limiting examples of Cas enzymes include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2c1 (e.g., SEQ ID NO: 232), Cas12c/C2c3, Cas12d/CasY, Cas12e/CasX, Cas12g, Cas12h, Cas12i, Cas12j/CasΦ, Cpf1, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, homologues thereof, or modified or engineered versions thereof. Other nucleic acid programmable DNA binding proteins are also within the scope of this disclosure, although they may not be specifically listed in this disclosure. See, e.g., Makarova et al. “Classification and Nomenclature of CRISPR-Cas Systems: Where from Here?” CRISPR J. 2018 October; 1:325-336. doi: 10.1089/crispr.2018.0033; Yan et al., “Functionally diverse type V CRISPR-Cas systems” Science. 2019 Jan. 4; 363(6422):88-91. doi: 10.1126/science.aav7271, the entire contents of each are hereby incorporated by reference. Exemplary nucleic acid programmable DNA binding proteins and nucleic acid sequences encoding nucleic acid programmable DNA binding proteins are provided in the Sequence Listing as SEQ ID NOs: 197-230.

The terms “nucleobase editing domain” or “nucleobase editing protein,” as used herein, refers to a protein or enzyme that can catalyze a nucleobase modification in RNA or DNA, such as cytosine (or cytidine) to uracil (or uridine) or thymine (or thymidine), and adenine (or adenosine) to hypoxanthine (or inosine) deaminations, as well as non-templated nucleotide additions and insertions. In some embodiments, the nucleobase editing domain is a deaminase domain (e.g., an adenine deaminase or an adenosine deaminase; or a cytidine deaminase or a cytosine deaminase).

As used herein, “obtaining” as in “obtaining an agent” includes synthesizing, purchasing, or otherwise acquiring the agent.

A “patient” or “subject” as used herein refers to a mammalian subject or individual diagnosed with, at risk of having or developing, or suspected of having or developing a disease or a disorder. In some embodiments, the term “patient” refers to a mammalian subject with a higher than average likelihood of developing a disease or a disorder. Exemplary patients can be humans, non-human primates, cats, dogs, pigs, cattle, cats, horses, camels, llamas, goats, sheep, rodents (e.g., mice, rabbits, rats, or guinea pigs) and other mammalians that can benefit from the therapies disclosed herein. Exemplary human patients can be male and/or female.

“Patient in need thereof” or “subject in need thereof” is referred to herein as a patient diagnosed with, at risk or having, predetermined to have, or suspected of having a disease or disorder, for instance, but not restricted to Glycogen Storage Disease Type 1 (GSD1 or Von Gierke Disease).

The terms “pathogenic mutation,” “pathogenic variant,” “disease casing mutation,” “disease causing variant,” “deleterious mutation,” or “predisposing mutation” refers to a genetic alteration or mutation that increases an individual's susceptibility or predisposition to a certain disease or disorder. In some embodiments, the pathogenic mutation comprises at least one wild-type amino acid substituted by at least one pathogenic amino acid in a protein encoded by a gene.

The term “pharmaceutically-acceptable carrier” means a pharmaceutically-acceptable material, composition, or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). The terms such as “excipient,” “carrier,” “pharmaceutically acceptable carrier,” “vehicle,” or the like are used interchangeably herein.

The term “pharmaceutical composition” means a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

The terms “protein”, “peptide”, “polypeptide”, and their grammatical equivalents are used interchangeably herein, and refer to a polymer of amino acid residues linked together by peptide (amide) bonds. A protein, peptide, or polypeptide can be naturally occurring, recombinant, or synthetic, or any combination thereof.

The term “fusion protein” as used herein refers to a hybrid polypeptide which comprises protein domains from at least two different proteins.

By “promoter” is meant an array of nucleic acid control sequences, which direct transcription of a nucleic acid. A promoter includes necessary nucleic acid sequences near the start site of transcription. A promoter also optionally includes distal enhancer or repressor sequence elements. A “constitutive promoter” is a promoter that is continuously active and is not subject to regulation by external signals or molecules. In contrast, the activity of an “inducible promoter” is regulated by an external signal or molecule (for example, a transcription factor). By way of example, a promoter may be a CMV promoter.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

By “reduces” is meant a negative alteration of at least 10%, 25%, 50%, 75%, or 100%.

By “reference” is meant a standard or control condition. In one embodiment, the reference is a wild-type or healthy cell. For example, in some embodiments the reference is a cell of a subject not inflicted with GSD1a. In some embodiments, the reference is a cell with normal or wild-type glucose-6-phosphatase activity. In other embodiments and without limitation, a reference is an untreated cell that is not subjected to a test condition, or is subjected to placebo or normal saline, medium, buffer, and/or a control vector that does not harbor a polynucleotide of interest.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides or about 300 nucleotides or any integer thereabout or therebetween. In some embodiments, a reference sequence is a wild-type sequence of a protein of interest. In other embodiments, a reference sequence is a polynucleotide sequence encoding a wild-type protein.

The term “RNA-programmable nuclease,” and “RNA-guided nuclease” are used with (e.g., binds or associates with) one or more RNA(s) that is not a target for cleavage. In some embodiments, an RNA-programmable nuclease, when in a complex with an RNA, may be referred to as a nuclease:RNA complex. Typically, the bound RNA(s) is referred to as a guide RNA (gRNA). In some embodiments, the RNA-programmable nuclease is the (CRISPR-associated system) Cas9 endonuclease, for example, Cas9 (Csn1) from Streptococcus pyogenes.

The term “single nucleotide polymorphism (SNP)” is a variation in a single nucleotide that occurs at a specific position in the genome, where each variation is present to some appreciable degree within a population (e.g., >1%).

By “specifically binds” is meant a nucleic acid molecule, polypeptide, or complex thereof (e.g., a nucleic acid programmable DNA binding protein, a guide nucleic acid), compound, or molecule that recognizes and binds a polypeptide and/or nucleic acid molecule as described herein, but which does not substantially recognize and bind other molecules in a sample, for example, a biological sample.

By “substantially identical” is meant a polypeptide or nucleic acid molecule exhibiting at least 50% identity to a reference amino acid sequence. In one embodiment, a reference sequence is a wild-type amino acid or nucleic acid sequence. In another embodiment, a reference sequence is any one of the amino acid or nucleic acid sequences described herein. In one embodiment, such a sequence is at least 60%, 80%, 85%, 90%, 95% or even 99% identical at the amino acid level or nucleic acid level to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

COBALT is used, for example, with the following parameters:
a) alignment parameters: Gap penalties-11,-1 and End-Gap penalties-5,-1,
b) CDD Parameters: Use RPS BLAST on; Blast E-value 0.003; Find Conserved columns and Recompute on, and
c) Query Clustering Parameters: Use query clusters on; Word Size 4; Max cluster distance 0.8; Alphabet Regular.
   EMBOSS Needle is used, for example, with the following parameters:
a) Matrix: BLOSUM62;
b) GAP OPEN: 10;
c) GAP EXTEND: 0.5;
d) OUTPUT FORMAT: pair;
e) END GAP PENALTY: false;
f) END GAP OPEN: 10; and
g) END GAP EXTEND: 0.5.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In an embodiment, wash steps will occur at 25° C. in 30 mM NaCl, 3 mM trisodium citrate, and 0.1% SDS. In another embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “split” is meant divided into two or more fragments.

A “split Cas9 protein” or “split Cas9” refers to a Cas9 protein that is provided as an N-terminal fragment and a C-terminal fragment encoded by two separate nucleotide sequences. The polypeptides corresponding to the N-terminal portion and the C-terminal portion of the Cas9 protein may be spliced to form a “reconstituted” Cas9 protein.

By “subject” is meant a mammal, including, but not limited to, a human or non-human mammal, such as a bovine, equine, canine, ovine, or feline. Subjects include livestock, domesticated animals raised to produce labor and to provide commodities, such as food, including without limitation, cattle, goats, chickens, horses, pigs, rabbits, and sheep.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In one embodiment, such a sequence is at least 60%, 80% or 85%, 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, COBALT, EMBOSS Needle, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

COBALT is used, for example, with the following parameters:
a) alignment parameters: Gap penalties-11,-1 and End-Gap penalties-5,-1,
b) CDD Parameters: Use RPS BLAST on; Blast E-value 0.003; Find Conserved columns and Recompute on, and
c) Query Clustering Parameters: Use query clusters on; Word Size 4; Max cluster distance 0.8; Alphabet Regular.
   EMBOSS Needle is used, for example, with the following parameters:
a) Matrix: BLOSUM62;
b) GAP OPEN: 10;
c) GAP EXTEND: 0.5;
d) OUTPUT FORMAT: pair;
e) END GAP PENALTY: false;
f) END GAP OPEN: 10; and
g) END GAP EXTEND: 0.5.

The term “target site” refers to a sequence within a nucleic acid molecule that is modified by a nucleobase editor. In one embodiment, the target site is deaminated by a deaminase or a fusion protein comprising a deaminase (e.g., adenine deaminase).

As used herein “transduction” means to transfer a gene or genetic material to a cell via a viral vector.

“Transformation,” as used herein refers to the process of introducing a genetic change in a cell produced by the introduction of exogenous nucleic acid.

“Transfection” refers to the transfer of a gene or genetical material to a cell via a chemical or physical means.

By “translocation” is meant the rearrangement of nucleic acid segments between non-homologous chromosomes.

As used herein, the terms “treat,” treating,” “treatment,” and the like refer to reducing or ameliorating a disorder and/or symptom(s) associated therewith or obtaining a desired pharmacologic and/or physiologic effect. It will be appreciated that, although not precluded, treating a disorder or condition does not require that the disorder, condition or symptoms associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease. In some embodiments, the effect is preventative, i.e., the effect protects or prevents an occurrence or reoccurrence of a disease or condition. To this end, the presently disclosed methods comprise administering a therapeutically effective amount of a compositions as described herein.

By “uracil glycosylase inhibitor” or “UGI” is meant an agent that inhibits the uracil-excision repair system. In one embodiment, the agent is a protein or fragment thereof that binds a host uracil-DNA glycosylase and prevents removal of uracil residues from DNA. In an embodiment, a UGI is a protein, a fragment thereof, or a domain that is capable of inhibiting a uracil-DNA glycosylase base-excision repair enzyme. In some embodiments, a UGI domain comprises a wild-type UGI or a modified version thereof. In some embodiments, a UGI domain comprises a fragment of the exemplary amino acid sequence set forth below. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or 100% of the exemplary UGI sequence provided below. In some embodiments, a UGI comprises an amino acid sequence that is homologous to the exemplary UGI amino acid sequence or fragment thereof, as set forth below. In some embodiments, the UGI, or a portion thereof, is at least 70%, at least 75%, at least 80%, at least 85%, at least 90%, at least 95%, at least 96%, at least 97%, at least 98%, at least 99%, at least 99.5%, at least 99.9%, or 100% identical to a wild-type UGI or a UGI sequence, or portion thereof, as set forth below. An exemplary UGI comprises an amino acid sequence as follows: >sp1P14739IUNGI_BPPB2 Uracil-DNA glycosylase inhibitor

(SEQ ID NO: 231)
MTNLSDIIEKETGKOLVIQESILMLPEEVEEVIGNKPESDILVHTAYDES
TDENVMLLTSDAPEYKPWALVIQDSNGENKIKML.

The term “vector” refers to a means of introducing a nucleic acid sequence into a cell, resulting in a transformed cell. Vectors include plasmids, transposons, phages, viruses, liposomes, and episome. “Expression vectors” are nucleic acid sequences comprising the nucleotide sequence to be expressed in the recipient cell. Expression vectors may include additional nucleic acid sequences to promote and/or facilitate the expression of the of the introduced sequence such as start, stop, enhancer, promoter, and secretion sequences.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

DNA editing has emerged as a viable means to modify disease states by correcting pathogenic mutations at the genetic level. Until recently, all DNA editing platforms have functioned by inducing a DNA double strand break (DSB) at a specified genomic site and relying on endogenous DNA repair pathways to determine the product outcome in a semi-stochastic manner, resulting in complex populations of genetic products. Though precise, user-defined repair outcomes can be achieved through the homology directed repair (HDR) pathway, a number of challenges have prevented high efficiency repair using HDR in therapeutically-relevant cell types. In practice, this pathway is inefficient relative to the competing, error-prone non-homologous end joining pathway. Further, HDR is tightly restricted to the G1 and S phases of the cell cycle, preventing precise repair of DSBs in post-mitotic cells. As a result, it has proven difficult or impossible to alter genomic sequences in a user-defined, programmable manner with high efficiencies in these populations.

### BRIEF DESCRIPTION OF THE DRAWINGS

FIG. 1 provides a schematic depicting a G6PC nucleotide target sequence (ATTCTCTTTGGACAGTGTCCATACTGGTGG; SEQ ID NO 399) and corresponding amino acid sequence (ILFGQCPYWW; SEQ ID NO: 401) indicating bystander and on target A>G bases for correction of the GSD1a R83C mutation.

FIGS. 2A and 2B depict in vivo correction of GSD1a mutations in liver extracts of transgenic mouse models heterozygous for huG6PC-R83C. FIG. 2A is a schematic depicting in vivo workflow. Lipid nanoparticles (LNP) carrying base editor mRNA and gRNA were dosed via IV injection in transgenic mice heterozygous for huG6PC (huR83C TET), harboring the R83C mutation. FIG. 2B is a bar graph depicting A to G base editing efficiency of the GSD1a R83C mutation using MSP828 comparing on-target to bystander editing.

FIG. 3 is a bar graph depicting correction of the GSD1a R83C mutation in a transgenic mouse model heterozygous for huG6PC, harboring the R83C mutation, using TadA adenosine deaminase variants MSP605, MSP824, MSP825, MSP680, MSP828, and MSP820. In vitro screens were run to select desirable base-editors for R83C correction. LNP co-formulations of gRNA and representative base-editors were dosed (at a sub-saturating dose of 1 mpk), in vivo, in transgenic mice heterozygous for huG6PC-R83C. The base-editing potency of the variants for the R83C correction in livers of the LNP-treated, huG6PC-R83C heterozygote, transgenic animals are shown in FIG. 3. Variant MSP828 yielded a high level of on-target activity under these conditions. A to G base editing efficiency is shown for on-target and bystander editing.

FIG. 4 shows schematics depicting normal and loss-of-function g6pc function and related outcomes. GSD-Ia (or GSD1a herein) is an autosomal recessive disorder caused by mutations in the g6pc gene. R83C, located in the active site of the enzyme, is the most prevalent pathogenic mutation identified in Caucasian GSD-Ia patients and is associated with inactivation of G6Pase. A loss of G6Pase function can result in life-threatening hypoglycemia, seizures and even death. To mitigate hypoglycemia, patients must maintain strict and frequent adherence to glucose supplementation through day and night, by way of a slow glucose release formula. One missed or delayed dose can result in emergency hypoglycemia. Among many complications, enlarged liver, accumulation of uric acid, lactate, and lipids are common in GSD-Ia patients.

FIG. 5 shows a schematic illustrating that base editors as described herein generate permanent, predicted nucleotide substitutions in an editing window. The R83C mutation introduces a single G>A conversion in the g6pc gene. Adenine base editors (ABEs) enable the programmable conversion of A to G in genomic DNA and thus may be used to correct this mutation. FIG. 5 depicts the utility of ABEs and base editing as described herein. ABE binds to target DNA that is complementary to the guide-RNA and exposes a stretch of single-stranded DNA. The deaminase converts the target adenine into inosine, and the Cas enzyme nicks the opposite strand, which is then repaired, completing the base pair conversion. The direct repair of a point mutation has the potential for restoration of gene function.

FIGS. 6A and 6B provide a depiction of the target nucleotide site, and bystander and PAM nucleotides and a bar graph showing that ABEs used in immortalized HEK293 cells yield a significant rate of precise correction of R83C. Base-editors for A>G conversion in the g6pc gene were optimized for correction of R83C. Shown in FIG. 6A is the target DNA sequence (CCACCAGTATGGACACTGTCCAAAGAGAAT (SEQ ID NO: 402)) and underlying amino acid translation (WWYPCQGFLI; SEQ ID NO: 403) for the GSD-Ia R83C mutation. The target edit is shown by double-underlining, at position 12. The editing window also includes a possible bystander, shown by single-underlining at position 6, and an edit that may result in a synonymous conversion is shown at position 10. For screening, a HEK293 cell line was generated to express the g6pc transgene harboring the R83C mutation and was transfected with base-editor mRNA and gRNA. Allele frequencies were assessed by high-throughput targeted amplicon Next-Generation Sequencing (NGS). Variants 1-5 represent a combination of gRNA and base-editor RNA, engineered for optimized target correction. Variant 5 yielded approximately 60% targeted base-editing efficiency for R83C correction with limited bystander editing (FIG. 6B).

FIG. 7 presents a photographic image and bar graphs demonstrating that 3-week-old homozygous huR83C (Hom huR83C) mice exhibited expected growth impairment and metabolic defects characteristic of GSD-1a. For the experients, a GSD-Ia mouse that expresses the human G6PC-R83C transgene in place of mouse G6PC was generated to validate base-editing in vivo. The results shown confirmed that mice homozygous for huR83C exhibited postnatal lethality—they were either stillborn or died within 24 hours. On glucose supplementation therapy, the animals survived to at least 3 weeks of age and revealed characteristic pathological signatures of GSD-Ia, with reduced body weight, enlarged livers, significant G6Pase inhibition, and abnormal serum metabolites as compared to littermate controls, a phenotype that is consistent with clinical and published reports.

FIGS. 8A and 8B show dot plots of in vivo correction achieved by the base editors (ABEs) described herein. FIG. 8A illustrates efficient lipid nanoparticle (LNP)-mediated base editing (huG6PC-R83C correction) in livers of adult and newborn heterozygous huR83C mice. To validate base-editing efficiency for R83C correction in vivo, LNP-mediated delivery was first optimized in less fragile transgenic mice heterozygous for huR83C. The schematic in FIG. 2A depicts in vivo workflow for these experiments, with lipid nanoparticle (LNP), or LNP co-formulations of base-editor mRNA and gRNA dosed via IV injection.

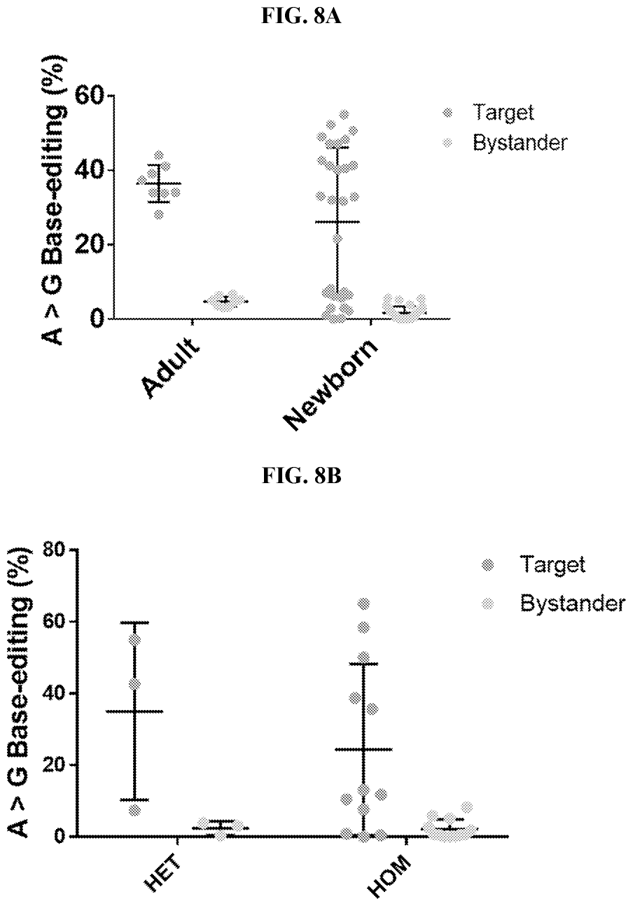

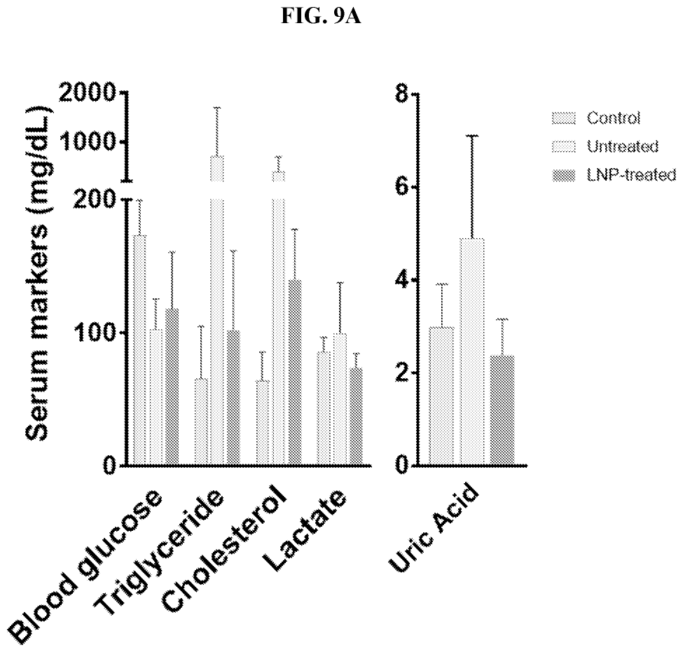

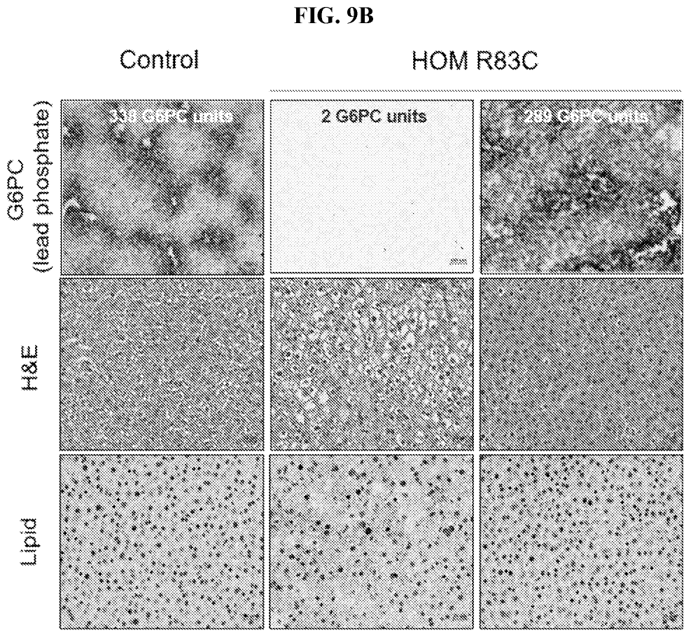

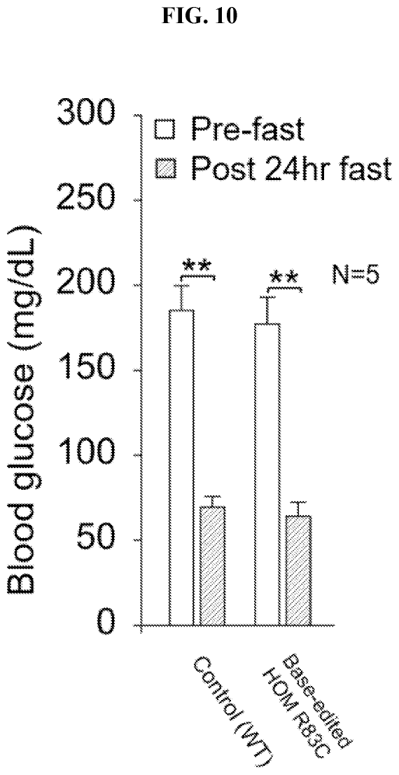

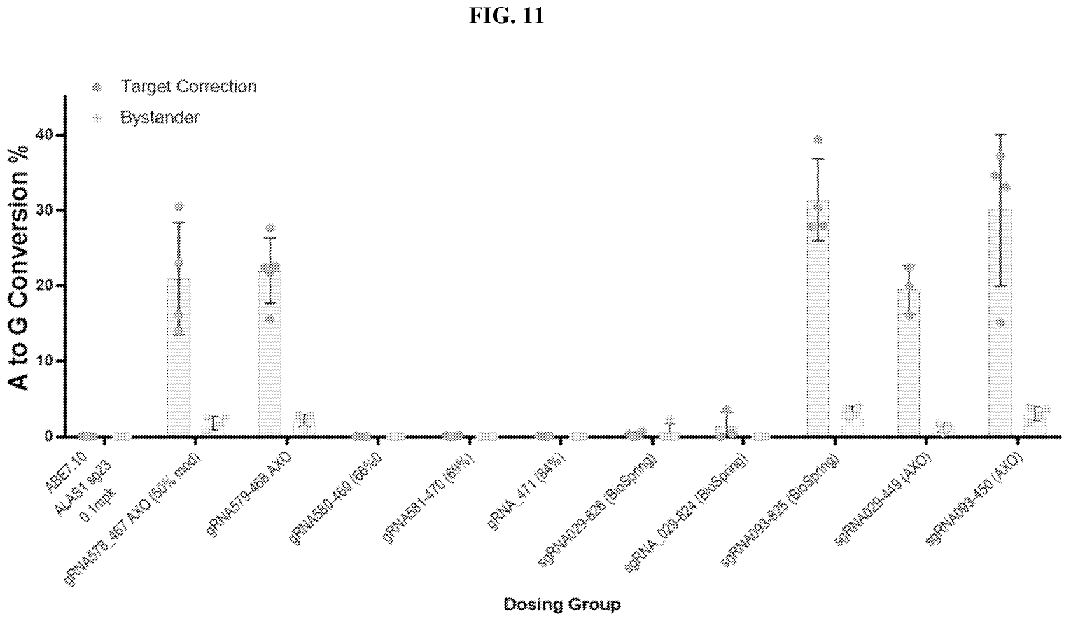

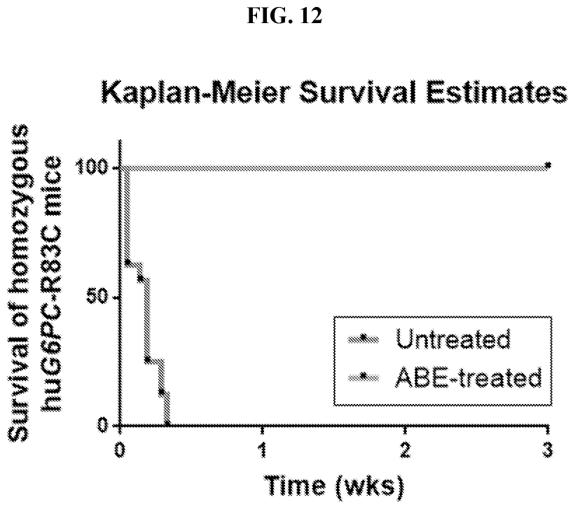

Given neonatal lethality of the homozygous mice, LNP-dosing was employed via the temporal vein of heterozygous huR83C mice shortly post birth, and activity was compared to that seen in adult heterozygous huR83C mice that had received LNP administered via the tail vein. NGS analysis of whole liver extracts revealed approximately 40% base-editing efficiency in adults and up to ˜60% efficiency in newborns, with a broader range in efficiencies. Bystander editing remained low in adults and newborns. FIG. 8B shows that LNP-mediated R83C correction in livers is associated with survival of newborn homozygous huR83C mice and littermate heterozygous huR83C mice. Briefly, newborn mice homozygous for huR83C were treated with LNP containing guide RNA and mRNA encoding ABE. The treated mice grew normally to 3 weeks of age, without hypoglycemia-induced seizures, in the absence of glucose therapy. The treated homozygous huR83C mice displayed editing efficiencies up to ˜60% in total liver extracts (i.e., ˜60% R83C correction), consistent with littermate controls that were heterozygous for huR83C.

FIGS. 9A and 9B show bar graphs and immunohistochemical staining images demonstrating the base editing as described herein in mice homozygous for huG6PC-R83C restores near-normal metabolic function to reverse GSD-Ia pathology. At 3 weeks, it was validated that the treated homozygous huR83C mice displayed proper metabolic function, with restoration of near-normal serum metabolite markers, including glucose, triglycerides, cholesterol, lactate, and uric acid, as shown by the darkest bars in the graph in FIG. 9A. Moreover, biochemical assays of G6PC activity (as assessed biochemically and via lead-phosphate staining) in LNP-treated homozygous huR83C mice were consistent with that of litter-mate controls. Hepatomegaly, another clinical presentation of GSD-Ia, is caused primarily by excess glycogen and lipid deposition. Immuno-histochemical analysis revealed normal hepatocyte size and lipid deposition in LNP-treated mice (FIG. 9B). The results demonstrate the potential of base-editing to correct the R83C mutation and the metabolic defects associated with GSD-Ia.

FIG. 10 shows a bar graph demonstrating that a single LNP dose administration in homozygous huG6PC-R83C mice maintained euglycemia during a 24-hour fasting challenge via base-editing as described herein.

FIG. 11 shows a bar graph demonstrating the results of experiments in which representative heavy mods gRNAs (heavy mod series 1 gRNAs for R83C (saCas9), Example 5) were used in experiments in which adult transgenic mice heterozygous for huG6PC-R83C were dosed at a sub-saturating dose of 1 mpk of 1:1 ratio of gRNA:editor mRNA.

FIG. 12 presents a graph illustrating Kaplan-Meier survival curves generated to estimate the survival of newborn transgenic mice homozygous for huG6PC-R83C, either post base-editing via ABE mRNA (ABE-treated) or untreated (Untreated). The top line plot on the graph represents the survival of animals following base-editing via ABE mRNA (ABE-treated), (100% survival over time (3 wks)), as described herein (Example 2). The leftmost line plot on the graph represents the survival of untreated animals over time (poor to no survival at less than 1 week).

### DETAILED DESCRIPTION OF THE EMBODIMENTS

Provided and featured herein are compositions comprising novel adenosine base editors that have increased efficiency and methods of using base editors comprising adenosine deaminase variants for altering mutations associated with Glycogen Storage Disease Type 1a (GSD1a).

The embodiments described herein are based, at least in part, on the discovery that a base editor featuring adenosine deaminase variants (e.g., adenosine deaminase variants that comprise a combination of alterations in a TadA*7.10 amino acid sequence, where the combination of alterations is V82G, Y147T/D, Q154S, and one or more of L36H, I76Y, F149Y, N157K, and D167N, or a corresponding combination of alterations in another adenosine deaminase) precisely corrects single nucleotide polymorphisms in the endogenous glucose-6-phosphatase (G6PC) gene (e.g. R83C, Q347X).

The GSD1a mutations, R83C and Q347X, are cytidine to thymidine (C→T) transition mutations, resulting in a C·G to T·A base pair substitution. These substitutions may be reverted back to a wild-type, non-pathogenic genomic sequence with an adenosine base editor (ABE) which catalyzes A·T to G·C substitutions. By extension, GSD1a-causing mutations are potential targets for reversion to wild-type sequence using ABEs without the risks of inducing G6PC gene overexpression, as may occur using gene therapy. Accordingly, A·T to G·C DNA base editing precisely corrects one or more of the most prevalent GSD1a-causing mutations in the G6PC gene.

### Nucleobase Editors

Useful in the methods and compositions described herein are nucleobase editors that edit, modify or alter a target nucleotide sequence of a polynucleotide. Nucleobase editors described herein typically include a polynucleotide programmable nucleotide binding domain and a nucleobase editing domain (e.g., adenosine deaminase or cytidine deaminase). A polynucleotide programmable nucleotide binding domain, when in conjunction with a bound guide polynucleotide (e.g., gRNA), can specifically bind to a target polynucleotide sequence and thereby localize the base editor to the target nucleic acid sequence desired to be edited.

In certain embodiments, the nucleobase editors provided herein comprise one or more features that improve base editing activity. For example, any of the nucleobase editors provided herein may comprise a Cas9 domain that has reduced nuclease activity. In some embodiments, any of the nucleobase editors provided herein may have a Cas9 domain that does not have nuclease activity (dCas9), or a Cas9 domain that cuts one strand of a duplexed DNA molecule, referred to as a Cas9 nickase (nCas9). Without wishing to be bound by any particular theory, the presence of the catalytic residue (e.g., H840) maintains the activity of the Cas9 to cleave the non-edited (e.g., non-deaminated) strand opposite the targeted nucleobase. Mutation of the catalytic residue (e.g., D10 to A10) prevents cleavage of the edited (e.g., deaminated) strand containing the targeted residue (e.g., A or C). Such Cas9 variants can generate a single-strand DNA break (nick) at a specific location based on the gRNA-defined target sequence, leading to repair of the non-edited strand, ultimately resulting in a nucleobase change on the non-edited strand.

### Polynucleotide Programmable Nucleotide Binding Domain

Polynucleotide programmable nucleotide binding domains bind polynucleotides (e.g., RNA, DNA). A polynucleotide programmable nucleotide binding domain of a base editor can itself comprise one or more domains (e.g., one or more nuclease domains). In some embodiments, the nuclease domain of a polynucleotide programmable nucleotide binding domain can comprise an endonuclease or an exonuclease. An endonuclease can cleave a single strand of a double-stranded nucleic acid or both strands of a double-stranded nucleic acid molecule. In some embodiments, a nuclease domain of a polynucleotide programmable nucleotide binding domain can cut zero, one, or two strands of a target polynucleotide.

Non-limiting examples of a polynucleotide programmable nucleotide binding domain which can be incorporated into a base editor include a CRISPR protein-derived domain, a restriction nuclease, a meganuclease, TAL nuclease (TALEN), and a zinc finger nuclease (ZFN). In some embodiments, a base editor comprises a polynucleotide programmable nucleotide binding domain comprising a natural or modified protein or portion thereof which via a bound guide nucleic acid is capable of binding to a nucleic acid sequence during CRISPR (i.e., Clustered Regularly Interspaced Short Palindromic Repeats)-mediated modification of a nucleic acid. Such a protein is referred to herein as a “CRISPR protein.” Accordingly, disclosed herein is a base editor comprising a polynucleotide programmable nucleotide binding domain comprising all or a portion of a CRISPR protein (i.e. a base editor comprising as a domain all or a portion of a CRISPR protein, also referred to as a “CRISPR protein-derived domain” of the base editor). A CRISPR protein-derived domain incorporated into a base editor can be modified compared to a wild-type or natural version of the CRISPR protein. For example, as described below a CRISPR protein-derived domain can comprise one or more mutations, insertions, deletions, rearrangements and/or recombinations relative to a wild-type or natural version of the CRISPR protein.

Cas proteins that can be used herein include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, Cas12a/Cpf1, Cas12b/C2c1 (e.g., SEQ ID NO: 232), Cas12c/C2c3, Cas12d/CasY, Cas12e/CasX, Cas12g, Cas12h, Cas12i, and Cas12j/CasΦ, CARF, DinG, homologues thereof, or modified versions thereof. A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

A vector that encodes a CRISPR enzyme that is mutated to with respect, to a corresponding wild-type enzyme such that the mutated CRISPR enzyme lacks the ability to cleave one or both strands of a target polynucleotide containing a target sequence can be used. A Cas protein (e.g., Cas9, Cas12) or a Cas domain (e.g., Cas9, Cas12) can refer to a polypeptide or domain with at least or at least about 50%, 60%, 70%, 80%, 90%, 91%, 92%, 93%, 94%, 95%, 96%, 97%, 98%, 99%, or 100% sequence identity and/or sequence homology to a wild-type exemplary Cas polypeptide or Cas domain. Cas (e.g., Cas9, Cas12) can refer to the wild-type or a modified form of the Cas protein that can comprise an amino acid change such as a deletion, insertion, substitution, variant, mutation, fusion, chimera, or any combination thereof.

In some embodiments, a CRISPR protein-derived domain of a base editor can include all or a portion of Cas9 from Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); Neisseria meningitidis (NCBI Ref: YP_002342100.1), Streptococcus pyogenes, or Staphylococcus aureus.

Cas9 nuclease sequences and structures are well known to those of skill in the art (See, e.g., “Complete genome sequence of an Ml strain of Streptococcus pyogenes.” Ferretti et al., Proc. Natl. Acad. Sci. U.S.A. 98:4658-4663(2001); “CRISPR RNA maturation by trans-encoded small RNA and host factor RNase III.” Deltcheva E., et al., Nature 471:602-607(2011); and “A programmable dual-RNA-guided DNA endonuclease in adaptive bacterial immunity.” Jinek M., et al., Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

In some embodiments, the gRNA scaffold sequence is as follows:

(SEQ ID NO: 317)
GUUUUAGAGCUAGAAAUAGCAAGUUAAAAUAAGGCUAGUCCGUUAUCAAC
UUGAAAAAGUGGCACCGAGUCGGUGCUUUU

In an embodiment, the RNA scaffold comprises a stem loop. In an embodiment, the RNA scaffold comprises the nucleic acid sequence:

(SEQ ID NO: 389)
GUUUUUGUACUCUCAAGAUUUAAGUAACUGUACAACGAAACUUACACAGU
UACUUAAAUCUUGCAGAAGCUACAAAGAUAAGGCUUCAUGCCGAAAUCAA
CACCCUGUCAUUUUAUGGCAGGGUG.

In an embodiment, the RNA scaffold comprises a canonical stem loop. In an embodiment, the RNA scaffold comprises the nucleic acid sequence:

(SEQ ID NO: 324)
GUUUUAGAGCUAGAAAUAGCAAGUUAAAAUAAGGCUAGUCCGUUAUCAAC
UUGAAAAAGUGGCACCGAGUCGGUGCU*mU*mU*mU

where m=2′-O-methyl modification and *=3′ phosphorothioate internucleotide linkages (i.e., at the first 3′ terminal RNA residues as shown here).

In an embodiment, the RNA scaffold comprises the nucleic acid sequence:

(SEQ ID NO: 390)
GUUUUAGAGCUAGAAAUAGCAAGUUAAAAUAAGGCUAGUCCGUUAUCAAC
UUGAAAAAGUGGCACCGAGUCGGUGCUUUU

In an embodiment, an S. pyogenes sgRNA scaffold polynucleotide sequence is as follows:

(SEQ ID NO: 319)
GUUUUAGAGCUAGAAAUAGCAAGUUAAAAUAAGGCUAGUCCGUUAUCAAC
UUGAAAAAGUGGCACCGAGUCGGUGC

In an embodiment, an S. aureus sgRNA scaffold polynucleotide sequence is as follows:

(SEQ ID NO: 320)
GUUUUAGUACUCUGUAAUGAAAAUUACAGAAUCUACUAAAACAAGGCAAA
AUGCCGUGUUUAUCUCGUCAACUUGUUGGCGAGA

In an embodiment, the RNA scaffold comprises a non-canonical sequence. In an embodiment, the RNA scaffold comprises the nucleic acid sequence:

(SEQ ID NO: 323)
GUUUUAGAGCUAGAAAUAGCAAGUUAAAAUAAGGCUAGUCCGUUAUCAAC
UUGAAAAAGUGGGACCGAGUCGGUGCU*mU*mU*mU

where m=2′-O-methyl modification and *=3′ phosphorothioate internucleotide linkages (i.e., at the first 3′ terminal RNA residues as shown here).

In some embodiments, wild-type Cas9 corresponds to Cas9 from Streptococcus pyogenes (NCBI Reference Sequence: NC_017053.1). An exemplary Streptococcus pyogenes Cas9 (spCas9) nucleic acid sequence is provided below:

(SEQ ID NO: 198)
ATGGATAAGAAATACTCAATAGGCTTAGATATCGGCACAAATAGCGTCGGATGGGCGGTGAT
CACTGATGATTATAAGGTTCCGTCTAAAAAGTTCAAGGTTCTGGGAAATACAGACCGCCACA
GTATCAAAAAAAATCTTATAGGGGCTCTTTTATTTGGCAGTGGAGAGACAGCGGAAGCGACT
CGTCTCAAACGGACAGCTCGTAGAAGGTATACACGTCGGAAGAATCGTATTTGTTATCTACA
GGAGATTTTTTCAAATGAGATGGCGAAAGTAGATGATAGTTTCTTTCATCGACTTGAAGAGT
CTTTTTTGGTGGAAGAAGACAAGAAGCATGAACGTCATCCTATTTTTGGAAATATAGTAGAT
GAAGTTGCTTATCATGAGAAATATCCAACTATCTATCATCTGCGAAAAAAATTGGCAGATTC
TACTGATAAAGCGGATTTGCGCTTAATCTATTTGGCCTTAGCGCATATGATTAAGTTTCGTG
GTCATTTTTTGATTGAGGGAGATTTAAATCCTGATAATAGTGATGTGGACAAACTATTTATC
CAGTTGGTACAAATCTACAATCAATTATTTGAAGAAAACCCTATTAACGCAAGTAGAGTAGA
TGCTAAAGCGATTCTTTCTGCACGATTGAGTAAATCAAGACGATTAGAAAATCTCATTGCTC
AGCTCCCCGGTGAGAAGAGAAATGGCTTGTTTGGGAATCTCATTGCTTTGTCATTGGGATTG
ACCCCTAATTTTAAATCAAATTTTGATTTGGCAGAAGATGCTAAATTACAGCTTTCAAAAGA
TACTTACGATGATGATTTAGATAATTTATTGGCGCAAATTGGAGATCAATATGCTGATTTGT
TTTTGGCAGCTAAGAATTTATCAGATGCTATTTTACTTTCAGATATCCTAAGAGTAAATAGT
GAAATAACTAAGGCTCCCCTATCAGCTTCAATGATTAAGCGCTACGATGAACATCATCAAGA
CTTGACTCTTTTAAAAGCTTTAGTTCGACAACAACTTCCAGAAAAGTATAAAGAAATCTTTT
TTGATCAATCAAAAAACGGATATGCAGGTTATATTGATGGGGGAGCTAGCCAAGAAGAATTT
TATAAATTTATCAAACCAATTTTAGAAAAAATGGATGGTACTGAGGAATTATTGGTGAAACT
AAATCGTGAAGATTTGCTGCGCAAGCAACGGACCTTTGACAACGGCTCTATTCCCCATCAAA
TTCACTTGGGTGAGCTGCATGCTATTTTGAGAAGACAAGAAGACTTTTATCCATTTTTAAAA
GACAATCGTGAGAAGATTGAAAAAATCTTGACTTTTCGAATTCCTTATTATGTTGGTCCATT
GGCGCGTGGCAATAGTCGTTTTGCATGGATGACTCGGAAGTCTGAAGAAACAATTACCCCAT
GGAATTTTGAAGAAGTTGTCGATAAAGGTGCTTCAGCTCAATCATTTATTGAACGCATGACA
AACTTTGATAAAAATCTTCCAAATGAAAAAGTACTACCAAAACATAGTTTGCTTTATGAGTA
TTTTACGGTTTATAACGAATTGACAAAGGTCAAATATGTTACTGAGGGAATGCGAAAACCAG
CATTTCTTTCAGGTGAACAGAAGAAAGCCATTGTTGATTTACTCTTCAAAACAAATCGAAAA
GTAACCGTTAAGCAATTAAAAGAAGATTATTTCAAAAAAATAGAATGTTTTGATAGTGTTGA
AATTTCAGGAGTTGAAGATAGATTTAATGCTTCATTAGGCGCCTACCATGATTTGCTAAAAA
TTATTAAAGATAAAGATTTTTTGGATAATGAAGAAAATGAAGATATCTTAGAGGATATTGTT
TTAACATTGACCTTATTTGAAGATAGGGGGATGATTGAGGAAAGACTTAAAACATATGCTCA
CCTCTTTGATGATAAGGTGATGAAACAGCTTAAACGTCGCCGTTATACTGGTTGGGGACGTT
TGTCTCGAAAATTGATTAATGGTATTAGGGATAAGCAATCTGGCAAAACAATATTAGATTTT
TTGAAATCAGATGGTTTTGCCAATCGCAATTTTATGCAGCTGATCCATGATGATAGTTTGAC
ATTTAAAGAAGATATTCAAAAAGCACAGGTGTCTGGACAAGGCCATAGTTTACATGAACAGA
TTGCTAACTTAGCTGGCAGTCCTGCTATTAAAAAAGGTATTTTACAGACTGTAAAAATTGTT
GATGAACTGGTCAAAGTAATGGGGCATAAGCCAGAAAATATCGTTATTGAAATGGCACGTGA
AAATCAGACAACTCAAAAGGGCCAGAAAAATTCGCGAGAGCGTATGAAACGAATCGAAGAAG
GTATCAAAGAATTAGGAAGTCAGATTCTTAAAGAGCATCCTGTTGAAAATACTCAATTGCAA
AATGAAAAGCTCTATCTCTATTATCTACAAAATGGAAGAGACATGTATGTGGACCAAGAATT
AGATATTAATCGTTTAAGTGATTATGATGTCGATCACATTGTTCCACAAAGTTTCATTAAAG
ACGATTCAATAGACAATAAGGTACTAACGCGTTCTGATAAAAATCGTGGTAAATCGGATAAC
GTTCCAAGTGAAGAAGTAGTCAAAAAGATGAAAAACTATTGGAGACAACTTCTAAACGCCAA
GTTAATCACTCAACGTAAGTTTGATAATTTAACGAAAGCTGAACGTGGAGGTTTGAGTGAAC
TTGATAAAGCTGGTTTTATCAAACGCCAATTGGTTGAAACTCGCCAAATCACTAAGCATGTG
GCACAAATTTTGGATAGTCGCATGAATACTAAATACGATGAAAATGATAAACTTATTCGAGA
GGTTAAAGTGATTACCTTAAAATCTAAATTAGTTTCTGACTTCCGAAAAGATTTCCAATTCT
ATAAAGTACGTGAGATTAACAATTACCATCATGCCCATGATGCGTATCTAAATGCCGTCGTT
GGAACTGCTTTGATTAAGAAATATCCAAAACTTGAATCGGAGTTTGTCTATGGTGATTATAA
AGTTTATGATGTTCGTAAAATGATTGCTAAGTCTGAGCAAGAAATAGGCAAAGCAACCGCAA
AATATTTCTTTTACTCTAATATCATGAACTTCTTCAAAACAGAAATTACACTTGCAAATGGA
GAGATTCGCAAACGCCCTCTAATCGAAACTAATGGGGAAACTGGAGAAATTGTCTGGGATAA
AGGGCGAGATTTTGCCACAGTGCGCAAAGTATTGTCCATGCCCCAAGTCAATATTGTCAAGA
AAACAGAAGTACAGACAGGCGGATTCTCCAAGGAGTCAATTTTACCAAAAAGAAATTCGGAC
AAGCTTATTGCTCGTAAAAAAGACTGGGATCCAAAAAAATATGGTGGTTTTGATAGTCCAAC
GGTAGCTTATTCAGTCCTAGTGGTTGCTAAGGTGGAAAAAGGGAAATCGAAGAAGTTAAAAT
CCGTTAAAGAGTTACTAGGGATCACAATTATGGAAAGAAGTTCCTTTGAAAAAAATCCGATT
GACTTTTTAGAAGCTAAAGGATATAAGGAAGTTAAAAAAGACTTAATCATTAAACTACCTAA
ATATAGTCTTTTTGAGTTAGAAAACGGTCGTAAACGGATGCTGGCTAGTGCCGGAGAATTAC
AAAAAGGAAATGAGCTGGCTCTGCCAAGCAAATATGTGAATTTTTTATATTTAGCTAGTCAT
TATGAAAAGTTGAAGGGTAGTCCAGAAGATAACGAACAAAAACAATTGTTTGTGGAGCAGCA
TAAGCATTATTTAGATGAGATTATTGAGCAAATCAGTGAATTTTCTAAGCGTGTTATTTTAG
CAGATGCCAATTTAGATAAAGTTCTTAGTGCATATAACAAACATAGAGACAAACCAATACGT
GAACAAGCAGAAAATATTATTCATTTATTTACGTTGACGAATCTTGGAGCTCCCGCTGCTTT
TAAATATTTTGATACAACAATTGATCGTAAACGATATACGTCTACAAAAGAAGTTTTAGATG
CCACTCTTATCCATCAATCCATCACTGGTCTTTATGAAACACGCATTGATTTGAGTCAGCTA
GGAGGTGACTGA.

An exemplary Streptococcus pyogenes Cas9 (spCas9) amino acid sequence is provided below:

(SEQ ID NO: 199)
MDKKYSIGLDIGTNSVGWAVITDDYKVPSKKFKVLGNTDRHSIKKNLIGA
LLFGSGETAEATRLKRTARRRYTRRKNRICYLQEIFSNEMAKVDDSFFHR
LEESFLVEEDKKHERHPIFGNIVDEVAYHEKYPTIYHLRKKLADSTDKAD
LRLIYLALAHMIKFRGHFLIEGDLNPDNSDVDKLFIQLVQIYNQLFEENP
INASRVDAKAILSARLSKSRRLENLIAQLPGEKRNGLFGNLIALSLGLTP
NFKSNFDLAEDAKLQLSKDTYDDDLDNLLAQIGDQYADLFLAAKNLSDAI
LLSDILRVNSEITKAPLSASMIKRYDEHHQDLTLLKALVROQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGAYHDLLKI
IKDKDFLDNEENEDILEDIVLTLTLFEDRGMIEERLKTYAHLFDDKVMKQ
LKRRRYTGWGRLSRKLINGIRDKQSGKTILDFLKSDGFANRNFMQLIHDD
SLTFKEDIQKAQVSGQGHSLHEQIANLAGSPAIKKGILQTVKIVDELVKV
MGHKPENIVIEMARENQTTQKGQKNSRERMKRIEEGIKELGSQILKEHPV
ENTQLQNEKLYLYYLQNGRDMYVDQELDINRLSDYDVDHIVPQSFIKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIYEKLKGSPEDNEQKQLFVEQHK
HYLDEIIEQISEFSKRVILADANLDKVLSAYNKHRDKPIREQAENIIHLF
TLTNLGAPAAFKYFDTTIDRKRYTSTKEVLDATLIHQSITGLYETRIDLS
QLGGD

(single underline: HNH domain; double underline: RuvC domain)

In some embodiments, wild-type Cas9 corresponds to, or comprises the following nucleotide sequences:

(SEQ ID NO: 200)
ATGGATAAAAAGTATTCTATTGGTTTAGACATCGGCACTAATTCCGTTGGATGGGCTGTCAT
AACCGATGAATACAAAGTACCTTCAAAGAAATTTAAGGTGTTGGGGAACACAGACCGTCATT
CGATTAAAAAGAATCTTATCGGTGCCCTCCTATTCGATAGTGGCGAAACGGCAGAGGCGACT
CGCCTGAAACGAACCGCTCGGAGAAGGTATACACGTCGCAAGAACCGAATATGTTACTTACA
AGAAATTTTTAGCAATGAGATGGCCAAAGTTGACGATTCTTTCTTTCACCGTTTGGAAGAGT
CCTTCCTTGTCGAAGAGGACAAGAAACATGAACGGCACCCCATCTTTGGAAACATAGTAGAT
GAGGTGGCATATCATGAAAAGTACCCAACGATTTATCACCTCAGAAAAAAGCTAGTTGACTC
AACTGATAAAGCGGACCTGAGGTTAATCTACTTGGCTCTTGCCCATATGATAAAGTTCCGTG
GGCACTTTCTCATTGAGGGTGATCTAAATCCGGACAACTCGGATGTCGACAAACTGTTCATC
CAGTTAGTACAAACCTATAATCAGTTGITTGAAGAGAACCCTATAAATGCAAGTGGCGTGGA
TGCGAAGGCTATTCTTAGCGCCCGCCTCTCTAAATCCCGACGGCTAGAAAACCTGATCGCAC
AATTACCCGGAGAGAAGAAAAATGGGTTGTTCGGTAACCTTATAGCGCTCTCACTAGGCCTG
ACACCAAATTTTAAGTCGAACTTCGACTTAGCTGAAGATGCCAAATTGCAGCTTAGTAAGGA
CACGTACGATGACGATCTCGACAATCTACTGGCACAAATTGGAGATCAGTATGCGGACTTAT
TTTTGGCTGCCAAAAACCTTAGCGATGCAATCCTCCTATCTGACATACTGAGAGTTAATACT
GAGATTACCAAGGCGCCGTTATCCGCTTCAATGATCAAAAGGTACGATGAACATCACCAAGA
CTTGACACTTCTCAAGGCCCTAGTCCGTCAGCAACTGCCTGAGAAATATAAGGAAATATTCT
TTGATCAGTCGAAAAACGGGTACGCAGGTTATATTGACGGCGGAGCGAGTCAAGAGGAATTC
TACAAGTTTATCAAACCCATATTAGAGAAGATGGATGGGACGGAAGAGTTGCTTGTAAAACT
CAATCGCGAAGATCTACTGCGAAAGCAGCGGACTTTCGACAACGGTAGCATTCCACATCAAA
TCCACTTAGGCGAATTGCATGCTATACTTAGAAGGCAGGAGGATTTTTATCCGTTCCTCAAA
GACAATCGTGAAAAGATTGAGAAAATCCTAACCTTTCGCATACCTTACTATGTGGGACCCCT
GGCCCGAGGGAACTCTCGGTTCGCATGGATGACAAGAAAGTCCGAAGAAACGATTACTCCAT
GGAATTTTGAGGAAGTTGTCGATAAAGGTGCGTCAGCTCAATCGTTCATCGAGAGGATGACC
AACTTTGACAAGAATTTACCGAACGAAAAAGTATTGCCTAAGCACAGTTTACTTTACGAGTA
TTTCACAGTGTACAATGAACTCACGAAAGTTAAGTATGTCACTGAGGGCATGCGTAAACCCG
CCTTTCTAAGCGGAGAACAGAAGAAAGCAATAGTAGATCTGTTATTCAAGACCAACCGCAAA
GTGACAGTTAAGCAATTGAAAGAGGACTACTTTAAGAAAATTGAATGCTTCGATTCTGTCGA
GATCTCCGGGGTAGAAGATCGATTTAATGCGTCACTTGGTACGTATCATGACCTCCTAAAGA
TAATTAAAGATAAGGACTTCCTGGATAACGAAGAGAATGAAGATATCTTAGAAGATATAGTG
TTGACTCTTACCCTCTTTGAAGATCGGGAAATGATTGAGGAAAGACTAAAAACATACGCTCA
CCTGTTCGACGATAAGGTTATGAAACAGTTAAAGAGGCGTCGCTATACGGGCTGGGGACGAT
TGTCGCGGAAACTTATCAACGGGATAAGAGACAAGCAAAGTGGTAAAACTATTCTCGATTTT
CTAAAGAGCGACGGCTTCGCCAATAGGAACTTTATGCAGCTGATCCATGATGACTCTTTAAC
CTTCAAAGAGGATATACAAAAGGCACAGGTTTCCGGACAAGGGGACTCATTGCACGAACATA
TTGCGAATCTTGCTGGTTCGCCAGCCATCAAAAAGGGCATACTCCAGACAGTCAAAGTAGTG
GATGAGCTAGTTAAGGTCATGGGACGTCACAAACCGGAAAACATTGTAATCGAGATGGCACG
CGAAAATCAAACGACTCAGAAGGGGCAAAAAAACAGTCGAGAGCGGATGAAGAGAATAGAAG
AGGGTATTAAAGAACTGGGCAGCCAGATCTTAAAGGAGCATCCTGTGGAAAATACCCAATTG
CAGAACGAGAAACTTTACCTCTATTACCTACAAAATGGAAGGGACATGTATGTTGATCAGGA
ACTGGACATAAACCGTTTATCTGATTACGACGTCGATCACATTGTACCCCAATCCTTTTTGA
AGGACGATTCAATCGACAATAAAGTGCTTACACGCTCGGATAAGAACCGAGGGAAAAGTGAC
AATGTTCCAAGCGAGGAAGTCGTAAAGAAAATGAAGAACTATTGGCGGCAGCTCCTAAATGC
GAAACTGATAACGCAAAGAAAGTTCGATAACTTAACTAAAGCTGAGAGGGGTGGCTTGTCTG
AACTTGACAAGGCCGGATTTATTAAACGTCAGCTCGTGGAAACCCGCCAAATCACAAAGCAT
GTTGCACAGATACTAGATTCCCGAATGAATACGAAATACGACGAGAACGATAAGCTGATTCG
GGAAGTCAAAGTAATCACTTTAAAGTCAAAATTGGTGTCGGACTTCAGAAAGGATTTTCAAT
TCTATAAAGTTAGGGAGATAAATAACTACCACCATGCGCACGACGCTTATCTTAATGCCGTC
GTAGGGACCGCACTCATTAAGAAATACCCGAAGCTAGAAAGTGAGTTTGTGTATGGTGATTA
CAAAGTTTATGACGTCCGTAAGATGATCGCGAAAAGCGAACAGGAGATAGGCAAGGCTACAG
CCAAATACTTCTTTTATTCTAACATTATGAATTTCTTTAAGACGGAAATCACTCTGGCAAAC
GGAGAGATACGCAAACGACCTTTAATTGAAACCAATGGGGAGACAGGTGAAATCGTATGGGA
TAAGGGCCGGGACTTCGCGACGGTGAGAAAAGTTTTGTCCATGCCCCAAGTCAACATAGTAA
AGAAAACTGAGGTGCAGACCGGAGGGTTTTCAAAGGAATCGATTCTTCCAAAAAGGAATAGT
GATAAGCTCATCGCTCGTAAAAAGGACTGGGACCCGAAAAAGTACGGTGGCTTCGATAGCCC
TACAGTTGCCTATTCTGTCCTAGTAGTGGCAAAAGTTGAGAAGGGAAAATCCAAGAAACTGA
AGTCAGTCAAAGAATTATTGGGGATAACGATTATGGAGCGCTCGTCTTTTGAAAAGAACCCC
ATCGACTTCCTTGAGGCGAAAGGTTACAAGGAAGTAAAAAAGGATCTCATAATTAAACTACC
AAAGTATAGTCTGTTTGAGTTAGAAAATGGCCGAAAACGGATGTTGGCTAGCGCCGGAGAGC
TTCAAAAGGGGAACGAACTCGCACTACCGTCTAAATACGTGAATTTCCTGTATTTAGCGTCC
CATTACGAGAAGTTGAAAGGTTCACCTGAAGATAACGAACAGAAGCAACTTTTTGTTGAGCA
GCACAAACATTATCTCGACGAAATCATAGAGCAAATTTCGGAATTCAGTAAGAGAGTCATCC
TAGCTGATGCCAATCTGGACAAAGTATTAAGCGCATACAACAAGCACAGGGATAAACCCATA
CGTGAGCAGGCGGAAAATATTATCCATTTGTTTACTCTTACCAACCTCGGCGCTCCAGCCGC
ATTCAAGTATTTTGACACAACGATAGATCGCAAACGATACACTTCTACCAAGGAGGTGCTAG
ACGCGACACTGATTCACCAATCCATCACGGGATTATATGAAACTCGGATAGATTTGTCACAG
CTTGGGGGTGACGGATCCCCCAAGAAGAAGAGGAAAGTCTCGAGCGACTACAAAGACCATGA
CGGTGATTATAAAGATCATGACATCGATTACAAGGATGACGATGACAAGGCTGCAGGA

In some embodiments, wild-type Cas9 corresponds to, or comprises the following amino acid sequence:

(SEQ ID NO: 201)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQYVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAYVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD.

(single underline: HNH domain; double underline: RuvC domain).

In some embodiments, wild-type Cas9 corresponds to Cas9 from Streptococcus pyogenes (NCBI Reference Sequence: NC_002737.2. In some embodiments, wild-type Cas9 corresponds to Cas9 from Streptococcus pyogenes (Uniprot Reference Sequence: Q99ZW2).

The amino acid sequence of an exemplary catalytically inactive Cas9 (dCas9) is as follows:

(SEQ ID NO: 203)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVQNEKLY
LYYLQNGRDMYVDQELDINRLSDYDVDAIVPQSFLKDDSIDNKVLTRSDK
NRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKL
VSDFRKDFQFYKVREINNYHHAHDAYLNAVVGTALIKKYPKLESEFVYGD
YKVYDVRKMIAKSEQEIGKATAKYFFYSNIMNFFKTEITLANGEIRKRPL
IETNGETGEIVWDKGRDFATVRKVLSMPQVNIVKKTEVQTGGFSKESILP
KRNSDKLIARKKDWDPKKYGGFDSPTVAYSVLVVAKVEKGKSKKLKSVKE
LLGITIMERSSFEKNPIDFLEAKGYKEVKKDLIIKLPKYSLFELENGRKR
MLASAGELQKGNELALPSKYVNFLYLASHYEKLKGSPEDNEQKQLFVEQH
KHYLDEIIEQISEFSKRVILADANLDKVLSAYNKHRDKPIREQAENIIHL
FTLTNLGAPAAFKYFDTTIDRKRYTSTKEVLDATLIHQSITGLYETRIDL
SQLGGD

(see, e.g., Qi et al., “Repurposing CRISPR as an RNA-guided platform for sequence-specific control of gene expression.” Cell. 2013; 152(5):1173-83, the entire contents of which are incorporated herein by reference).

In some embodiments, a Cas9 nuclease has an inactive (e.g., an inactivated) DNA cleavage domain, that is, the Cas9 is a nickase, referred to as an “nCas9” protein (for “nickase” Cas9). A nuclease-inactivated Cas9 protein may interchangeably be referred to as a “dCas9” protein (for nuclease-“dead” Cas9) or catalytically inactive Cas9. Methods for generating a Cas9 protein (or a fragment thereof) having an inactive DNA cleavage domain are known (See, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)).

Additional suitable nuclease-inactive dCas9 domains will be apparent to those of skill in the art based on this disclosure and knowledge in the field, and are within the scope of this disclosure. Such additional exemplary suitable nuclease-inactive Cas9 domains include, but are not limited to, D10A/H840A, D10A/D839A/H840A, and D10A/D839A/H840A/N863A mutant domains (See, e.g., Prashant et al., CAS9 transcriptional activators for target specificity screening and paired nickases for cooperative genome engineering. Nature Biotechnology. 2013; 31(9): 833-838, the entire contents of which are incorporated herein by reference).

In some embodiments, a Cas9 nuclease has an inactive (e.g., an inactivated) DNA cleavage domain, that is, the Cas9 is a nickase, referred to as an “nCas9” protein (for “nickase” Cas9). The Cas9 nickase may be a Cas9 protein that is capable of cleaving only one strand of a duplexed nucleic acid molecule (e.g., a duplexed DNA molecule). In some embodiments the Cas9 nickase cleaves the target strand of a duplexed nucleic acid molecule, meaning that the Cas9 nickase cleaves the strand that is base paired to (complementary to) a gRNA (e.g., an sgRNA) that is bound to the Cas9. In some embodiments, a Cas9 nickase comprises a D10A mutation and has a histidine at position 840.

The amino acid sequence of an exemplary catalytically Cas9 nickase (nCas9) is as follows:

(SEQ ID NO: 233)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVROQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD.

In some embodiments, Cas9 is a variant Cas9 protein. A variant Cas9 polypeptide has an amino acid sequence that is different by one amino acid (e.g., has a deletion, insertion, substitution, fusion) when compared to the amino acid sequence of a wild-type Cas9 protein. In some instances, the variant Cas9 polypeptide has an amino acid change (e.g., deletion, insertion, or substitution) that reduces the nuclease activity of the Cas9 polypeptide. For example, in some instances, the variant Cas9 polypeptide has less than 50%, less than 40%, less than 30%, less than 20%, less than 10%, less than 5%, or less than 1% of the nuclease activity of the corresponding wild-type Cas9 protein. In some embodiments, the variant Cas9 protein has no substantial nuclease activity.

In some embodiments the Cas9 nickase cleaves the non-target, non-base-edited strand of a duplexed nucleic acid molecule, meaning that the Cas9 nickase cleaves the strand that is not base paired to a gRNA (e.g., an sgRNA) that is bound to the Cas9. In some embodiments, a Cas9 nickase comprises an H840A mutation and has an aspartic acid residue at position 10, or a corresponding mutation. In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field, and are within the scope of this disclosure.

In some embodiments, Cas9 is a modified Cas9. A given gRNA targeting sequence can have additional sites throughout the genome where partial homology exists. These sites are called off-targets and need to be considered when designing a gRNA. In addition to optimizing gRNA design, CRISPR specificity can also be increased through modifications to Cas9. Cas9 generates double-strand breaks (DSBs) through the combined activity of two nuclease domains, RuvC and HNH. Cas9 nickase, a D10A mutant of SpCas9, retains one nuclease domain and generates a DNA nick rather than a DSB. The nickase system can also be combined with HDR-mediated gene editing for specific gene edits.

### Catalytically Dead Nucleases

Also provided herein are base editors comprising a polynucleotide programmable nucleotide binding domain which is catalytically dead (i.e., incapable of cleaving a target polynucleotide sequence). Herein the terms “catalytically dead” and “nuclease dead” are used interchangeably to refer to a polynucleotide programmable nucleotide binding domain which has one or more mutations and/or deletions resulting in its inability to cleave a strand of a nucleic acid. In some embodiments, a catalytically dead polynucleotide programmable nucleotide binding domain base editor can lack nuclease activity as a result of specific point mutations in one or more nuclease domains. For example, in the case of a base editor comprising a Cas9 domain, the Cas9 can comprise both a D10A mutation and an H840A mutation. Such mutations inactivate both nuclease domains, thereby resulting in the loss of nuclease activity. In other embodiments, a catalytically dead polynucleotide programmable nucleotide binding domain can comprise one or more deletions of all or a portion of a catalytic domain (e.g., RuvC1 and/or HNH domains). In further embodiments, a catalytically dead polynucleotide programmable nucleotide binding domain comprises a point mutation (e.g., D10A or H840A) as well as a deletion of all or a portion of a nuclease domain. dCas9 domains are known in the art and described, for example, in Qi et al., “Repurposing CRISPR as an RNA-guided platform for sequence-specific control of gene expression.” Cell. 2013; 152(5):1173-83, the entire contents of which are incorporated herein by reference.

Additional suitable nuclease-inactive dCas9 domains will be apparent to those of skill in the art based on this disclosure and knowledge in the field, and are within the scope of this disclosure. Such additional exemplary suitable nuclease-inactive Cas9 domains include, but are not limited to, D10A/H840A, D10A/D839A/H840A, and D10A/D839A/H840A/N863A mutant domains (See, e.g., Prashant et al., CAS9 transcriptional activators for target specificity screening and paired nickases for cooperative genome engineering. Nature Biotechnology. 2013; 31(9): 833-838, the entire contents of which are incorporated herein by reference).

In some embodiments, dCas9 corresponds to, or comprises in part or in whole, a Cas9 amino acid sequence having one or more mutations that inactivate the Cas9 nuclease activity. In some embodiments, the nuclease-inactive dCas9 domain comprises a D10X mutation and a H840X mutation of the amino acid sequence set forth herein, or a corresponding mutation in any of the amino acid sequences provided herein, wherein X is any amino acid change. In some embodiments, the nuclease-inactive dCas9 domain comprises a D10A mutation and a H840A mutation of the amino acid sequence set forth herein, or a corresponding mutation in any of the amino acid sequences provided herein. In some embodiments, a nuclease-inactive Cas9 domain comprises the amino acid sequence set forth in Cloning vector pPlatTET-gRNA2 (Accession No. BAV54124).

In some embodiments, a variant Cas9 protein can cleave the complementary strand of a guide target sequence but has reduced ability to cleave the non-complementary strand of a double stranded guide target sequence. For example, the variant Cas9 protein can have a mutation (amino acid substitution) that reduces the function of the RuvC domain. As a non-limiting example, in some embodiments, a variant Cas9 protein has a D10A (aspartate to alanine at amino acid position 10) and can therefore cleave the complementary strand of a double stranded guide target sequence but has reduced ability to cleave the non-complementary strand of a double stranded guide target sequence (thus resulting in a single strand break (SSB) instead of a double strand break (DSB) when the variant Cas9 protein cleaves a double stranded target nucleic acid) (see, for example, Jinek et al., Science. 2012 Aug. 17; 337(6096):816-21).

In some embodiments, a variant Cas9 protein can cleave the non-complementary strand of a double stranded guide target sequence but has reduced ability to cleave the complementary strand of the guide target sequence. For example, the variant Cas9 protein can have a mutation (amino acid substitution) that reduces the function of the HNH domain (RuvC/HNH/RuvC domain motifs). As a non-limiting example, in some embodiments, the variant Cas9 protein has an H840A (histidine to alanine at amino acid position 840) mutation and can therefore cleave the non-complementary strand of the guide target sequence but has reduced ability to cleave the complementary strand of the guide target sequence (thus resulting in a SSB instead of a DSB when the variant Cas9 protein cleaves a double stranded guide target sequence). Such a Cas9 protein has a reduced ability to cleave a guide target sequence (e.g., a single stranded guide target sequence) but retains the ability to bind a guide target sequence (e.g., a single stranded guide target sequence).

In some embodiments, a variant Cas9 protein can cleave the non-complementary strand of a double stranded guide target sequence but has reduced ability to cleave the complementary strand of the guide target sequence. For example, the variant Cas9 protein can have a mutation (amino acid substitution) that reduces the function of the HNH domain (RuvC/HNH/RuvC domain motifs). As a non-limiting example, in some embodiments, the variant Cas9 protein has an H840A (histidine to alanine at amino acid position 840) mutation and can therefore cleave the non-complementary strand of the guide target sequence but has reduced ability to cleave the complementary strand of the guide target sequence (thus resulting in a SSB instead of a DSB when the variant Cas9 protein cleaves a double stranded guide target sequence). Such a Cas9 protein has a reduced ability to cleave a guide target sequence (e.g., a single stranded guide target sequence) but retains the ability to bind a guide target sequence (e.g., a single stranded guide target sequence).

As another non-limiting example, in some embodiments, the variant Cas9 protein harbors W476A and W1126A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA).

As another non-limiting example, in some embodiments, the variant Cas9 protein harbors P475A, W476A, N477A, D1125A, W1126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA).

As another non-limiting example, in some embodiments, the variant Cas9 protein harbors H840A, W476A, and W1126A, mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). As another non-limiting example, in some embodiments, the variant Cas9 protein harbors H840A, D10A, W476A, and W1126A, mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). In some embodiments, the variant Cas9 has restored catalytic His residue at position 840 in the Cas9 HNH domain (A840H).

As another non-limiting example, in some embodiments, the variant Cas9 protein harbors D10A, H840A, P475A, W476A, N477A, D1125A, W1126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). In some embodiments, when a variant Cas9 protein harbors W476A and W1126A mutations or when the variant Cas9 protein harbors P475A, W476A, N477A, D1125A, W1126A, and D1127A mutations, the variant Cas9 protein does not bind efficiently to a PAM sequence. Thus, in some such embodiments, when such a variant Cas9 protein is used in a method of binding, the method does not require a PAM sequence. In other words, in some embodiments, when such a variant Cas9 protein is used in a method of binding, the method can include a guide RNA, but the method can be performed in the absence of a PAM sequence (and the specificity of binding is therefore provided by the targeting segment of the guide RNA). Other residues can be mutated to achieve the above effects (i.e., inactivate one or the other nuclease portions). As non-limiting examples, residues D10, G12, G17, E762, H840, N854, N863, H982, H983, A984, D986, and/or A987 can be altered (i.e., substituted). Also, mutations other than alanine substitutions are suitable.

In some embodiments, the variant Cas protein can be spCas9, spCas9-VRQR, spCas9-VRER, xCas9 (sp), saCas9, saCas9-KKH, spCas9-MQKSER, spCas9-LRKIQK, or spCas9-LRVSQL.

In some embodiments, a modified SpCas9 including amino acid substitutions D1135M, S1136Q, G1218K, E1219F, A1322R, D1332A, R1335E, and T1337R (SpCas9-MQKFRAER) and having specificity for the altered PAM 5′-NGC-3′ was used.

In some embodiments, the Cas9 is a Cas9 variant having specificity for an altered PAM sequence. In some embodiments, the Additional Cas9 variants and PAM sequences are described in Miller, S. M., et al. Continuous evolution of SpCas9 variants compatible with non-G PAMs, Nat. Biotechnol. (2020), the entirety of which is incorporated herein by reference. in some embodiments, a Cas9 variate have no specific PAM requirements. In some embodiments, a Cas9 variant, e.g. a SpCas9 variant has specificity for a NRNH PAM, wherein R is A or G and H is A, C, or T. In some embodiments, the SpCas9 variant has specificity for a PAM sequence AAA, TAA, CAA, GAA, TAT, GAT, or CAC. In some embodiments, the SpCas9 variant comprises an amino acid substitution at position 1114, 1134, 1135, 1137, 1139, 1151, 1180, 1188, 1211, 1218, 1219, 1221, 1249, 1256, 1264, 1290, 1318, 1317, 1320, 1321, 1323, 1332, 1333, 1335, 1337, or 1339 or a corresponding position thereof. In some embodiments, the SpCas9 variant comprises an amino acid substitution at position 1114, 1135, 1218, 1219, 1221, 1249, 1320, 1321, 1323, 1332, 1333, 1335, or 1337 or a corresponding position thereof. In some embodiments, the SpCas9 variant comprises an amino acid substitution at position 1114, 1134, 1135, 1137, 1139, 1151, 1180, 1188, 1211, 1219, 1221, 1256, 1264, 1290, 1318, 1317, 1320, 1323, 1333 or a corresponding position thereof. In some embodiments, the SpCas9 variant comprises an amino acid substitution at position 1114, 1131, 1135, 1150, 1156, 1180, 1191, 1218, 1219, 1221, 1227, 1249, 1253, 1286, 1293, 1320, 1321, 1332, 1335, 1339 or a corresponding position thereof. In some embodiments, the SpCas9 variant comprises an amino acid substitution at position 1114, 1127, 1135, 1180, 1207, 1219, 1234, 1286, 1301, 1332, 1335, 1337, 1338, 1349 or a corresponding position thereof. Exemplary amino acid substitutions and PAM specificity of SpCas9 variants are shown in Tables 1A-1D.

TABLE 1A

| | SpCas9 amino acid position | | | | | | | | | | | | |
|---|---|---|---|---|---|---|---|---|---|---|---|---|---|
| | 1114 | 1135 | 1218 | 1219 | 1221 | 1249 | 1320 | 1321 | 1323 | 1332 | 1333 | 1335 | 1337 |
| SpCas9 | R | D | G | E | Q | P | A | P | A | D | R | R | T |
| AAA | | N | | V | H | | | | | | G | | |
| AAA | | N | | V | H | | | | | | G | | |
| AAA | | | | V | | | | | | | G | | |
| TAA | G | N | | V | | | | | | | I | | |
| TAA | | N | | V | | | | | | | I | | A |
| TAA | G | N | | V | | | | | | | I | | A |
| CAA | | | | V | | | | | | | K | | |
| CAA | | N | | V | | | | | | | K | | |
| CAA | | N | | V | | | | | | | K | | |
| GAA | | | | V | H | | V | | | | K | | |
| GAA | | N | | V | | | V | | | | K | | |
| GAA | | | | V | H | | V | | | | K | | |
| TAT | | | S | V | H | S | | S | | | | L | |
| TAT | | | S | V | H | S | | S | | | | L | |
| TAT | | | S | V | H | S | | S | | | | L | |
| GAT | | | | V | | | | | | | I | | |
| GAT | | | | V | | | | | D | | | Q | |
| GAT | | | | V | | | | | D | | | Q | |
| CAC | | | | V | | | | | | N | | Q | N |
| CAC | | N | | V | | | | | | | | Q | N |
| CAC | | | | V | | | | | | N | | Q | N |

TABLE 1B

| | SpCas9 amino acid position | | | | | | | | | | | | | | | | | | |
|---|---|---|---|---|---|---|---|---|---|---|---|---|---|---|---|---|---|---|---|
| | 11 | 11 | 11 | 11 | 11 | 11 | 11 | 11 | 12 | 12 | 12 | 12 | 12 | 12 | 13 | 13 | 13 | 13 | 13 |
| | 14 | 34 | 35 | 37 | 39 | 51 | 80 | 88 | 11 | 19 | 21 | 56 | 64 | 90 | 18 | 17 | 20 | 23 | 33 |
| SpCas9 | R | F | D | P | V | K | D | K | K | E | Q | Q | H | V | L | N | A | A | R |
| GAA | | | | | | | | | | V | H | | | | | | V | | K |
| GAA | | | N | S | | | | | | V | | | | | | | V | D | K |
| GAA | | | N | | | | | | | V | H | | Y | | | | V | | K |
| CAA | | | N | | | | | | | V | H | | Y | | | | V | | K |
| CAA | G | | N | S | | | | | | V | H | | Y | | | | V | | K |
| CAA | | | N | | | | | R | | V | H | | | | | | V | | K |
| CAA | | | N | | | | G | | R | V | H | | Y | | | | V | | K |
| CAA | | | N | | | | | | | V | H | | Y | | | | V | | K |
| AAA | | | N | | | | G | | | V | H | R | Y | | | | V | D | K |
| CAA | G | | N | | | | G | | | V | H | | Y | | | | V | D | K |
| CAA | | L | N | | | | G | | | V | H | | Y | | | T | V | D | K |
| TAA | G | | N | | | | G | | | V | H | | Y | G | S | | V | D | K |
| TAA | G | | N | | | E | G | | | V | H | | Y | | S | | V | | K |
| TAA | G | | N | | | | G | | | V | H | | Y | | S | | V | D | K |
| TAA | G | | N | | | | G | | R | V | H | | | | | | V | | K |
| TAA | | | N | | | | G | | R | V | H | | Y | | | | V | | K |
| TAA | G | | N | | A | | G | | | V | H | | | | | | V | | K |
| TAA | G | | N | | | | | | | V | H | | | | | | V | | K |

TABLE 1C

| | SpCas9 amino acid position | | | | | | | | | | | | | | | | | | | |
|---|---|---|---|---|---|---|---|---|---|---|---|---|---|---|---|---|---|---|---|---|
| | 11 | 11 | 11 | 11 | 11 | 11 | 11 | 12 | 12 | 12 | 12 | 12 | 12 | 12 | 12 | 13 | 13 | 13 | 13 | 13 |
| | 14 | 31 | 35 | 50 | 56 | 80 | 91 | 18 | 19 | 21 | 27 | 49 | 53 | 86 | 93 | 20 | 21 | 32 | 35 | 39 |
| SpCas9 | R | Y | D | E | K | D | K | G | E | Q | A | P | E | N | A | A | P | D | R | T |
| SacB. | | | N | | | | N | | V | H | | | | | | V | S | | L | |
| TAT | | | | | | | | | | | | | | | | | | | | |
| SacB. | | | N | | | | | S | V | H | | S | | | | | S | G | L | |
| TAT | | | | | | | | | | | | | | | | | | | | |
| AAT | | | N | | | | | S | V | H | V | S | | K | T | | S | G | L | I |
| TAT | G | | N | | | G | | S | V | H | | S | K | | | | S | G | L | |
| TAT | G | | N | | | G | | S | V | H | | S | | | | | S | G | L | |
| TAT | G | C | N | | | G | | S | V | H | | S | | | | | S | G | L | |
| TAT | G | C | N | | | G | | S | V | H | | S | | | | | S | G | L | |
| TAT | G | C | N | | | G | | S | V | H | | S | | | | | S | G | L | |
| TAT | G | C | N | | E | G | | S | V | H | | S | | | | | S | G | L | |
| TAT | G | C | N | V | | G | | S | V | H | | S | | | | | S | G | L | |
| TAT | | C | N | | | G | | S | V | H | | S | | | | | S | G | L | |
| TAT | G | C | N | | | G | | S | V | H | | S | | | | | S | G | L | |

TABLE 1D

| | SpCas9 amino acid position | | | | | | | | | | | | | |
|---|---|---|---|---|---|---|---|---|---|---|---|---|---|---|
| | 111 | 112 | 113 | 118 | 120 | 121 | 123 | 128 | 130 | 133 | 133 | 133 | 133 | 134 |
| | 4 | 7 | 5 | 0 | 7 | 9 | 4 | 6 | 1 | 2 | 5 | 7 | 8 | 9 |
| SpCas9 | R | D | D | D | E | E | N | N | P | D | R | T | S | H |
| SacB. | | | N | | | V | | | | N | Q | N | | |
| CAC | | | | | | | | | | | | | | |
| AAC | G | | N | | | V | | | | N | Q | N | | |
| AAC | G | | N | | | V | | | | N | Q | N | | |
| TAC | G | | N | | | V | | | | N | Q | N | | |
| TAC | G | | N | | | V | | H | | N | Q | N | | |
| TAC | G | | N | | G | V | D | H | | N | Q | N | | |
| TAC | G | | N | | | V | | | | N | Q | N | | |
| TAC | G | G | N | E | | V | | H | | N | Q | N | | |
| TAC | G | | N | | | V | | H | | N | Q | N | | |
| TAC | G | | N | | | | | | | N | Q | N | T | R |

### Nucleic Acid Programmable DNA Binding Proteins

Some aspects of the disclosure provide fusion proteins comprising domains that act as nucleic acid programmable DNA binding proteins, which may be used to guide a protein, such as a base editor, to a specific nucleic acid (e.g., DNA or RNA) sequence. In particular embodiments, a fusion protein comprises a nucleic acid programmable DNA binding protein domain and a deaminase domain. Non-limiting examples of nucleic acid programmable DNA binding proteins include, Cas9 (e.g., dCas9 and nCas9),

In some embodiments, one of the Cas9 domains present in the fusion protein may be replaced with a guide nucleotide sequence-programmable DNA-binding protein domain that has no requirements for a PAM sequence.

In some embodiments, the Cas9 domain is a Cas9 domain from Staphylococcus aureus (SaCas9). In some embodiments, the SaCas9 domain is a nuclease active SaCas9, a nuclease inactive SaCas9 (SaCas9d), or a SaCas9 nickase (SaCas9n). In some embodiments, the SaCas9 comprises a N579A mutation, or a corresponding mutation in any of the amino acid sequences provided herein.

In some embodiments, the SaCas9 domain, the SaCas9d domain, or the SaCas9n domain can bind to a nucleic acid sequence having a non-canonical PAM. In some embodiments, the SaCas9 domain, the SaCas9d domain, or the SaCas9n domain can bind to a nucleic acid sequence having a NNGRRT or a NNGRRT PAM sequence. The PAM sequence can be any PAM sequence known in the art. Suitable PAM sequences include, but are not limited to, NGG, NGA, NGC, NGN, NGT, NGCG, NGAG, NGAN, NGNG, NGCN, NGCG, NGTN, NNGRRT, NNNRRT, NNGRR(N), TTTV, TYCV, TYCV, TATV, NNNNGATT, NNAGAAW, or NAAAAC. Y is a pyrimidine; N is any nucleotide base; W is A or T.

In some embodiments, the SaCas9 domain comprises one or more of a E781X, a N967X, and a R1014X mutation, or a corresponding mutation in any of the amino acid sequences provided herein, wherein X is any amino acid. In some embodiments, the SaCas9 domain comprises one or more of a E781K, a N967K, and a R1014H mutation, or one or more corresponding mutation in any of the amino acid sequences provided herein. In some embodiments, the SaCas9 domain comprises a E781K, a N967K, or a R1014H mutation, or corresponding mutations in any of the amino acid sequences provided herein.

### Exemplary SaCas9 Sequence

(SEQ ID NO: 218)
KRNYILGLDIGITSVGYGIIDYETRDVIDAGVRLFKEANVENNEGRRSKR
GARRLKRRRRHRIQRVKKLLFDYNLLTDHSELSGINPYEARVKGLSQKLS
EEEFSAALLHLAKRRGVHNVNEVEEDTGNELSTKEQISRNSKALEEKYVA
ELQLERLKKDGEVRGSINRFKTSDYVKEAKQLLKVQKAYHOLDQSFIDTY
IDLLETRRTYYEGPGEGSPFGWKDIKEWYEMLMGHCTYFPEELRSVKYAY
NADLYNALNDLNNLVITRDENEKLEYYEKFQIIENVFKQKKKPTLKQIAK
EILVNEEDIKGYRVTSTGKPEFTNLKVYHDIKDITARKEITENAELLDQI
AKILTIYQSSEDIQEELTNLNSELTQEEIEQISNLKGYTGTHNLSLKAIN
LILDELWHTNDNQIAIFNRLKLVPKKVDLSQQKEIPTTLVDDFILSPVVK
RSFIQSIKVINAIIKKYGLPNDIIIELAREKNSKDAQKMINEMQKRNRQT
NERIEEIIRTTGKENAKYLIEKIKLHDMQEGKCLYSLEAIPLEDLLNNPF
NYEVDHIIPRSVSFDNSFNNKVLVKQEENSKKGNRTPFQYLSSSDSKISY
ETFKKHILNLAKGKGRISKTKKEYLLEERDINRFSVQKDFINRNLVDTRY
ATRGLMNLLRSYFRVNNLDVKVKSINGGFTSFLRRKWKFKKERNKGYKHH
AEDALIIANADFIFKEWKKLDKAKKVMENQMFEEKQAESMPEIETEQEYK
EIFITPHQIKHIKDFKDYKYSHRVDKKPNRELINDTLYSTRKDDKGNTLI
VNNLNGLYDKDNDKLKKLINKSPEKLLMYHHDPQTYQKLKLIMEQYGDEK
NPLYKYYEETGNYLTKYSKKDNGPVIKKIKYYGNKLNAHLDITDDYPNSR
NKVVKLSLKPYRFDVYLDNGVYKFVTVKNLDVIKKENYYEVNSKCYEEAK
KLKKISNQAEFIASFYNNDLIKINGELYRVIGVNNDLLNRIEVNMIDITY
REYLENMNDKRPPRIIKTIASKTQSIKKYSTDILGNLYEVKSKKHPQIIK
KG

Residue N579 above, which is underlined and in bold, may be mutated (e.g., to a A579) to yield a SaCas9 nickase.

### Exemplary SaCas9n Sequence

(SEQ ID NO: 219)
KRNYILGLDIGITSVGYGIIDYETRDVIDAGVRLFKEANVENNEGRRSKR
GARRLKRRRRHRIQRVKKLLFDYNLLTDHSELSGINPYEARVKGLSQKLS
EEEFSAALLHLAKRRGVHNVNEVEEDTGNELSTKEQISRNSKALEEKYVA
ELQLERLKKDGEVRGSINRFKTSDYVKEAKQLLKVQKAYHQLDQSFIDTY
IDLLETRRTYYEGPGEGSPFGWKDIKEWYEMLMGHCTYFPEELRSVKYAY
NADLYNALNDLNNLVITRDENEKLEYYEKFQIIENVFKQKKKPTLKQIAK
EILVNEEDIKGYRVTSTGKPEFTNLKVYHDIKDITARKEITENAELLDQI
AKILTIYQSSEDIQEELTNLNSELTQEEIEQISNLKGYTGTHNLSLKAIN
LILDELWHTNDNQIAIFNRLKLVPKKVDLSQQKEIPTTLVDDFILSPVVK
RSFIQSIKVINAIIKKYGLPNDIIIELAREKNSKDAQKMINEMQKRNRQT
NERIEEIIRTTGKENAKYLIEKIKLHDMQEGKCLYSLEAIPLEDLLNNPF
NYEVDHIIPRSVSFDNSFNNKVLVKQEEASKKGNRTPFQYLSSSDSKISY
ETFKKHILNLAKGKGRISKTKKEYLLEERDINRFSVQKDFINRNLVDTRY
ATRGLMNLLRSYFRVNNLDVKVKSINGGFTSFLRRKWKFKKERNKGYKHH
AEDALIIANADFIFKEWKKLDKAKKVMENQMFEEKQAESMPEIETEQEYK
EIFITPHQIKHIKDFKDYKYSHRVDKKPNRELINDTLYSTRKDDKGNTLI
VNNLNGLYDKDNDKLKKLINKSPEKLLMYHHDPQTYQKLKLIMEQYGDEK
NPLYKYYEETGNYLTKYSKKDNGPVIKKIKYYGNKLNAHLDITDDYPNSR
NKVVKLSLKPYRFDVYLDNGVYKFVTVKNLDVIKKENYYEVNSKCYEEAK
KLKKISNQAEFIASFYNNDLIKINGELYRVIGVNNDLLNRIEVNMIDITY
REYLENMNDKRPPRIIKTIASKTQSIKKYSTDILGNLYEVKSKKHPQIIK
KG.

Residue A579 above, which can be mutated from N579 to yield a SaCas9 nickase, is underlined and in bold.

In some embodiments, the napDNAbp is a circular permutant (e.g., SEQ ID NO: 238). In the following sequence, the plain text denotes an adenosine deaminase sequence, bold sequence indicates sequence derived from Cas9, the italicized sequence denotes a linker sequence, and the underlined sequence denotes a bipartite nuclear localization sequence, and double underlined sequence indicates mutations. The asterisk (*) denotes a STOP codon. CP5 (with MSP “NGC=Pam Variant with mutations Regular Cas9 likes NGG” PID=Protein Interacting Domain and “D10A” nickase):

(SEQ ID NO: 238)
EIGKATAKYFFYSNIMNFFKTEITLANGEIRKRPLIETNGETGEIVWDKG
RDFATVRKVLSMPQVNIVKKTEVQTGGFSKESILPKRNSDKLIARKKDWD
PKKYGGFMQPTVAYSVLVVAKVEKGKSKKLKSVKELLGITIMERSSFEKN
PIDFLEAKGYKEVKKDLIIKLPKYSLFELENGRKRMLASAKFLQKGNELA
LPSKYVNFLYLASHYEKLKGSPEDNEQKQLFVEQHKHYLDEIIEQISEFS
KRVILADANLDKVLSAYNKHRDKPIREQAENIIHLFTLTNLGAPRAFKYF
DTTIARKEYRSTKEVLDATLIHQSITGLYETRIDLSQLGGDGGSGGSGGS
GGSGGSGGSGGMDKKYSIGLAIGTNSVGWAVITDEYKVPSKKFKVLGNTD
RHSIKKNLIGALLFDSGETAEATRLKRTARRRYTRRKNRICYLQEIFSNE
MAKVDDSFFHRLEESFLVEEDKKHERHPIFGNIVDEVAYHEKYPTIYHLR
KKLVDSTDKADLRLIYLALAHMIKFRGHFLIEGDLNPDNSDVDKLFIQLV
QTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADL
FLAAKNLSDAILLSDILRVNTEITKAPLSASMIKRYDEHHQDLTLLKALV
RQQLPEKYKEIFFDQSKNGYAGYIDGGASQEEFYKFIKPILEKMDGTEEL
LVKLNREDLLRKQRTFDNGSIPHQIHLGELHAILRRQEDFYPFLKDNREK
IEKILTFRIPYYVGPLARGNSRFAWMTRKSEETITPWNFEEVVDKGASAQ
SFIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFNA
SLGTYHDLLKIIKDKDFLDNEENEDILEDIVLTLTLFEDREMIEERLKTY
AHLFDDKVMKQLKRRRYTGWGRLSRKLINGIRDKQSGKTILDFLKSDGFA
NRNFMQLIHDDSLTFKEDIQKAQVSGQGDSLHEHIANLAGSPAIKKGILQ
TVKVVDELVKVMGRHKPENIVIEMARENQTTQKGQKNSRERMKRIEEGIK
ELGSQILKEHPVENTQLQNEKLYLYYLQNGRDMYVDQELDINRLSDYDVD
HIVPQSFLKDDSIDNKVLTRSDKNRGKSDNVPSEEVVKKMKNYWRQLLNA
KLITQRKFDNLTKAERGGLSELDKAGFIKRQLVETRQITKHVAQILDSRM
NTKYDENDKLIREVKVITLKSKLVSDFRKDFQFYKVREINNYHHAHDAYL
NAVVGTALIKKYPKLESEFVYGDYKVYDVRKMIAKSEQEGADKRTADGSE
FESPKKKRKV*.

Single effectors of microbial CRISPR-Cas systems include, without limitation, Cas9, Cpf1, Cas12b/C2c1, and Cas12c/C2c3. Typically, microbial CRISPR-Cas systems are divided into Class 1 and Class 2 systems. Class 1 systems have multisubunit effector complexes, while Class 2 systems have a single protein effector. For example, Cas9 and Cpf1 are Class 2 effectors. In addition to Cas9 and Cpf1, three distinct Class 2 CRISPR-Cas systems (Cas12b/C2c1, and Cas12c/C2c3) have been described by Shmakov et al., “Discovery and Functional Characterization of Diverse Class 2 CRISPR Cas Systems”, Mol. Cell, 2015 Nov. 5; 60(3): 385-397, the entire contents of which is hereby incorporated by reference. Effectors of two of the systems, Cas12b/C2c1, and Cas12c/C2c3, contain RuvC-like endonuclease domains related to Cpf1. A third system contains an effector with two predicated HEPN RNase domains. Production of mature CRISPR RNA is tracrRNA-independent, unlike production of CRISPR RNA by Cas12b/C2c1. Cas12b/C2c1 depends on both CRISPR RNA and tracrRNA for DNA cleavage.

In some embodiments, the napDNAbp is a circular permutant (e.g., SEQ ID NO: 238).

The crystal structure of Alicyclobaccillus acidoterrastris Cas12b/C2c1 (AacC2c1) has been reported in complex with a chimeric single-molecule guide RNA (sgRNA). See e.g., Liu et al., “C2c1-sgRNA Complex Structure Reveals RNA-Guided DNA Cleavage Mechanism”, Mol. Cell, 2017 Jan. 19; 65(2):310-322, the entire contents of which are hereby incorporated by reference. The crystal structure has also been reported in Alicyclobacillus acidoterrestris C2c1 bound to target DNAs as ternary complexes. See e.g., Yang et al., “PAM-dependent Target DNA Recognition and Cleavage by C2C1 CRISPR-Cas endonuclease”, Cell, 2016 Dec. 15; 167(7):1814-1828, the entire contents of which are hereby incorporated by reference. Catalytically competent conformations of AacC2c1, both with target and non-target DNA strands, have been captured independently positioned within a single RuvC catalytic pocket, with Cas12b/C2c1-mediated cleavage resulting in a staggered seven-nucleotide break of target DNA. Structural comparisons between Cas12b/C2c1 ternary complexes and previously identified Cas9 and Cpf1 counterparts demonstrate the diversity of mechanisms used by CRISPR-Cas9 systems.

In some embodiments, the nucleic acid programmable DNA binding protein (napDNAbp) of any of the fusion proteins provided herein may be a Cas12b/C2c1, or a Cas12c/C2c3 protein. In some embodiments, the napDNAbp is a Cas12b/C2c1 protein. In some embodiments, the napDNAbp is a Cas12c/C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to a naturally-occurring Cas12b/C2c1 or Cas12c/C2c3 protein. In some embodiments, the napDNAbp is a naturally-occurring Cas12b/C2c1 or Cas12c/C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any one of the napDNAbp sequences provided herein. It should be appreciated that Cas12b/C2c1 or Cas12c/C2c3 from other bacterial species may also be used in accordance with the present disclosure.

In some embodiments, a napDNAbp refers to Cas12c. In some embodiments, the Cas12c protein is a Cas12c1 (SEQ ID NO: 239) or a variant of Cas12c1. In some embodiments, the Cas12 protein is a Cas12c2 (SEQ ID NO: 240) or a variant of Cas12c2. In some embodiments, the Cas12 protein is a Cas12c protein from Oleiphilus sp. HI0009 (i.e., OspCas12c; SEQ ID NO: 241) or a variant of OspCas12c. These Cas12c molecules have been described in Yan et al., “Functionally Diverse Type V CRISPR-Cas Systems,” Science, 2019 Jan. 4; 363: 88-91; the entire contents of which is hereby incorporated by reference. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring Cas12c1, Cas12c2, or OspCas12c protein. In some embodiments, the napDNAbp is a naturally-occurring Cas12c1, Cas12c2, or OspCas12c protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any Cas12c1, Cas12c2, or OspCas12c protein described herein. It should be appreciated that Cas12c1, Cas12c2, or OspCas12c from other bacterial species may also be used in accordance with the present disclosure.

In some embodiments, a napDNAbp refers to Cas12g, Cas12h, or Cas12i, which have been described in, for example, Yan et al., “Functionally Diverse Type V CRISPR-Cas Systems,” Science, 2019 Jan. 4; 363: 88-91; the entire contents of each is hereby incorporated by reference. Exemplary Cas12g, Cas12h, and Cas12i polypeptide sequences are provided in the Sequence Listing as SEQ ID NOs: 242-245. By aggregating more than 10 terabytes of sequence data, new classifications of Type V Cas proteins were identified that showed weak similarity to previously characterized Class V protein, including Cas12g, Cas12h, and Cas12i. In some embodiments, the Cas12 protein is a Cas12g or a variant of Cas12g. In some embodiments, the Cas12 protein is a Cas12h or a variant of Cas12h. In some embodiments, the Cas12 protein is a Cas12i or a variant of Cas12i. It should be appreciated that other RNA-guided DNA binding proteins may be used as a napDNAbp, and are within the scope of this disclosure. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring Cas12g, Cas12h, or Cas12i protein. In some embodiments, the napDNAbp is a naturally-occurring Cas12g, Cas12h, or Cas12i protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any Cas12g, Cas12h, or Cas12i protein described herein. It should be appreciated that Cas12g, Cas12h, or Cas12i from other bacterial species may also be used in accordance with the present disclosure. In some embodiments, the Cas12i is a Cas12i1 or a Cas12i2.

In some embodiments, the nucleic acid programmable DNA binding protein (napDNAbp) of any of the fusion proteins provided herein may be a Cas12j/CasΦ protein. Cas12j/CasΦ is described in Pausch et al., “CRISPR-CasΦ from huge phages is a hypercompact genome editor,” Science, 17 Jul. 2020, Vol. 369, Issue 6501, pp. 333-337, which is incorporated herein by reference in its entirety. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to a naturally-occurring Cas12j/CasΦ protein. In some embodiments, the napDNAbp is a naturally-occurring Cas12j/CasΦ protein. In some embodiments, the napDNAbp is a nuclease inactive (“dead”) Cas12j/CasΦ protein. It should be appreciated that Cas12j/CasΦ from other species may also be used in accordance with the present disclosure.

In some embodiments, the nucleic acid programmable DNA binding protein (napDNAbp) of any of the fusion proteins provided herein may be a Cas12j/CasΦ protein. Cas12j/CasΦ is described in Pausch et al., “CRISPR-CasΦ from huge phages is a hypercompact genome editor,” Science, 17 Jul. 2020, Vol. 369, Issue 6501, pp. 333-337, which is incorporated herein by reference in its entirety. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to a naturally-occurring Cas12j/CasΦ protein. In some embodiments, the napDNAbp is a naturally-occurring Cas12j/CasΦ protein. In some embodiments, the napDNAbp is a nuclease inactive (“dead”) Cas12j/CasΦ protein. It should be appreciated that Cas12j/CasΦ from other species may also be used in accordance with the present disclosure.

### Guide Polynucleotides

A polynucleotide programmable nucleotide binding domain, when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid.

In an embodiment, the guide polynucleotide is a guide RNA. Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA,” or simply “gRNA”) can be engineered so as to incorporate aspects of both the crRNA and tracrRNA into a single RNA species. See, e.g., Jinek M. et al., Science 337:816-821(2012), the entire contents of which is hereby incorporated by reference.

In some embodiments, the guide polynucleotide is at least one single guide RNA (“sgRNA” or “gRNA”). In some embodiments, the guide polynucleotide is at least one tracrRNA. In some embodiments, the guide polynucleotide does not require PAM sequence to guide the polynucleotide-programmable DNA-binding domain (e.g., Cas9) to the target nucleotide sequence.

A guide polynucleotide can be DNA. A guide polynucleotide can be RNA. As will be appreciated by one having skill in the art, in a guide polynucleotide sequence uracil (U) replaces thymine (T) in the sequence. In some embodiments, the guide polynucleotide comprises natural nucleotides (e.g., adenosine). In some embodiments, the guide polynucleotide comprises non-natural (or unnatural) nucleotides (e.g., peptide nucleic acid or nucleotide analogs). In some embodiments, the targeting region of a guide nucleic acid sequence can be at least 15, 16, 17, 18, 19, 20, 21, 22, 23, 24, 25, 26, 27, 28, 29, or 30 nucleotides in length. A targeting region of a guide nucleic acid can be between 10-30 nucleotides in length, or between 15-25 nucleotides in length, or between 15-20 nucleotides in length. In some embodiments, a guide polynucleotide may be truncated by 1, 2, 3, 4, etc. nucleotides, particularly at the 5′ end. By way of nonlimiting example, a guide polynucleotide of 20 nucleotides in length may be truncated by 1, 2, 3, 4, etc. nucleotides, particularly at the 5′ end.

In some embodiments, a guide polynucleotide comprises two or more individual polynucleotides, which can interact with one another via for example complementary base pairing (e.g., a dual guide polynucleotide). For example, a guide polynucleotide can comprise a CRISPR RNA (crRNA) and a trans-activating CRISPR RNA (tracrRNA). For example, a guide polynucleotide can comprise one or more trans-activating CRISPR RNA (tracrRNA).

In type II CRISPR systems, targeting of a nucleic acid by a CRISPR protein (e.g., Cas9) typically requires complementary base pairing between a first RNA molecule (crRNA) comprising a sequence that recognizes the target sequence and a second RNA molecule (trRNA) comprising repeat sequences which forms a scaffold region that stabilizes the guide RNA-CRISPR protein complex. Such dual guide RNA systems can be employed as a guide polynucleotide to direct the base editors disclosed herein to a target polynucleotide sequence.

In some embodiments, the base editor provided herein utilizes a single guide polynucleotide (e.g., sgRNA). In some embodiments, the base editor provided herein utilizes a dual guide polynucleotide (e.g., dual gRNAs). In some embodiments, the base editor provided herein utilizes one or more guide polynucleotide (e.g., multiple gRNA). In some embodiments, a single guide polynucleotide is utilized for different base editors described herein. For example, a single guide polynucleotide can be utilized for an adenosine base editor.

In some embodiments, the methods described herein can utilize an engineered Cas protein. A guide RNA (gRNA) is a short synthetic RNA composed of a scaffold sequence necessary for Cas-binding and a user-defined ˜20 nucleotide spacer that defines the genomic target to be modified. Exemplary gRNA scaffold sequences are provided in the sequence listing as SEQ ID NOs: 317-327. Thus, a skilled artisan can change the genomic target of the Cas protein specificity is partially determined by how specific the gRNA targeting sequence is for the genomic target compared to the rest of the genome.

In other embodiments, a guide polynucleotide can comprise both the polynucleotide targeting portion of the nucleic acid and the scaffold portion of the nucleic acid in a single molecule (i.e., a single-molecule guide nucleic acid). For example, a single-molecule guide polynucleotide can be a single guide RNA (sgRNA or gRNA). Herein the term guide polynucleotide sequence contemplates any single, dual or multi-molecule nucleic acid capable of interacting with and directing a base editor to a target polynucleotide sequence.

Typically, a guide polynucleotide (e.g., crRNA/trRNA complex or a gRNA) comprises a “polynucleotide-targeting segment” that includes a sequence capable of recognizing and binding to a target polynucleotide sequence, and a “protein-binding segment” that stabilizes the guide polynucleotide within a polynucleotide programmable nucleotide binding domain component of a base editor. In some embodiments, the polynucleotide targeting segment of the guide polynucleotide recognizes and binds to a DNA polynucleotide, thereby facilitating the editing of a base in DNA. In other embodiments, the polynucleotide targeting segment of the guide polynucleotide recognizes and binds to an RNA polynucleotide, thereby facilitating the editing of a base in RNA. Herein a “segment” refers to a section or region of a molecule, e.g., a contiguous stretch of nucleotides in the guide polynucleotide. A segment can also refer to a region/section of a complex such that a segment can comprise regions of more than one molecule. For example, where a guide polynucleotide comprises multiple nucleic acid molecules, the protein-binding segment of can include all or a portion of multiple separate molecules that are for instance hybridized along a region of complementarity. In some embodiments, a protein-binding segment of a DNA-targeting RNA that comprises two separate molecules can comprise (i) base pairs 40-75 of a first RNA molecule that is 100 base pairs in length; and (ii) base pairs 10-25 of a second RNA molecule that is 50 base pairs in length. The definition of “segment,” unless otherwise specifically defined in a particular context, is not limited to a specific number of total base pairs, is not limited to any particular number of base pairs from a given RNA molecule, is not limited to a particular number of separate molecules within a complex, and can include regions of RNA molecules that are of any total length and can include regions with complementarity to other molecules.

A guide RNA or a guide polynucleotide can comprise two or more RNAs, e.g., CRISPR RNA (crRNA) and transactivating crRNA (tracrRNA). A guide RNA or a guide polynucleotide can sometimes comprise a single-chain RNA, or single guide RNA (sgRNA) formed by fusion of a portion (e.g., a functional portion) of crRNA and tracrRNA. A guide RNA or a guide polynucleotide can also be a dual RNA comprising a crRNA and a tracrRNA. Furthermore, a crRNA can hybridize with a target DNA.

As discussed above, a guide RNA or a guide polynucleotide can be an expression product. For example, a DNA that encodes a guide RNA can be a vector comprising a sequence coding for the guide RNA. A guide RNA or a guide polynucleotide can be transferred into a cell by transfecting the cell with an isolated guide RNA or plasmid DNA comprising a sequence coding for the guide RNA and a promoter. A guide RNA or a guide polynucleotide can also be transferred into a cell in other way, such as using virus-mediated gene delivery.

A guide RNA or a guide polynucleotide can be isolated. For example, a guide RNA can be transfected in the form of an isolated RNA into a cell or organism. A guide RNA can be prepared by in vitro transcription using any in vitro transcription system known in the art. A guide RNA can be transferred to a cell in the form of isolated RNA rather than in the form of plasmid comprising encoding sequence for a guide RNA.

A guide RNA or a guide polynucleotide can comprise three regions: a first region at the 5′ end that can be complementary to a target site in a chromosomal sequence, a second internal region that can form a stem loop structure, and a third 3′ region that can be single-stranded. A first region of each guide RNA can also be different such that each guide RNA guides a fusion protein to a specific target site. Further, second and third regions of each guide RNA can be identical in all guide RNAs.

A first region of a guide RNA or a guide polynucleotide can be complementary to sequence at a target site in a chromosomal sequence such that the first region of the guide RNA can base pair with the target site. In some embodiments, a first region of a guide RNA can comprise from or from about 10 nucleotides to 25 nucleotides (i.e., from 10 nucleotides to nucleotides; or from about 10 nucleotides to about 25 nucleotides; or from 10 nucleotides to about 25 nucleotides; or from about 10 nucleotides to 25 nucleotides) or more. For example, a region of base pairing between a first region of a guide RNA and a target site in a chromosomal sequence can be or can be about 10, 11, 12, 13, 14, 15, 16, 17, 18, 19, 20, 22, 23, 24, 25, or more nucleotides in length. In some embodiments, a first region of a guide RNA can be or can be about 19, 20, or 21 nucleotides in length.

A guide RNA or a guide polynucleotide can also comprise a second region that forms a secondary structure. For example, a secondary structure formed by a guide RNA can comprise a stem (or hairpin) and a loop. A length of a loop and a stem can vary. For example, a loop can range from or from about 3 to 10 nucleotides in length, and a stem can range from or from about 6 to 20 base pairs in length. A stem can comprise one or more bulges of 1 to 10 or about 10 nucleotides. The overall length of a second region can range from or from about 16 to 60 nucleotides in length. For example, a loop can be or can be about 4 nucleotides in length and a stem can be or can be about 12 base pairs.

A guide RNA or a guide polynucleotide can also comprise a third region at the 3′ end that can be essentially single-stranded. For example, a third region is sometimes not complementarity to any chromosomal sequence in a cell of interest and is sometimes not complementarity to the rest of a guide RNA. Further, the length of a third region can vary. A third region can be more than or more than about 4 nucleotides in length. For example, the length of a third region can range from or from about 5 to 60 nucleotides in length.

A guide RNA or a guide polynucleotide can target any exon or intron of a gene target. In some embodiments, a guide can target exon 1 or 2 of a gene; in other embodiments, a guide can target exon 3 or 4 of a gene. A composition can comprise multiple guide RNAs that all target the same exon or in some embodiments, multiple guide RNAs that can target different exons. An exon and an intron of a gene can be targeted.

A guide RNA or a guide polynucleotide can target a nucleic acid sequence of or of about 20 nucleotides. A target nucleic acid can be less than or less than about 20 nucleotides. A target nucleic acid can be at least or at least about 5, 10, 15, 16, 17, 18, 19, 20, 21, 22, 23, 24, 25, 30, or anywhere between 1-100 nucleotides in length. A target nucleic acid can be at most or at most about 5, 10, 15, 16, 17, 18, 19, 20, 21, 22, 23, 24, 25, 30, 40, 50, or anywhere between 1-100 nucleotides in length. A target nucleic acid sequence can be or can be about 20 bases immediately 5′ of the first nucleotide of the PAM. A guide RNA can target a nucleic acid sequence. A target nucleic acid can be at least or at least about 1-10, 1-20, 1-30, 1-40, 1-50, 1-60, 1-70, 1-80, 1-90, or 1-100 nucleotides.

A guide polynucleotide, for example, a guide RNA, can refer to a nucleic acid that can hybridize to another nucleic acid, for example, the target nucleic acid or protospacer in a genome of a cell. A guide polynucleotide can be RNA. A guide polynucleotide can be DNA. The guide polynucleotide can be programmed or designed to bind to a sequence of nucleic acid site-specifically. A guide polynucleotide can comprise a polynucleotide chain and can be called a single guide polynucleotide. A guide polynucleotide can comprise two polynucleotide chains and can be called a double guide polynucleotide. A guide RNA can be introduced into a cell or embryo as an RNA molecule. For example, an RNA molecule can be transcribed in vitro and/or can be chemically synthesized. An RNA can be transcribed from a synthetic DNA molecule, e.g., a gBlocks® gene fragment. A guide RNA can then be introduced into a cell or embryo as an RNA molecule. A guide RNA can also be introduced into a cell or embryo in the form of a non-RNA nucleic acid molecule, e.g., a DNA molecule. For example, a DNA encoding a guide RNA can be operably linked to promoter control sequence for expression of the guide RNA in a cell or embryo of interest. An RNA coding sequence can be operably linked to a promoter sequence that is recognized by RNA polymerase III (Pol III). Plasmid vectors that can be used to express guide RNA include, but are not limited to, px330 vectors and px333 vectors. In some embodiments, a plasmid vector (e.g., px333 vector) can comprise at least two guide RNA-encoding DNA sequences.

Methods for selecting, designing, and validating guide polynucleotides, e.g., guide RNAs and targeting sequences are described herein and known to those skilled in the art. For example, to minimize the impact of potential substrate promiscuity of a deaminase domain in the nucleobase editor system (e.g., an AID domain), the number of residues that could unintentionally be targeted for deamination (e.g., off-target C residues that could potentially reside on ssDNA within the target nucleic acid locus) may be minimized. In addition, software tools can be used to optimize the gRNAs corresponding to a target nucleic acid sequence, e.g., to minimize total off-target activity across the genome. For example, for each possible targeting domain choice using S. pyogenes Cas9, all off-target sequences (preceding selected PAMs, e.g., NAG or NGG) may be identified across the genome that contain up to certain number (e.g., 1, 2, 3, 4, 5, 6, 7, 8, 9, or 10) of mismatched base-pairs. First regions of gRNAs complementary to a target site can be identified, and all first regions (e.g., crRNAs) can be ranked according to its total predicted off-target score; the top-ranked targeting domains represent those that are likely to have the greatest on-target and the least off-target activity. Candidate targeting gRNAs can be functionally evaluated by using methods known in the art and/or as set forth herein.

As a non-limiting example, target DNA hybridizing sequences in crRNAs of a guide RNA for use with Cas9s may be identified using a DNA sequence searching algorithm. gRNA design may be carried out using custom gRNA design software based on the public tool cas-offinder as described in Bae S., Park J., & Kim J.-S. Cas-OFFinder: A fast and versatile algorithm that searches for potential off-target sites of Cas9 RNA-guided endonucleases. Bioinformatics 30, 1473-1475 (2014). This software scores guides after calculating their genome-wide off-target propensity. Typically matches ranging from perfect matches to 7 mismatches are considered for guides ranging in length from 17 to 24. Once the off-target sites are computationally-determined, an aggregate score is calculated for each guide and summarized in a tabular output using a web-interface. In addition to identifying potential target sites adjacent to PAM sequences, the software also identifies all PAM adjacent sequences that differ by 1, 2, 3 or more than 3 nucleotides from the selected target sites. Genomic DNA sequences for a target nucleic acid sequence, e.g., a target gene may be obtained and repeat elements may be screened using publicly available tools, for example, the RepeatMasker program. RepeatMasker searches input DNA sequences for repeated elements and regions of low complexity. The output is a detailed annotation of the repeats present in a given query sequence.

Following identification, first regions of guide RNAs, e.g., crRNAs, may be ranked into tiers based on their distance to the target site, their orthogonality and presence of 5′ nucleotides for close matches with relevant PAM sequences (for example, a 5′ G based on identification of close matches in the human genome containing a relevant PAM e.g., NGG PAM for S. pyogenes, NNGRRT or NNGRRV PAM for S. aureus). As used herein, orthogonality refers to the number of sequences in the human genome that contain a minimum number of mismatches to the target sequence. A “high level of orthogonality” or “good orthogonality” may, for example, refer to 20-mer targeting domains that have no identical sequences in the human genome besides the intended target, nor any sequences that contain one or two mismatches in the target sequence. Targeting domains with good orthogonality may be selected to minimize off-target DNA cleavage.

In some embodiments, a reporter system may be used for detecting base-editing activity and testing candidate guide polynucleotides. In some embodiments, a reporter system may comprise a reporter gene based assay where base editing activity leads to expression of the reporter gene. For example, a reporter system may include a reporter gene comprising a deactivated start codon, e.g., a mutation on the template strand from 3′-TAC-5′ to 3′-CAC-5′. Upon successful deamination of the target C, the corresponding mRNA will be transcribed as 5′-AUG-3′ instead of 5′-GUG-3′, enabling the translation of the reporter gene. Suitable reporter genes will be apparent to those of skill in the art. Non-limiting examples of reporter genes include gene encoding green fluorescence protein (GFP), red fluorescence protein (RFP), luciferase, secreted alkaline phosphatase (SEAP), or any other gene whose expression are detectable and apparent to those skilled in the art. The reporter system can be used to test many different gRNAs, e.g., in order to determine which nucleotide residue(s) with respect to the target DNA sequence the respective deaminase will target. sgRNAs that target non-template strand nucleotide residues can also be tested in order to assess off-target effects of a specific base editing protein, e.g., a Cas9 deaminase fusion protein. In some embodiments, such gRNAs can be designed such that the mutated start codon will not be base-paired with the gRNA. The guide polynucleotides can comprise standard nucleotides, modified nucleotides (e.g., pseudouridine), nucleotide isomers, and/or nucleotide analogs. In some embodiments, the guide polynucleotide can comprise at least one detectable label. The detectable label can be a fluorophore (e.g., FAM, TMR, Cy3, Cy5, Texas Red, Oregon Green, Alexa Fluors, Halo tags, or any other suitable fluorescent dye), a detection tag (e.g., biotin, digoxigenin, and the like), quantum dots, or gold particles.

The guide polynucleotides can be synthesized chemically and/or enzymatically. For example, the guide RNA can be synthesized using standard phosphoramidite-based solid-phase synthesis methods. Alternatively, the guide RNA can be synthesized in vitro by operably linking DNA encoding the guide RNA to a promoter control sequence that is recognized by a phage RNA polymerase. Examples of suitable phage promoter sequences include T7, T3, SP6 promoter sequences, or variations thereof. In embodiments in which the guide RNA comprises two separate molecules (e.g., crRNA and tracrRNA), the crRNA can be chemically synthesized and the tracrRNA can be enzymatically synthesized.

In some embodiments, a base editor system may comprise multiple guide polynucleotides, e.g., gRNAs. For example, the gRNAs may target the base editor to one or more target loci (e.g., at least one (1) gRNA, at least 2 gRNA, at least 5 gRNA, at least 10 gRNA, at least 20 gRNA, at least 30 g RNA, or at least 50 gRNA). In some embodiments, multiple gRNA sequences can be tandemly arranged and are preferably separated by a direct repeat.

A DNA sequence encoding a guide RNA or a guide polynucleotide can also be part of a vector. In some embodiments, a vector comprises additional expression control sequences (e.g., enhancer sequences, Kozak sequences, polyadenylation sequences, transcriptional termination sequences, etc.), selectable marker sequences (e.g., GFP or antibiotic resistance genes such as puromycin), origins of replication, and the like. A DNA molecule encoding a guide RNA or a guide polynucleotide can be circular or linear.

In some embodiments, one or more components of a base editor system may be encoded by DNA sequences. Such DNA sequences may be introduced into an expression system, e.g., a cell, together or separately. For example, DNA sequences encoding a polynucleotide programmable nucleotide binding domain and a guide RNA may be introduced into a cell, each DNA sequence can be part of a separate molecule (e.g., one vector containing the polynucleotide programmable nucleotide binding domain coding sequence and a second vector containing the guide RNA coding sequence) or both can be part of a same molecule (e.g., one vector containing coding (and regulatory) sequence for both the polynucleotide programmable nucleotide binding domain and the guide RNA).

A guide polynucleotide can comprise one or more modifications to provide a nucleic acid with a new or enhanced feature. A guide polynucleotide can comprise a nucleic acid affinity tag. A guide polynucleotide can comprise synthetic nucleotide, synthetic nucleotide analog, nucleotide derivatives, and/or modified nucleotides.

In some embodiments, a gRNA or a guide polynucleotide can comprise modifications. A modification can be made at any location of a gRNA or a guide polynucleotide. More than one modification can be made to a single gRNA or a guide polynucleotide. A gRNA or a guide polynucleotide can undergo quality control after a modification. In some embodiments, quality control can include PAGE, HPLC, MS, or any combination thereof.

A modification of a gRNA or a guide polynucleotide can be a substitution, insertion, deletion, chemical modification, physical modification, stabilization, purification, or any combination thereof.

A gRNA or a guide polynucleotide can also be modified by 5′adenylate, 5′ guanosine-triphosphate cap, 5′N7-Methylguanosine-triphosphate cap, 5′triphosphate cap, 3′phosphate, 3′thiophosphate, 5′phosphate, 5′thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencer 2, DABCYL SE, dT-DABCYL, IRDye QC-1, QSY-21, QSY-35, QSY-7, QSY-9, carboxyl linker, thiol linkers, 2′-deoxyribonucleoside analog purine, 2′-deoxyribonucleoside analog pyrimidine, ribonucleoside analog, 2′-O-methyl ribonucleoside analog, sugar modified analogs, wobble/universal bases, fluorescent dye label, 2′-fluoro RNA, 2′-O-methyl RNA, methylphosphonate, phosphodiester DNA, phosphodiester RNA, phosphothioate DNA, phosphorothioate RNA, UNA, pseudouridine-5′-triphosphate, 5′-methylcytidine-5′-triphosphate, or any combination thereof. In some embodiments, a modification is one or more of 2′-O-methyl (2′-OMe), phosphorothioate (PS), 2′-O-methyl thioPACE (MSP), 2′O-methyl-PACE (MP), 2′-fluoro RNA (2′-F-RNA), and constrained ethyl (S-cEt).

In some embodiments, a modification is permanent. In other embodiments, a modification is transient. In some embodiments, multiple modifications are made to a gRNA or a guide polynucleotide. A gRNA or a guide polynucleotide modification can alter physiochemical properties of a nucleotide, such as their conformation, polarity, hydrophobicity, chemical reactivity, base-pairing interactions, or any combination thereof.

A modification can also be a phosphorothioate substitute. In some embodiments, a natural phosphodiester bond can be susceptible to rapid degradation by cellular nucleases and; a modification of internucleotide linkage using phosphorothioate (PS) bond substitutes can be more stable towards hydrolysis by cellular degradation. A modification can increase stability in a gRNA or a guide polynucleotide. A modification can also enhance biological activity. In some embodiments, a phosphorothioate enhanced RNA gRNA can inhibit RNase A, RNase T1, calf serum nucleases, or any combinations thereof. These properties can allow the use of PS-RNA gRNAs to be used in applications where exposure to nucleases is of high probability in vivo or in vitro. For example, phosphorothioate (PS) bonds can be introduced between the last 3-5 nucleotides at the 5′- or ″-end of a gRNA which can inhibit exonuclease degradation. In some embodiments, phosphorothioate bonds can be added throughout an entire gRNA to reduce attack by endonucleases. In some embodiments, the guide RNA is designed to disrupt a splice site (i.e., a splice acceptor (SA) or a splice donor (SD). In some embodiments, the guide RNA is designed such that the base editing results in a premature STOP codon.

### Protospacer Adjacent Motif

The term “protospacer adjacent motif (PAM)” or PAM-like motif refers to a 2-6 base pair DNA sequence immediately following the DNA sequence targeted by the Cas9 nuclease in the CRISPR bacterial adaptive immune system. In some embodiments, the PAM can be a 5′ PAM (i.e., located upstream of the 5′ end of the protospacer). In other embodiments, the PAM can be a 3′ PAM (i.e., located downstream of the 5′ end of the protospacer). The PAM sequence is essential for target binding, but the exact sequence depends on a type of Cas protein. The PAM sequence can be any PAM sequence known in the art. Suitable PAM sequences include, but are not limited to, NGG, NGA, NGC, NGN, NGT, NGTT, NGCG, NGAG, NGAN, NGNG, NGCN, NGCG, NGTN, NNGRRT, NNNRRT, NNGRR(N), TTTV, TYCV, TYCV, TATV, NNNNGATT, NNAGAAW, or NAAAAC. Y is a pyrimidine; N is any nucleotide base; W is A or T.

A base editor provided herein can comprise a CRISPR protein-derived domain that is capable of binding a nucleotide sequence that contains a canonical or non-canonical protospacer adjacent motif (PAM) sequence. A PAM site is a nucleotide sequence in proximity to a target polynucleotide sequence. Some aspects of the disclosure provide for base editors comprising all or a portion of CRISPR proteins that have different PAM specificities.

For example, typically Cas9 proteins, such as Cas9 from S. pyogenes (spCas9), require a canonical NGG PAM sequence to bind a particular nucleic acid region, where the “N” in “NGG” is adenine (A), thymine (T), guanine (G), or cytosine (C), and the G is guanine. A PAM can be CRISPR protein-specific and can be different between different base editors comprising different CRISPR protein-derived domains. A PAM can be 5′ or 3′ of a target sequence. A PAM can be upstream or downstream of a target sequence. A PAM can be 1, 2, 3, 4, 5, 6, 7, 8, 9, 10 or more nucleotides in length. Often, a PAM is between 2-6 nucleotides in length.

In some embodiments, the PAM is an “NRN” PAM where the “N” in “NRN” is adenine (A), thymine (T), guanine (G), or cytosine (C), and the R is adenine (A) or guanine (G); or the PAM is an “NYN” PAM, wherein the “N” in NYN is adenine (A), thymine (T), guanine (G), or cytosine (C), and the Y is cytidine (C) or thymine (T), for example, as described in R. T. Walton et al., 2020, Science, 10.1126/science.aba8853 (2020), the entire contents of which are incorporated herein by reference.

Several PAM variants are described in Table 2 below.

TABLE 2
Cas9 proteins and corresponding PAM sequences

| Variant | PAM |
|---|---|
| spCas9 | NGG |
| spCas9-VRQR | NGA |
| spCas9-VRER | NGCG |
| xCas9 (sp) | NGN |
| saCas9 | NNGRRT |
| saCas9-KKH | NNNRRT |
| spCas9-MQKSER | NGCG |
| spCas9-MQKSER | NGCN |
| spCas9-LRKIQK | NGTN |
| spCas9-LRVSQK | NGTN |
| spCas9-LRVSQL | NGTN |
| spCas9-MQKFRAER | NGC |
| Cpf1 | 5′ (TTTV) |
| SpyMac | 5′ -NAA-3′ |

In some embodiments, the PAM is NGC. In some embodiments, the NGC PAM is recognized by a Cas9 variant. In some embodiments, the NGC PAM variant includes one or more amino acid substitutions selected from D1135M, S1136Q, G1218K, E1219F, A1322R, D1332A, R1335E, and T1337R (collectively termed “MQKFRAER”).

In some embodiments, the PAM is NGT. In some embodiments, the NGT PAM is recognized by a Cas9 variant. In some embodiments, the NGT PAM variant is generated through targeted mutations at one or more residues 1335, 1337, 1135, 1136, 1218, and/or 1219. In some embodiments, the NGT PAM variant is created through targeted mutations at one or more residues 1219, 1335, 1337, 1218. In some embodiments, the NGT PAM variant is created through targeted mutations at one or more residues 1135, 1136, 1218, 1219, and 1335. In some embodiments, the NGT PAM variant is selected from the set of targeted mutations provided in Tables 3A and 3B below.

TABLE 3A
NGT PAM Variant Mutations at residues 1219, 1335, 1337, 1218

| Variant | E1219V | R1335Q | T1337 | G1218 |
|---|---|---|---|---|
| 1 | F | V | T | |
| 2 | F | V | R | |
| 3 | F | V | Q | |
| 4 | F | V | L | |
| 5 | F | V | T | R |
| 6 | F | V | R | R |
| 7 | F | V | Q | R |
| 8 | F | V | L | R |
| 9 | L | L | T | |
| 10 | L | L | R | |
| 11 | L | L | Q | |
| 12 | L | L | L | |
| 13 | F | I | T | |
| 14 | F | I | R | |
| 15 | F | I | Q | |
| 16 | F | I | L | |
| 17 | F | G | C | |
| 18 | H | L | N | |
| 19 | F | G | C | A |
| 20 | H | L | N | V |
| 21 | L | A | W | |
| 22 | L | A | F | |
| 23 | L | A | Y | |
| 24 | I | A | W | |
| 25 | I | A | F | |
| 26 | I | A | Y | |

TABLE 3B
NGT PAM Variant Mutations at residues 1135, 1136, 1218, 1219,
and 1335

| Variant | D1135L | S1136R | G1218S | E1219V | R1335Q |
|---|---|---|---|---|---|
| 27 | G | | | | |
| 28 | V | | | | |
| 29 | I | | | | |
| 30 | | A | | | |
| 31 | | W | | | |
| 32 | | H | | | |
| 33 | | K | | | |
| 34 | | | K | | |
| 35 | | | R | | |
| 36 | | | Q | | |
| 37 | | | T | | |
| 38 | | | N | | |
| 39 | | | | I | |
| 40 | | | | A | |
| 41 | | | | N | |
| 42 | | | | Q | |
| 43 | | | | G | |
| 44 | | | | L | |
| 45 | | | | S | |
| 46 | | | | T | |
| 47 | | | | | L |
| 48 | | | | | I |
| 49 | | | | | V |
| 50 | | | | | N |
| 51 | | | | | S |
| 52 | | | | | T |
| 53 | | | | | F |
| 54 | | | | | Y |
| 55 | N1286Q | I1331F | | | |

In some embodiments, the NGT PAM variant is selected from variant 5, 7, 28, 31, or 36 in Table 3A and Table 3B. In some embodiments, the variants have improved NGT PAM recognition.

In some embodiments, the NGT PAM variants have mutations at residues 1219, 1335, 1337, and/or 1218. In some embodiments, the NGT PAM variant is selected with mutations for improved recognition from the variants provided in Table 4 below.

TABLE 4
NGT PAM Variant Mutations at residues 1219, 1335, 1337, and 1218

| Variant | E1219V | R1335Q | T1337 | G1218 |
|---|---|---|---|---|
| 1 | F | V | T | |
| 2 | F | V | R | |
| 3 | F | V | Q | |
| 4 | F | V | L | |
| 5 | F | V | T | R |
| 6 | F | V | R | R |
| 7 | F | V | Q | R |
| 8 | F | V | L | R |

In some embodiments, the NGT PAM is selected from the variants provided in Table 5 below.

TABLE 5
NGT PAM variants

| | NGTN | | | | | | | |
|---|---|---|---|---|---|---|---|---|
| | variant | D1135 | S1136 | G1218 | E1219 | A1322R | R1335 | T1337 |
| Variant 1 | LRKIQK | L | R | K | I | — | Q | K |
| Variant 2 | LRSVQK | L | R | S | V | — | Q | K |
| Variant 3 | LRSVQL | L | R | S | V | — | Q | L |
| Variant 4 | LRKIRQK | L | R | K | I | R | Q | K |
| Variant 5 | LRSVRQK | L | R | S | V | R | Q | K |
| Variant 6 | LRSVRQL | L | R | S | V | R | Q | L |

In some embodiments the NGTN variant is variant 1. In some embodiments, the NGTN variant is variant 2. In some embodiments, the NGTN variant is variant 3. In some embodiments, the NGTN variant is variant 4. In some embodiments, the NGTN variant is variant 5. In some embodiments, the NGTN variant is variant 6.

In some embodiments, the Cas9 domain is a Cas9 domain from Streptococcus pyogenes (SpCas9). In some embodiments, the SpCas9 domain is a nuclease active SpCas9, a nuclease inactive SpCas9 (SpCas9d), or a SpCas9 nickase (SpCas9n). In some embodiments, the SpCas9 comprises a D9X mutation, or a corresponding mutation in any of the amino acid sequences provided herein, wherein X is any amino acid except for D. In some embodiments, the SpCas9 comprises a D9A mutation, or a corresponding mutation in any of the amino acid sequences provided herein. In some embodiments, the SpCas9 domain, the SpCas9d domain, or the SpCas9n domain can bind to a nucleic acid sequence having a non-canonical PAM. In some embodiments, the SpCas9 domain, the SpCas9d domain, or the SpCas9n domain can bind to a nucleic acid sequence having an NGG, a NGA, or a NGCG PAM sequence.

In some embodiments, the SpCas9 domain comprises one or more of a D1135X, a R1335X, and a T1337X mutation, or a corresponding mutation in any of the amino acid sequences provided herein, wherein X is any amino acid. In some embodiments, the SpCas9 domain comprises one or more of a D1135E, R1335Q, and T1337R mutation, or a corresponding mutation in any of the amino acid sequences provided herein. In some embodiments, the SpCas9 domain comprises a D1135E, a R1335Q, and a T1337R mutation, or corresponding mutations in any of the amino acid sequences provided herein. In some embodiments, the SpCas9 domain comprises one or more of a D1135X, a R1335X, and a T1337X mutation, or a corresponding mutation in any of the amino acid sequences provided herein, wherein X is any amino acid. In some embodiments, the SpCas9 domain comprises one or more of a D1135V, a R1335Q, and a T1337R mutation, or a corresponding mutation in any of the amino acid sequences provided herein. In some embodiments, the SpCas9 domain comprises a D1135V, a R1335Q, and a T1337R mutation, or corresponding mutations in any of the amino acid sequences provided herein. In some embodiments, the SpCas9 domain comprises one or more of a D1135X, a G1218X, a R1335X, and a T1337X mutation, or a corresponding mutation in any of the amino acid sequences provided herein, wherein X is any amino acid. In some embodiments, the SpCas9 domain comprises one or more of a D1135V, a G1218R, a R1335Q, and a T1337R mutation, or a corresponding mutation in any of the amino acid sequences provided herein. In some embodiments, the SpCas9 domain comprises a D1135V, a G1218R, a R1335Q, and a T1337R mutation, or corresponding mutations in any of the amino acid sequences provided herein.

In some examples, a PAM recognized by a CRISPR protein-derived domain of a base editor disclosed herein can be provided to a cell on a separate oligonucleotide to an insert (e.g., an AAV insert) encoding the base editor. In such embodiments, providing PAM on a separate oligonucleotide can allow cleavage of a target sequence that otherwise would not be able to be cleaved, because no adjacent PAM is present on the same polynucleotide as the target sequence.

In an embodiment, S. pyogenes Cas9 (SpCas9) can be used as a CRISPR endonuclease for genome engineering. However, others can be used. In some embodiments, a different endonuclease can be used to target certain genomic targets. In some embodiments, synthetic SpCas9-derived variants with non-NGG PAM sequences can be used. Additionally, other Cas9 orthologues from various species have been identified and these “non-SpCas9s” can bind a variety of PAM sequences that can also be useful for the present disclosure. For example, the relatively large size of SpCas9 (approximately 4 kb coding sequence) can lead to plasmids carrying the SpCas9 cDNA that cannot be efficiently expressed in a cell. Conversely, the coding sequence for Staphylococcus aureus Cas9 (SaCas9) is approximately 1 kilobase shorter than SpCas9, possibly allowing it to be efficiently expressed in a cell. Similar to SpCas9, the SaCas9 endonuclease is capable of modifying target genes in mammalian cells in vitro and in mice in vivo. In some embodiments, a Cas protein can target a different PAM sequence. In some embodiments, a target gene can be adjacent to a Cas9 PAM, 5′-NGG, for example. In other embodiments, other Cas9 orthologs can have different PAM requirements. For example, other PAMs such as those of S. thermophilus (5′-NNAGAA for CRISPR1 and 5′-NGGNG for CRISPR3) and Neisseria meningitidis (5′-NNNNGATT) can also be found adjacent to a target gene.

In some embodiments, for a S. pyogenes system, a target gene sequence can precede (i.e., be 5′ to) a 5′-NGG PAM, and a 20-nt guide RNA sequence can base pair with an opposite strand to mediate a Cas9 cleavage adjacent to a PAM. In some embodiments, an adjacent cut can be or can be about 3 base pairs upstream of a PAM. In some embodiments, an adjacent cut can be or can be about 10 base pairs upstream of a PAM. In some embodiments, an adjacent cut can be or can be about 0-20 base pairs upstream of a PAM. For example, an adjacent cut can be next to, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 base pairs upstream of a PAM. An adjacent cut can also be downstream of a PAM by 1 to 30 base pairs.

In some embodiments, the Cas9 domain is a recombinant Cas9 domain. In some embodiments, the recombinant Cas9 domain is a SpyMacCas9 domain. In some embodiments, the SpyMacCas9 domain is a nuclease active SpyMacCas9, a nuclease inactive SpyMacCas9 (SpyMacCas9d), or a SpyMacCas9 nickase (SpyMacCas9n). In some embodiments, the SaCas9 domain, the SaCas9d domain, or the SaCas9n domain can bind to a nucleic acid sequence having a non-canonical PAM. In some embodiments, the SpyMacCas9 domain, the SpCas9d domain, or the SpCas9n domain can bind to a nucleic acid sequence having a NAA PAM sequence.

The sequence of an exemplary Cas9 A homolog of Spy Cas9 in Streptococcus macacae with native 5′-NAAN-3′ PAM specificity is known in the art and described, for example, by Chatterjee, et al., “A Cas9 with PAM recognition for adenine dinucleotides”, Nature Communications, vol. 11, article no. 2474 (2020), and is in the Sequence Listing as SEQ ID NO: 237.

The sequence of an exemplary Cas9 A homolog of Spy Cas9 in Streptococcus macacae with native 5′-NAAN-3′ PAM specificity is known in the art and described, for example, by Jakimo et al. (biorxiv.org/content/biorxiv/early/2018/09/27/429654.full.pdf), In some embodiments, a variant Cas9 protein harbors, H840A, P475A, W476A, N477A, D1125A, W1126A, and D1218A mutations such that the polypeptide has a reduced ability to cleave a target DNA or RNA. Such a Cas9 protein has a reduced ability to cleave a target DNA (e.g., a single stranded target DNA) but retains the ability to bind a target DNA (e.g., a single stranded target DNA). As another non-limiting example, in some embodiments, the variant Cas9 protein harbors D10A, H840A, P475A, W476A, N477A, D1125A, W1126A, and D1218A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). In some embodiments, when a variant Cas9 protein harbors W476A and W1126A mutations or when the variant Cas9 protein harbors P475A, W476A, N477A, D1125A, W1126A, and D1218A mutations, the variant Cas9 protein does not bind efficiently to a PAM sequence. Thus, in some such cases, when such a variant Cas9 protein is used in a method of binding, the method does not require a PAM sequence. In other words, in some embodiments, when such a variant Cas9 protein is used in a method of binding, the method can include a guide RNA, but the method can be performed in the absence of a PAM sequence (and the specificity of binding is therefore provided by the targeting segment of the guide RNA). Other residues can be mutated to achieve the above effects (i.e., inactivate one or the other nuclease portions). As non-limiting examples, residues D10, G12, G17, E762, H840, N854, N863, H982, H983, A984, D986, and/or A987 can be altered (i.e., substituted). Also, mutations other than alanine substitutions are suitable.

In some embodiments, a CRISPR protein-derived domain of a base editor can comprise all or a portion of a Cas9 protein with a canonical PAM sequence (NGG). In other embodiments, a Cas9-derived domain of a base editor can employ a non-canonical PAM sequence. Such sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); R. T. Walton et al. “Unconstrained genome targeting with near-PAMless engineered CRISPR-Cas9 variants” Science 10.1126/science.aba8853 (2020); Hu et al. “Evolved Cas9 variants with broad PAM compatibility and high DNA specificity,” Nature, 2018 Apr. 5, 556(7699), 57-63; Miller et al., “Continuous evolution of SpCas9 variants compatible with non-G PAMs” Nat. Biotechnol., 2020 April; 38(4):471-481; the entire contents of each are hereby incorporated by reference.

Cas9 Domains with Reduced Exclusivity

Typically, Cas9 proteins, such as Cas9 from S. pyogenes (spCas9), require a canonical NGG PAM sequence to bind a particular nucleic acid region, where the “N” in “NGG” is adenosine (A), thymidine (T), or cytosine (C), and the G is guanosine. This may limit the ability to edit desired bases within a genome. In some embodiments, the base editing fusion proteins provided herein may need to be placed at a precise location, for example a region comprising a target base that is upstream of the PAM. See e.g., Komor, A. C., et al., “Programmable editing of a target base in genomic DNA without double-stranded DNA cleavage” Nature 533, 420-424 (2016), the entire contents of which are hereby incorporated by reference. Exemplary polypeptide sequences for spCas9 proteins capable of binding a PAM sequence are provided in the Sequence Listing as SEQ ID NOs: 197, 201, and 234-237. Accordingly, in some embodiments, any of the fusion proteins provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

Fusion Proteins with Internal Insertions

Provided herein are fusion proteins comprising a heterologous polypeptide fused to a nucleic acid programmable nucleic acid binding protein, for example, a napDNAbp. A heterologous polypeptide can be a polypeptide that is not found in the native or wild-type napDNAbp polypeptide sequence. The heterologous polypeptide can be fused to the napDNAbp at a C-terminal end of the napDNAbp, an N-terminal end of the napDNAbp, or inserted at an internal location of the napDNAbp.

In some embodiments, the heterologous polypeptide is inserted at an internal location of the napDNAbp. In some embodiments, the heterologous polypeptide is a deaminase (e.g., adenosine deaminase) or a functional fragment thereof. For example, a fusion protein can comprise a deaminase (e.g., adenosine deaminase) flanked by an N-terminal fragment and a C-terminal fragment of a Cas9 polypeptide. The deaminase in a fusion protein can be an adenosine deaminase. In some embodiments, the adenosine deaminase is a TadA (e.g., TadA*7.10 or a variant thereof).

In some embodiments, the fusion protein comprises the structure:
NH2-[N-terminal fragment of a napDNAbp]-[deaminase]-[C-terminal fragment of a napDNAbp]-COOH;
NH2-[N-terminal fragment of a Cas9]-[adenosine deaminase]-[C-terminal fragment of a Cas9]-COOH;
wherein each instance of “]-[” is an optional linker.

The deaminase can be a circular permutant deaminase. For example, the deaminase can be a circular permutant adenosine deaminase. In some embodiments, the deaminase is a circular permutant TadA, circularly permutated at amino acid residue 116 as numbered in the TadA reference sequence. In some embodiments, the deaminase is a circular permutant TadA, circularly permutated at amino acid residue 136 as numbered in the TadA reference sequence. In some embodiments, the deaminase is a circular permutant TadA, circularly permutated at amino acid residue 65 as numbered in the TadA reference sequence.

The fusion protein can comprise more than one deaminase. The fusion protein can comprise, for example, 1, 2, 3, 4, 5 or more deaminases. In some embodiments, the fusion protein comprises one deaminase. In some embodiments, the fusion protein comprises two deaminases. The two or more deaminases in a fusion protein can be an adenosine deaminase, cytidine deaminase, or a combination thereof. The two or more deaminases can be homodimers. The two or more deaminases can be heterodimers. The two or more deaminases can be inserted in tandem in the napDNAbp. In some embodiments, the two or more deaminases may not be in tandem in the napDNAbp.

In some embodiments, the napDNAbp in the fusion protein is a Cas9 polypeptide or a fragment thereof. The Cas9 polypeptide can be a variant Cas9 polypeptide. In some embodiments, the Cas9 polypeptide is a Cas9 nickase (nCas9) polypeptide or a fragment thereof. In some embodiments, the Cas9 polypeptide is a nuclease dead Cas9 (dCas9) polypeptide or a fragment thereof. The Cas9 polypeptide in a fusion protein can be a full-length Cas9 polypeptide. In some cases, the Cas9 polypeptide in a fusion protein may not be a full length Cas9 polypeptide. The Cas9 polypeptide can be truncated, for example, at a N-terminal or C-terminal end relative to a naturally-occurring Cas9 protein. The Cas9 polypeptide can be a circularly permuted Cas9 protein. The Cas9 polypeptide can be a fragment, a portion, or a domain of a Cas9 polypeptide, that is still capable of binding the target polynucleotide and a guide nucleic acid sequence.

In some embodiments, the Cas9 polypeptide is a Streptococcus pyogenes Cas9 (SpCas9), Staphylococcus aureus Cas9 (SaCas9), Streptococcus thermophilus 1 Cas9 (St1Cas9), or fragments or variants thereof.

Fusion proteins comprising a heterologous catalytic domain flanked by N- and C-terminal fragments of a Cas9 polypeptide are also useful for base editing in the methods as described herein. Fusion proteins comprising Cas9 and one or more deaminase domains, e.g., adenosine deaminase, or comprising an adenosine deaminase domain flanked by Cas9 sequences are also useful for highly specific and efficient base editing of target sequences. In an embodiment, a chimeric Cas9 fusion protein contains a heterologous catalytic domain (e.g., adenosine deaminase, cytidine deaminase, or adenosine deaminase and cytidine deaminase) inserted within a Cas9 polypeptide. In some embodiments, the fusion protein comprises an adenosine deaminase domain and a cytidine deaminase domain inserted within a Cas9. In some embodiments, an adenosine deaminase is fused within a Cas9 and a cytidine deaminase is fused to the C-terminus. In some embodiments, an adenosine deaminase is fused within Cas9 and a cytidine deaminase fused to the N-terminus. In some embodiments, a cytidine deaminase is fused within Cas9 and an adenosine deaminase is fused to the C-terminus. In some embodiments, a cytidine deaminase is fused within Cas9 and an adenosine deaminase fused to the N-terminus.

Exemplary structures of a fusion protein with an adenosine deaminase and a cytidine deaminase and a Cas9 are provided as follows:
NH₂-[Cas9(adenosine deaminase)]-[cytidine deaminase]-COOH;
NH₂-[cytidine deaminase]-[Cas9(adenosine deaminase)]-COOH;
NH₂-[Cas9(cytidine deaminase)]-[adenosine deaminase]-COOH; or
NH₂-[adenosine deaminase]-[Cas9(cytidine deaminase)]-COOH.

In some embodiments, the “-” used in the general architecture above indicates the presence of an optional linker.

In various embodiments, the catalytic domain has DNA modifying activity (e.g., deaminase activity), such as adenosine deaminase activity. In some embodiments, the adenosine deaminase is a TadA (e.g., TadA*7.10). In some embodiments, the TadA is a TadA variant. In some embodiments, a TadA variant is fused within Cas9 and a cytidine deaminase is fused to the C-terminus. In some embodiments, a TadA variant is fused within Cas9 and a cytidine deaminase fused to the N-terminus. In some embodiments, a cytidine deaminase is fused within Cas9 and a TadA variant is fused to the C-terminus. In some embodiments, a cytidine deaminase is fused within Cas9 and a TadA variant fused to the N-terminus. Exemplary structures of a fusion protein with a TadA variant and a cytidine deaminase and a Cas9 are provided as follows:
NH₂-[Cas9(TadA variant)]-[cytidine deaminase]-COOH;
NH₂-[cytidine deaminase]-[Cas9(TadA variant)]-COOH;
NH₂-[Cas9(cytidine deaminase)]-[TadA variant]-COOH; or
NH₂-[TadA variant]-[Cas9(cytidine deaminase)]-COOH.

In some embodiments, the “-” used in the general architecture above indicates the presence of an optional linker.

In other embodiments, the fusion protein contains one or more catalytic domains. In other embodiments, at least one of the one or more catalytic domains is inserted within the Cas12 polypeptide or is fused at the Cas12 N-terminus or C-terminus. In other embodiments, at least one of the one or more catalytic domains is inserted within a loop, an alpha helix region, an unstructured portion, or a solvent accessible portion of the Cas12 polypeptide. In other embodiments, the Cas12 polypeptide is Cas12a, Cas12b, Cas12c, Cas12d, Cas12e, Cas12g, Cas12h, Cas12i, or Cas12j/CasΦ. In other embodiments, the Cas12 polypeptide has at least about 85% amino acid sequence identity to Bacillus hisashii Cas12b, Bacillus thermoamylovorans Cas12b, Bacillus sp. V3-13 Cas12b, or Alicyclobacillus acidiphilus Cas12b (SEQ ID NO: 254). In other embodiments, the Cas12 polypeptide has at least about 90% amino acid sequence identity to Bacillus hisashii Cas12b (SEQ ID NO: 255), Bacillus thermoamylovorans Cas12b, Bacillus sp. V3-13 Cas12b, or Alicyclobacillus acidiphilus Cas12b. In other embodiments, the Cas12 polypeptide has at least about 95% amino acid sequence identity to Bacillus hisashii Cas12b, Bacillus thermoamylovorans Cas12b (SEQ ID NO: 256), Bacillus sp. V3-13 Cas12b (SEQ ID NO: 257), or Alicyclobacillus acidiphilus Cas12b.

In other embodiments, the Cas12 polypeptide contains or consists essentially of a fragment of Bacillus hisashii Cas12b, Bacillus thermoamylovorans Cas12b, Bacillus sp. V3-13 Cas12b, or Alicyclobacillus acidiphilus Cas12b. In embodiments, the Cas12 polypeptide contains BvCas12b (V4), which in some embodiments is expressed as 5′ mRNA Cap-5′ UTR-bhCas12b-STOP sequence-3′ UTR-120polyA tail (SEQ ID NOs: 258-260).

In other embodiments, the catalytic domain is inserted between amino acid positions 153-154, 255-256, 306-307, 980-981, 1019-1020, 534-535, 604-605, or 344-345 of BhCas12b or a corresponding amino acid residue of Cas12a, Cas12c, Cas12d, Cas12e, Cas12g, Cas12h, Cas12i, or Cas12j/CasΦ. In other embodiments, the catalytic domain is inserted between amino acids P153 and S154 of BhCas12b. In other embodiments, the catalytic domain is inserted between amino acids K255 and E256 of BhCas12b. In other embodiments, the catalytic domain is inserted between amino acids D980 and G981 of BhCas12b. In other embodiments, the catalytic domain is inserted between amino acids K1019 and L1020 of BhCas12b. In other embodiments, the catalytic domain is inserted between amino acids F534 and P535 of BhCas12b. In other embodiments, the catalytic domain is inserted between amino acids K604 and G605 of BhCas12b. In other embodiments, the catalytic domain is inserted between amino acids H344 and F345 of BhCas12b. In other embodiments, catalytic domain is inserted between amino acid positions 147 and 148, 248 and 249, 299 and 300, 991 and 992, or 1031 and 1032 of BvCas12b or a corresponding amino acid residue of Cas12a, Cas12c, Cas12d, Cas12e, Cas12g, Cas12h, Cas12i, or Cas12j/CasΦ. In other embodiments, the catalytic domain is inserted between amino acids P147 and D148 of BvCas12b. In other embodiments, the catalytic domain is inserted between amino acids G248 and G249 of BvCas12b. In other embodiments, the catalytic domain is inserted between amino acids P299 and E300 of BvCas12b. In other embodiments, the catalytic domain is inserted between amino acids G991 and E992 of BvCas12b. In other embodiments, the catalytic domain is inserted between amino acids K1031 and M1032 of BvCas12b. In other embodiments, the catalytic domain is inserted between amino acid positions 157 and 158, 258 and 259, 310 and 311, 1008 and 1009, or 1044 and 1045 of AaCas12b or a corresponding amino acid residue of Cas12a, Cas12c, Cas12d, Cas12e, Cas12g, Cas12h, Cas12i, or Cas12j/CasΦ. In other embodiments, the catalytic domain is inserted between amino acids P157 and G158 of AaCas12b. In other embodiments, the catalytic domain is inserted between amino acids V258 and G259 of AaCas12b. In other embodiments, the catalytic domain is inserted between amino acids D310 and P311 of AaCas12b. In other embodiments, the catalytic domain is inserted between amino acids G1008 and E1009 of AaCas12b. In other embodiments, the catalytic domain is inserted between amino acids G1044 and K1045 at of AaCas12b.

In other embodiments, the fusion protein contains a nuclear localization signal (e.g., a bipartite nuclear localization signal). In other embodiments, the amino acid sequence of the nuclear localization signal is MAPKKKRKVGIHGVPAA (SEQ ID NO: 261). In other embodiments of the above aspects, the nuclear localization signal is encoded by the following sequence:

ATGGCCCCAAAGAAGAAGCGGAAGGTCGGTATCCACGGAGTCCCAGCAGCC (SEQ ID NO: 262). In other embodiments, the Cas12b polypeptide contains a mutation that silences the catalytic activity of a RuvC domain. In other embodiments, the Cas12b polypeptide contains D574A, D829A and/or D952A mutations. In other embodiments, the fusion protein further contains a tag (e.g., an influenza hemagglutinin tag).

In some embodiments, the fusion protein comprises a napDNAbp domain (e.g., Cas12-derived domain) with an internally fused nucleobase editing domain (e.g., all or a portion of a deaminase domain, e.g., an adenosine deaminase domain). In some embodiments, the napDNAbp is a Cas12b.

By way of nonlimiting example, an adenosine deaminase (e.g., TadA*8.13) may be inserted into a BhCas12b to produce a fusion protein (e.g., TadA*8.13-BhCas12b) that effectively edits a nucleic acid sequence.

In some embodiments, the base editing system described herein is an ABE with TadA inserted into a Cas9. Polypeptide sSequences of relevant ABEs with TadA inserted into a Cas9 are provided in the attached Ssequence Llisting as SEQ ID NOs: 263-308.

In some embodiments, adenosine base editors were generated to insert TadA or variants thereof into the Cas9 polypeptide at the identified positions.

Exemplary, yet nonlimiting, fusion proteins are described in International PCT Application Nos. PCT/US2020/016285 and U.S. Provisional Application Nos. 62/852,228 and 62/852,224, the contents of which are incorporated by reference herein in their entireties.

The heterologous polypeptide (e.g., deaminase) can be inserted in the napDNAbp (e.g., Cas9) at a suitable location, for example, such that the napDNAbp retains its ability to bind the target polynucleotide and a guide nucleic acid. A deaminase (e.g., adenosine deaminase, cytidine deaminase, or adenosine deaminase and cytidine deaminase) can be inserted into a napDNAbp without compromising function of the deaminase (e.g., base editing activity) or the napDNAbp (e.g., ability to bind to target nucleic acid and guide nucleic acid). A deaminase (e.g., adenosine deaminase, cytidine deaminase, or adenosine deaminase and cytidine deaminase) can be inserted in the napDNAbp at, for example, a disordered region or a region comprising a high temperature factor or B-factor as shown by crystallographic studies. Regions of a protein that are less ordered, disordered, or unstructured, for example solvent exposed regions and loops, can be used for insertion without compromising structure or function. A deaminase (e.g., adenosine deaminase, cytidine deaminase, or adenosine deaminase and cytidine deaminase) can be inserted in the napDNAbp in a flexible loop region or a solvent-exposed region. In some embodiments, the deaminase (e.g., adenosine deaminase, cytidine deaminase, or adenosine deaminase and cytidine deaminase) is inserted in a flexible loop of the Cas9 polypeptide.

In some embodiments, the insertion location of a deaminase (e.g., adenosine deaminase, cytidine deaminase, or adenosine deaminase and cytidine deaminase) is determined by B-factor analysis of the crystal structure of Cas9 polypeptide. In some embodiments, the deaminase (e.g., adenosine deaminase, cytidine deaminase, or adenosine deaminase and cytidine deaminase) is inserted in regions of the Cas9 polypeptide comprising higher than average B-factors (e.g., higher B factors compared to the total protein or the protein domain comprising the disordered region). B-factor or temperature factor can indicate the fluctuation of atoms from their average position (for example, as a result of temperature-dependent atomic vibrations or static disorder in a crystal lattice). A high B-factor (e.g., higher than average B-factor) for backbone atoms can be indicative of a region with relatively high local mobility. Such a region can be used for inserting a deaminase without compromising structure or function. A deaminase (e.g., adenosine deaminase, cytidine deaminase, or adenosine deaminase and cytidine deaminase) can be inserted at a location with a residue having a Ca atom with a B-factor that is 50%, 60%, 70%, 80%, 90%, 100%, 110%, 120%, 130%, 140%, 150%, 160%, 170%, 180%, 190%, 200%, or greater than 200% more than the average B-factor for the total protein. A deaminase (e.g., adenosine deaminase, cytidine deaminase, or adenosine deaminase and cytidine deaminase) can be inserted at a location with a residue having a Ca atom with a B-factor that is 50%, 60%, 70%, 80%, 90%, 100%, 110%, 120%, 130%, 140%, 150%, 160%, 170%, 180%, 190%, 200% or greater than 200% more than the average B-factor for a Cas9 protein domain comprising the residue. Cas9 polypeptide positions comprising a higher than average B-factor can include, for example, residues 768, 792, 1052, 1015, 1022, 1026, 1029, 1067, 1040, 1054, 1068, 1246, 1247, and 1248 as numbered in the above Cas9 reference sequence. Cas9 polypeptide regions comprising a higher than average B-factor can include, for example, residues 792-872, 792-906, and 2-791 as numbered in the above Cas9 reference sequence.

A heterologous polypeptide (e.g., deaminase) can be inserted in the napDNAbp at an amino acid residue selected from the group consisting of: 768, 791, 792, 1015, 1016, 1022, 1023, 1026, 1029, 1040, 1052, 1054, 1067, 1068, 1069, 1246, 1247, and 1248 as numbered in the above Cas9 reference sequence, or a corresponding amino acid residue in another Cas9 polypeptide. In some embodiments, the heterologous polypeptide is inserted between amino acid positions 768-769, 791-792, 792-793, 1015-1016, 1022-1023, 1026-1027, 1029-1030, 1040-1041, 1052-1053, 1054-1055, 1067-1068, 1068-1069, 1247-1248, or 1248-1249 as numbered in the above Cas9 reference sequence or corresponding amino acid positions thereof. In some embodiments, the heterologous polypeptide is inserted between amino acid positions 769-770, 792-793, 793-794, 1016-1017, 1023-1024, 1027-1028, 1030-1031, 1041-1042, 1053-1054, 1055-1056, 1068-1069, 1069-1070, 1248-1249, or 1249-1250 as numbered in the above Cas9 reference sequence or corresponding amino acid positions thereof. In some embodiments, the heterologous polypeptide replaces an amino acid residue selected from the group consisting of: 768, 791, 792, 1015, 1016, 1022, 1023, 1026, 1029, 1040, 1052, 1054, 1067, 1068, 1069, 1246, 1247, and 1248 as numbered in the above Cas9 reference sequence, or a corresponding amino acid residue in another Cas9 polypeptide. It should be understood that the reference to the above Cas9 reference sequence with respect to insertion positions is for illustrative purposes. The insertions as discussed herein are not limited to the Cas9 polypeptide sequence of the above Cas9 reference sequence, but include insertion at corresponding locations in variant Cas9 polypeptides, for example a Cas9 nickase (nCas9), nuclease dead Cas9 (dCas9), a Cas9 variant lacking a nuclease domain, a truncated Cas9, or a Cas9 domain lacking partial or complete HNH domain.

A heterologous polypeptide (e.g., deaminase) can be inserted in the napDNAbp at an amino acid residue selected from the group consisting of: 768, 792, 1022, 1026, 1040, 1068, and 1247 as numbered in the above Cas9 reference sequence, or a corresponding amino acid residue in another Cas9 polypeptide. In some embodiments, the heterologous polypeptide is inserted between amino acid positions 768-769, 792-793, 1022-1023, 1026-1027, 1029-1030, 1040-1041, 1068-1069, or 1247-1248 as numbered in the above Cas9 reference sequence or corresponding amino acid positions thereof. In some embodiments, the heterologous polypeptide is inserted between amino acid positions 769-770, 793-794, 1023-1024, 1027-1028, 1030-1031, 1041-1042, 1069-1070, or 1248-1249 as numbered in the above Cas9 reference sequence or corresponding amino acid positions thereof. In some embodiments, the heterologous polypeptide replaces an amino acid residue selected from the group consisting of: 768, 792, 1022, 1026, 1040, 1068, and 1247 as numbered in the above Cas9 reference sequence, or a corresponding amino acid residue in another Cas9 polypeptide.

A heterologous polypeptide (e.g., deaminase) can be inserted in the napDNAbp at an amino acid residue as described herein, or a corresponding amino acid residue in another Cas9 polypeptide. In an embodiment, a heterologous polypeptide (e.g., deaminase) can be inserted in the napDNAbp at an amino acid residue selected from the group consisting of: 1002, 1003, 1025, 1052-1056, 1242-1247, 1061-1077, 943-947, 686-691, 569-578, 530-539, and 1060-1077 as numbered in the above Cas9 reference sequence, or a corresponding amino acid residue in another Cas9 polypeptide. The deaminase (e.g., adenosine deaminase, cytidine deaminase, or adenosine deaminase and cytidine deaminase) can be inserted at the N-terminus or the C-terminus of the residue or replace the residue. In some embodiments, the deaminase (e.g., adenosine deaminase, cytidine deaminase, or adenosine deaminase and cytidine deaminase) is inserted at the C-terminus of the residue.

In some embodiments, an adenosine deaminase (e.g., TadA) is inserted at an amino acid residue selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide. In some embodiments, an adenosine deaminase (e.g., TadA) is inserted in place of residues 792-872, 792-906, or 2-791 as numbered in the above Cas9 reference sequence, or a corresponding amino acid residue in another Cas9 polypeptide. In some embodiments, the adenosine deaminase is inserted at the N-terminus of an amino acid selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide. In some embodiments, the adenosine deaminase is inserted at the C-terminus of an amino acid selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide. In some embodiments, the adenosine deaminase is inserted to replace an amino acid selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 768 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 768 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 768 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 768 as numbered in the above Cas9 reference sequence, or a corresponding amino acid residue in another Cas9 polypeptide.

In some embodiments, the deaminase (e.g., adenosine deaminase) is inserted at amino acid residue 791 or is inserted at amino acid residue 792, as numbered in the above Cas9 reference sequence, or a corresponding amino acid residue in another Cas9 polypeptide. In some embodiments, the deaminase (e.g., adenosine deaminase) is inserted at the N-terminus of amino acid residue 791 or is inserted at the N-terminus of amino acid 792, as numbered in the above Cas9 reference sequence, or a corresponding amino acid residue in another Cas9 polypeptide. In some embodiments, the deaminase (e.g., adenosine deaminase) is inserted at the C-terminus of amino acid 791 or is inserted at the N-terminus of amino acid 792, as numbered in the above Cas9 reference sequence, or a corresponding amino acid residue in another Cas9 polypeptide. In some embodiments, the deaminase (e.g., adenosine deaminase) is inserted to replace amino acid 791, or is inserted to replace amino acid 792, as numbered in the above Cas9 reference sequence, or a corresponding amino acid residue in another Cas9 polypeptide.

In some embodiments, the deaminase (e.g., adenosine deaminase) is inserted at amino acid residue 1016 as numbered in the above Cas9 reference sequence, or a corresponding amino acid residue in another Cas9 polypeptide. In some embodiments, the deaminase (e.g., adenosine deaminase) is inserted at the N-terminus of amino acid residue 1016 as numbered in the above Cas9 reference sequence, or a corresponding amino acid residue in another Cas9 polypeptide. In some embodiments, the deaminase (e.g., adenosine deaminase) is inserted at the C-terminus of amino acid residue 1016 as numbered in the above Cas9 reference sequence, or a corresponding amino acid residue in another Cas9 polypeptide. In some embodiments, the deaminase (e.g., adenosine deaminase) is inserted to replace amino acid residue 1016 as numbered in the above Cas9 reference sequence, or a corresponding amino acid residue in another Cas9 polypeptide.

In some embodiments, the deaminase (e.g., adenosine deaminase) is inserted at amino acid residue 1022, or is inserted at amino acid residue 1023, as numbered in the above Cas9 reference sequence, or a corresponding amino acid residue in another Cas9 polypeptide. In some embodiments, the deaminase (e.g., adenosine deaminase) is inserted at the N-terminus of amino acid residue 1022 or is inserted at the N-terminus of amino acid residue 1023, as numbered in the above Cas9 reference sequence, or a corresponding amino acid residue in another Cas9 polypeptide. In some embodiments, the deaminase (e.g., adenosine deaminase) is inserted at the C-terminus of amino acid residue 1022 or is inserted at the C-terminus of amino acid residue 1023, as numbered in the above Cas9 reference sequence, or a corresponding amino acid residue in another Cas9 polypeptide. In some embodiments, the deaminase (e.g., adenosine deaminase) is inserted to replace amino acid residue 1022, or is inserted to replace amino acid residue 1023, as numbered in the above Cas9 reference sequence, or a corresponding amino acid residue in another Cas9 polypeptide.

In some embodiments, the deaminase (e.g., adenosine deaminase) is inserted at amino acid residue 1026, or is inserted at amino acid residue 1029, as numbered in the above Cas9 reference sequence, or a corresponding amino acid residue in another Cas9 polypeptide. In some embodiments, the deaminase (e.g., adenosine deaminase) is inserted at the N-terminus of amino acid residue 1026 or is inserted at the N-terminus of amino acid residue 1029, as numbered in the above Cas9 reference sequence, or a corresponding amino acid residue in another Cas9 polypeptide. In some embodiments, the deaminase (e.g., adenosine deaminase) is inserted at the C-terminus of amino acid residue 1026 or is inserted at the C-terminus of amino acid residue 1029, as numbered in the above Cas9 reference sequence, or a corresponding amino acid residue in another Cas9 polypeptide. In some embodiments, the deaminase (e.g., adenosine deaminase) is inserted to replace amino acid residue 1026, or is inserted to replace amino acid residue 1029, as numbered in the above Cas9 reference sequence, or corresponding amino acid residue in another Cas9 polypeptide.

In some embodiments, the deaminase (e.g., adenosine deaminase) is inserted at amino acid residue 1040 as numbered in the above Cas9 reference sequence, or a corresponding amino acid residue in another Cas9 polypeptide. In some embodiments, the deaminase (e.g., adenosine deaminase,) is inserted at the N-terminus of amino acid residue 1040 as numbered in the above Cas9 reference sequence, or a corresponding amino acid residue in another Cas9 polypeptide. In some embodiments, the deaminase (e.g., adenosine deaminase) is inserted at the C-terminus of amino acid residue 1040 as numbered in the above Cas9 reference sequence, or a corresponding amino acid residue in another Cas9 polypeptide. In some embodiments, the deaminase (e.g., adenosine deaminase) is inserted to replace amino acid residue 1040 as numbered in the above Cas9 reference sequence, or a corresponding amino acid residue in another Cas9 polypeptide.

In some embodiments, the deaminase (e.g., adenosine deaminase) is inserted at amino acid residue 1052, or is inserted at amino acid residue 1054, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1052 or is inserted at the N-terminus of amino acid residue 1054, as numbered in the above Cas9 reference sequence, or a corresponding amino acid residue in another Cas9 polypeptide. In some embodiments, the deaminase (e.g., adenosine deaminase) is inserted at the C-terminus of amino acid residue 1052 or is inserted at the C-terminus of amino acid residue 1054, as numbered in the above Cas9 reference sequence, or a corresponding amino acid residue in another Cas9 polypeptide. In some embodiments, the deaminase (e.g., adenosine deaminase) is inserted to replace amino acid residue 1052, or is inserted to replace amino acid residue 1054, as numbered in the above Cas9 reference sequence, or a corresponding amino acid residue in another Cas9 polypeptide.

In some embodiments, the deaminase (e.g., adenosine deaminase) is inserted at amino acid residue 1067, or is inserted at amino acid residue 1068, or is inserted at amino acid residue 1069, as numbered in the above Cas9 reference sequence, or a corresponding amino acid residue in another Cas9 polypeptide. In some embodiments, the deaminase (e.g., adenosine deaminase) is inserted at the N-terminus of amino acid residue 1067 or is inserted at the N-terminus of amino acid residue 1068 or is inserted at the N-terminus of amino acid residue 1069, as numbered in the above Cas9 reference sequence, or a corresponding amino acid residue in another Cas9 polypeptide. In some embodiments, the deaminase (e.g., adenosine deaminase) is inserted at the C-terminus of amino acid residue 1067 or is inserted at the C-terminus of amino acid residue 1068 or is inserted at the C-terminus of amino acid residue 1069, as numbered in the above Cas9 reference sequence, or a corresponding amino acid residue in another Cas9 polypeptide. In some embodiments, the deaminase (e.g., adenosine deaminase) is inserted to replace amino acid residue 1067, or is inserted to replace amino acid residue 1068, or is inserted to replace amino acid residue 1069, as numbered in the above Cas9 reference sequence, or a corresponding amino acid residue in another Cas9 polypeptide.

In some embodiments, the deaminase (e.g., adenosine deaminase) is inserted at amino acid residue 1246, or is inserted at amino acid residue 1247, or is inserted at amino acid residue 1248, as numbered in the above Cas9 reference sequence, or a corresponding amino acid residue in another Cas9 polypeptide. In some embodiments, the deaminase (e.g., adenosine deaminase) is inserted at the N-terminus of amino acid residue 1246 or is inserted at the N-terminus of amino acid residue 1247 or is inserted at the N-terminus of amino acid residue 1248, as numbered in the above Cas9 reference sequence, or a corresponding amino acid residue in another Cas9 polypeptide. In some embodiments, the deaminase (e.g., adenosine deaminase is inserted at the C-terminus of amino acid residue 1246 or is inserted at the C-terminus of amino acid residue 1247 or is inserted at the C-terminus of amino acid residue 1248, as numbered in the above Cas9 reference sequence, or a corresponding amino acid residue in another Cas9 polypeptide. In some embodiments, the deaminase (e.g., adenosine deaminase) is inserted to replace amino acid residue 1246, or is inserted to replace amino acid residue 1247, or is inserted to replace amino acid residue 1248, as numbered in the above Cas9 reference sequence, or a corresponding amino acid residue in another Cas9 polypeptide.

In some embodiments, a heterologous polypeptide (e.g., deaminase) is inserted in a flexible loop of a Cas9 polypeptide. The flexible loop portions can be selected from the group consisting of 530-537, 569-570, 686-691, 943-947, 1002-1025, 1052-1077, 1232-1247, or 1298-1300 as numbered in the above Cas9 reference sequence, or a corresponding amino acid residue in another Cas9 polypeptide. The flexible loop portions can be selected from the group consisting of: 1-529, 538-568, 580-685, 692-942, 948-1001, 1026-1051, 1078-1231, or 1248-1297 as numbered in the above Cas9 reference sequence, or a corresponding amino acid residue in another Cas9 polypeptide.

A heterologous polypeptide (e.g., adenine deaminase) can be inserted into a Cas9 polypeptide region corresponding to amino acid residues: 1017-1069, 1242-1247, 1052-1056, 1060-1077, 1002-1003, 943-947, 530-537, 568-579, 686-691, 1242-1247, 1298-1300, 1066-1077, 1052-1056, or 1060-1077 as numbered in the above Cas9 reference sequence, or a corresponding amino acid residue in another Cas9 polypeptide.

A heterologous polypeptide (e.g., adenine deaminase) can be inserted in place of a deleted region of a Cas9 polypeptide. The deleted region can correspond to an N-terminal or C-terminal portion of the Cas9 polypeptide. In some embodiments, the deleted region corresponds to residues 792-872 as numbered in the above Cas9 reference sequence, or a corresponding amino acid residue in another Cas9 polypeptide. In some embodiments, the deleted region corresponds to residues 792-906 as numbered in the above Cas9 reference sequence, or a corresponding amino acid residue in another Cas9 polypeptide. In some embodiments, the deleted region corresponds to residues 2-791 as numbered in the above Cas9 reference sequence, or a corresponding amino acid residue in another Cas9 polypeptide. In some embodiments, the deleted region corresponds to residues 1017-1069 as numbered in the above Cas9 reference sequence, or corresponding amino acid residues thereof.

Exemplary internal fusions base editors are provided in Table 6 below:

TABLE 6
Insertion loci in Cas9 proteins

| BE ID | Modification | Other ID |
|---|---|---|
| IBE001 | Cas9 TadA ins 1015 | ISLAY01 |
| IBE002 | Cas9 TadA ins 1022 | ISLAY02 |
| IBE003 | Cas9 TadA ins 1029 | ISLAY03 |
| IBE004 | Cas9 TadA ins 1040 | ISLAY04 |
| IBE005 | Cas9 TadA ins 1068 | ISLAY05 |
| IBE006 | Cas9 TadA ins 1247 | ISLAY06 |
| IBE007 | Cas9 TadA ins 1054 | ISLAY07 |
| IBE008 | Cas9 TadA ins 1026 | ISLAY08 |
| IBE009 | Cas9 TadA ins 768 | ISLAY09 |
| IBE020 | delta HNH TadA 792 | ISLAY20 |
| IBE021 | N-term fusion single TadA helix truncated 165-end | ISLAY21 |
| IBE029 | TadA-Circular Permutant 116 ins 1067 | ISLAY29 |
| IBE031 | TadA-Circular Permutant 136 ins 1248 | ISLAY31 |
| IBE032 | TadA-Circular Permutant 136 ins 1052 | ISLAY32 |
| IBE035 | delta 792-872 TadA ins | ISLAY35 |
| IBE036 | delta 792-906 TadA ins | ISLAY36 |
| IBE043 | TadA-Circular Permutant 65 ins 1246 | ISLAY43 |
| IBE044 | TadA ins C-term truncate2 791 | ISLAY44 |

A heterologous polypeptide (e.g., deaminase) can be inserted within a structural or functional domain of a Cas9 polypeptide. A heterologous polypeptide (e.g., deaminase) can be inserted between two structural or functional domains of a Cas9 polypeptide. A heterologous polypeptide (e.g., deaminase) can be inserted in place of a structural or functional domain of a Cas9 polypeptide, for example, after deleting the domain from the Cas9 polypeptide. The structural or functional domains of a Cas9 polypeptide can include, for example, RuvC I, RuvC II, RuvC III, Rec1, Rec2, PI, or HNH.

In some embodiments, the Cas9 polypeptide lacks one or more domains selected from the group consisting of: RuvC I, RuvC II, RuvC III, Rec1, Rec2, PI, or HNH domain. In some embodiments, the Cas9 polypeptide lacks a nuclease domain. In some embodiments, the Cas9 polypeptide lacks an HNH domain. In some embodiments, the Cas9 polypeptide lacks a portion of the HNH domain such that the Cas9 polypeptide has reduced or abolished HNH activity. In some embodiments, the Cas9 polypeptide comprises a deletion of the nuclease domain, and the deaminase is inserted to replace the nuclease domain. In some embodiments, the HNH domain is deleted and the deaminase is inserted in its place. In some embodiments, one or more of the RuvC domains is deleted and the deaminase is inserted in its place.

A fusion protein comprising a heterologous polypeptide can be flanked by a N-terminal and a C-terminal fragment of a napDNAbp. In some embodiments, the fusion protein comprises a deaminase flanked by a N-terminal fragment and a C-terminal fragment of a Cas9 polypeptide. The N terminal fragment or the C terminal fragment can bind the target polynucleotide sequence. The C-terminus of the N terminal fragment or the N-terminus of the C terminal fragment can comprise a part of a flexible loop of a Cas9 polypeptide. The C-terminus of the N terminal fragment or the N-terminus of the C terminal fragment can comprise a part of an alpha-helix structure of the Cas9 polypeptide. The N-terminal fragment or the C-terminal fragment can comprise a DNA binding domain. The N-terminal fragment or the C-terminal fragment can comprise a RuvC domain. The N-terminal fragment or the C-terminal fragment can comprise an HNH domain. In some embodiments, neither of the N-terminal fragment and the C-terminal fragment comprises an HNH domain.

In some embodiments, the C-terminus of the N terminal Cas9 fragment comprises an amino acid that is in proximity to a target nucleobase when the fusion protein deaminates the target nucleobase. In some embodiments, the N-terminus of the C terminal Cas9 fragment comprises an amino acid that is in proximity to a target nucleobase when the fusion protein deaminates the target nucleobase. The insertion location of different deaminases can be different in order to have proximity between the target nucleobase and an amino acid in the C-terminus of the N terminal Cas9 fragment or the N-terminus of the C terminal Cas9 fragment. For example, the insertion position of a deaminase can be at an amino acid residue selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide.

The N-terminal Cas9 fragment of a fusion protein (i.e. the N-terminal Cas9 fragment flanking the deaminase in a fusion protein) can comprise the N-terminus of a Cas9 polypeptide. The N-terminal Cas9 fragment of a fusion protein can comprise a length of at least about: 100, 200, 300, 400, 500, 600, 700, 800, 900, 1000, 1100, 1200, or 1300 amino acids. The N-terminal Cas9 fragment of a fusion protein can comprise a sequence corresponding to amino acid residues: 1-56, 1-95, 1-200, 1-300, 1-400, 1-500, 1-600, 1-700, 1-718, 1-765, 1-780, 1-906, 1-918, or 1-1100 as numbered in the above Cas9 reference sequence, or a corresponding amino acid residue in another Cas9 polypeptide. The N-terminal Cas9 fragment can comprise a sequence comprising at least: 85%, at least 90%, at least 91%, at least 92%, at least 93%, at least 94%, at least 95%, at least 96%, at least 97%, at least 98%, at least 99%, or at least 99.5% sequence identity to amino acid residues: 1-56, 1-95, 1-200, 1-300, 1-400, 1-500, 1-600, 1-700, 1-718, 1-765, 1-780, 1-906, 1-918, or 1-1100 as numbered in the above Cas9 reference sequence, or a corresponding amino acid residue in another Cas9 polypeptide.

The C-terminal Cas9 fragment of a fusion protein (i.e. the C-terminal Cas9 fragment flanking the deaminase in a fusion protein) can comprise the C-terminus of a Cas9 polypeptide. The C-terminal Cas9 fragment of a fusion protein can comprise a length of at least about: 100, 200, 300, 400, 500, 600, 700, 800, 900, 1000, 1100, 1200, or 1300 amino acids. The C-terminal Cas9 fragment of a fusion protein can comprise a sequence corresponding to amino acid residues: 1099-1368, 918-1368, 906-1368, 780-1368, 765-1368, 718-1368, 94-1368, or 56-1368 as numbered in the above Cas9 reference sequence, or a corresponding amino acid residue in another Cas9 polypeptide. The N-terminal Cas9 fragment can comprise a sequence comprising at least: 85%, at least 90%, at least 91%, at least 92%, at least 93%, at least 94%, at least 95%, at least 96%, at least 97%, at least 98%, at least 99%, or at least 99.5% sequence identity to amino acid residues: 1099-1368, 918-1368, 906-1368, 780-1368, 765-1368, 718-1368, 94-1368, or 56-1368 as numbered in the above Cas9 reference sequence, or a corresponding amino acid residue in another Cas9 polypeptide.

The N-terminal Cas9 fragment and C-terminal Cas9 fragment of a fusion protein taken together may not correspond to a full-length naturally occurring Cas9 polypeptide sequence, for example, as set forth in the above Cas9 reference sequence.

The fusion protein described herein can effect targeted deamination with reduced deamination at non-target sites (e.g., off-target sites), such as reduced genome wide spurious deamination. The fusion protein described herein can effect targeted deamination with reduced bystander deamination at non-target sites. The undesired deamination or off-target deamination can be reduced by at least 30%, at least 40%, at least 50%, at least 60%, at least 70%, at least 80%, at least 90%, at least 95%, or at least 99% compared with, for example, an end terminus fusion protein comprising the deaminase fused to a N terminus or a C terminus of a Cas9 polypeptide. The undesired deamination or off-target deamination can be reduced by at least one-fold, at least two-fold, at least three-fold, at least four-fold, at least five-fold, at least tenfold, at least fifteen fold, at least twenty fold, at least thirty fold, at least forty fold, at least fifty fold, at least 60 fold, at least 70 fold, at least 80 fold, at least 90 fold, or at least hundred fold, compared with, for example, an end terminus fusion protein comprising the deaminase fused to a N terminus or a C terminus of a Cas9 polypeptide.

In some embodiments, the deaminase (e.g., adenosine deaminase) of the fusion protein deaminates no more than two nucleobases within the range of an R-loop. In some embodiments, the deaminase of the fusion protein deaminates no more than three nucleobases within the range of the R-loop. In some embodiments, the deaminase of the fusion protein deaminates no more than 2, 3, 4, 5, 6, 7, 8, 9, or 10 nucleobases within the range of the R-loop. An R-loop is a three-stranded nucleic acid structure including a DNA:RNA hybrid, a DNA:DNA or an RNA: RNA complementary structure and the associated with single-stranded DNA. As used herein, an R-loop may be formed when a target polynucleotide is contacted with a CRISPR complex or a base editing complex, wherein a portion of a guide polynucleotide, e.g. a guide RNA, hybridizes with and displaces with a portion of a target polynucleotide, e.g. a target DNA. In some embodiments, an R-loop comprises a hybridized region of a spacer sequence and a target DNA complementary sequence. An R-loop region may be of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobase pairs in length. In some embodiments, the R-loop region is about 20 nucleobase pairs in length. It should be understood that, as used herein, an R-loop region is not limited to the target DNA strand that hybridizes with the guide polynucleotide. For example, editing of a target nucleobase within an R-loop region may be to a DNA strand that comprises the complementary strand to a guide RNA, or may be to a DNA strand that is the opposing strand of the strand complementary to the guide RNA. In some embodiments, editing in the region of the R-loop comprises editing a nucleobase on non-complementary strand (protospacer strand) to a guide RNA in a target DNA sequence.

The fusion protein described herein can effect target deamination in an editing window different from canonical base editing. In some embodiments, a target nucleobase is from about 1 to about 20 bases upstream of a PAM sequence in the target polynucleotide sequence. In some embodiments, a target nucleobase is from about 2 to about 12 bases upstream of a PAM sequence in the target polynucleotide sequence. In some embodiments, a target nucleobase is from about 1 to 9 base pairs, about 2 to 10 base pairs, about 3 to 11 base pairs, about 4 to 12 base pairs, about 5 to 13 base pairs, about 6 to 14 base pairs, about 7 to 15 base pairs, about 8 to 16 base pairs, about 9 to 17 base pairs, about 10 to 18 base pairs, about 11 to 19 base pairs, about 12 to 20 base pairs, about 1 to 7 base pairs, about 2 to 8 base pairs, about 3 to 9 base pairs, about 4 to 10 base pairs, about 5 to 11 base pairs, about 6 to 12 base pairs, about 7 to 13 base pairs, about 8 to 14 base pairs, about 9 to 15 base pairs, about 10 to 16 base pairs, about 11 to 17 base pairs, about 12 to 18 base pairs, about 13 to 19 base pairs, about 14 to 20 base pairs, about 1 to 5 base pairs, about 2 to 6 base pairs, about 3 to 7 base pairs, about 4 to 8 base pairs, about 5 to 9 base pairs, about 6 to 10 base pairs, about 7 to 11 base pairs, about 8 to 12 base pairs, about 9 to 13 base pairs, about 10 to 14 base pairs, about 11 to 15 base pairs, about 12 to 16 base pairs, about 13 to 17 base pairs, about 14 to 18 base pairs, about 15 to 19 base pairs, about 16 to 20 base pairs, about 1 to 3 base pairs, about 2 to 4 base pairs, about 3 to 5 base pairs, about 4 to 6 base pairs, about 5 to 7 base pairs, about 6 to 8 base pairs, about 7 to 9 base pairs, about 8 to 10 base pairs, about 9 to 11 base pairs, about 10 to 12 base pairs, about 11 to 13 base pairs, about 12 to 14 base pairs, about 13 to 15 base pairs, about 14 to 16 base pairs, about 15 to 17 base pairs, about 16 to 18 base pairs, about 17 to 19 base pairs, about 18 to 20 base pairs away or upstream of the PAM sequence. In some embodiments, a target nucleobase is about 1, 2, 3, 4, 5, 6, 7, 8, 9, 10, 11, 12, 13, 14, 15, 16, 17, 18, 19, 20, or more base pairs away from or upstream of the PAM sequence. In some embodiments, a target nucleobase is about 1, 2, 3, 4, 5, 6, 7, 8, or 9 base pairs upstream of the PAM sequence. In some embodiments, a target nucleobase is about 2, 3, 4, or 6 base pairs upstream of the PAM sequence.

The fusion protein can comprise more than one heterologous polypeptide. For example, the fusion protein can additionally comprise one or more UGI domains and/or one or more nuclear localization signals. The two or more heterologous domains can be inserted in tandem. The two or more heterologous domains can be inserted at locations such that they are not in tandem in the NapDNAbp.

A fusion protein can comprise a linker between the deaminase and the napDNAbp polypeptide. The linker can be a peptide or a non-peptide linker. For example, the linker can be an XTEN, (GGGS)n (SEQ ID NO: 246), (GGGGS)n (SEQ ID NO: 247), (G)n, (EAAAK)n (SEQ ID NO: 248), (GGS)n, SGSETPGTSESATPES (SEQ ID NO: 249).

In some embodiments, the napDNAbp in the fusion protein is a Cas12 polypeptide, e.g., Cas12b/C2c1, or a fragment thereof. The Cas12 polypeptide can be a variant Cas12 polypeptide. In other embodiments, the N- or C-terminal fragments of the Cas12 polypeptide comprise a nucleic acid programmable DNA binding domain or a RuvC domain. In other embodiments, the fusion protein contains a linker between the Cas12 polypeptide and the catalytic domain. In other embodiments, the amino acid sequence of the linker is GGSGGS (SEQ ID NO: 250) or GSSGSETPGTSESATPESSG (SEQ ID NO: 251). In other embodiments, the linker is a rigid linker. In other embodiments of the above aspects, the linker is encoded by GGAGGCTCTGGAGGAAGC (SEQ ID NO: 252) or GGCTCTTCTGGATCTGAAACACCTGGCACAAGCGAGAGCGCCACCCCTGAGAGCTCTGGC (SEQ ID NO: 253).

In some embodiments, the fusion protein comprises a linker between the N-terminal Cas9 fragment and the deaminase. In some embodiments, the fusion protein comprises a linker between the C-terminal Cas9 fragment and the deaminase. In some embodiments, the N-terminal and C-terminal fragments of napDNAbp are connected to the deaminase with a linker. In some embodiments, the N-terminal and C-terminal fragments are joined to the deaminase domain without a linker. In some embodiments, the fusion protein comprises a linker between the N-terminal Cas9 fragment and the deaminase, but does not comprise a linker between the C-terminal Cas9 fragment and the deaminase. In some embodiments, the fusion protein comprises a linker between the C-terminal Cas9 fragment and the deaminase, but does not comprise a linker between the N-terminal Cas9 fragment and the deaminase.

In some embodiments, the base editing system described herein is an ABE with TadA inserted into a Cas9. Sequences of relevant ABEs with TadA inserted into a Cas9 are provided. In some embodiments, adenosine deaminase base editors were generated to insert TadA or variants thereof into the Cas9 polypeptide at the identified positions.

Fusion proteins comprising a heterologous catalytic domain flanked by N- and C-terminal fragments of a Cas12 polypeptide are also useful for base editing in the methods as described herein. Fusion proteins comprising Cas12 and one or more deaminase domains, e.g., adenosine deaminase, or comprising an adenosine deaminase domain flanked by Cas12 sequences are also useful for highly specific and efficient base editing of target sequences. In an embodiment, a chimeric Cas12 fusion protein contains a heterologous catalytic domain (e.g., adenosine deaminase, cytidine deaminase, or adenosine deaminase and cytidine deaminase) inserted within a Cas12 polypeptide. In some embodiments, the fusion protein comprises an adenosine deaminase domain and a cytidine deaminase domain inserted within a Cas12. In some embodiments, an adenosine deaminase is fused within Cas12 and a cytidine deaminase is fused to the C-terminus. In some embodiments, an adenosine deaminase is fused within Cas12 and a cytidine deaminase fused to the N-terminus. In some embodiments, a cytidine deaminase is fused within Cas12 and an adenosine deaminase is fused to the C-terminus. In some embodiments, a cytidine deaminase is fused within Cas12 and an adenosine deaminase fused to the N-terminus. Exemplary structures of a fusion protein with an adenosine deaminase and a cytidine deaminase and a Cas12 are provided as follows:
NH2-[Cas12(adenosine deaminase)]-[cytidine deaminase]-COOH;
NH2-[cytidine deaminase]-[Cas12(adenosine deaminase)]-COOH;
NH2-[Cas12(cytidine deaminase)]-[adenosine deaminase]-COOH; or
NH2-[adenosine deaminase]-[Cas12(cytidine deaminase)]-COOH;

In some embodiments, the “-” used in the general architecture above indicates the presence of an optional linker.

In various embodiments, the catalytic domain has DNA modifying activity (e.g., deaminase activity), such as adenosine deaminase activity. In some embodiments, the adenosine deaminase is a TadA (e.g., TadA*7.10). In some embodiments, the TadA is a TadA*8. In some embodiments, a TadA*8 is fused within Cas12 and a cytidine deaminase is fused to the C-terminus. In some embodiments, a TadA*8 is fused within Cas12 and a cytidine deaminase fused to the N-terminus. In some embodiments, a cytidine deaminase is fused within Cas12 and a TadA*8 is fused to the C-terminus. In some embodiments, a cytidine deaminase is fused within Cas12 and a TadA*8 fused to the N-terminus. Exemplary structures of a fusion protein with a TadA*8 and a cytidine deaminase and a Cas12 are provided as follows:
N-[Cas12(TadA*8)]-[cytidine deaminase]-C;
N-[cytidine deaminase]-[Cas12(TadA*8)]-C;
N-[Cas12(cytidine deaminase)]-[TadA*8]-C; or
N-[TadA*8]-[Cas12(cytidine deaminase)]-C.

In some embodiments, the “-” used in the general architecture above indicates the presence of an optional linker.

In other embodiments, the fusion protein contains one or more catalytic domains. In other embodiments, at least one of the one or more catalytic domains is inserted within the Cas12 polypeptide or is fused at the Cas12 N-terminus or C-terminus. In other embodiments, at least one of the one or more catalytic domains is inserted within a loop, an alpha helix region, an unstructured portion, or a solvent accessible portion of the Cas12 polypeptide. In other embodiments, the Cas12 polypeptide is Cas12a, Cas12b, Cas12c, Cas12d, Cas12e, Cas12g, Cas12h, Cas12i, or Cas12j/CasΦ. In other embodiments, the Cas12 polypeptide has at least about 85% amino acid sequence identity to Bacillus hisashii Cas12b, Bacillus thermoamylovorans Cas12b, Bacillus sp. V3-13 Cas12b, or Alicyclobacillus acidiphilus Cas12b (SEQ ID NO: 254). In other embodiments, the Cas12 polypeptide has at least about 90% amino acid sequence identity to Bacillus hisashii Cas12b (SEQ ID NO: 255), Bacillus thermoamylovorans Cas12b, Bacillus sp. V3-13 Cas12b, or Alicyclobacillus acidiphilus Cas12b. In other embodiments, the Cas12 polypeptide has at least about 95% amino acid sequence identity to Bacillus hisashii Cas12b, Bacillus thermoamylovorans Cas12b (SEQ ID NO: 256), Bacillus sp. V3-13 Cas12b (SEQ ID NO: 257), or Alicyclobacillus acidiphilus Cas12b. In other embodiments, the Cas12 polypeptide contains or consists essentially of a fragment of Bacillus hisashii Cas12b, Bacillus thermoamylovorans Cas12b, Bacillus sp. V3-13 Cas12b, or Alicyclobacillus acidiphilus Cas12b. In embodiments, the Cas12 polypeptide contains BvCas12b (V4), which in some embodiments is expressed as 5′ mRNA Cap-5′ UTR-bhCas12b-STOP sequence-3′ UTR-120polyA tail (SEQ ID NOs: 258-260).

In other embodiments, the catalytic domain is inserted between amino acid positions 153-154, 255-256, 306-307, 980-981, 1019-1020, 534-535, 604-605, or 344-345 of BhCas12b or a corresponding amino acid residue of Cas12a, Cas12c, Cas12d, Cas12e, Cas12g, Cas12h, Cas12i, or Cas12j/CasΦ. In other embodiments, the catalytic domain is inserted between amino acids P153 and S154 of BhCas12b. In other embodiments, the catalytic domain is inserted between amino acids K255 and E256 of BhCas12b. In other embodiments, the catalytic domain is inserted between amino acids D980 and G981 of BhCas12b. In other embodiments, the catalytic domain is inserted between amino acids K1019 and L1020 of BhCas12b. In other embodiments, the catalytic domain is inserted between amino acids F534 and P535 of BhCas12b. In other embodiments, the catalytic domain is inserted between amino acids K604 and G605 of BhCas12b. In other embodiments, the catalytic domain is inserted between amino acids H344 and F345 of BhCas12b. In other embodiments, catalytic domain is inserted between amino acid positions 147 and 148, 248 and 249, 299 and 300, 991 and 992, or 1031 and 1032 of BvCas12b or a corresponding amino acid residue of Cas12a, Cas12c, Cas12d, Cas12e, Cas12g, Cas12h, Cas12i, or Cas12j/CasΦ. In other embodiments, the catalytic domain is inserted between amino acids P147 and D148 of BvCas12b. In other embodiments, the catalytic domain is inserted between amino acids G248 and G249 of BvCas12b. In other embodiments, the catalytic domain is inserted between amino acids P299 and E300 of BvCas12b. In other embodiments, the catalytic domain is inserted between amino acids G991 and E992 of BvCas12b. In other embodiments, the catalytic domain is inserted between amino acids K1031 and M1032 of BvCas12b. In other embodiments, the catalytic domain is inserted between amino acid positions 157 and 158, 258 and 259, 310 and 311, 1008 and 1009, or 1044 and 1045 of AaCas12b or a corresponding amino acid residue of Cas12a, Cas12c, Cas12d, Cas12e, Cas12g, Cas12h, Cas12i, or Cas12j/CasΦ. In other embodiments, the catalytic domain is inserted between amino acids P157 and G158 of AaCas12b. In other embodiments, the catalytic domain is inserted between amino acids V258 and G259 of AaCas12b. In other embodiments, the catalytic domain is inserted between amino acids D310 and P311 of AaCas12b. In other embodiments, the catalytic domain is inserted between amino acids G1008 and E1009 of AaCas12b. In other embodiments, the catalytic domain is inserted between amino acids G1044 and K1045 at of AaCas12b.

In other embodiments, the fusion protein contains a nuclear localization signal (e.g., a bipartite nuclear localization signal). In other embodiments, the amino acid sequence of the nuclear localization signal is MAPKKKRKVGIHGVPAA (SEQ ID NO: 261). In other embodiments of the above aspects, the nuclear localization signal is encoded by the following sequence:

ATGGCCCCAAAGAAGAAGCGGAAGGTCGGTATCCACGGAGTCCCAGCAGCC (SEQ ID NO: 262). In other embodiments, the Cas12b polypeptide contains a mutation that silences the catalytic activity of a RuvC domain. In other embodiments, the Cas12b polypeptide contains D574A, D829A and/or D952A mutations. In other embodiments, the fusion protein further contains a tag (e.g., an influenza hemagglutinin tag).

In some embodiments, the fusion protein comprises a napDNAbp domain (e.g., Cas12-derived domain) with an internally fused nucleobase editing domain (e.g., all or a portion of a deaminase domain, e.g., an adenosine deaminase domain). In some embodiments, the napDNAbp is a Cas12b. In some embodiments, the base editor comprises a BhCas12b domain with an internally fused TadA*8 domain inserted at the loci provided in Table 7 below.

TABLE 7
Insertion loci in Cas 12b proteins

| | Insertion site | Inserted between aa |
|---|---|---|
| BhCas12b | | |
| position 1 | 153 | PS |
| position 2 | 255 | KE |
| position 3 | 306 | DE |
| position 4 | 980 | DG |
| position 5 | 1019 | KL |
| position 6 | 534 | FP |
| position 7 | 604 | KG |
| position 8 | 344 | HF |
| BvCas12b | | |
| position 1 | 147 | PD |
| position 2 | 248 | GG |
| position 3 | 299 | PE |
| position 4 | 991 | GE |
| position 5 | 1031 | KM |
| AaCas12b | | |
| position 1 | 157 | PG |
| position 2 | 258 | VG |
| position 3 | 310 | DP |
| position 4 | 1008 | GE |
| position 5 | 1044 | GK |

By way of nonlimiting example, an adenosine deaminase (e.g., TadA*8.13) may be inserted into a BhCas12b to produce a fusion protein (e.g., TadA*8.13-BhCas12b) that effectively edits a nucleic acid sequence.

Exemplary, yet nonlimiting, fusion proteins are described in International PCT Application Nos. PCT/US2020/016285, PCT/US2020/018073, PCT/US2020/018107, PCT/US2020/018124, PCT/US2020/018132, PCT/US2020/018169, PCT/US2020/018178, PCT/US2020/018192, PCT/US2020/018193, and PCT/US2020/018195, the contents of which are incorporated by reference herein in their entireties.

### A to G Editing

In some embodiments, a base editor described herein comprises an adenosine deaminase domain. Such an adenosine deaminase domain of a base editor can facilitate the editing of an adenine (A) nucleobase to a guanine (G) nucleobase by deaminating the A to form inosine (I), which exhibits base pairing properties of G. Adenosine deaminase is capable of deaminating (i.e., removing an amine group) adenine of a deoxyadenosine residue in deoxyribonucleic acid (DNA). In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

A base editor comprising an adenosine deaminase can act on any polynucleotide, including DNA, RNA and DNA-RNA hybrids. In certain embodiments, a base editor comprising an adenosine deaminase can deaminate a target A of a polynucleotide comprising RNA. For example, the base editor can comprise an adenosine deaminase domain capable of deaminating a target A of an RNA polynucleotide and/or a DNA-RNA hybrid polynucleotide. In an embodiment, an adenosine deaminase incorporated into a base editor comprises all or a portion of adenosine deaminase acting on RNA (ADAR, e.g., ADAR1 or ADAR2) or tRNA (ADAT). A base editor comprising an adenosine deaminase domain can also be capable of deaminating an A nucleobase of a DNA polynucleotide. In an embodiment an adenosine deaminase domain of a base editor comprises all or a portion of an ADAT comprising one or more mutations which permit the ADAT to deaminate a target A in DNA. For example, the base editor can comprise all or a portion of an ADAT from Escherichia coli (EcTadA) comprising one or more of the following mutations: D108N, A106V, D147Y, E155V, L84F, H123Y, I156F, or a corresponding mutation in another adenosine deaminase. Exemplary ADAT homolog polypeptide sequences are provided in the Sequence Listing as SEQ ID NOs: 1 and 309-315.

In some embodiments, a base editor described herein comprises a fusion protein comprising an adenosine deaminase domain (e.g., adenosine deaminase variant domain). In some embodiments, an adenosine deaminase variant domain contains a combination of alterations in a TadA*7.10 amino acid sequence, where the combinations are V82G, Y147T/D, Q154S, and one or more of L36H, I76Y, F149Y, N157K, and D167N. In some embodiments, the combinations of alterations in a TadA*7.10 amino acid sequence are V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; or L36H+I76Y+V82G+Y147D+F149Y+Q154S+N157K+D167N or a corresponding alteration in another adenosine deaminase. Such an adenosine deaminase domain of a base editor can facilitate the editing of an adenine (A) nucleobase to a guanine (G) nucleobase by deaminating the A to form inosine (I), which exhibits base pairing properties of G. Adenosine deaminase is capable of deaminating (i.e., removing an amine group) adenine of a deoxyadenosine residue in deoxyribonucleic acid (DNA).

In some embodiments, the nucleobase editors provided herein can be made by fusing together one or more protein domains, thereby generating a fusion protein. In certain embodiments, the fusion proteins provided herein comprise one or more features that improve the base editing activity (e.g., efficiency, selectivity, and specificity) of the fusion proteins. For example, the fusion proteins provided herein can comprise a Cas9 domain that has reduced nuclease activity. In some embodiments, the fusion proteins provided herein can have a Cas9 domain that does not have nuclease activity (dCas9), or a Cas9 domain that cuts one strand of a duplexed DNA molecule, referred to as a Cas9 nickase (nCas9). Without wishing to be bound by any particular theory, the presence of the catalytic residue (e.g., H840) maintains the activity of the Cas9 to cleave the non-edited (e.g., non-deaminated) strand containing a T opposite the targeted A. Mutation of the catalytic residue (e.g., D10 to A10) of Cas9 prevents cleavage of the edited strand containing the targeted A residue. Such Cas9 variants are able to generate a single-strand DNA break (nick) at a specific location based on the gRNA-defined target sequence, leading to repair of the non-edited strand, ultimately resulting in a T to C change on the non-edited strand. In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

A base editor comprising an adenosine deaminase can act on any polynucleotide, including DNA, RNA and DNA-RNA hybrids. In certain embodiments, a base editor comprising an adenosine deaminase can deaminate a target A of a polynucleotide comprising RNA. For example, the base editor can comprise an adenosine deaminase domain capable of deaminating a target A of an RNA polynucleotide and/or a DNA-RNA hybrid polynucleotide. In an embodiment, an adenosine deaminase incorporated into a base editor comprises all or a portion of adenosine deaminase acting on RNA (ADAR, e.g., ADAR1 or ADAR2). In another embodiment, an adenosine deaminase incorporated into a base editor comprises all or a portion of adenosine deaminase acting on tRNA (ADAT). A base editor comprising an adenosine deaminase domain can also be capable of deaminating an A nucleobase of a DNA polynucleotide. In an embodiment an adenosine deaminase domain of a base editor comprises all or a portion of an ADAT comprising one or more mutations which permit the ADAT to deaminate a target A in DNA. For example, the base editor can comprise all or a portion of an ADAT from Escherichia coli (EcTadA) comprising one or more of the following mutations: D108N, A106V, D147Y, E155V, L84F, H123Y, I156F, or a corresponding mutation in another adenosine deaminase.

The adenosine deaminase can be derived from any suitable organism (e.g., E. coli). In some embodiments, the adenosine deaminase is from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that correspond to any of the mutations described herein (e.g., any of the mutations identified in ecTadA) can be generated accordingly.

### Adenosine Deaminases

In some embodiments, the fusion proteins as described herein comprise one or more adenosine deaminase domains. In some embodiments, the adenosine deaminases provided herein are capable of deaminating adenine. In some embodiments, the adenosine deaminases provided herein are capable of deaminating adenine in a deoxyadenosine residue of DNA. The adenosine deaminase may be derived from any suitable organism (e.g., E. coli). In some embodiments, the adenine deaminase is a naturally-occurring adenosine deaminase that includes one or more mutations corresponding to any of the mutations provided herein (e.g., mutations in ecTadA). One of skill in the art will be able to identify the corresponding residue in any homologous protein, e.g., by sequence alignment and determination of homologous residues. Accordingly, one of skill in the art would be able to generate mutations in any naturally-occurring adenosine deaminase (e.g., having homology to ecTadA) that corresponds to any of the mutations described herein, e.g., any of the mutations identified in ecTadA. In some embodiments, the adenosine deaminase is from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli.

Provided and described herein are adenosine deaminase variants that have increased efficiency (>50-60%) and specificity. In particular, the adenosine deaminase variants described herein are more likely to edit a desired base within a polynucleotide, and are less likely to edit bases that are not intended to be altered (i.e., “bystanders”).

In some embodiments, the adenosine deaminase is a TadA deaminase. In particular embodiments, the TadA is any one of the TadA described in PCT/US2017/045381 (WO 2018/027078), which is incorporated herein by reference in its entirety.

A wild type TadA(wt) adenosine deaminase has the following sequence (also termed TadA reference sequence):

(SEQ ID NO: 391)
MSEVEFSHEYWMRHALTLAKRAWDEREVPVGAVLVHNNRVIGEGWNRPIG
RHDPTAHAEIMALRQGGLVMQNYRLIDATLYVTLEPCVMCAGAMIHSRIG
RVVFGARDAKTGAAGSLMDVLHHPGMNHRVEITEGILADECAALLSDFFR
MRRQEIKAQKKAQSSTD

In some embodiments the adenosine deaminase is a full-length E. coli TadA deaminase. For example, in certain embodiments, the adenosine deaminase comprises the amino acid sequence:

(SEQ ID NO: 392)
MRRAFITGVFFLSEVEFSHEYWMRHALTLAKRAWDEREVPVGAVLVHNNR
VIGEGWNRPIGRHDPTAHAEIMALRQGGLVMQNYRLIDATLYVTLEPCVM
CAGAMIHSRIGRVVFGARDAKTGAAGSLMDVLHHPGMNHRVEITEGILAD
ECAALLSDFFRMRRQEIKAQKKAQSSTD.

In some embodiments, the adenosine deaminase is from a prokaryote. In some embodiments, the adenosine deaminase is from a bacterium. In some embodiments, the adenosine deaminase is from Escherichia coli (E. coli), Staphylococcus aureus (S. aureus), Salmonella typhimurium (S. typhimurium), Shewanella putrefaciens (S. putrefaciens), Haemophilus influenzae (H. influenzae), Caulobacter crescentus (C. crescentus), Geobacter sulfurreducens (G. sulfurreducens), or Bacillus subtilis. In some embodiments, the adenosine deaminase is from E. coli.

It should be appreciated, however, that additional adenosine deaminases useful in the present application would be apparent to the skilled artisan and are within the scope of this disclosure. For example, the adenosine deaminase may be a homolog of adenosine deaminase acting on tRNA (ADAT). Without limitation, the amino acid sequences of exemplary AD AT homologs include the following:

Staphylococcus aureus (S. aureus) TadA:
(SEQ ID NO: 309)
MGSHMTNDIYFMTLAIEEAKKAAQLGEVPIGAIITKDDEVIARAHNLRET
LQQPTAHAEHIAIERAAKVLGSWRLEGCTLYVTLEPCVMCAGTIVMSRIP
RVVYGADDPKGGCSGSLMNLLQQSNFNHRAIVDKGVLKEACSTLLTTFFK
NLRANKKSTN
Bacillus subtilis (B. subtilis) TadA:
(SEQ ID NO: 310)
MTQDELYMKEAIKEAKKAEEKGEVPIGAVLVINGEIIARAHNLRETEQRS
IAHAEMLVIDEACKALGTWRLEGATLYVTLEPCPMCAGAVVLSRVEKVVF
GAFDPKGGCSGTLMNLLQEERFNHQAEVVSGVLEEECGGMLSAFFRELRK
KKKAARKNLSE
Salmonella typhimurium (S. typhimurium) TadA:
(SEQ ID NO: 311)
MPPAFITGVTSLSDVELDHEYWMRHALTLAKRAWDEREVPVGAVLVHNHR
VIGEGWNRPIGRHDPTAHAEIMALRQGGLVLQNYRLLDTTLYVTLEPCVM
CAGAMVHSRIGRVVFGARDAKTGAAGSLIDVLHHPGMNHRVEIIEGVLRD
ECATLLSDFFRMRRQEIKALKKADRAEGAGPAV
Shewanella putrefaciens (S. putrefaciens) TadA:
(SEQ ID NO: 312)
MDEYWMQVAMQMAEKAEAAGEVPVGAVLVKDGQQIATGYNLSISQHDPTA
HAEILCLRSAGKKLENYRLLDATLYITLEPCAMCAGAMVHSRIARVVYGA
RDEKTGAAGTVVNLLQHPAFNHQVEVTSGVLAEACSAQLSRFFKRRRDEK
KALKLAQRAQQGIE
Haemophilus influenzae F3031 (H. influenzae) TadA:
(SEQ ID NO: 313)
MDAAKVRSEFDEKMMRYALELADKAEALGEIPVGAVLVDDARNIIGEGWN
LSIVQSDPTAHAEIIALRNGAKNIQNYRLLNSTLYVTLEPCTMCAGAILH
SRIKRLVFGASDYKTGAIGSRFHFFDDYKMNHTLEITSGVLAEECSQKLS
TFFQKRREEKKIEKALLKSLSDK
Caulobacter crescentus (C. crescentus) TadA:
(SEQ ID NO: 314)
MRTDESEDQDHRMMRLALDAARAAAEAGETPVGAVILDPSTGEVIATAGN
GPIAAHDPTAHAEIAAMRAAAAKLGNYRLTDLTLVVTLEPCAMCAGAISH
ARIGRVVFGADDPKGGAVVHGPKFFAQPTCHWRPEVTGGVLADESADLLR
GFFRARRKAKI
Geobacter sulfurreducens (G. sulfurreducens) TadA:
(SEQ ID NO: 315)
MSSLKKTPIRDDAYWMGKAIREAAKAAARDEVPIGAVIVRDGAVIGRGHN
LREGSNDPSAHAEMIAIRQAARRSANWRLTGATLYVTLEPCLMCMGAIIL
ARLERVVFGCYDPKGGAAGSLYDLSADPRLNHQVRLSPGVCQEECGTMLS
DFFRDLRRRKKAKATPALFIDERKVPPEP
An embodiment of E. Coli TadA (ecTadA) includes
the following:
(SEQ ID NO: 1)
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCYFFR
MPRQVFNAQKKAQSSTD

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identity plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences known in the art or described herein.

It should be appreciated that any of the mutations provided herein (e.g., based on the TadA reference sequence) can be introduced into other adenosine deaminases, such as E. coli TadA (ecTadA), S. aureus TadA (saTadA), or other adenosine deaminases (e.g., bacterial adenosine deaminases). It would be apparent to the skilled artisan that additional deaminases may similarly be aligned to identify homologous amino acid residues that can be mutated as provided herein. Thus, any of the mutations identified in the TadA reference sequence can be made in other adenosine deaminases (e.g., ecTada) that have homologous amino acid residues. It should also be appreciated that any of the mutations provided herein can be made individually or in any combination in the TadA reference sequence or another adenosine deaminase.

In some embodiments, the adenosine deaminase comprises a D108X mutation in the TadA reference sequence, or a corresponding mutation in another adenosine deaminase, where X indicates any amino acid other than the corresponding amino acid in the wild-type adenosine deaminase. In some embodiments, the adenosine deaminase comprises a D108G, D108N, D108V, D108A, or D108Y mutation in TadA reference sequence, or a corresponding mutation in another adenosine deaminase. It should be appreciated, however, that additional deaminases may similarly be aligned to identify homologous amino acid residues that can be mutated as provided herein.

In some embodiments, the adenosine deaminase comprises an A106X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an A106V mutation in TadA reference sequence, or a corresponding mutation in another adenosine deaminase (e.g., ecTadA).

In some embodiments, the adenosine deaminase comprises a E155X mutation in TadA reference sequence, or a corresponding mutation in another adenosine deaminase, where the presence of X indicates any amino acid other than the corresponding amino acid in the wild-type adenosine deaminase. In some embodiments, the adenosine deaminase comprises a E155D, E155G, or E155V mutation in TadA reference sequence, or a corresponding mutation in another adenosine deaminase (e.g., ecTadA).

In some embodiments, the adenosine deaminase comprises a D147X mutation in TadA reference sequence, or a corresponding mutation in another adenosine deaminase, where the presence of X indicates any amino acid other than the corresponding amino acid in the wild-type adenosine deaminase. In some embodiments, the adenosine deaminase comprises a D147Y, mutation in TadA reference sequence, or a corresponding mutation in another adenosine deaminase (e.g., ecTadA).

In some embodiments, the adenosine deaminase comprises an A106X, E155X, or D147X, mutation in the TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises an E155D, E155G, or E155V mutation. In some embodiments, the adenosine deaminase comprises a D147Y.

It should be appreciated that any of the mutations provided herein (e.g., based on the ecTadA amino acid sequence of TadA reference sequence) may be introduced into other adenosine deaminases, such as S. aureus TadA (saTadA), or other adenosine deaminases (e.g., bacterial adenosine deaminases). It would be apparent to the skilled artisan how to are homologous to the mutated residues in ecTadA. Thus, any of the mutations identified in ecTadA may be made in other adenosine deaminases that have homologous amino acid residues. It should also be appreciated that any of the mutations provided herein may be made individually or in any combination in ecTadA or another adenosine deaminase.

For example, an adenosine deaminase contains a combination of mutations (e.g., V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; or L36H+I76Y+V82G+Y147D+F149Y+Q154S+N157K+D167N), and may contain one or more additional mutations. Additional mutations include, for example, a D108N, a A106V, a E155V, and/or a D147Y mutation in TadA reference sequence, or a corresponding mutation in another adenosine deaminase (e.g., ecTadA). In some embodiments, an adenosine deaminase comprises the following group of mutations (groups of mutations are separated by a “;”) in TadA reference sequence, or corresponding mutations in another adenosine deaminase: D108N and A106V; D108N and E155V; D108N and D147Y; A106V and E155V; A106V and D147Y; E155V and D147Y; D108N, A106V, and E155V; D108N, A106V, and D147Y; D108N, E155V, and D147Y; A106V, E155V, and D147Y; and D108N, A106V, E155V, and D147Y. It should be appreciated, however, that any combination of corresponding mutations provided herein may be made in an adenosine deaminase (e.g., ecTadA).

In some embodiments, the adenosine deaminase comprises one or more of a H8X, T17X, L18X, W23X, L34X, W45X, R51X, A56X, E59X, E85X, M94X, I95X, V102X, F104X, A106X, R107X, D108X, K110X, M118X, N127X, A138X, F149X, M151X, R153X, Q154X, I156X, and/or K157X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of H8Y, T17S, L18E, W23L, L34S, W45L, R51H, A56E, or A56S, E59G, E85K, or E85G, M94L, I95L, V102A, F104L, A106V, R107C, or R107H, or R107P, D108G, or D108N, or D108V, or D108A, or D108Y, K110I, M118K, N127S, A138V, F149Y, M151V, R153C, Q154L, I156D, and/or K157R mutation in TadA reference sequence, or one or more corresponding mutations in another adenosine deaminase.

In some embodiments, the adenosine deaminase comprises one or more of a H8X, D108X, and/or N127X mutation in TadA reference sequence, or one or more corresponding mutations in another adenosine deaminase, where X indicates the presence of any amino acid. In some embodiments, the adenosine deaminase comprises one or more of a H8Y, D108N, and/or N127S mutation in TadA reference sequence, or one or more corresponding mutations in another adenosine deaminase.

In some embodiments, the adenosine deaminase comprises one or more of H8X, R26X, M61X, L68X, M70X, A106X, D108X, A109X, N127X, D147X, R152X, Q154X, E155X, K161X, Q163X, and/or T166X mutation in TadA reference sequence, or one or more corresponding mutations in another adenosine deaminase, where X indicates the presence of any amino acid other than the corresponding amino acid in the wild-type adenosine deaminase. In some embodiments, the adenosine deaminase comprises one or more of H8Y, R26W, M61I, L68Q, M70V, A106T, D108N, A109T, N127S, D147Y, R152C, Q154H or Q154R, E155G or E155V or E155D, K161Q, Q163H, and/or T166P mutation in TadA reference sequence, or one or more corresponding mutations in another adenosine deaminase.

In some embodiments, the adenosine deaminase comprises one, two, three, four, five, or six mutations selected from the group consisting of H8X, D108X, N127X, D147X, R152X, and Q154X in TadA reference sequence, or a corresponding mutation or mutations in another adenosine deaminase (e.g., ecTadA), where X indicates the presence of any amino acid other than the corresponding amino acid in the wild-type adenosine deaminase. In some embodiments, the adenosine deaminase comprises one, two, three, four, five, six, seven, or eight mutations selected from the group consisting of H8X, M61X, M70X, D108X, N127X, Q154X, E155X, and Q163X in TadA reference sequence, or a corresponding mutation or mutations in another adenosine deaminase (e.g., ecTadA), where X indicates the presence of any amino acid other than the corresponding amino acid in the wild-type adenosine deaminase. In some embodiments, the adenosine deaminase comprises one, two, three, four, or five, mutations selected from the group consisting of H8X, D108X, N127X, E155X, and T166X in TadA reference sequence, or a corresponding mutation or mutations in another adenosine deaminase (e.g., ecTadA), where X indicates the presence of any amino acid other than the corresponding amino acid in the wild-type adenosine deaminase. In some embodiments, the adenosine deaminase comprises one, two, three, four, five, or six mutations selected from the group consisting of H8X, A106X, and D108X, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, five, six, seven, or eight mutations selected from the group consisting of H8X, R26X, L68X, D108X, N127X, D147X, and E155X, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, six, or seven mutations selected from the group consisting of H8X, R126X, L68X, D108X, N127X, D147X, and E155X in TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, or five mutations selected from the group consisting of H8X, D108X, A109X, N127X, and E155X in TadA reference sequence, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, or six mutations selected from the group consisting of H8Y, D108N, N127S, D147Y, R152C, and Q154H in TadA reference sequence, or a corresponding mutation or mutations in another adenosine deaminase (e.g., ecTadA). In some embodiments, the adenosine deaminase comprises one, two, three, four, five, six, seven, or eight mutations selected from the group consisting of H8Y, M61I, M70V, D108N, N127S, Q154R, E155G and Q163H in TadA reference sequence, or a corresponding mutation or mutations in another adenosine deaminase (e.g., ecTadA). In some embodiments, the adenosine deaminase comprises one, two, three, four, or five, mutations selected from the group consisting of H8Y, D108N, N127S, E155V, and T166P in TadA reference sequence, or a corresponding mutation or mutations in another adenosine deaminase (e.g., ecTadA). In some embodiments, the adenosine deaminase comprises one, two, three, four, five, or six mutations selected from the group consisting of H8Y, A106T, D108N, N127S, E155D, and K161Q in TadA reference sequence, or a corresponding mutation or mutations in another adenosine deaminase (e.g., ecTadA). In some embodiments, the adenosine deaminase comprises one, two, three, four, five, six, seven, or eight mutations selected from the group consisting of H8Y, R26W, L68Q, D108N, N127S, D147Y, and E155V in TadA reference sequence, or a corresponding mutation or mutations in another adenosine deaminase (e.g., ecTadA). In some embodiments, the adenosine deaminase comprises one, two, three, four, or five, mutations selected from the group consisting of H8Y, D108N, A109T, N127S, and E155G in TadA reference sequence, or a corresponding mutation or mutations in another adenosine deaminase (e.g., ecTadA).

In some embodiments, the adenosine deaminase comprises one or more of the or one or more corresponding mutations in another adenosine deaminase. In some embodiments, the adenosine deaminase comprises a D108N, D108G, or D108V mutation in TadA reference sequence, or corresponding mutations in another adenosine deaminase. In some embodiments, the adenosine deaminase comprises a A106V and D108N mutation in TadA reference sequence, or corresponding mutations in another adenosine deaminase. In some embodiments, the adenosine deaminase comprises R107C and D108N mutations in TadA reference sequence, or corresponding mutations in another adenosine deaminase. In some embodiments, the adenosine deaminase comprises a H8Y, D108N, N127S, D147Y, and Q154H mutation in TadA reference sequence, or corresponding mutations in another adenosine deaminase. In some embodiments, the adenosine deaminase comprises a H8Y, D108N, N127S, D147Y, and E155V mutation in TadA reference sequence, or corresponding mutations in another adenosine deaminase. In some embodiments, the adenosine deaminase comprises a D108N, D147Y, and E155V mutation in TadA reference sequence, or corresponding mutations in another adenosine deaminase. In some embodiments, the adenosine deaminase comprises a H8Y, D108N, and N127S mutation in TadA reference sequence, or corresponding mutations in another adenosine deaminase. In some embodiments, the adenosine deaminase comprises a A106V, D108N, D147Y, and E155V mutation in TadA reference sequence, or corresponding mutations in another adenosine deaminase (e.g., ecTadA).

In some embodiments, the adenosine deaminase comprises one or more of S2X, H8X, I49X, L84X, H123X, N127X, I156X, and/or K160X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of S2A, H8Y, I49F, L84F, H123Y, N127S, I156F, and/or K160S mutation in TadA reference sequence, or one or more corresponding mutations in another adenosine deaminase (e.g., ecTadA).

In some embodiments, the adenosine deaminase comprises an L84X mutation adenosine deaminase, where X indicates any amino acid other than the corresponding amino acid in the wild-type adenosine deaminase. In some embodiments, the adenosine deaminase comprises an L84F mutation in TadA reference sequence, or a corresponding mutation in another adenosine deaminase (e.g., ecTadA).

In some embodiments, the adenosine deaminase comprises an H123X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an H123Y mutation in TadA reference sequence, or a corresponding mutation in another adenosine deaminase.

In some embodiments, the adenosine deaminase comprises an I156X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an I156F mutation in TadA reference sequence, or a corresponding mutation in another adenosine deaminase.

In some embodiments, the adenosine deaminase comprises one, two, three, four, five, six, or seven mutations selected from the group consisting of L84X, A106X, D108X, H123X, D147X, E155X, and I156X in TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, five, or six mutations selected from the group consisting of S2X, I49X, A106X, D108X, D147X, and E155X in TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, or five mutations selected from the group consisting of H8X, A106X, D108X, N127X, and K160X in TadA reference sequence, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, six, or seven mutations selected from the group consisting of L84F, A106V, D108N, H123Y, D147Y, E155V, and I156F in TadA reference sequence, or a corresponding mutation or mutations in another adenosine deaminase. In some embodiments, the adenosine deaminase comprises one, two, three, four, five, or six mutations selected from the group consisting of S2A, I49F, A106V, D108N, D147Y, and E155V in TadA reference sequence.

In some embodiments, the adenosine deaminase comprises one, two, three, four, or five mutations selected from the group consisting of H8Y, A106T, D108N, N127S, and K160S in TadA reference sequence, or a corresponding mutation or mutations in another adenosine deaminase.

In some embodiments, the adenosine deaminase comprises one or more of a E25X, R26X, R107X, A142X, and/or A143X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of E25M, E25D, E25A, E25R, E25V, E25S, E25Y, R26G, R26N, R26Q, R26C, R26L, R26K, R107P, R107K, R107A, R107N, R107W, R107H, R107S, A142N, A142D, A142G, A143D, A143G, A143E, A143L, A143W, A143M, A143S, A143Q, and/or A143R mutation in TadA reference sequence, or one or more corresponding mutations in another adenosine deaminase. In some embodiments, the adenosine deaminase comprises one or more of the mutations described herein corresponding to TadA reference sequence, or one or more corresponding mutations in another adenosine deaminase.

In some embodiments, the adenosine deaminase comprises an E25X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an E25M, E25D, E25A, E25R, E25V, E25S, or E25Y mutation in TadA reference sequence, or a corresponding mutation in another adenosine deaminase (e.g., ecTadA).

In some embodiments, the adenosine deaminase comprises an R26X mutation in TadA reference sequence, or a corresponding mutation in another adenosine deaminase, where X indicates any amino acid other than the corresponding amino acid in the wild-type adenosine deaminase. In some embodiments, the adenosine deaminase comprises R26G, R26N, R26Q, R26C, R26L, or R26K mutation in TadA reference sequence, or a corresponding mutation in another adenosine deaminase (e.g., ecTadA).

In some embodiments, the adenosine deaminase comprises an R107X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an R107P, R107K, R107A, R107N, R107W, R107H, or R107S mutation in TadA reference sequence, or a corresponding mutation in another adenosine deaminase (e.g., ecTadA).

In some embodiments, the adenosine deaminase comprises an A142X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an A142N, A142D, A142G, mutation in TadA reference sequence, or a corresponding mutation in another adenosine deaminase (e.g., ecTadA).

In some embodiments, the adenosine deaminase comprises an A143X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an A143D, A143G, A143E, A143L, A143W, A143M, A143S, A143Q, and/or A143R mutation in TadA reference sequence, or a corresponding mutation in another adenosine deaminase (e.g., ecTadA).

In some embodiments, the adenosine deaminase comprises one or more of a H36X, N37X, P48X, I49X, R51X, M70X, N72X, D77X, E134X, S146X, Q154X, K157X, and/or K161X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of H36L, N37T, N37S, P48T, P48L, I49V, R51H, R51L, M70L, N72S, D77G, E134G, S146R, S146C, Q154H, K157N, and/or K161T mutation in TadA reference sequence, or one or more corresponding mutations in another adenosine deaminase (e.g., ecTadA).

In some embodiments, the adenosine deaminase comprises an H36X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an H36L mutation in TadA reference sequence, or a corresponding mutation in another adenosine deaminase.

In some embodiments, the adenosine deaminase comprises an N37X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an N37T or N37S mutation in TadA reference sequence, or a corresponding mutation in another adenosine deaminase.

In some embodiments, the adenosine deaminase comprises an P48X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an P48T or P48L mutation in TadA reference sequence, or a corresponding mutation in another adenosine deaminase.

In some embodiments, the adenosine deaminase comprises an R51X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an R51H or R51L mutation in TadA reference sequence, or a corresponding mutation in another adenosine deaminase.

In some embodiments, the adenosine deaminase comprises an S146X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an S146R or S146C mutation in TadA reference sequence, or a corresponding mutation in another adenosine deaminase.

In some embodiments, the adenosine deaminase comprises an K157X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a K157N mutation in TadA reference sequence, or a corresponding mutation in another adenosine deaminase.

In some embodiments, the adenosine deaminase comprises an P48X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a P48S, P48T, or P48A mutation in TadA reference sequence, or a corresponding mutation in another adenosine deaminase.

In some embodiments, the adenosine deaminase comprises an A142X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a A142N mutation in TadA reference sequence, or a corresponding mutation in another adenosine deaminase.

In some embodiments, the adenosine deaminase comprises an W23X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a W23R or W23L mutation in TadA reference sequence, or a corresponding mutation in another adenosine deaminase.

In some embodiments, the adenosine deaminase comprises an R152X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a R152P or R52H mutation in TadA reference sequence, or a corresponding mutation in another adenosine deaminase.

In one embodiment, the adenosine deaminase may comprise the mutations H36L, R51L, L84F, A106V, D108N, H123Y, S146C, D147Y, E155V, I156F, and K157N. In some embodiments, the adenosine deaminase comprises the following combination of mutations relative to TadA reference sequence, where each mutation of a combination is separated by a “_” and each combination of mutations is between parentheses:
(A106V_D108N),
(R107C_D108N),
(H8Y_D108N_N127S_D147Y_Q154H),
(H8Y_D108N_N127S_D147Y_E155V),
(D108N_D147Y_E155V),
(H8Y_D108N_N127S),
(H8Y_D108N_N127S_D147Y_Q154H),
(A106V_D108N_D147Y_E155V),
(D108Q_D147Y_E155V),
(D108M_D147Y_E155V),
(D108L_D147Y_E155V),
(D108K_D147Y_E155V),
(D108I_D147Y_E155V),
(D108F_D147Y_E155V),
(A106V_D108N_D147Y),
(A106V_D108M_D147Y_E155V),
(E59A_A106V_D108N_D147Y_E155V),
(E59A cat dead_A106V_D108N_D147Y_E155V),
(L84F_A106V_D108N_H123Y_D147Y_E155V_I156Y),
(L84F_A106V_D108N_H123Y_D147Y_E155V_I156F),
(D103A_D104N),
(G22P_D103A_D104N),
(D103A_D104N_S138A),
(R26G_L84F_A106V_R107H_D108N_H123Y_A142N_A143D_D147Y_E155V_I156F),
(E25G_R26G_L84F_A106V_R107H_D108N_H123Y_A142N_A143D_D147Y_E155V_I156F),
(E25D_R26G_L84F_A106V_R107K_D108N_H123Y_A142N_A143G_D147Y_E155V_I156F),
(R26Q_L84F_A106V_D108N_H123Y_A142N_D147Y_E155V_I156F),
(E25M_R26G_L84F_A106V_R107P_D108N_H123Y_A142N_A143D_D147Y_E155V_I156F),
(R26C_L84F_A106V_R107H_D108N_H123Y_A142N_D147Y_E155V_I156F),
(L84F_A106V_D108N_H123Y_A142N_A143L_D147Y_E155V_I156F),
(R26G_L84F_A106V_D108N_H123Y_A142N_D147Y_E155V_I156F),
(E25A_R26G_L84F_A106V_R107N_D108N_H123Y_A142N_A143E_D147Y_E155V_I156F),
(R26G_L84F_A106V_R107H_D108N_H123Y_A142N_A143D_D147Y_E155V_I156F),
(A106V_D108N_A142N_D147Y_E155V),
(R26G_A106V_D108N_A142N_D147Y_E155V),
(E25D_R26G_A106V_R107K_D108N_A142N_A143G_D147Y_E155V),
(R26G_A106V_D108N_R107H_A142N_A143D_D147Y_E155V),
(E25D_R26G_A106V_D108N_A142N_D147Y_E155V),
(A106V_R107K_D108N_A142N_D147Y_E155V),
(A106V_D108N_A142N_A143G_D147Y_E155V),
(A106V_D108N_A142N_A143L_D147Y_E155V),
(H36L_R51L_L84F_A106V_D108N_H123Y_S146C_D147Y_E155V_I156F_K157N),
(N37T_P48T_M70L_L84F_A106V_D108N_H123Y_D147Y_149V_E155V_I156F),
(N37S_L84F_A106V_D108N_H123Y_D147Y_E155V_I156F_K161T),
(H36L_L84F_A106V_D108N_H123Y_D147Y_Q154H_E155V_I156F),
(N72S_L84F_A106V_D108N_H123Y_S146R_D147Y_E155V_I156F),
(H36L_P48L_L84F_A106V_D108N_H123Y_E134G_D147Y_E155V_I156F),
(H36L_L84F_A106V_D108N_H123Y_D147Y_E155V_I156F_K157N)
(H36L_L84F_A106V_D108N_H123Y_S146C_D147Y_E155V_I156F),
(L84F_A106V_D108N_H123Y_S146R_D147Y_E155V_I156F_K161T),
(N37S_R51H_D77G_L84F_A106V_D108N_H123Y_D147Y_E155V_I156F),
(R51L_L84F_A106V_D108N_H123Y_D147Y_E155V_I156F_K157N),
(D24G_Q71R_L84F_H96L_A106V_D108N_H123Y_D147Y_E155V_I156F_K160E),
(H36L_G67V_L84F_A106V_D108N_H123Y_S146T_D147Y_E155V_I156F),
(Q71L_L84F_A106V_D108N_H123Y_L137M_A143E_D147Y_E155V_I156F),
(E25G_L84F_A106V_D108N_H123Y_D147Y_E155V_I156F_Q159L),
(L84F_A91T_F1041_A106V_D108N_H123Y_D147Y_E155V_I156F),
(N72D_L84F_A106V_D108N_H123Y_G125A_D147Y_E155V_I156F),
(P48S_L84F_S97C_A106V_D108N_H123Y_D147Y_E155V_I156F),
(W23G_L84F_A106V_D108N_H123Y_D147Y_E155V_I156F),
(D24G_P48L_Q71R_L84F_A106V_D108N_H123Y_D147Y_E155V_I156F_Q159L),
(L84F_A106V_D108N_H123Y_A142N_D147Y_E155V_I156F),
(H36L_R51L_L84F_A106V_D108N_H123Y_A142N_S146C_D147Y_E155V_I156F_K157N),
(N37S_L84F_A106V_D108N_H123Y_A142N_D147Y_E155V_I156F_K161T),
(L84F_A106V_D108N_D147Y_E155V_I156F),
(R51L_L84F_A106V_D108N_H123Y_S146C_D147Y_E155V_I156F_K157N_K161T),
(L84F_A106V_D108N_H123Y_S146C_D147Y_E155V_I156F_K161T),
(L84F_A106V_D108N_H123Y_S146C_D147Y_E155V_I156F_K157N_K160E_K161T),
(L84F_A106V_D108N_H123Y_S146C_D147Y_E155V_I156F_K157N_K160E),
(R74Q_L84F_A106V_D108N_H123Y_D147Y_E155V_I156F),
(R74A_L84F_A106V_D108N_H123Y_D147Y_E155V_I156F),
(L84F_A106V_D108N_H123Y_D147Y_E155V_I156F),
(R74Q_L84F_A106V_D108N_H123Y_D147Y_E155V_I156F),
(L84F_R98Q_A106V_D108N_H123Y_D147Y_E155V_I156F),
(L84F_A106V_D108N_H123Y_R129Q_D147Y_E155V_I156F),
(P48S_L84F_A106V_D108N_H123Y_A142N_D147Y_E155V_I156F),
(P48S_A142N),
(P48T_I49V_L84F_A106V_D108N_H123Y_A142N_D147Y_E155V_I156F_L157N),
(P48T_I49V_A142N),
(H36L_P48S_R51L_L84F_A106V_D108N_H123Y_S146C_D147Y_E155V_I156F_K157N),
(H36L_P48S_R51L_L84F_A106V_D108N_H123Y_S146C_A142N_D147Y_E155V_I156F
(H36L_P48T_I49V_R51L_L84F_A106V_D108N_H123Y_S146C_D147Y_E155V_I156F_K157N),
(H36L_P48T_I49V_R51L_L84F_A106V_D108N_H123Y_A142N_S146C_D147Y_E155V_I156F_K157N),
(H36L_P48A_R51L_L84F_A106V_D108N_H123Y_S146C_D147Y_E155V_I156F_K157N),
(H36L_P48A_R51L_L84F_A106V_D108N_H123Y_A142N_S146C_D147Y_E155V_I156F_K157N),
(H36L_P48A_R51L_L84F_A106V_D108N_H123Y_S146C_A142N_D147Y_E155V_I156F_K157N),
(W23L_H36L_P48A_R51L_L84F_A106V_D108N_H123Y_S146C_D147Y_E155V_I156F_K157N),
(W23R_H36L_P48A_R51L_L84F_A106V_D108N_H123Y_S146C_D147Y_E155V_I156F_K157N),
(W23L_H36L_P48A_R51L_L84F_A106V_D108N_H123Y_S146R_D147Y_E155V_I156F_K161T),
(H36L_P48A_R51L_L84F_A106V_D108N_H123Y_S146C_D147Y_R152H_E155V_I156F_K157N),
(H36L_P48A_R51L_L84F_A106V_D108N_H123Y_S146C_D147Y_R152P_E155V_I156F_K157N),
(W23L_H36L_P48A_R51L_L84F_A106V_D108N_H123Y_S146C_D147Y_R152P_E155V_I156F_K157N),
(W23L_H36L_P48A_R51L_L84F_A106V_D108N_H123Y_A142A_S146C_D147Y_E155V_I156F_K157N),
(W23L_H36L_P48A_R51L_L84F_A106V_D108N_H123Y_A142A_S146C_D147Y_R152P_E155V_I156F_K157N),
(W23L_H36L_P48A_R51L_L84F_A106V_D108N_H123Y_S146R_D147Y_E155V_I156F_K161T),
(W23R_H36L_P48A_R51L_L84F_A106V_D108N_H123Y_S146C_D147Y_R152P_E155V_I156F_K157N),
(H36L_P48A_R51L_L84F_A106V_D108N_H123Y_A142N_S146C_D147Y_R152P_E155V_I156F_K157N).

In some embodiments, the TadA deaminase is TadA variant. In some embodiments, the TadA variant is TadA*7.10. In particular embodiments, the fusion proteins comprise a single TadA*7.10 domain (e.g., provided as a monomer). In other embodiments, the fusion protein comprises TadA*7.10 and TadA(wt), which are capable of forming heterodimers. In one embodiment, a fusion protein as described herein comprises a wild-type TadA linked to TadA*7.10, which is linked to Cas9 nickase.

In some embodiments, TadA*7.10 comprises at least one alteration. In some embodiments, the adenosine deaminase comprises an alteration in the following sequence:

TadA*7.10
(SEQ ID NO: 1)
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCYFFR
MPRQVFNAQKKAQSSTD

In some embodiments, TadA*7.10 comprises an alteration at amino acid 82 and/or 166. In particular embodiments, TadA*7.10 comprises one or more of the following alterations: Y147T, Y147R, Q154S, Y123H, V82S, T166R, and/or Q154R. In other embodiments, a variant of TadA*7.10 comprises a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R.

In some embodiments, a variant of TadA*7.10 comprises one or more of alterations selected from the group of L36H, I76Y, V82G, Y147T, Y147D, F149Y, Q154S, N157K, and/or D167N. In some embodiments, a variant of TadA*7.10 comprises V82G, Y147T/D, Q154S, and one or more of L36H, I76Y, F149Y, N157K, and D167N. In other embodiments, a variant of TadA*7.10 comprises a combination of alterations selected from the group of: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; L36H+I76Y+V82G+Y147D+F149Y+Q154S+N157K+D167N.

In some embodiments, an adenosine deaminase variant (e.g., TadA variant) comprises a deletion. In some embodiments, an adenosine deaminase variant comprises a deletion of the C terminus. In particular embodiments, an adenosine deaminase variant comprises a deletion of the C terminus beginning at residue 149, 150, 151, 152, 153, 154, 155, 156, and 157, relative to TadA*7.10, the TadA reference sequence, or a corresponding mutation in another TadA.

In other embodiments, an adenosine deaminase variant (e.g., TadA*8) is a monomer comprising one or more of the following alterations: Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant (TadA*8) is a monomer comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In other embodiments, a base editor of the disclosure comprising an adenosine deaminase variant (e.g., TadA*8) monomer comprising one or more of the following alterations: R26C, V88A, A109S, T111R, D119N, H122N, Y147D, F149Y, T166I and/or D167N, relative to TadA*7.10, the TadA reference sequence, or a corresponding mutation in another TadA. In other embodiments, the adenosine deaminase variant (TadA*8) monomer comprises a combination of alterations selected from the group of: R26C+A109S+T111R+D119N+H122N+Y147D+F149Y+T166I+D167N; V88A+A109S+T111R+D119N+H122N+F149Y+T166I+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to TadA*7.10, the TadA reference sequence, or a corresponding mutation in another TadA.

In some embodiments, an adenosine deaminase variant (e.g., MSP828) is a monomer comprising one or more of the following alterations L36H, I76Y, V82G, Y147T, Y147D, F149Y, Q154S, N157K, and/or D167N, relative to TadA*7.10, the TadA reference sequence, or a corresponding mutation in another TadA. In some embodiments, an adenosine deaminase variant (e.g., MSP828) is a monomer comprising V82G, Y147T/D, Q154S, and one or more of L36H, I76Y, F149Y, N157K, and D167N, relative to TadA*7.10, the TadA reference sequence, or a corresponding mutation in another TadA. In other embodiments, the adenosine deaminase variant (TadA variant) is a monomer comprising a combination of alterations selected from the group of: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; L36H+I76Y+V82G+Y147D+F149Y+Q154S+N157K+D167N, relative to TadA*7.10, the TadA reference sequence, or a corresponding mutation in another TadA.

In other embodiments, the adenosine deaminase variant is a homodimer comprising two adenosine deaminase domains (e.g., TadA*8) each having one or more of the following alterations Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant is a homodimer comprising two adenosine deaminase domains (e.g., TadA*8) each having a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In other embodiments, a base editor of the disclosure comprising an adenosine deaminase variant (e.g., TadA*8) homodimer comprising two adenosine deaminase domains (e.g., TadA*8) each having one or more of the following alterations R26C, V88A, A109S, T111R, D119N, H122N, Y147D, F149Y, T166I and/or D167N, relative to TadA*7.10, the TadA reference sequence, or a corresponding mutation in another TadA. In other embodiments, the adenosine deaminase variant is a homodimer comprising two adenosine deaminase domains (e.g., TadA*8) each having a combination of alterations selected from the group of: R26C+A109S+T111R+D119N+H122N+Y147D+F149Y+T166I+D167N; V88A+A109S+T111R+D119N+H122N+F149Y+T166I+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to TadA*7.10, the TadA reference sequence, or a corresponding mutation in another TadA.

In some embodiments, an adenosine deaminase variant is a homodimer comprising two adenosine deaminase domains (e.g., TadA*7.10) each having one or more of the following alterations L36H, I76Y, V82G, Y147T, Y147D, F149Y, Q154S, N157K, and/or D167N, relative to TadA*7.10, the TadA reference sequence, or a corresponding mutation in another TadA. In some embodiments, an adenosine deaminase variant is a homodimer comprising two adenosine deaminase variant domains (e.g., MSP828) each having the following alterations V82G, Y147T/D, Q154S, and one or more of L36H, I76Y, F149Y, N157K, and D167N, relative to TadA*7.10, the TadA reference sequence, or a corresponding mutation in another TadA. In other embodiments, the adenosine deaminase variant is a homodimer comprising two adenosine deaminase domains (e.g., TadA*7.10) each having a combination of alterations selected from the group of: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; L36H+I76Y+V82G+Y147D+F149Y+Q154S+N157K+D167N, relative to TadA*7.10, the TadA reference sequence, or a corresponding mutation in another TadA.

In other embodiments, the adenosine deaminase variant is a heterodimer of a wild-type adenosine deaminase domain and an adenosine deaminase variant domain (e.g., TadA*8) comprising one or more of the following alterations Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant is a heterodimer of a wild-type adenosine deaminase domain and an adenosine deaminase variant domain (e.g., TadA*8) comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In other embodiments, a base editor comprises a heterodimer of a wild-type adenosine deaminase domain and an adenosine deaminase variant domain (e.g., TadA*8) comprising one or more of the following alterations R26C, V88A, A109S, T111R, D119N, H122N, Y147D, F149Y, T166I and/or D167N, relative to TadA*7.10, the TadA reference sequence, or a corresponding mutation in another TadA. In other embodiments, the base editor comprises a heterodimer of a wild-type adenosine deaminase domain and an adenosine deaminase variant domain (e.g., TadA*8) comprising a combination of alterations selected from the group of: R26C+A109S+T111R+D119N+H122N+Y147D+F149Y+T166I+D167N; V88A+A109S+T111R+D119N+H122N+F149Y+T166I+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to TadA*7.10, the TadA reference sequence, or a corresponding mutation in another TadA.

In other embodiments, the adenosine deaminase variant is a heterodimer of a wild-type adenosine deaminase domain and an adenosine deaminase variant domain (e.g., TadA*7.10) comprising one or more of the following alterations L36H, I76Y, V82G, Y147T, Y147D, F149Y, Q154S, N157K, and/or D167N, relative to TadA*7.10, the TadA reference sequence, or a corresponding mutation in another TadA. In some embodiments, an adenosine deaminase variant is a heterodimer comprising a wild-type adenosine deaminase domain and an adenosine deaminase variant domain (e.g., MSP828) having the following alterations V82G, Y147T/D, Q154S, and one or more of L36H, I76Y, F149Y, N157K, and D167N, relative to TadA*7.10, the TadA reference sequence, or a corresponding mutation in another TadA. In other embodiments, the adenosine deaminase variant is a heterodimer of a wild-type adenosine deaminase domain and an adenosine deaminase variant domain (e.g., TadA*7.10) comprising a combination of alterations selected from the group of: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; L36H+I76Y+V82G+Y147D+F149Y+Q154S+N157K+D167N, relative to TadA*7.10, the TadA reference sequence, or a corresponding mutation in another TadA.

In other embodiments, the adenosine deaminase variant is a heterodimer of a TadA*7.10 domain and an adenosine deaminase variant domain (e.g., TadA*8) comprising one or more of the following alterations Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant is a heterodimer of a TadA*7.10 domain and an adenosine deaminase variant domain (e.g., TadA*8) comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In other embodiments, a base editor comprises a heterodimer of a TadA*7.10 domain and an adenosine deaminase variant domain (e.g., TadA*8) comprising one or more of the following alterations R26C, V88A, A109S, T111R, D119N, H122N, Y147D, F149Y, T166I and/or D167N, relative to TadA*7.10, the TadA reference sequence, or a corresponding mutation in another TadA. In other embodiments, the base editor comprises a heterodimer of a TadA*7.10 domain and an adenosine deaminase variant domain (e.g., TadA*8) comprising a combination of alterations selected from the group of: R26C+A109S+T111R+D119N+H122N+Y147D+F149Y+T166I+D167N; V88A+A109S+T111R+D119N+H122N+F149Y+T166I+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to TadA*7.10, the TadA reference sequence, or a corresponding mutation in another TadA.

In other embodiments, the adenosine deaminase variant is a heterodimer of a TadA*7.10 domain and an adenosine deaminase variant domain (e.g., TadA*7.10) comprising one or more of the following alterations L36H, I76Y, V82G, Y147T, Y147D, F149Y, Q154S, N157K, and/or D167N, relative to TadA*7.10, the TadA reference sequence, or a corresponding mutation in another TadA. In some embodiments, an adenosine deaminase variant is a heterodimer comprising a TadA*7.10 domain and an adenosine deaminase variant domain (e.g., MSP828) having the following alterations V82G, Y147T/D, Q154S, and one or more of L36H, I76Y, F149Y, N157K, and D167N, relative to TadA*7.10, the TadA reference sequence, or a corresponding mutation in another TadA. In other embodiments, the adenosine deaminase variant is a heterodimer of a TadA*7.10 domain and an adenosine deaminase variant domain (e.g., TadA*7.10) comprising a combination of alterations selected from the group of: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; L36H+I76Y+V82G+Y147D+F149Y+Q154S+N157K+D167N, relative to TadA*7.10, the TadA reference sequence, or a corresponding mutation in another TadA.

In some embodiments, the TadA*8 is a variant as shown in Tables 8A, 10, 11, or 13. Tables 8A, 10, 11, and 13 show certain amino acid position numbers in the TadA amino acid sequence and the amino acids present in those positions in the TadA-7.10 adenosine deaminase. Tables 8A, 10, 11, and 13 also show amino acid changes in TadA variants relative to TadA-7.10 following phage-assisted non-continuous evolution (PANCE) and phage-assisted continuous evolution (PACE), as described in M. Richter et al., 2020, Nature Biotechnology, doi.org/10.1038/s41587-020-0453-z, the entire contents of which are incorporated by reference herein. In some embodiments, the TadA*8 is TadA*8a, TadA*8b, TadA*8c, TadA*8d, or TadA*8e. In some embodiments, the TadA*8 is TadA*8e.

In particular embodiments, an adenosine deaminase heterodimer can comprise a TadA*8 domain and an adenosine deaminase domain selected from Staphylococcus aureus (S. aureus) TadA, Bacillus subtilis (B. subtilis) TadA, Salmonella typhimurium (S. typhimurium) TadA, Shewanella putrefaciens (S. putrefaciens) TadA, Haemophilus influenzae F3031 (H. influenzae) TadA, Caulobacter crescentus (C. crescentus) TadA, Geobacter sulfurreducens (G. sulfurreducens) TadA, or TadA*7.10.

In some embodiments, an adenosine deaminase is a TadA*8. In one embodiment, an adenosine deaminase is a TadA*8 that comprises or consists essentially of the following sequence or a fragment thereof having adenosine deaminase activity:

(SEQ ID NO: 316)
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCTFFR
MPRQVFNAQKKAQSSTD

In some embodiments, the TadA*8 is truncated. In some embodiments, the truncated TadA*8 is missing 1, 2, 3, 4, 5, 6, 7, 8, 9, 10, 11, 12, 13, 14, 15, 6, 17, 18, 19, or 20 N-terminal amino acid residues relative to the full length TadA*8. In some embodiments, the truncated TadA*8 is missing 1, 2, 3, 4, 5, 6, 7, 8, 9, 10, 11, 12, 13, 14, 15, 6, 17, 18, 19, or 20 C-terminal amino acid residues relative to the full length TadA*8. In some embodiments the adenosine deaminase variant is a full-length TadA*8.

In one embodiment, a fusion protein as described and/or exemplified herein comprises a wild-type TadA is linked to an adenosine deaminase variant described herein (e.g., TadA*8), which is linked to Cas9 nickase. In particular embodiments, the fusion proteins comprise a single TadA*8 domain (e.g., provided as a monomer). In other embodiments, the base editor comprises TadA*8 and TadA(wt), which are capable of forming heterodimers.

In some embodiments the TadA*8 is TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24

TABLE 8A
Additional TadA*8 Variants

| | | TadA amino acid number | | | | | | | | | |
|---|---|---|---|---|---|---|---|---|---|---|---|
| | TadA | 26 | 88 | 109 | 111 | 119 | 122 | 147 | 149 | 166 1 | 167 |
| | TadA-7.10 | R | V | A | T | D | H | Y | F | T | D |
| PANCE 1 | | | | | R | | | | | | |
| PANCE 2 | | | | S/T | R | | | | | | |
| PACE | TadA-8a | C | | S | R | N | N | D | Y | I | N |
| | TadA-8b | | A | S | R | N | N | | Y | I | N |
| | TadA-8c | C | | S | R | N | N | | Y | I | N |
| | TadA-8d | | A | | R | N | | | Y | | |
| | TadA-8e | | | S | R | N | N | D | Y | I | N |

In some embodiments, the TadA variant is a variant as shown in Table 8B. Table 8B shows certain amino acid position numbers in the TadA amino acid sequence and the amino acids present in those positions in the TadA*7.10 adenosine deaminase. In some embodiments, the TadA variant is MSP605, MSP680, MSP823, MSP824, MSP825, MSP827, MSP828, or MSP829. In some embodiments, the TadA variant is MSP828. In some embodiments, the TadA variant is MSP829.

TABLE 8B
TadA Variants

| | TadA Amino Acid Number | | | | | | | |
|---|---|---|---|---|---|---|---|---|
| Variant | 36 | 76 | 82 | 147 | 149 | 154 | 157 | 167 |
| TadA-7.10 | L | I | V | Y | F | Q | N | D |
| MSP605 | | | G | T | | S | | |
| MSP680 | | Y | G | T | | S | | |
| MSP823 | H | | G | T | | S | K | |
| MSP824 | | | G | D | Y | S | | N |
| MSP825 | H | | G | D | Y | S | K | N |
| MSP827 | H | Y | G | T | | S | K | |
| MSP828 | | Y | G | D | Y | S | | N |
| MSP829 | H | Y | G | D | Y | S | K | N |

In one embodiment, a fusion protein as described herein comprises a wild-type TadA is linked to an adenosine deaminase variant described herein, which is linked to Cas9 nickase. In particular embodiments, the fusion proteins comprise a single variant TadA domain (e.g., provided as a monomer). In other embodiments, the fusion protein comprises a variant TadA and TadA(wt), which are capable of forming heterodimers.

In some embodiments, the TadA variant is truncated. In some embodiments, the truncated TadA is missing 1, 2, 3, 4, 5, 6, 7, 8, 9, 10, 11, 12, 13, 14, 15, 6, 17, 18, 19, or 20 N-terminal amino acid residues relative to the full length TadA variant. In some embodiments, the truncated TadA variant is missing 1, 2, 3, 4, 5, 6, 7, 8, 9, 10, 11, 12, 13, 14, 15, 6, 17, 18, 19, or 20 C-terminal amino acid residues relative to the full length TadA variant. In some embodiments the adenosine deaminase variant is a full-length TadA variant.

In particular embodiments, a TadA*8 comprises one or more mutations at any of the following positions shown in bold. In other embodiments, a TadA*8 comprises one or more mutations at any of the positions shown with underlining:

(SEQ ID NO: 1)
MSEVEFSHEY WMRHALTLAK RARDEREVPV GAVLVLNNRV
IGEGWNRAIG⁵⁰
LHDPTAHAEI MALRQGGLVM QNYRLIDATL YVTFEPCVMC
AGAMIHSRIG¹⁰⁰
RVVFGVRNAK TGAAGSLMDV LHYPGMNHRV EITEGILADE
CAALLCYFFR¹⁵⁰
MPRQVFNAQK KAQSSTD

For example, the TadA*8 comprises alterations at amino acid position 82 and/or 166 (e.g., V82S, T166R) alone or in combination with any one or more of the following Y147T, Y147R, Q154S, Y123H, and/or Q154R, relative to TadA*7.10, the TadA reference sequence, or a corresponding mutation in another TadA.

In particular embodiments, a combination of alterations is selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA. In some embodiments, an adenosine deaminase comprises one or more of the following alterations: R21N, R23H, E25F, N38G, L51W, P54C, M70V, Q71M, N72K, Y73S, V82T, M94V, P124W, T133K, D139L, D139M, C146R, and A158K.

In some embodiments, an adenosine deaminase comprises one or more of the following combinations of alterations: V82S+Q154R+Y147R; V82S+Q154R+Y123H; V82S+Q154R+Y147R+Y123H; Q154R+Y147R+Y123H+I76Y+V82S; V82S+I76Y; V82S+Y147R; V82S+Y147R+Y123H; V82S+Q154R+Y123H; Q154R+Y147R+Y123H+I76Y; V82S+Y147R; V82S+Y147R+Y123H; V82S+Q154R+Y123H; V82S+Q154R+Y147R; V82S+Q154R+Y147R; Q154R+Y147R+Y123H+I76Y; Q154R+Y147R+Y123H+I76Y+V82S; I76Y_V82S_Y123H_Y147R_Q154R; Y147R+Q154R+H123H; and V82S+Q154R.

In some embodiments, an adenosine deaminase comprises one or more of the following combinations of alterations: E25F+V82S+Y123H, T133K+Y147R+Q154R; E25F+V82S+Y123H+Y147R+Q154R; L51W+V82S+Y123H+C146R+Y147R+Q154R; Y73S+V82S+Y123H+Y147R+Q154R; P54C+V82S+Y123H+Y147R+Q154R; N38G+V82T+Y123H+Y147R+Q154R; N72K+V82S+Y123H+D139L+Y147R+Q154R; E25F+V82S+Y123H+D139M+Y147R+Q154R; Q71M+V82S+Y123H+Y147R+Q154R; E25F+V82S+Y123H+T133K+Y147R+Q154R; E25F+V82S+Y123H+Y147R+Q154R; V82S+Y123H+P124W+Y147R+Q154R; L51W+V82S+Y123H+C146R+Y147R+Q154R; P54C+V82S+Y123H+Y147R+Q154R; Y73S+V82S+Y123H+Y147R+Q154R; N38G+V82T+Y123H+Y147R+Q154R; R23H+V82S+Y123H+Y147R+Q154R; R21N+V82S+Y123H+Y147R+Q154R; V82S+Y123H+Y147R+Q154R+A158K; N72K+V82S+Y123H+D139L+Y147R+Q154R; E25F+V82S+Y123H+D139M+Y147R+Q154R; and M70V+V82S+M94V+Y123H+Y147R+Q154R

In some embodiments, an adenosine deaminase comprises one or more of the following combinations of alterations: Q71M+V82S+Y123H+Y147R+Q154R; E25F+I76Y+V82S+Y123H+Y147R+Q154R; I76Y+V82T+Y123H+Y147R+Q154R; N38G+I76Y+V82S+Y123H+Y147R+Q154R; R23H+I76Y+V82S+Y123H+Y147R+Q154R; P54C+I76Y+V82S+Y123H+Y147R+Q154R; R21N+I76Y+V82S+Y123H+Y147R+Q154R; I76Y+V82S+Y123H+D139M+Y147R+Q154R; Y73S+I76Y+V82S+Y123H+Y147R+Q154R; E25F+I76Y+V82S+Y123H+Y147R+Q154R; I76Y+V82T+Y123H+Y147R+Q154R; N38G+I76Y+V82S+Y123H+Y147R+Q154R; R23H+I76Y+V82S+Y123H+Y147R+Q154R; P54C+I76Y+V82S+Y123H+Y147R+Q154R; R21N+I76Y+V82S+Y123H+Y147R+Q154R; I76Y+V82S+Y123H+D139M+Y147R+Q154R; Y73S+I76Y+V82S+Y123H+Y147R+Q154R; and V82S+Q154R; N72K_V82S+Y123H+Y147R+Q154R; Q71M_V82S+Y123H+Y147R+Q154R; V82S+Y123H+T133K+Y147R+Q154R; V82S+Y123H+T133K+Y147R+Q154R+A158K; M70V+Q71M+N72K+V82S+Y123H+Y147R+Q154R; N72K_V82S+Y123H+Y147R+Q154R; Q71M_V82S+Y123H+Y147R+Q154R; M70V+V82S+M94V+Y123H+Y147R+Q154R; V82S+Y123H+T133K+Y147R+Q154R; V82S+Y123H+T133K+Y147R+Q154R+A158K; and M70V+Q71M+N72K+V82S+Y123H+Y147R+Q154R. In some embodiments, the adenosine deaminase is expressed as a monomer. In other embodiments, the adenosine deaminase is expressed as a heterodimer. In some embodiments, the deaminase or other polypeptide sequence lacks a methionine, for example when included as a component of a fusion protein. This can alter the numbering of positions. However, the skilled person will understand that such corresponding mutations refer to the same mutation, e.g., Y73S and Y72S and D139M and D138M.

In some embodiments, the TadA*9 variant is a monomer. In some embodiments, the TadA*9 variant is a heterodimer with a wild-type TadA adenosine deaminase. In some embodiments, the TadA*9 variant is a heterodimer with another TadA variant (e.g., TadA*8, TadA*9). Additional details of TadA*9 adenosine deaminases are described in International PCT Application No. PCT/2020/049975, which is incorporated herein by reference in its entirety. In one embodiment, a fusion protein as described herein comprises a wild-type TadA is linked to an adenosine deaminase variant described herein (e.g., TadA variant), which is linked to Cas9 nickase. In particular embodiments, the fusion proteins comprise a single TadA variant domain (e.g., provided as a monomer). In other embodiments, the base editor comprises TadA*8 and TadA(wt), which are capable of forming heterodimers.

In particular embodiments, the fusion proteins comprise a single (e.g., provided as a monomer) TadA variant domain. In some embodiments, the TadA variant is linked to a Cas9 nickase. In some embodiments, the fusion proteins described herein comprise as a heterodimer of a wild-type TadA (TadA(wt)) linked to a TadA variant. In other embodiments, the fusion proteins described herein comprise as a heterodimer of a TadA*7.10 linked to a TadA variant. In some embodiments, the fusion protein comprises a TadA variant monomer. In some embodiments, the fusion protein comprises a heterodimer of a TadA variant and a TadA(wt). In some embodiments, the fusion protein comprises a heterodimer of a TadA variant and TadA*7.10. In some embodiments, the fusion protein comprises a heterodimer of two TadA variants. In some embodiments, the TadA variant is selected from Table 8A, 8B, 9, 10, 11, 12, 13, 14A, 14B, 18, or 20 infra or any other TadA variant provided herein.

In some embodiments, the deaminase or other polypeptide sequence lacks a methionine, for example when included as a component of a fusion protein. This can alter the numbering of positions. However, the skilled person will understand that such corresponding mutations refer to the same mutation.

Any of the mutations provided herein and any additional mutations (e.g., based on the ecTadA amino acid sequence) can be introduced into any other adenosine deaminases. Any of the mutations provided herein can be made individually or in any combination in TadA reference sequence or another adenosine deaminase (e.g., ecTadA).

Details of A to G nucleobase editing proteins are described in International PCT Application No. PCT/2017/045381 (WO2018/027078) and Gaudelli, N. M., et al., “Programmable base editing of A·T to G·C in genomic DNA without DNA cleavage” Nature, 551, 464-471 (2017), the entire contents of which are hereby incorporated by reference.

High Fidelity Cas9 Domains Some aspects of the disclosure provide high fidelity Cas9 domains. In some embodiments, high fidelity Cas9 domains are engineered Cas9 domains comprising one or more mutations that decrease electrostatic interactions between the Cas9 domain and a sugar-phosphate backbone of a DNA, as compared to a corresponding wild-type Cas9 domain. Without wishing to be bound by any particular theory, high fidelity Cas9 domains that have decreased electrostatic interactions with a sugar-phosphate backbone of DNA may have less off-target effects. In some embodiments, a Cas9 domain (e.g., a wild type Cas9 domain) comprises one or more mutations that decreases the association between the Cas9 domain and a sugar-phosphate backbone of a DNA. In some embodiments, a Cas9 domain comprises one or more mutations that decreases the association between the Cas9 domain and a sugar-phosphate backbone of a DNA by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, or at least 70%.

In some embodiments, any of the Cas9 fusion proteins provided herein comprise one or more of a N497X, a R661X, a Q695X, and/or a Q926X mutation, or a corresponding mutation in any of the amino acid sequences provided herein, wherein X is any amino acid. In some embodiments, any of the Cas9 fusion proteins provided herein comprise one or more of a N497A, a R661A, a Q695A, and/or a Q926A mutation, or a corresponding mutation in any of the amino acid sequences provided herein. In some embodiments, the Cas9 domain comprises a D10A mutation, or a corresponding mutation in any of the amino acid sequences provided herein. Cas9 domains with high fidelity are known in the art and would be apparent to the skilled artisan. For example, Cas9 domains with high fidelity have been described in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each are incorporated herein by reference. An Exemplary high fidelity Cas9 domain is provided in the Sequence Listing as SEQ ID NO: 233. In some embodiments, high fidelity Cas9 domains are engineered Cas9 domains comprising one or more mutations that decrease electrostatic interactions between the Cas9 domain and the sugar-phosphate backbone of a DNA, relative to a corresponding wild-type Cas9 domain. High fidelity Cas9 domains that have decreased electrostatic interactions with the sugar-phosphate backbone of DNA have less off-target effects. In some embodiments, the Cas9 domain (e.g., a wild type Cas9 domain (SEQ ID NOs: 197 and 200)) comprises one or more mutations that decrease the association between the Cas9 domain and the sugar-phosphate backbone of a DNA. In some embodiments, a Cas9 domain comprises one or more mutations that decreases the association between the Cas9 domain and the sugar-phosphate backbone of DNA by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, or at least 70%.

In some embodiments, the modified Cas9 is a high fidelity Cas9 enzyme. In some embodiments, the high fidelity Cas9 enzyme is SpCas9(K855A), eSpCas9(1.1), SpCas9-HF1, or hyper accurate Cas9 variant (HypaCas9). The modified Cas9 eSpCas9(1.1) contains alanine substitutions that weaken the interactions between the HNH/RuvC groove and the non-target DNA strand, preventing strand separation and cutting at off-target sites. Similarly, SpCas9-HF1 lowers off-target editing through alanine substitutions that disrupt Cas9's interactions with the DNA phosphate backbone. HypaCas9 contains mutations (SpCas9 N692A/M694A/Q695A/H698A) in the REC3 domain that increase Cas9 proofreading and target discrimination. All three high fidelity enzymes generate less off-target editing than wildtype Cas9.

An exemplary high fidelity Cas9 is provided below. High Fidelity Cas9 domain 15 mutations relative to Cas9 are shown in bold and underlined.

(SEQ ID NO: 233)
DKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTAFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGALSRKLINGIRDKQSGKTILDFLKSDGFANRNFMALIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRAITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD

Fusion Proteins Comprising a NapDNAbp and a Cytidine Deaminase and/or Adenosine Deaminase

Some aspects of the disclosure provide fusion proteins comprising a Cas9 domain or other nucleic acid programmable DNA binding protein (e.g., Cas12) and one or more cytidine deaminase and/or adenosine deaminase domains. It should be appreciated that the Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the cytidine deaminases and/or adenosine deaminases provided herein. The domains of the base editors disclosed herein can be arranged in any order.

In some embodiments, the fusion protein comprises the following domains A-C, A-D, or A-E:
NH₂-[A-B-C]-COOH;
NH₂-[A-B-C-D]-COOH; or
NH₂-[A-B-C-D-E]-COOH;
   wherein A and C or A, C, and E, each comprises one or more of the following:
an adenosine deaminase domain or an active fragment thereof,
a cytidine deaminase domain or an active fragment thereof, and
wherein B or B and D, each comprises one or more domains having nucleic acid sequence specific binding activity.

In some embodiments, the fusion protein comprises the following structure:
NH₂-[Aₙ-Bₒ-Cₙ]-COOH;
NH₂-[Aₙ-Bₒ-Cₙ-Dₒ]-COOH; or
NH₂-[Aₙ-Bₒ-Cₚ-Dₒ-E_{q}]-COOH;
   wherein A and C or A, C, and E, each comprises one or more of the following:
an adenosine deaminase domain or an active fragment thereof,
a cytidine deaminase domain or an active fragment thereof, and
wherein n is an integer: 1, 2, 3, 4, or 5, wherein p is an integer: 0, 1, 2, 3, 4, or 5; wherein q is an integer 0, 1, 2, 3, 4, or 5; and wherein B or B and D each comprises a domain having nucleic acid sequence specific binding activity; and wherein o is an integer: 1, 2, 3, 4, or 5.

For example, and without limitation, in some embodiments, the fusion protein comprises the structure:
NH2-[adenosine deaminase]-[Cas9 domain]-COOH;
NH2-[Cas9 domain]-[adenosine deaminase]-COOH;
NH2-[cytidine deaminase]-[Cas9 domain]-COOH;
NH2-[Cas9 domain]-[cytidine deaminase]-COOH;
NH2-[cytidine deaminase]-[Cas9 domain]-[adenosine deaminase]-COOH;
NH2-[adenosine deaminase]-[Cas9 domain]-[cytidine deaminase]-COOH;
NH2-[adenosine deaminase]-[cytidine deaminase]-[Cas9 domain]-COOH;
NH2-[cytidine deaminase]-[adenosine deaminase]-[Cas9 domain]-COOH;
NH2-[Cas9 domain]-[adenosine deaminase]-[cytidine deaminase]-COOH; or
NH2-[Cas9 domain]-[cytidine deaminase]-[adenosine deaminase]-COOH.

In some embodiments, any of the Cas12 domains or Cas12 proteins provided herein may be fused with any of the cytidine or adenosine deaminases provided herein. For example, and without limitation, in some embodiments, the fusion protein comprises the structure:
NH2-[adenosine deaminase]-[Cas12 domain]-COOH;
NH2-[Cas12 domain]-[adenosine deaminase]-COOH;
NH2-[cytidine deaminase]-[Cas12 domain]-COOH;
NH2-[Cas12 domain]-[cytidine deaminase]-COOH;
NH2-[cytidine deaminase]-[Cas12 domain]-[adenosine deaminase]-COOH;
NH2-[adenosine deaminase]-[Cas12 domain]-[cytidine deaminase]-COOH;
NH2-[adenosine deaminase]-[cytidine deaminase]-[Cas12 domain]-COOH;
NH2-[cytidine deaminase]-[adenosine deaminase]-[Cas12 domain]-COOH;
NH2-[Cas12 domain]-[adenosine deaminase]-[cytidine deaminase]-COOH; or
NH2-[Cas12 domain]-[cytidine deaminase]-[adenosine deaminase]-COOH.

In some embodiments, the adenosine deaminase is a TadA*8. Exemplary fusion protein structures include the following:
NH2-[TadA*8]-[Cas9 domain]-COOH;
NH2-[Cas9 domain]-[TadA*8]-COOH;
NH2-[TadA*8]-[Cas12 domain]-COOH; or
NH2-[Cas12 domain]-[TadA*8]-COOH.

In some embodiments, the adenosine deaminase of the fusion protein comprises a TadA*8 and a cytidine deaminase and/or an adenosine deaminase. In some embodiments, the TadA*8 is TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24.

Exemplary fusion protein structures include the following:
NH2-[TadA*8]-[Cas9/Cas12]-[adenosine deaminase]-COOH;
NH2-[adenosine deaminase]-[Cas9/Cas12]-[TadA*8]-COOH;
NH2-[TadA*8]-[Cas9/Cas12]-[cytidine deaminase]-COOH; or
NH2-[cytidine deaminase]-[Cas9/Cas12]-[TadA*8]-COOH.

In some embodiments, the adenosine deaminase of the fusion protein comprises a TadA*9 and a cytidine deaminase and/or an adenosine deaminase. Exemplary fusion protein structures include the following:
NH2-[TadA*9]-[Cas9/Cas12]-[adenosine deaminase]-COOH;
NH2-[adenosine deaminase]-[Cas9/Cas12]-[TadA*9]-COOH;
NH2-[TadA*9]-[Cas9/Cas12]-[cytidine deaminase]-COOH; or
NH2-[cytidine deaminase]-[Cas9/Cas12]-[TadA*9]-COOH.

In some embodiments, the fusion protein can comprise a deaminase flanked by an N-terminal fragment and a C-terminal fragment of a Cas9 or Cas12 polypeptide. In some embodiments, the fusion protein comprises a cytidine deaminase flanked by an N-terminal fragment and a C-terminal fragment of a Cas9 or Cas12 polypeptide. In some embodiments, the fusion protein comprises an adenosine deaminase flanked by an N-terminal fragment and a C-terminal fragment of a Cas9 or Cas12 polypeptide.

In some embodiments, the fusion proteins comprising a cytidine deaminase or adenosine deaminase and a napDNAbp (e.g., Cas9 or Cas12 domain) do not include a linker sequence. In some embodiments, a linker is present between the cytidine or adenosine deaminase and the napDNAbp. In some embodiments, the “-” used in the general architecture above indicates the presence of an optional linker. In some embodiments, cytidine or adenosine deaminase and the napDNAbp are fused via any of the linkers provided herein. For example, in some embodiments the cytidine or adenosine deaminase and the napDNAbp are fused via any of the linkers provided herein.

It should be appreciated that the fusion proteins of the present disclosure may comprise one or more additional features. For example, in some embodiments, the fusion protein may comprise inhibitors, cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

Exemplary, yet nonlimiting, fusion proteins are described in International PCT Application Nos. PCT/2017/044935, PCT/US2019/044935, and PCT/US2020/016288, each of which is incorporated herein by reference for its entirety.

### Fusion Proteins Comprising a Nuclear Localization Sequence (NLS)

In some embodiments, the fusion proteins provided herein further comprise one or more (e.g., 2, 3, 4, 5) nuclear targeting sequences, for example a nuclear localization sequence (NLS). In one embodiment, a bipartite NLS is used. In some embodiments, a NLS comprises an amino acid sequence that facilitates the importation of a protein, that comprises an NLS, into the cell nucleus (e.g., by nuclear transport). In some embodiments, any of the fusion proteins provided herein further comprise a nuclear localization sequence (NLS). In some embodiments, the NLS is fused to the N-terminus of the fusion protein. In some embodiments, the NLS is fused to the C-terminus of the fusion protein. In some embodiments, the NLS is fused to the N-terminus of the Cas9 domain. In some embodiments, the NLS is fused to the C-terminus of the Cas9 domain. In some embodiments, the NLS is fused to the C-terminus of an nCas9 domain or a dCas9 domain. In some embodiments, the NLS is fused to the N-terminus of the adenosine deaminase. In some embodiments, the NLS is fused to the C-terminus of the adenosine deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences. In some embodiments, an NLS comprises the amino acid sequence PKKKRKVEGADKRTADGSEFESPKKKRKV (SEQ ID NO: 328), KRTADGSEFESPKKKRKV (SEQ ID NO: 190), KRPAATKKAGQAKKKK (SEQ ID NO: 191), KKTELQTTNAENKTKKL (SEQ ID NO: 192), KRGINDRNFWRGENGRKTR (SEQ ID NO: 193), RKSGKIAAIVVKRPRKPKKKRKV (SEQ ID NO: 329), or MDSLLMNRRKFLYQFKNVRWAKGRRETYLC (SEQ ID NO: 196).

In some embodiments, the fusion proteins comprising an adenosine deaminase, a Cas9 domain, and an NLS do not comprise a linker sequence. In some embodiments, linker sequences between one or more of the domains or proteins (e.g., adenosine deaminase, Cas9 domain or NLS) are present. In some embodiments, a linker is present between the adenosine deaminase domains and the napDNAbp. In some embodiments, the “-” used in the general architecture below indicates the presence of an optional linker. In some embodiments, the adenosine deaminase and the napDNAbp are fused via any of the linkers provided herein. For example, in some embodiments the adenosine deaminase and the napDNAbp are fused via any of the linkers provided herein.

In some embodiments, the general architecture of exemplary napDNAbp (e.g., Cas9) fusion proteins with an adenosine deaminase and a napDNAbp (e.g., Cas9) domain comprises any one of the following structures, where NLS is a nuclear localization sequence (e.g., any NLS provided herein), NH₂ is the N-terminus of the fusion protein, and COOH is the C-terminus of the fusion protein:
NH₂′-NLS-[adenosine deaminase]-[napDNAbp domain]-COOH;
NH₂′-NLS [napDNAbp domain]-[adenosine deaminase]-COOH;
NH₂-[adenosine deaminase]-[napDNAbp domain]-NLS-COOH;
NH₂-[napDNAbp domain]-[adenosine deaminase]-NLS-COOH;

In some embodiments, the NLS is present in a linker or the NLS is flanked by linkers, for example described herein. A bipartite NLS comprises two basic amino acid clusters, which are separated by a relatively short spacer sequence (hence bipartite—2 parts, while monopartite NLSs are not). The NLS of nucleoplasmin, KR [PAATKKAGQA] KKKK (SEQ ID NO: 191), is the prototype of the ubiquitous bipartite signal: two clusters of basic amino acids, separated by a spacer of about 10 amino acids. The sequence of an exemplary bipartite NLS follows: PKKKRKVEGADKRTADGSEFESPKKKRKV (SEQ ID NO: 328).

A vector that encodes a CRISPR enzyme comprising one or more nuclear localization sequences (NLSs) can be used. For example, there can be or be about 1, 2, 3, 4, 5, 6, 7, 8, 9, 10 NLSs used. A CRISPR enzyme can comprise the NLSs at or near the amino-terminus, about or more than about 1, 2, 3, 4, 5, 6, 7, 8, 9, 10 NLSs at or near the carboxy-terminus, or any combination thereof (e.g., one or more NLS at the amino-terminus and one or more NLS at the carboxy terminus). When more than one NLS is present, each can be selected independently of others, such that a single NLS can be present in more than one copy and/or in combination with one or more other NLSs present in one or more copies.

CRISPR enzymes used in the methods can comprise about 6 NLSs. An NLS is considered near the N- or C-terminus when the nearest amino acid to the NLS is within about 50 amino acids along a polypeptide chain from the N- or C-terminus, e.g., within 1, 2, 3, 4, 5, 10, 15, 20, 25, 30, 40, or 50 amino acids.

### Base Editor System

Provided herein are systems, compositions, and methods for editing a nucleobase using a base editor system. In some embodiments, the base editor system comprises (1) a base editor (BE) comprising a polynucleotide programmable nucleotide binding domain and a nucleobase editing domain (e.g., a deaminase domain) for editing the nucleobase; and (2) a guide polynucleotide (e.g., guide RNA) in conjunction with the polynucleotide programmable nucleotide binding domain. In some embodiments, the base editor system is an adenosine base editor (ABE). In some embodiments, the polynucleotide programmable nucleotide binding domain is a polynucleotide programmable DNA binding domain. In some embodiments, the polynucleotide programmable nucleotide binding domain is a polynucleotide programmable RNA binding domain. In some embodiments, the nucleobase editing domain is a deaminase domain. In some embodiments, a deaminase domain can be an adenine deaminase or an adenosine deaminase. In some embodiments, the adenosine base editor can deaminate adenine in DNA.

In some embodiments, a base editing system as provided herein provides a new approach to genome editing that uses a fusion protein containing a catalytically defective Streptococcus pyogenes Cas9, a deaminase (e.g., adenosine deaminase), and an inhibitor of base excision repair to induce programmable, single nucleotide (C→T or A→G) changes in DNA without generating double-strand DNA breaks, without requiring a donor DNA template, and without inducing an excess of stochastic insertions and deletions.

Details of nucleobase editing proteins are described in International PCT Application Nos. PCT/2017/045381 (WO2018/027078) and PCT/US2016/058344 (WO2017/070632), each of which is incorporated herein by reference for its entirety. Also see Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference.

Use of the base editor system provided herein comprises the steps of: (a) contacting a target nucleotide sequence of a polynucleotide (e.g., double- or single stranded DNA or RNA) of a subject with a base editor system comprising a nucleobase editor (e.g., an adenosine base editor) and a guide polynucleic acid (e.g., gRNA), wherein the target nucleotide sequence comprises a targeted nucleobase pair; (b) inducing strand separation of said target region; (c) converting a first nucleobase of said target nucleobase pair in a single strand of the target region to a second nucleobase; and (d) cutting no more than one strand of said target region, where a third nucleobase complementary to the first nucleobase base is replaced by a fourth nucleobase complementary to the second nucleobase. It should be appreciated that in some embodiments, step (b) is omitted. In some embodiments, said targeted nucleobase pair is a plurality of nucleobase pairs in one or more genes. In some embodiments, the base editor system provided herein is capable of multiplex editing of a plurality of nucleobase pairs in one or more genes. In some embodiments, the plurality of nucleobase pairs is located in the same gene. In some embodiments, the plurality of nucleobase pairs is located in one or more genes, wherein at least one gene is located in a different locus.

In some embodiments, the cut single strand (nicked strand) is hybridized to the guide nucleic acid. In some embodiments, the cut single strand is opposite to the strand comprising the first nucleobase. In some embodiments, the base editor comprises a Cas9 domain. In some embodiments, the first base is adenine, and the second base is not a G, C, A, or T. In some embodiments, the second base is inosine.

In some embodiments, a single guide polynucleotide may be utilized to target a deaminase to a target nucleic acid sequence. In some embodiments, a single pair of guide polynucleotides may be utilized to target different deaminases to a target nucleic acid sequence.

The nucleobase components and the polynucleotide programmable nucleotide binding component of a base editor system may be associated with each other covalently or non-covalently. For example, in some embodiments, the deaminase domain can be targeted to a target nucleotide sequence by a polynucleotide programmable nucleotide binding domain. In some embodiments, the polynucleotide programmable nucleotide binding domain is non-covalently associated with or attached to the deaminase domain. In some embodiments, a polynucleotide programmable nucleotide binding domain can be fused or linked to a deaminase domain. In some embodiments, a polynucleotide programmable nucleotide binding domain can target a deaminase domain to a target nucleotide sequence by non-covalently interacting with or associating with the deaminase domain. For example, in some embodiments, the nucleobase editing component, e.g., the deaminase component can comprise an additional heterologous portion or domain that is capable of interacting with, associating with, or capable of forming a complex with an additional heterologous portion or domain that is part of a polynucleotide programmable nucleotide binding domain. In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion may be capable of binding to a polynucleotide linker. The additional heterologous portion may be a protein domain. In some embodiments, the additional heterologous portion may be a K Homology (KH) domain, a MS2 coat protein domain, a PP7 coat protein domain, a SfMu Com coat protein domain, a steril alpha motif, a telomerase Ku binding motif and Ku protein, a telomerase Sm7 binding motif and Sm7 protein, or an RNA recognition motif.

A base editor system may further comprise a guide polynucleotide component. It should be appreciated that components of the base editor system may be associated with each other via covalent bonds, noncovalent interactions, or any combination of associations and interactions thereof. In some embodiments, a deaminase domain can be targeted to a target nucleotide sequence by a guide polynucleotide. For example, in some embodiments, the nucleobase editing component of the base editor system, e.g., the deaminase component, can comprise an additional heterologous portion or domain (e.g., polynucleotide binding domain such as an RNA or DNA binding protein) that is capable of interacting with, associating with, or capable of forming a complex with a portion or segment (e.g., a polynucleotide motif) of a guide polynucleotide. In some embodiments, the additional heterologous portion or domain (e.g., polynucleotide binding domain such as an RNA or DNA binding protein) can be fused or linked to the deaminase domain. In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion may be capable of binding to a polynucleotide linker. The additional heterologous portion may be a protein domain. In some embodiments, the additional heterologous portion may be a K Homology (KH) domain, a MS2 coat protein domain, a PP7 coat protein domain, a SfMu Com coat protein domain, a sterile alpha motif, a telomerase Ku binding motif and Ku protein, a telomerase Sm7 binding motif and Sm7 protein, or an RNA recognition motif.

In some embodiments, a base editor system can further comprise an inhibitor of base excision repair (BER) component. It should be appreciated that components of the base editor system may be associated with each other via covalent bonds, noncovalent interactions, or any combination of associations and interactions thereof. The inhibitor of BER component may comprise a base excision repair inhibitor. In some embodiments, the inhibitor of base excision repair can be a uracil DNA glycosylase inhibitor (UGI). In some embodiments, the inhibitor of base excision repair can be an inosine base excision repair inhibitor. In some embodiments, the inhibitor of base excision repair can be targeted to the target nucleotide sequence by the polynucleotide programmable nucleotide binding domain. In some embodiments, a polynucleotide programmable nucleotide binding domain can be fused or linked to an inhibitor of base excision repair. In some embodiments, a polynucleotide programmable nucleotide binding domain can be fused or linked to a deaminase domain and an inhibitor of base excision repair. In some embodiments, a polynucleotide programmable nucleotide binding domain can target an inhibitor of base excision repair to a target nucleotide sequence by non-covalently interacting with or associating with the inhibitor of base excision repair. For example, in some embodiments, the inhibitor of base excision repair component can comprise an additional heterologous portion or domain that is capable of interacting with, associating with, or capable of forming a complex with an additional heterologous portion or domain that is part of a polynucleotide programmable nucleotide binding domain. In some embodiments, the inhibitor of base excision repair can be targeted to the target nucleotide sequence by the guide polynucleotide. For example, in some embodiments, the inhibitor of base excision repair can comprise an additional heterologous portion or domain (e.g., polynucleotide binding domain such as an RNA or DNA binding protein) that is capable of interacting with, associating with, or capable of forming a complex with a portion or segment (e.g., a polynucleotide motif) of a guide polynucleotide. In some embodiments, the additional heterologous portion or domain of the guide polynucleotide (e.g., polynucleotide binding domain such as an RNA or DNA binding protein) can be fused or linked to the inhibitor of base excision repair. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion may be capable of binding to a polynucleotide linker. The additional heterologous portion may be a protein domain. In some embodiments, the additional heterologous portion may be a K Homology (KH) domain, a MS2 coat protein domain, a PP7 coat protein domain, a SfMu Com coat protein domain, a sterile alpha motif, a telomerase Ku binding motif and Ku protein, a telomerase Sm7 binding motif and Sm7 protein, or an RNA recognition motif.

In some embodiments, the base editor inhibits base excision repair (BER) of the edited strand. In some embodiments, the base editor protects or binds the non-edited strand. In some embodiments, the base editor comprises UGI activity. In some embodiments, the base editor comprises a catalytically inactive inosine-specific nuclease. In some embodiments, the base editor comprises nickase activity. In some embodiments, the intended edit of base pair is upstream of a PAM site. In some embodiments, the intended edit of base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edit of base-pair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site.

In some embodiments, the method does not require a canonical (e.g., NGG) PAM site. In some embodiments, the nucleobase editor comprises a linker or a spacer. In some embodiments, the linker or spacer is 1-25 amino acids in length. In some embodiments, the linker or spacer is 5-20 amino acids in length. In some embodiments, the linker or spacer is 10, 11, 12, 13, 14, 15, 16, 17, 18, 19, or 20 amino acids in length.

In some embodiments, the base editing fusion proteins provided herein need to be positioned at a precise location, for example, where a target base is placed within a defined region (e.g., a “deamination window”). In some embodiments, a target can be within a 4 base region. In some embodiments, such a defined target region can be approximately 15 bases upstream of the PAM. See Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference.

In some embodiments, the target region comprises a target window, wherein the target window comprises the target nucleobase pair. In some embodiments, the target window comprises 1-10 nucleotides. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edit of base pair is within the target window. In some embodiments, the target window comprises the intended edit of base pair. In some embodiments, the method is performed using any of the base editors provided herein. In some embodiments, a target window is a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a polynucleotide programmable nucleotide binding domain. In some embodiments, an NLS of the base editor is localized C-terminal to a polynucleotide programmable nucleotide binding domain.

Other exemplary features that can be present in a base editor as disclosed herein are localization sequences, such as cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

In some embodiments, the adenosine base editor (ABE) can deaminate adenine in DNA. In some embodiments, ABE is generated by replacing APOBEC1 component of BE3 with natural or engineered E. coli TadA, human ADAR2, mouse ADA, or human ADAT2. In some embodiments, ABE comprises evolved TadA variant. In some embodiments, the ABE is ABE 1.2 (TadA*-XTEN-nCas9-NLS). In some embodiments, TadA* comprises A106V and D108N mutations.

In some embodiments, the ABE is a second-generation ABE. In some embodiments, the ABE is ABE2.1, which comprises additional mutations D147Y and E155V in TadA* (TadA*2.1). In some embodiments, the ABE is ABE2.2, ABE2.1 fused to catalytically inactivated version of human alkyl adenine DNA glycosylase (AAG with E125Q mutation). In some embodiments, the ABE is ABE2.3, ABE2.1 fused to catalytically inactivated version of E. coli Endo V (inactivated with D35A mutation). In some embodiments, the ABE is ABE2.6 which has a linker twice as long (32 amino acids, (SGGS)2 (SEQ ID NO: 330)-XTEN-(SGGS)2 (SEQ ID NO: 330)) as the linker in ABE2.1. In some embodiments, the ABE is ABE2.7, which is ABE2.1 tethered with an additional wild-type TadA monomer. In some embodiments, the ABE is ABE2.8, which is ABE2.1 tethered with an additional TadA*2.1 monomer. In some embodiments, the ABE is ABE2.9, which is a direct fusion of evolved TadA (TadA*2.1) to the N-terminus of ABE2.1. In some embodiments, the ABE is ABE2.10, which is a direct fusion of wild-type TadA to the N-terminus of ABE2.1. In some embodiments, the ABE is ABE2.11, which is ABE2.9 with an inactivating E59A mutation at the N-terminus of TadA* monomer. In some embodiments, the ABE is ABE2.12, which is ABE2.9 with an inactivating E59A mutation in the internal TadA* monomer.

In some embodiments, the ABE is a fifth generation ABE. In some embodiments, the ABE is ABE5.1, which is generated by importing a consensus set of mutations from surviving clones (H36L, R51L, S146C, and K157N) into ABE3.1. In some embodiments, the ABE is ABE5.3, which has a heterodimeric construct containing wild-type E. coli TadA fused to an internal evolved TadA*. In some embodiments, the ABE is ABE5.2, ABE5.4, ABE5.5, ABE5.6, ABE5.7, ABE5.8, ABE5.9, ABE5.10, ABE5.11, ABE5.12, ABE5.13, or ABE5.14, as shown in Table 9 below. In some embodiments, the ABE is a sixth generation ABE. In some embodiments, the ABE is ABE6.1, ABE6.2, ABE6.3, ABE6.4, ABE6.5, or ABE6.6, as shown in Table 9 below. In some embodiments, the ABE is a seventh generation ABE. In some embodiments, the ABE is ABE7.1, ABE7.2, ABE7.3, ABE7.4, ABE7.5, ABE7.6, ABE7.7, ABE7.8, ABE 7.9, or ABE7.10, as shown in Table 9 below.

In some embodiments, the adenosine base editor (ABE) can deaminate adenine in DNA. In some embodiments, the ABE is a an ABE as shown in Table 9 below.

TABLE 9
Genotypes of ABEs

| | 23 | 26 | 36 | 37 | 48 | 49 | 51 | 72 | 84 | 87 | 106 | 108 | 123 | 125 | 142 | 146 | 147 | 152 | 155 | 156 | 157 | 161 |
|---|---|---|---|---|---|---|---|---|---|---|---|---|---|---|---|---|---|---|---|---|---|---|
| ABE0.1 | W | R | H | N | P | | R | N | L | S | A | D | H | G | A | S | D | R | E | I | K | K |
| ABE0.2 | W | R | H | N | P | | R | N | L | S | A | D | H | G | A | S | D | R | E | I | K | K |
| ABE1.1 | W | R | H | N | P | | R | N | L | S | A | N | H | G | A | S | D | R | E | I | K | K |
| ABE1.2 | W | R | H | N | P | | R | N | L | S | V | N | H | G | A | S | D | R | E | I | K | K |
| ABE2.1 | W | R | H | N | P | | R | N | L | S | V | N | H | G | A | S | Y | R | V | I | K | K |
| ABE2.2 | W | R | H | N | P | | R | N | L | S | V | N | H | G | A | S | Y | R | V | I | K | K |
| ABE2.3 | W | R | H | N | P | | R | N | L | S | V | N | H | G | A | S | Y | R | V | I | K | K |
| ABE2.4 | W | R | H | N | P | | R | N | L | S | V | N | H | G | A | S | Y | R | V | I | K | K |
| ABE2.5 | W | R | H | N | P | | R | N | L | S | V | N | H | G | A | S | Y | R | V | I | K | K |
| ABE2.6 | W | R | H | N | P | | R | N | L | S | V | N | H | G | A | S | Y | R | V | I | K | K |
| ABE2.7 | W | R | H | N | P | | R | N | L | S | V | N | H | G | A | S | Y | R | V | I | K | K |
| ABE2.8 | W | R | H | N | P | | R | N | L | S | V | N | H | G | A | S | Y | R | V | I | K | K |
| ABE2.9 | W | R | H | N | P | | R | N | L | S | V | N | H | G | A | S | Y | R | V | I | K | K |
| ABE2.10 | W | R | H | N | P | | R | N | L | S | V | N | H | G | A | S | Y | R | V | I | K | K |
| ABE2.11 | W | R | H | N | P | | R | N | L | S | V | N | H | G | A | S | Y | R | V | I | K | K |
| ABE2.12 | W | R | H | N | P | | R | N | L | S | V | N | H | G | A | S | Y | R | V | I | K | K |
| ABE3.1 | W | R | H | N | P | | R | N | F | S | V | N | Y | G | A | S | Y | R | V | F | K | K |
| ABE3.2 | W | R | H | N | P | | R | N | F | S | V | N | Y | G | A | S | Y | R | V | F | K | K |
| ABE3.3 | W | R | H | N | P | | R | N | F | S | V | N | Y | G | A | S | Y | R | V | F | K | K |
| ABE3.4 | W | R | H | N | P | | R | N | F | S | V | N | Y | G | A | S | Y | R | V | F | K | K |
| ABE3.5 | W | R | H | N | P | | R | N | F | S | V | N | Y | G | A | S | Y | R | V | F | K | K |
| ABE3.6 | W | R | H | N | P | | R | N | F | S | V | N | Y | G | A | S | Y | R | V | F | K | K |
| ABE3.7 | W | R | H | N | P | | R | N | F | S | V | N | Y | G | A | S | Y | R | V | F | K | K |
| ABE3.8 | W | R | H | N | P | | R | N | F | S | V | N | Y | G | A | S | Y | R | V | F | K | K |
| ABE4.1 | W | R | H | N | P | | R | N | L | S | V | N | H | G | N | S | Y | R | V | I | K | K |
| ABE4.2 | W | G | H | N | P | | R | N | L | S | V | N | H | G | N | S | Y | R | V | I | K | K |
| ABE4.3 | W | R | H | N | P | | R | N | F | S | V | N | Y | G | N | S | Y | R | V | F | K | K |
| ABE5.1 | W | R | L | N | P | | L | N | F | S | V | N | Y | G | A | C | Y | R | V | F | N | K |
| ABE5.2 | W | R | H | S | P | | R | N | F | S | V | N | Y | G | A | S | Y | R | V | F | K | T |
| ABE5.3 | W | R | L | N | P | | L | N | I | S | V | N | Y | G | A | C | Y | R | V | F | N | K |
| ABE5.4 | W | R | H | S | P | | R | N | F | S | V | N | Y | G | A | S | Y | R | V | F | K | T |
| ABE5.5 | W | R | L | N | P | | L | N | F | S | V | N | Y | G | A | C | Y | R | V | F | N | K |
| ABE5.6 | W | R | L | N | P | | L | N | F | S | V | N | Y | G | A | C | Y | R | V | F | N | K |
| ABE5.7 | W | R | L | N | P | | L | N | F | S | V | N | Y | G | A | C | Y | R | V | F | N | K |
| ABE5.8 | W | R | L | N | P | | L | N | F | S | V | N | Y | G | A | C | Y | R | V | F | N | K |
| ABE5.9 | W | R | L | N | P | | L | N | F | S | V | N | Y | G | A | C | Y | R | V | F | N | K |
| ABE5.10 | W | R | L | N | P | | L | N | F | S | V | N | Y | G | A | C | Y | R | V | F | N | K |
| ABE5.11 | W | R | L | N | P | | L | N | F | S | V | N | Y | G | A | C | Y | R | V | F | N | K |
| ABE5.12 | W | R | L | N | P | | L | N | F | S | V | N | Y | G | A | C | Y | R | V | F | N | K |
| ABE5.13 | W | R | H | N | P | | L | D | F | S | V | N | Y | A | A | S | Y | R | V | F | K | K |
| ABE5.14 | W | R | H | N | S | | L | N | F | C | V | N | Y | G | A | S | Y | R | V | F | K | K |
| ABE6.1 | W | R | H | N | S | | L | N | F | S | V | N | Y | G | N | S | Y | R | V | F | K | K |
| ABE6.2 | W | R | H | N | T | V | L | N | F | S | V | N | Y | G | N | S | Y | R | V | F | N | K |
| ABE6.3 | W | R | L | N | S | | L | N | F | S | V | N | Y | G | A | C | Y | R | V | F | N | K |
| ABE6.4 | W | R | L | N | S | | L | N | F | S | V | N | Y | G | N | C | Y | R | V | F | N | K |
| ABE6.5 | W | R | L | N | T | V | L | N | F | S | V | N | Y | G | A | C | Y | R | V | F | N | K |
| ABE6.6 | W | R | L | N | T | V | L | N | F | S | V | N | Y | G | N | C | Y | R | V | F | N | K |
| ABE7.1 | W | R | L | N | A | | L | N | F | S | V | N | Y | G | A | C | Y | R | V | F | N | K |
| ABE7.2 | W | R | L | N | A | | L | N | F | S | V | N | Y | G | N | C | Y | R | V | F | N | K |
| ABE7.3 | L | R | L | N | A | | L | N | F | S | V | N | Y | G | A | C | Y | R | V | F | N | K |
| ABE7.4 | R | R | L | N | A | | L | N | F | S | V | N | Y | G | A | C | Y | R | V | F | N | K |
| ABE7.5 | W | R | L | N | A | | L | N | F | S | V | N | Y | G | A | C | Y | H | V | F | N | K |
| ABE7.6 | W | R | L | N | A | | L | N | I | S | V | N | Y | G | A | C | Y | P | V | F | N | K |
| ABE7.7 | L | R | I | N | A | | L | N | F | S | V | N | Y | G | A | C | Y | P | V | I | N | K |
| ABE7.8 | L | R | L | N | A | | L | N | F | S | V | N | Y | G | N | C | Y | R | V | F | N | K |
| ABE7.9 | L | R | L | N | A | | L | N | F | S | V | N | Y | G | N | C | Y | P | V | F | N | K |
| ABE7.10 | R | R | L | N | A | | L | N | F | S | V | N | Y | G | A | C | Y | P | V | F | N | K |

In some embodiments, the base editor is an eighth generation ABE (ABE8). In some embodiments, the ABE8 contains a TadA*8 variant. In some embodiments, the ABE8 has a monomeric construct containing a TadA*8 variant (“ABE8.x-m”). In some embodiments, the ABE8 is ABE8.1-m, which has a monomeric construct containing TadA*7.10 with a Y147T mutation (TadA*8.1). In some embodiments, the ABE8 is ABE8.2-m, which has a monomeric construct containing TadA*7.10 with a Y147R mutation (TadA*8.2). In some embodiments, the ABE8 is ABE8.3-m, which has a monomeric construct containing TadA*7.10 with a Q154S mutation (TadA*8.3).

In some embodiments, the ABE8 is ABE8.4-m, which has a monomeric construct containing TadA*7.10 with a Y123H mutation (TadA*8.4). In some embodiments, the ABE8 is ABE8.5-m, which has a monomeric construct containing TadA*7.10 with a V82S mutation (TadA*8.5). In some embodiments, the ABE8 is ABE8.6-m, which has a monomeric construct containing TadA*7.10 with a T166R mutation (TadA*8.6). In some embodiments, the ABE8 is ABE8.7-m, which has a monomeric construct containing TadA*7.10 with a Q154R mutation (TadA*8.7). In some embodiments, the ABE8 is ABE8.8-m, which has a monomeric construct containing TadA*7.10 with Y147R, Q154R, and Y123H mutations (TadA*8.8). In some embodiments, the ABE8 is ABE8.9-m, which has a monomeric construct containing TadA*7.10 with Y147R, Q154R and I76Y mutations (TadA*8.9). In some embodiments, the ABE8 is ABE8.10-m, which has a monomeric construct containing TadA*7.10 with Y147R, Q154R, and T166R mutations (TadA*8.10). In some embodiments, the ABE8 is ABE8.11-m, which has a monomeric construct containing TadA*7.10 with Y147T and Q154R mutations (TadA*8.11). In some embodiments, the ABE8 is ABE8.12-m, which has a monomeric construct containing TadA*7.10 with Y147T and Q154S mutations (TadA*8.12).

In some embodiments, the ABE8 is ABE8.13-m, which has a monomeric construct containing TadA*7.10 with Y123H (Y123H reverted from H123Y), Y147R, Q154R and I76Y mutations (TadA*8.13). In some embodiments, the ABE8 is ABE8.14-m, which has a monomeric construct containing TadA*7.10 with I76Y and V82S mutations (TadA*8.14). In some embodiments, the ABE8 is ABE8.15-m, which has a monomeric construct containing TadA*7.10 with V82S and Y147R mutations (TadA*8.15). In some embodiments, the ABE8 is ABE8.16-m, which has a monomeric construct containing TadA*7.10 with V82S, Y123H (Y123H reverted from H123Y) and Y147R mutations (TadA*8.16). In some embodiments, the ABE8 is ABE8.17-m, which has a monomeric construct containing TadA*7.10 with V82S and Q154R mutations (TadA*8.17). In some embodiments, the ABE8 is ABE8.18-m, which has a monomeric construct containing TadA*7.10 with V82S, Y123H (Y123H reverted from H123Y) and Q154R mutations (TadA*8.18). In some embodiments, the ABE8 is ABE8.19-m, which has a monomeric construct containing TadA*7.10 with V82S, Y123H (Y123H reverted from H123Y), Y147R and Q154R mutations (TadA*8.19). In some embodiments, the ABE8 is ABE8.20-m, which has a monomeric construct containing TadA*7.10 with I76Y, V82S, Y123H (Y123H reverted from H123Y), Y147R and Q154R mutations (TadA*8.20). In some embodiments, the ABE8 is ABE8.21-m, which has a monomeric construct containing TadA*7.10 with Y147R and Q154S mutations (TadA*8.21). In some embodiments, the ABE8 is ABE8.22-m, which has a monomeric construct containing TadA*7.10 with V82S and Q154S mutations (TadA*8.22). In some embodiments, the ABE8 is ABE8.23-m, which has a monomeric construct containing TadA*7.10 with V82S and Y123H (Y123H reverted from H123Y) mutations (TadA*8.23). In some embodiments, the ABE8 is ABE8.24-m, which has a monomeric construct containing TadA*7.10 with V82S, Y123H (Y123H reverted from H123Y), and Y147T mutations (TadA*8.24).

In some embodiments, the ABE8 has a heterodimeric construct containing wild-type E. coli TadA fused to a TadA*8 variant (“ABE8.x-d”). In some embodiments, the ABE8 is ABE8.1-d, which has a heterodimeric construct containing wild-type E. coli TadA fused to TadA*7.10 with a Y147T mutation (TadA*8.1). In some embodiments, the ABE8 is ABE8.2-d, which has a heterodimeric construct containing wild-type E. coli TadA fused to TadA*7.10 with a Y147R mutation (TadA*8.2). In some embodiments, the ABE8 is ABE8.3-d, which has a heterodimeric construct containing wild-type E. coli TadA fused to TadA*7.10 with a Q154S mutation (TadA*8.3). In some embodiments, the ABE8 is ABE8.4-d, which has a heterodimeric construct containing wild-type E. coli TadA fused to TadA*7.10 with a Y123H mutation (TadA*8.4). In some embodiments, the ABE8 is ABE8.5-d, which has a heterodimeric construct containing wild-type E. coli TadA fused to TadA*7.10 with a V82S mutation (TadA*8.5). In some embodiments, the ABE8 is ABE8.6-d, which has a heterodimeric construct containing wild-type E. coli TadA fused to TadA*7.10 with a T166R mutation (TadA*8.6). In some embodiments, the ABE8 is ABE8.7-d, which has a heterodimeric construct containing wild-type E. coli TadA fused to TadA*7.10 with a Q154R mutation (TadA*8.7). In some embodiments, the ABE8 is ABE8.8-d, which has a heterodimeric construct containing wild-type E. coli TadA fused to TadA*7.10 with Y147R, Q154R, and Y123H mutations (TadA*8.8). In some embodiments, the ABE8 is ABE8.9-d, which has a heterodimeric construct containing wild-type E. coli TadA fused to TadA*7.10 with Y147R, Q154R and I76Y mutations (TadA*8.9). In some embodiments, the ABE8 is ABE8.10-d, which has a heterodimeric construct containing wild-type E. coli TadA fused to TadA*7.10 with Y147R, Q154R, and T166R mutations (TadA*8.10). In some embodiments, the ABE8 is ABE8.11-d, which has a heterodimeric construct containing wild-type E. coli TadA fused to TadA*7.10 with Y147T and Q154R mutations (TadA*8.11). In some embodiments, the ABE8 is ABE8.12-d, which has heterodimeric construct containing wild-type E. coli TadA fused to TadA*7.10 with Y147T and Q154S mutations (TadA*8.12). In some embodiments, the ABE8 is ABE8.13-d, which has a heterodimeric construct containing wild-type E. coli TadA fused to TadA*7.10 with Y123H (Y123H reverted from H123Y), Y147R, Q154R and I76Y mutations (TadA*8.13). In some embodiments, the ABE8 is ABE8.14-d, which has a heterodimeric construct containing wild-type E. coli TadA fused to TadA*7.10 with I76Y and V82S mutations (TadA*8.14). In some embodiments, the ABE8 is ABE8.15-d, which has a heterodimeric construct containing wild-type E. coli TadA fused to TadA*7.10 with V82S and Y147R mutations (TadA*8.15). In some embodiments, the ABE8 is ABE8.16-d, which has a heterodimeric construct containing wild-type E. coli TadA fused to TadA*7.10 with V82S, Y123H (Y123H reverted from H123Y) and Y147R mutations (TadA*8.16). In some embodiments, the ABE8 is ABE8.17-d, which has a heterodimeric construct containing wild-type E. coli TadA fused to TadA*7.10 with V82S and Q154R mutations (TadA*8.17). In some embodiments, the ABE8 is ABE8.18-d, which has a heterodimeric construct containing wild-type E. coli TadA fused to TadA*7.10 with V82S, Y123H (Y123H reverted from H123Y) and Q154R mutations (TadA*8.18). In some embodiments, the ABE8 is ABE8.19-d, which has a heterodimeric construct containing wild-type E. coli TadA fused to TadA*7.10 with V82S, Y123H (Y123H reverted from H123Y), Y147R and Q154R mutations (TadA*8.19). In some embodiments, the ABE8 is ABE8.20-d, which has a heterodimeric construct containing wild-type E. coli TadA fused to TadA*7.10 with I76Y, V82S, Y123H (Y123H reverted from H123Y), Y147R and Q154R mutations (TadA*8.20). In some embodiments, the ABE8 is ABE8.21-d, which has a heterodimeric construct containing wild-type E. coli TadA fused to TadA*7.10 with Y147R and Q154S mutations (TadA*8.21). In some embodiments, the ABE8 is ABE8.22-d, which has a heterodimeric construct containing wild-type E. coli TadA fused to TadA*7.10 with V82S and Q154S mutations (TadA*8.22). In some embodiments, the ABE8 is ABE8.23-d, which has a heterodimeric construct containing wild-type E. coli TadA fused to TadA*7.10 with V82S and Y123H (Y123H reverted from H123Y) mutations (TadA*8.23). In some embodiments, the ABE8 is ABE8.24-d, which has a heterodimeric construct containing wild-type E. coli TadA fused to TadA*7.10 with V82S, Y123H (Y123H reverted from H123Y), and Y147T mutations (TadA*8.24).

In some embodiments, the ABE8 has a heterodimeric construct containing TadA*7.10 fused to a TadA*8 variant (“ABE8.x-7”). In some embodiments, the ABE8 is ABE8.1-7, which has a heterodimeric construct containing TadA*7.10 fused to TadA*7.10 with a Y147T mutation (TadA*8.1). In some embodiments, the ABE8 is ABE8.2-7, which has a heterodimeric construct containing TadA*7.10 fused to TadA*7.10 with a Y147R mutation (TadA*8.2). In some embodiments, the ABE8 is ABE8.3-7, which has a heterodimeric construct containing TadA*7.10 fused to TadA*7.10 with a Q154S mutation (TadA*8.3). In some embodiments, the ABE8 is ABE8.4-7, which has a heterodimeric construct containing TadA*7.10 fused to TadA*7.10 with a Y123H mutation (TadA*8.4). In some embodiments, the ABE8 is ABE8.5-7, which has a heterodimeric construct containing TadA*7.10 fused to TadA*7.10 with a V82S mutation (TadA*8.5). In some embodiments, the ABE8 is ABE8.6-7, which has a heterodimeric construct containing TadA*7.10 fused to TadA*7.10 with a T166R mutation (TadA*8.6). In some embodiments, the ABE8 is ABE8.7-7, which has a heterodimeric construct containing TadA*7.10 fused to TadA*7.10 with a Q154R mutation (TadA*8.7). In some embodiments, the ABE8 is ABE8.8-7, which has a heterodimeric construct containing TadA*7.10 fused to TadA*7.10 with Y147R, Q154R, and Y123H mutations (TadA*8.8). In some embodiments, the ABE8 is ABE8.9-7, which has a heterodimeric construct containing TadA*7.10 fused to TadA*7.10 with Y147R, Q154R and I76Y mutations (TadA*8.9). In some embodiments, the ABE8 is ABE8.10-7, which has a heterodimeric construct containing TadA*7.10 fused to TadA*7.10 with Y147R, Q154R, and T166R mutations (TadA*8.10). In some embodiments, the ABE8 is ABE8.11-7, which has a heterodimeric construct containing TadA*7.10 fused to TadA*7.10 with Y147T and Q154R mutations (TadA*8.11). In some embodiments, the ABE8 is ABE8.12-7, which has a heterodimeric construct containing TadA*7.10 fused to TadA*7.10 with Y147T and Q154S mutations (TadA*8.12). In some embodiments, the ABE8 is ABE8.13-7, which has a heterodimeric construct containing TadA*7.10 fused to TadA*7.10 with Y123H (Y123H reverted from H123Y), Y147R, Q154R and I76Y mutations (TadA*8.13). In some embodiments, the ABE8 is ABE8.14-7, which has a heterodimeric construct containing TadA*7.10 fused to TadA*7.10 with I76Y and V82S mutations (TadA*8.14). In some embodiments, the ABE8 is ABE8.15-7, which has a heterodimeric construct containing TadA*7.10 fused to TadA*7.10 with V82S and Y147R mutations (TadA*8.15). In some embodiments, the ABE8 is ABE8.16-7, which has a heterodimeric construct containing TadA*7.10 fused to TadA*7.10 with V82S, Y123H (Y123H reverted from H123Y) and Y147R mutations (TadA*8.16). In some embodiments, the ABE8 is ABE8.17-7, which has a heterodimeric construct containing TadA*7.10 fused to TadA*7.10 with V82S and Q154R mutations (TadA*8.17). In some embodiments, the ABE8 is ABE8.18-7, which has a heterodimeric construct containing TadA*7.10 fused to TadA*7.10 with V82S, Y123H (Y123H reverted from H123Y) and Q154R mutations (TadA*8.18). In some embodiments, the ABE8 is ABE8.19-7, which has a heterodimeric construct containing TadA*7.10 fused to TadA*7.10 with V82S, Y123H (Y123H reverted from H123Y), Y147R and Q154R mutations (TadA*8.19). In some embodiments, the ABE8 is ABE8.20-7, which has a heterodimeric construct containing TadA*7.10 fused to TadA*7.10 with I76Y, V82S, Y123H (Y123H reverted from H123Y), Y147R and Q154R mutations (TadA*8.20). In some embodiments, the ABE8 is ABE8.21-7, which has a heterodimeric construct containing TadA*7.10 fused to TadA*7.10 with Y147R and Q154S mutations (TadA*8.21). In some embodiments, the ABE8 is ABE8.22-7, which has a heterodimeric construct containing TadA*7.10 fused to TadA*7.10 with V82S and Q154S mutations (TadA*8.22). In some embodiments, the ABE8 is ABE8.23-7, which has a heterodimeric construct containing TadA*7.10 fused to TadA*7.10 with V82S and Y123H (Y123H reverted from H123Y) mutations (TadA*8.23). In some embodiments, the ABE8 is ABE8.24-7, which has a heterodimeric construct containing TadA*7.10 fused to TadA*7.10 with V82S, Y123H (Y123H reverted from H123Y), and Y147T mutations (TadA*8.24).

In some embodiments, the ABE is ABE8.1-m, ABE8.2-m, ABE8.3-m, ABE8.4-m, ABE8.5-m, ABE8.6-m, ABE8.7-m, ABE8.8-m, ABE8.9-m, ABE8.10-m, ABE8.11-m, ABE8.12-m, ABE8.13-m, ABE8.14-m, ABE8.15-m, ABE8.16-m, ABE8.17-m, ABE8.18-m, ABE8.19-m, ABE8.20-m, ABE8.21-m, ABE8.22-m, ABE8.23-m, ABE8.24-m, ABE8.1-d, ABE8.2-d, ABE8.3-d, ABE8.4-d, ABE8.5-d, ABE8.6-d, ABE8.7-d, ABE8.8-d, ABE8.9-d, ABE8.10-d, ABE8.11-d, ABE8.12-d, ABE8.13-d, ABE8.14-d, ABE8.15-d, ABE8.16-d, ABE8.17-d, ABE8.18-d, ABE8.19-d, ABE8.20-d, ABE8.21-d, ABE8.22-d, ABE8.23-d, or ABE8.24-d as shown in Table 10 below.

TABLE 10
Adenosine Deaminase Base Editor 8 (ABE8) Variants

| | Adenosine | |
|---|---|---|
| ABE8 | Deaminase | Adenosine Deaminase Description |
| ABE8.1-m | TadA*8.1 | Monomer_TadA*7.10 + Y147T |
| ABE8.2-m | TadA*8.2 | Monomer_TadA*7.10 + Y147R |
| ABE8.3-m | TadA*8.3 | Monomer_TadA*7.10 + Q154S |
| ABE8.4-m | TadA*8.4 | Monomer_TadA*7.10 + Y123H |
| ABE8.5-m | TadA*8.5 | Monomer_TadA*7.10 + V82S |
| ABE8.6-m | TadA*8.6 | Monomer_TadA*7.10 + T166R |
| ABE8.7-m | TadA*8.7 | Monomer_TadA*7.10 + Q154R |
| ABE8.8-m | TadA*8.8 | Monomer_TadA7.10 + Y147R_Q154R_Y123H |
| ABE8.9-m | TadA*8.9 | Monomer_TadA*7.10 + Y147R_Q154R_I76Y |
| ABE8.10- | TadA*8.10 | Monomer_TadA*7.10 + Y147R_Q154R_T166R |
| m | | |
| ABE8.11- | TadA*8.11 | Monomer_TadA*7.10 + Y147T_Q154R |
| m | | |
| ABE8.12- | TadA*8.12 | Monomer_TadA*7.10 + Y147T_Q154S |
| m | | |
| ABE8.13- | TadA*8.13 | Monomer_TadA*7.10 + Y123H_Y147R_Q154R_176Y |
| m | | |
| ABE8.14- | TadA*8.14 | Monomer_TadA*7.10 + I76Y_V82S |
| m | | |
| ABE8.15- | TadA*8.15 | Monomer_TadA*7.10 + V82S_Y147R |
| m | | |
| ABE8.16- | TadA*8.16 | Monomer_TadA*7.10 + V82S_Y123H_Y147R |
| m | | |
| ABE8.17- | TadA*8.17 | Monomer_TadA*7.10 + V82S_Q154R |
| m | | |
| ABE8.18- | TadA*8.18 | Monomer_TadA*7.10 + V82S_Y123H_Q154R |
| m | | |
| ABE8.19- | TadA*8.19 | Monomer_TadA*7.10 + V82S_Y123H_Y147R_Q154R |
| m | | |
| ABE8.20- | TadA*8.20 | Monomer_TadA*7.10 + I76Y_V82S_Y123H_Y147R_Q154R |
| m | | |
| ABE8.21- | TadA*8.21 | Monomer_TadA*7.10 + Y147R_Q154S |
| m | | |
| ABE8.22- | TadA*8.22 | Monomer_TadA*7.10 + V82S_Q154S |
| m | | |
| ABE8.23- | TadA*8.23 | Monomer_TadA*7.10 + V82S_Y123H |
| m | | |
| ABE8.24- | TadA*8.24 | Monomer_TadA*7.10 + V82S_Y123H_Y147T |
| m | | |
| ABE8.1-d | TadA*8.1 | Heterodimer_(WT) + (TadA*7.10 + Y147T) |
| ABE8.2-d | TadA*8.2 | Heterodimer_(WT) + (TadA*7.10 + Y147R) |
| ABE8.3-d | TadA*8.3 | Heterodimer_(WT) + (TadA*7.10 + Q154S) |
| ABE8.4-d | TadA*8.4 | Heterodimer_(WT) + (TadA*7.10 + Y123H) |
| ABE8.5-d | TadA*8.5 | Heterodimer_(WT) + (TadA*7.10 + V82S) |
| ABE8.6-d | TadA*8.6 | Heterodimer_(WT) + (TadA*7.10 + T166R) |
| ABE8.7-d | TadA*8.7 | Heterodimer_(WT) + (TadA*7.10 + Q154R) |
| ABE8.8-d | TadA*8.8 | Heterodimer_(WT) + (TadA*7.10 + Y147R_Q154R_Y123H) |
| ABE8.9-d | TadA*8.9 | Heterodimer_(WT) + (TadA*7.10 + Y147R_Q154R_I76Y) |
| ABE8.10-d | TadA*8.10 | Heterodimer_(WT) + (TadA*7.10 + Y147R_Q154R_T166R) |
| ABE8.11-d | TadA*8.11 | Heterodimer_(WT) + (TadA*7.10 + Y147T_Q154R) |
| ABE8.12-d | TadA*8.12 | Heterodimer_(WT) + (TadA*7.10 + Y147T_Q154S) |
| ABE8.13-d | TadA*8.13 | Heterodimer_(WT) + (TadA*7.10 + |
| | | Y123H_Y147T_Q154R_176Y) |
| ABE8.14-d | TadA*8.14 | Heterodimer_(WT) + (TadA*7.10 + I76Y_V82S) |
| ABE8.15-d | TadA*8.15 | Heterodimer_(WT) + (TadA*7.10 + V82S_Y147R) |
| ABE8.16-d | TadA*8.16 | Heterodimer_(WT) + (TadA*7.10 + V82S_Y123H_Y147R) |
| ABE8.17-d | TadA*8.17 | Heterodimer_(WT) + (TadA*7.10 + V82S_Q154R) |
| ABE8.18-d | TadA*8.18 | Heterodimer_(WT) + (TadA*7.10 + V82S_Y123H_Q154R) |
| ABE8.19-d | TadA*8.19 | Heterodimer_(WT) + (TadA*7.10 + |
| | | V82S_Y123H_Y147R_Q154R) |
| ABE8.20-d | TadA*8.20 | Heterodimer_(WT) + (TadA*7.10 + |
| | | I76Y_V82S_Y123H_Y147R_Q154R) |
| ABE8.21-d | TadA*8.21 | Heterodimer_(WT) + (TadA*7.10 + Y147R_Q154S) |
| ABE8.22-d | TadA*8.22 | Heterodimer_(WT) + (TadA*7.10 + V82S_Q154S) |
| ABE8.23-d | TadA*8.23 | Heterodimer_(WT) + (TadA*7.10 + V82S_Y123H) |
| ABE8.24-d | TadA*8.24 | Heterodimer_(WT) + (TadA*7.10 + V82S_Y123H_Y147T) |

In some embodiments, the ABE8 is ABE8a-m, which has a monomeric construct containing TadA*7.10 with R26C, A109S, T111R, D119N, H122N, Y147D, F149Y, T166I, and D167N mutations (TadA*8a). In some embodiments, the ABE8 is ABE8b-m, which has a monomeric construct containing TadA*7.10 with V88A, A109S, T111R, D119N, H122N, F149Y, T166I, and D167N mutations (TadA*8b). In some embodiments, the ABE8 is ABE8c-m, which has a monomeric construct containing TadA*7.10 with R26C, A109S, T111R, D119N, H122N, F149Y, T166I, and D167N mutations (TadA*8c). In some embodiments, the ABE8 is ABE8d-m, which has a monomeric construct containing TadA*7.10 with V88A, T111R, D119N, and F149Y mutations (TadA*8d). In some embodiments, the ABE8 is ABE8e-m, which has a monomeric construct containing TadA*7.10 with A109S, T111R, D119N, H122N, Y147D, F149Y, T166I, and D167N mutations (TadA*8e).

In some embodiments, the ABE8 is ABE8a-d, which has a heterodimeric construct containing wild-type E. coli TadA fused to TadA*7.10 with R26C, A109S, T111R, D119, H122N, Y147D, F149Y, T166I, and D167N mutations (TadA*8a). In some embodiments, the ABE8 is ABE8b-d, which has a heterodimeric construct containing wild-type E. coli TadA fused to TadA*7.10 with V88A, A109S, T111R, D119N, H122N, F149Y, T166I, and D167N mutations (TadA*8b). In some embodiments, the ABE8 is ABE8c-d, which has a heterodimeric construct containing wild-type E. coli TadA fused to TadA*7.10 with R26C, A109S, T111R, D119N, H122N, F149Y, T166I, and D167N mutations (TadA*8c). In some embodiments, the ABE8 is ABE8d-d, which has a heterodimeric construct containing wild-type E. coli TadA fused to TadA*7.10 with V88A, T111R, D119N, and F149Y mutations (TadA*8d). In some embodiments, the ABE8 is ABE8e-d, which has a heterodimeric construct containing wild-type E. coli TadA fused to TadA*7.10 with A109S, T111R, D119N, H122N, Y147D, F149Y, T166I, and D167N mutations (TadA*8e).

In some embodiments, the ABE8 is ABE8a-7, which has a heterodimeric construct containing TadA*7.10 fused to TadA*7.10 with R26C, A109S, T111R, D119, H122N, Y147D, F149Y, T166I, and D167N mutations (TadA*8a). In some embodiments, the ABE8 is ABE8b-7, which has a heterodimeric construct containing TadA*7.10 fused to TadA*7.10 with V88A, A109S, T111R, D119N, H122N, F149Y, T166I, and D167N mutations (TadA*8b). In some embodiments, the ABE8 is ABE8c-7, which has a heterodimeric construct containing TadA*7.10 fused to TadA*7.10 with R26C, A109S, T111R, D119N, H122N, F149Y, T166I, and D167N mutations (TadA*8c). In some embodiments, the ABE8 is ABE8d-7, which has a heterodimeric construct containing TadA*7.10 fused to TadA*7.10 with V88A, T111R, D119N, and F149Y mutations (TadA*8d). In some embodiments, the ABE8 is ABE8e-7, which has a heterodimeric construct containing TadA*7.10 fused to TadA*7.10 with A109S, T111R, D119N, H122N, Y147D, F149Y, T166I, and D167N mutations (TadA*8e).

In some embodiments, the ABE is ABE8a-m, ABE8b-m, ABE8c-m, ABE8d-m, ABE8e-m, ABE8a-d, ABE8b-d, ABE8c-d, ABE8d-d, or ABE8e-d, as shown in Table 11 below. In some embodiments, the ABE is ABE8e-m or ABE8e-d. ABE8e shows efficient adenine base editing activity and low indel formation when used with Cas homologues other than SpCas9, for example, SaCas9, SaCas9-KKH, Cas12a homologues, e.g., LbCas12a, enAs-Cas12a, SpCas9-NG and circularly permuted CP1028-SpCas9 and CP1041-SpCas9. In addition to the mutations shown for ABE8e in Table 11, off-target RNA and DNA editing were reduced by introducing a V106W substitution into the TadA domain (as described in M. Richter et al., 2020, Nature Biotechnology, doi.org/10.1038/s41587-020-0453-z, the entire contents of which are incorporated by reference herein).

TABLE 11
Additional Adenosine Deaminase Base Editor 8 Variants

| ABE8 Base | Adenosine | |
|---|---|---|
| Editor | Deaminase | Adenosine Deaminase Description |
| ABE8a-m | TadA*8a | Monomer_*7.10 + R26C + A109S + T111R + D119N + |
| | | H122N + Y147D + F149Y + T166I + D167N |
| ABE8b-m | TadA*8b | Monomer_TadA*7.10 + V88A + A109S + T111R + D119N + |
| | | H122N + F149Y + T166I + D167N |
| ABE8c-m | TadA*8c | Monomer_TadA*7.10 + R26C + A109S + T111R + D119N + |
| | | H122N + F149Y + T166I + D167N |
| ABE8d-m | TadA*8d | Monomer_TadA*7.10 + V88A + T111R + D119N + F149Y |
| ABE8e-m | TadA*8e | Monomer_TadA*7.10 + A109S + T111R + D119N + H122N + |
| | | Y147D+ F149Y + T166I + D167N |
| ABE8a-d | TadA*8a | Heterodimer_(WT) + (TadA*7.10 + R26C + A109S + T111R + |
| | | D119N+ H122N+ Y147D + F149Y+ T166I+ D167N) |
| ABE8b-d | TadA*8b | Heterodimer_(WT) + (TadA*7.10 + V88A + A109S + T111R + |
| | | D119N + H122N + F149Y + T166I +D167N) |
| ABE8c-d | TadA*8c | Heterodimer_(WT) + (TadA*7.10 + R26C + A109S + T111R + |
| | | D119N + H122N + F149Y + T166I+ D167N) |
| ABE8d-d | TadA*8d | Heterodimer_(WT) + (TadA*7.10 + V88A + T111R + D119N + |
| | | F149Y) |
| ABE8e-d | TadA*8e | Heterodimer_(WT) + (TadA*7.10 + A109S + T111R + D119N + |
| | | H122N+Y147D+ F149Y + T166I + D167N) |

In some embodiments, base editors (e.g., ABE8) are generated by cloning an adenosine deaminase variant (e.g., TadA*8) into a scaffold that includes a circular permutant Cas9 (e.g., CP5 or CP6) and a bipartite nuclear localization sequence. In some embodiments, the base editor (e.g., ABE7.9, ABE7.10, or ABE8) is an NGC PAM CP5 variant (S. pyogenes Cas9 or spVRQR Cas9). In some embodiments, the base editor (e.g., ABE7.9, ABE7.10, or ABE8) is an AGA PAM CP5 variant (S. pyogenes Cas9 or spVRQR Cas9). In some embodiments, the base editor (e.g., ABE7.9, ABE7.10, or ABE8) is an NGC PAM CP6 variant (S. pyogenes Cas9 or spVRQR Cas9). In some embodiments, the base editor (e.g. ABE7.9, ABE7.10, or ABE8) is an AGA PAM CP6 variant (S. pyogenes Cas9 or spVRQR Cas9).

In some embodiments, the ABE has a genotype as shown in Table 12 below.

TABLE 12
Genotypes of ABEs

| | 23 | 26 | 36 | 37 | 48 | 49 | 51 | 72 | 84 | 87 | 105 | 108 | 123 | 125 | 142 | 145 | 147 | 152 | 155 | 156 | 157 | 161 |
|---|---|---|---|---|---|---|---|---|---|---|---|---|---|---|---|---|---|---|---|---|---|---|
| ABE7.9 | L | R | L | N | A | | L | N | F | S | V | N | Y | G | N | C | Y | P | V | F | N | K |
| ABE7.10 | R | R | L | N | A | | L | N | F | S | V | N | Y | G | A | C | Y | P | V | F | N | K |

As shown in Table 13 below, genotypes of 40 ABE8s are described. Residue positions in the evolved E. coli TadA portion of ABE are indicated. Mutational changes in ABE8 are shown when distinct from ABE7.10 mutations. In some embodiments, the ABE has a genotype of one of the ABEs as shown in Table 13 below.

TABLE 13
Residue Identity in Evolved TadA

| | 23 | 36 | 48 | 51 | 76 | 82 | 84 | 106 | 108 | 123 | 146 | 147 | 152 | 154 | 155 | 156 | 157 | 166 |
|---|---|---|---|---|---|---|---|---|---|---|---|---|---|---|---|---|---|---|
| ABE7.10 | R | L | A | L | I | V | F | V | N | Y | C | Y | P | Q | V | F | N | T |
| ABE8.1-m | | | | | | | | | | | | T | | | | | | |
| ABE8.2-m | | | | | | | | | | | | R | | | | | | |
| ABE8.3-m | | | | | | | | | | | | | | S | | | | |
| ABE8.4-m | | | | | | | | | | H | | | | | | | | |
| ABE8.5-m | | | | | | S | | | | | | | | | | | | |
| ABE8.6-m | | | | | | | | | | | | | | | | | | R |
| ABE8.7-m | | | | | | | | | | | | | | R | | | | |
| ABE8.8-m | | | | | | | | | | H | | R | | R | | | | |
| ABE8.9-m | | | | | Y | | | | | | | R | | R | | | | |
| ABE8.10-m | | | | | | | | | | | | R | | R | | | | R |
| ABE8.11-m | | | | | | | | | | | | T | | R | | | | |
| ABE8.12-m | | | | | | | | | | | | T | | S | | | | |
| ABE8.13-m | | | | | Y | | | | | H | | R | | R | | | | |
| ABE8.14-m | | | | | Y | S | | | | | | | | | | | | |
| ABE8.15-m | | | | | | S | | | | | | R | | | | | | |
| ABE8.16-m | | | | | | S | | | | H | | R | | | | | | |
| ABE8.17-m | | | | | | S | | | | | | | | R | | | | |
| ABE8.18-m | | | | | | S | | | | H | | | | R | | | | |
| ABE8.19-m | | | | | | S | | | | H | | R | | R | | | | |
| ABE8.20-m | | | | | Y | S | | | | H | | R | | R | | | | |
| ABE8.21-m | | | | | | | | | | | | R | | S | | | | |
| ABE8.22-m | | | | | | S | | | | | | | | S | | | | |
| ABE8.23-m | | | | | | S | | | | H | | | | | | | | |
| ABE8.24-m | | | | | | S | | | | H | | T | | | | | | |
| ABE8.1-d | | | | | | | | | | | | T | | | | | | |
| ABE8.2-d | | | | | | | | | | | | R | | | | | | |
| ABE8.3-d | | | | | | | | | | | | | | S | | | | |
| ABE8.4-d | | | | | | | | | | H | | | | | | | | |
| ABE8.5-d | | | | | | S | | | | | | | | | | | | |
| ABE8.6-d | | | | | | | | | | | | | | | | | | R |
| ABE8.7-d | | | | | | | | | | | | | | R | | | | |
| ABE8.8-d | | | | | | | | | | H | | R | | R | | | | |
| ABE8.9-d | | | | | Y | | | | | | | R | | R | | | | |
| ABE8.10-d | | | | | | | | | | | | R | | R | | | | R |
| ABE8.11-d | | | | | | | | | | | | T | | R | | | | |
| ABE8.12-d | | | | | | | | | | | | T | | S | | | | |
| ABE8.13-d | | | | | Y | | | | | H | | R | | R | | | | |
| ABE8.14-d | | | | | Y | S | | | | | | | | | | | | |
| ABE8.15-d | | | | | | S | | | | | | R | | | | | | |
| ABE8.16-d | | | | | | S | | | | H | | R | | | | | | |
| ABE8.17-d | | | | | | S | | | | | | | | R | | | | |
| ABE8.18-d | | | | | | S | | | | H | | | | R | | | | |
| ABE8.19-d | | | | | | S | | | | H | | R | | R | | | | |
| ABE8.20-d | | | | | Y | S | | | | H | | R | | R | | | | |
| ABE8.21-d | | | | | | | | | | | | R | | S | | | | |
| ABE8.22-d | | | | | | S | | | | | | | | S | | | | |
| ABE8.23-d | | | | | | S | | | | H | | | | | | | | |
| ABE8.24-d | | | | | | S | | | | H | | T | | | | | | |

In some embodiments, the base editor is ABE8.1, which comprises or consists essentially of the following sequence or a fragment thereof having adenosine deaminase activity:

ABE8.1 Y147T CP5 NGC PAM monomer
(SEQ ID NO: 331)
MSEVEFSHEYWMRHALTLAKRARDEREVPVGAVLVLNNRVIGEGWNRAIGLHDPTAHAEIMA
LRQGGLVMQNYRLIDATLYVTFEPCVMCAGAMIHSRIGRVVFGVRNAKTGAAGSLMDVLHYP
GMNHRVEITEGILADECAALLCTFFRMPRQVFNAQKKAQSSTD [symbol] [symbol]
EIGKATAKYFFYSNIMNFFKTEITLANGEIRKRPLIETNGETGEIVWDK
GRDFATVRKVLSMPQVNIVKKTEVQTGGFSKESILPKRNSDKLIARKKDWDPKKYGGFMQPT
VAYSVLVVAKVEKGKSKKLKSVKELLGITIMERSSFEKNPIDFLEAKGYKEVKKDLIIKLPK
YSLFELENGRKRMLASAKFLQKGNELALPSKYVNFLYLASHYEKLKGSPEDNEQKQLFVEQH
KHYLDEIIEQISEFSKRVILADANLDKVLSAYNKHRDKPIREQAENIIHLFTLTNLGAPRAF
KYFDTTIARKEYRSTKEVLDATLIHQSITGLYETRIDLSQLGGD [symbol] [symbol]
DKKYSIGLAIGTNSVGWAVITDEYKVPSKKFKVLGNTDRHSIKKNLIGALLFDSGETAE
ATRLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKNLSDAILLSDILRV
NTEITKAPLSASMIKRYDEHHQDLTLLKALVRQQLPEKYKEIFFDQSKNGYAGYIDGGASQE
EFYKFIKPILEKMDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVVDKGASAQSFIER
MTNFDKNLPNEKVLPKHSLLYEYFTVYNELTKVKYVTEGMRKPAFLSGEQKKAIVDLLFKTN
RKVTVKQLKEDYFKKIECFDSVEISGVEDRFNASLGTYHDLLKIIKDKDFLDNEENEDILED
IVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVK
VVDELVKVMGRHKPENIVIEMARENQTTQKGQKNSRERMKRIEEGIKELGSQILKEHPVENT
QLQNEKLYLYYLQNGRDMYVDQELDINRLSDYDVDHIVPQSFLKDDSIDNKVLTRSDKNRGK
SDNVPSEEVVKKMKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREINNYHHAHDAYLN
AVVGTALIKKYPKLESEFVYGDYKVYDVRKMIAKSEQEGADKRTADGSEFESPKKKRKV

In the above sequence, the plain text denotes an adenosine deaminase sequence, bold sequence indicates sequence derived from Cas9, the italicized sequence denotes a linker sequence, and the underlined sequence denotes a bipartite nuclear localization sequence. Other ABE8 sequences are provided in the attached sequence listing (SEQ ID NOs: 332-354).

In some embodiments, the base editor includes an adenosine deaminase variant comprising an amino acid sequence, which contains alterations relative to an ABE 7*10 reference sequence, as described herein. The term “monomer” as used in Table 14A refers to a monomeric form of TadA*7.10 comprising the alterations described. The term “heterodimer” as used in Table 14A refers to the specified wild-type E. coli TadA adenosine deaminase fused to a TadA*7.10 comprising the alterations as described.

TABLE 14A
Adenosine Deaminase Base Editor Variants

| | Adenosine | |
|---|---|---|
| ABE | Deaminase | Adenosine Deaminase Description |
| ABE-605m | MSP605 | monomer_TadA*7.10 + V82G + Y147T + Q154S |
| ABE-680m | MSP680 | monomer_TadA*7.10 + 176Y + V82G + Y147T + Q154S |
| ABE-823m | MSP823 | monomer_TadA*7.10 + L36H + V82G + Y147T + Q154S + |
| | | N157K |
| ABE-824m | MSP824 | monomer_TadA*7.10 + V82G + Y147D + F149Y + Q154S + |
| | | D167N |
| ABE-825m | MSP825 | monomer_TadA*7.10 + L36H + V82G + Y147D + F149Y + |
| | | Q154S + N157K + D167N |
| ABE-827m | MSP827 | monomer_TadA*7.10 + L36H + 176Y + V82G + Y147T + Q154S + |
| | | N157K |
| ABE-828m | MSP828 | monomer_TadA*7.10 + 176Y + V82G + Y147D + F149Y + Q154S + |
| | | D167N |
| ABE-829m | MSP829 | monomer_TadA*7.10 + L36H + 176Y + V82G + Y147D + F149Y + |
| | | Q154S + N157K + D167N |
| ABE-605d | MSP605 | heterodimer_(WT) + (TadA*7.10 + V82G + Y147T + Q154S) |
| ABE-680d | MSP680 | heterodimer_(WT) + (TadA*7.10 + I76Y + V82G + Y147T + |
| | | Q154S) |
| ABE-823d | MSP823 | heterodimer_(WT) + (TadA*7.10 + L36H + V82G + Y147T + |
| | | Q154S + N157K) |
| ABE-824d | MSP824 | heterodimer_(WT) + (TadA*7.10 + V82G + Y147D + F149Y + |
| | | Q154S + D167N) |
| ABE-825d | MSP825 | heterodimer_(WT) + (TadA*7.10 + L36H + V82G + Y147D + |
| | | F149Y + Q154S + N157K + D167N) |
| ABE-827d | MSP827 | heterodimer_(WT) + (TadA*7.10 + L36H + 176Y + V82G + Y147T + |
| | | Q154S + N157K) |
| ABE-828d | MSP828 | heterodimer_(WT) + (TadA*7.10 + I76Y + V82G + Y147D + |
| | | F149Y + Q154S + D167N) |
| ABE-829d | MSP829 | heterodimer_(WT) + (TadA*7.10 + L36H + 176Y + V82G + Y147D + |
| | | F149Y + Q154S + N157K + D167N) |

In some embodiments, the base editor is a ninth generation ABE (ABE9). In some embodiments, the ABE9 contains a TadA*9 variant. ABE9 base editors include an adenosine deaminase variant comprising an amino acid sequence, which contains alterations relative to an ABE 7*10 reference sequence, as described herein. Exemplary ABE9 variants are listed in Table 14A.

Details of ABE9 base editors are described in International PCT Application No. PCT/2020/049975, which is incorporated herein by reference for its entirety, and are listed in Table 14B. In Table 14B, “monomer” indicates an ABE comprising a single TadA*7.10 comprising the indicated alterations and “heterodimer” indicates an ABE comprising a TadA*7.10 comprising the indicated alterations fused to an E. coli TadA adenosine deaminase.

TABLE 14B
Adenosine Base Editor 9 (ABE9) Variants.

| ABE9 Description | Alterations |
|---|---|
| ABE9.1_monomer | E25F, V82S, Y123H, T133K, Y147R, Q154R |
| ABE9.2_monomer | E25F, V82S, Y123H, Y147R, Q154R |
| ABE9.3_monomer | V82S, Y123H, P124W, Y147R, Q154R |
| ABE9.4_monomer | L51W, V82S, Y123H, C146R, Y147R, Q154R |
| ABE9.5_ monomer | P54C, V82S, Y123H, Y147R, Q154R |
| ABE9.6_monomer | Y73S, V82S, Y123H, Y147R, Q154R |
| ABE9.7_monomer | N38G, V82T, Y123H, Y147R, Q154R |
| ABE9.8_monomer | R23H, V82S, Y123H, Y147R, Q154R |
| ABE9.9_monomer | R21N, V82S, Y123H, Y147R, Q154R |
| ABE9.10_monomer | V82S, Y123H, Y147R, Q154R, A158K |
| ABE9.11_monomer | N72K, V82S, Y123H, D139L, Y147R, Q154R, |
| ABE9.12_monomer | E25F, V82S, Y123H, D139M, Y147R, Q154R |
| ABE9.13_monomer | M70V, V82S, M94V, Y123H, Y147R, Q154R |
| ABE9.14_monomer | Q71M, V82S, Y123H, Y147R, Q154R |
| ABE9.15_heterodimer | E25F, V82S, Y123H, T133K, Y147R, Q154R |
| ABE9.16_heterodimer | E25F, V82S, Y123H, Y147R, Q154R |
| ABE9.17_heterodimer | V82S, Y123H, P124W, Y147R, Q154R |
| ABE9.18_heterodimer | L51W, V82S, Y123H, C146R, Y147R, Q154R |
| ABE9.19_heterodimer | P54C, V82S, Y123H, Y147R, Q154R |
| ABE9.2_heterodimer | Y73S, V82S, Y123H, Y147R, Q154R |
| ABE9.21_heterodimer | N38G, V82T, Y123H, Y147R, Q154R |
| ABE9.22_heterodimer | R23H, V82S, Y123H, Y147R, Q154R |
| ABE9.23_heterodimer | R2IN, V82S, Y123H, Y147R, Q154R |
| ABE9.24_heterodimer | V82S, Y123H, Y147R, Q154R, A158K |
| ABE9.25_heterodimer | N72K, V82S, Y123H, D139L, Y147R, Q154R, |
| ABE9.26_heterodimer | E25F, V82S, Y123H, D139M, Y147R, Q154R |
| ABE9.27_heterodimer | M70V, V82S, M94V, Y123H, Y147R, Q154R |
| ABE9.28_heterodimer | Q71M, V82S, Y123H, Y147R, Q154R |
| ABE9.29_monomer | E25F_I76Y_V82S_Y123H_Y147R_Q154R |
| ABE9.30_monomer | I76Y_V82T_Y123H_Y147R_Q154R |
| ABE9.31_monomer | N38G_I76Y_V82S_Y123H_Y147R_Q154R |
| ABE9.32_monomer | N38G_I76Y_V82T_Y123H_Y147R_Q154R |
| ABE9.33_monomer | R23H_I76Y_V82S_Y123H_Y147R_Q154R |
| ABE9.34_monomer | P54C_I76Y_V82S_Y123H_Y147R_Q154R |
| ABE9.35_monomer | R21N_I76Y_V82S_Y123H_Y147R_Q154R |
| ABE9.36_monomer | I76Y_V82S_Y123H_D138M_Y147R_Q154R |
| ABE9.37_monomer | Y72S_I76Y_V82S_Y123H_Y147R_Q154R |
| ABE9.38_heterodimer | E25F_I76Y_V82S_Y123H_Y147R_Q154R |
| ABE9.39_heterodimer | I76Y_V82T_Y123H_Y147R_Q154R |
| ABE9.40_heterodimer | N38G_I76YV82S_Y123H_Y147R_Q154R |
| ABE9.41_heterodimer | N38G_I76Y_V82T_Y123H_Y147R_Q154R |
| ABE9.42_heterodimer | R23H_I76Y_V82S_Y123H_Y147R_Q154R |
| ABE9.43_heterodimer | P54C_I76Y_V82S_Y123H_Y147R_Q154R |
| ABE9.44_heterodimer | R21N_I76Y_V82S_Y123H_Y147R_Q154R |
| ABE9.45_heterodimer | I76Y_V82S_Y123H_D138M_Y147R_Q154R |
| ABE9.46_heterodimer | Y72S_I76Y_V82S_Y123H_Y147R_Q154R |
| ABE9.47_monomer | N72K V82S, Y123H, Y147R, Q154R |
| ABE9.48_monomer | Q71M V82S, Y123H, Y147R, Q154R |
| ABE9.49_monomer | M70V, V82S, M94V, Y123H, Y147R, Q154R |
| ABE9.50_monomer | V82S, Y123H, T133K, Y147R, Q154R |
| ABE9.51_monomer | V82S, Y123H, T133K, Y147R, Q154R, |
| | A158K |
| ABE9.52_monomer | M70V, Q71M, N72K, V82S, Y123H, Y147R, |
| | Q154R |
| ABE9.53_heterodimer | N72K V82S, Y123H, Y147R, Q154R |
| ABE9.54_heterodimer | Q71M V82S, Y123H, Y147R, Q154R |
| ABE9.55_heterodimer | M70V,V82S, M94V, Y123H, Y147R, Q154R |
| ABE9.56_heterodimer | V82S, Y123H, T133K, Y147R, Q154R |
| ABE9.57_heterodimer | V82S, Y123H, T133K, Y147R, Q154R, |
| | A158K |
| ABE9.58_heterodimer | M70V, Q71M, N72K, V82S, Y123H, Y147R, |
| | Q154R |

In some embodiments, the base editor is a fusion protein comprising a polynucleotide programmable nucleotide binding domain (e.g., Cas9-derived domain) fused to a nucleobase editing domain (e.g., all or a portion of a deaminase domain). In certain embodiments, the fusion proteins provided herein comprise one or more features that improve the base editing activity of the fusion proteins. For example, any of the fusion proteins provided herein may comprise a Cas9 domain that has reduced nuclease activity. In some embodiments, any of the fusion proteins provided herein may have a Cas9 domain that does not have nuclease activity (dCas9), or a Cas9 domain that cuts one strand of a duplexed DNA molecule, referred to as a Cas9 nickase (nCas9).

In some embodiments, the base editor further comprises a domain comprising all or a portion of a uracil glycosylase inhibitor (UGI). In some embodiments, the base editor comprises a domain comprising all or a portion of a uracil binding protein (UBP), such as a uracil DNA glycosylase (UDG). In some embodiments, the base editor comprises a domain comprising all or a portion of a nucleic acid polymerase. In some embodiments, a base editor can comprise as a domain all or a portion of a nucleic acid polymerase (NAP). For example, a base editor can comprise all or a portion of a eukaryotic NAP. In some embodiments, a NAP or portion thereof incorporated into a base editor is a DNA polymerase. In some embodiments, a NAP or portion thereof incorporated into a base editor has translesion polymerase activity. In some embodiments, a NAP or portion thereof incorporated into a base editor is a translesion DNA polymerase. In some embodiments, a NAP or portion thereof incorporated into a base editor is a Rev7, Rev1 complex, polymerase iota, polymerase kappa, or polymerase eta. In some embodiments, a NAP or portion thereof incorporated into a base editor is a eukaryotic polymerase alpha, beta, gamma, delta, epsilon, gamma, eta, iota, kappa, lambda, mu, or nu component. In some embodiments, a NAP or portion thereof incorporated into a base editor comprises an amino acid sequence that is at least 75%, 80%, 85%, 90%, 95%, 96%, 97%, 98%, 99%, or 99.5% identical to a nucleic acid polymerase (e.g., a translesion DNA polymerase). In some embodiments, a nucleic acid polymerase or portion thereof incorporated into a base editor is a translesion DNA polymerase.

In some embodiments, a domain of the base editor can comprise multiple domains. For example, the base editor comprising a polynucleotide programmable nucleotide binding domain derived from Cas9 can comprise an REC lobe and an NUC lobe corresponding to the REC lobe and NUC lobe of a wild-type or natural Cas9. In another example, the base editor can comprise one or more of a RuvCI domain, BH domain, REC1 domain, REC2 domain, RuvCII domain, L1 domain, HNH domain, L2 domain, RuvCIII domain, WED domain, TOPO domain or CTD domain. In some embodiments, one or more domains of the base editor comprise a mutation (e.g., substitution, insertion, deletion) relative to a wild-type version of a polypeptide comprising the domain. For example, an HNH domain of a polynucleotide programmable DNA binding domain can comprise an H840A substitution. In another example, a RuvCI domain of a polynucleotide programmable DNA binding domain can comprise a D10A substitution.

Different domains (e.g., adjacent domains) of the base editor disclosed herein can be connected to each other with or without the use of one or more linker domains (e.g., an XTEN linker domain). In some embodiments, a linker domain can be a bond (e.g., covalent bond), chemical group, or a molecule linking two molecules or moieties, e.g., two domains of a fusion protein, such as, for example, a first domain (e.g., Cas9-derived domain) and a second domain (e.g., an adenosine deaminase domain). In some embodiments, a linker is a covalent bond (e.g., a carbon-carbon bond, disulfide bond, carbon-hetero atom bond, etc.). In certain embodiments, a linker is a carbon nitrogen bond of an amide linkage. In certain embodiments, a linker is a cyclic or acyclic, substituted or unsubstituted, branched or unbranched aliphatic or heteroaliphatic linker. In certain embodiments, a linker is polymeric (e.g., polyethylene, polyethylene glycol, polyamide, polyester, etc.). In certain embodiments, a linker comprises a monomer, dimer, or polymer of aminoalkanoic acid. In some embodiments, a linker comprises an aminoalkanoic acid (e.g., glycine, ethanoic acid, alanine, beta-alanine, 3-aminopropanoic acid, 4-aminobutanoic acid, 5-pentanoic acid, etc.). In some embodiments, a linker comprises a monomer, dimer, or polymer of aminohexanoic acid (Ahx). In certain embodiments, a linker is based on a carbocyclic moiety (e.g., cyclopentane, cyclohexane). In other embodiments, a linker comprises a polyethylene glycol moiety (PEG). In certain embodiments, a linker comprises an aryl or heteroaryl moiety. In certain embodiments, the linker is based on a phenyl ring. A linker can include functionalized moieties to facilitate attachment of a nucleophile (e.g., thiol, amino) from the peptide to the linker. Any electrophile can be used as part of the linker. Exemplary electrophiles include, but are not limited to, activated esters, activated amides, Michael acceptors, alkyl halides, aryl halides, acyl halides, and isothiocyanates. In some embodiments, a linker joins a gRNA binding domain of an RNA-programmable nuclease, including a Cas9 nuclease domain, and the catalytic domain of a nucleic acid editing protein. In some embodiments, a linker joins a dCas9 and a second domain (e.g., UGI, etc.).

### Linkers

In certain embodiments, linkers may be used to link any of the peptides or peptide domains as described herein. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

Typically, a linker is positioned between, or flanked by, two groups, molecules, or other moieties and connected to each one via a covalent bond, thus connecting the two. In some embodiments, a linker is an amino acid or a plurality of amino acids (e.g., a peptide or protein). In some embodiments, a linker is an organic molecule, group, polymer, or chemical moiety. In some embodiments, a linker is 2-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, the linker is about 3 to about 104 (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100) amino acids in length. Longer or shorter linkers are also contemplated.

In some embodiments, any of the fusion proteins provided herein, comprise a adenosine deaminase and a Cas9 domain that are fused to each other via a linker. Various linker lengths and flexibilities between the adenosine deaminase and the Cas9 domain can be employed (e.g., ranging from very flexible linkers of the form (GGGS)n (SEQ ID NO: 246), (GGGGS)n (SEQ ID NO: 247), and (G)n to more rigid linkers of the form (EAAAK)n (SEQ ID NO: 248), (SGGS)n (SEQ ID NO: 355), SGSETPGTSESATPES (SEQ ID NO: 249) (see, e.g., Guilinger JP, et al. Fusion of catalytically inactive Cas9 to FokI nuclease improves the specificity of genome modification. Nat. Biotechnol. 2014; 32(6): 577-82; the entire contents are incorporated herein by reference) and (XP)ₙ) in order to achieve the optimal length for activity for the adenosine deaminase nucleobase editor. In some embodiments, n is 1, 2, 3, 4, 5, 6, 7, 8, 9, 10, 11, 12, 13, 14, or 15. In some embodiments, the linker comprises a (GGS)n motif, wherein n is 1, 3, or 7. In some embodiments, adenosine deaminase and the Cas9 domain of any of the fusion proteins provided herein are fused via a linker comprising the amino acid sequence SGSETPGTSESATPES, which can also be referred to as the XTEN linker. In some embodiments, a linker comprises a plurality of proline residues and is 5-21, 5-14, 5-9, 5-7 amino acids in length, e.g., PAPAP (SEQ ID NO: 363), PAPAPA (SEQ ID NO: 364), PAPAPAP (SEQ ID NO: 365), PAPAPAPA (SEQ ID NO: 366), P(AP)4 (SEQ ID NO: 367), P(AP)7 (SEQ ID NO: 368), P(AP)10 (SEQ ID NO: 369) (see, e.g., Tan J, Zhang F, Karcher D, Bock R. Engineering of high-precision base editors for site-specific single nucleotide replacement. Nat Commun. 2019 Jan. 25; 10(1):439; the entire contents are incorporated herein by reference). Such proline-rich linkers are also termed “rigid” linkers. In some embodiments, adenosine deaminase and the Cas9 domain of any of the fusion proteins provided herein are fused via a linker comprising the amino acid sequence SGSETPGTSESATPES (SEQ ID NO: 249), which can also be referred to as the XTEN linker.

In some embodiments, the domains of the base editor are fused via a linker that comprises the amino acid sequence of:

(SEQ ID NO: 356)
SGGSSGSETPGTSESATPESSGGS,
(SEQ ID NO: 357)
SGGSSGGSSGSETPGTSESATPESSGGSSGGS,
or
(SEQ ID NO: 358)
GGSGGSPGSPAGSPTSTEEGTSESATPESGPGTSTEPSEGSAPGSPAGSP
TSTEEGTSTEPSEGSAPGTSTEPSEGSAPGTSESATPESGPGSEPATSGG
SGGS.

In some embodiments, domains of the base editor are fused via a linker comprising the amino acid sequence SGSETPGTSESATPES (SEQ ID NO: 249), which may also be referred to as the XTEN linker. In some embodiments, a linker comprises the amino acid sequence SGGS. In some embodiments, the linker is 24 amino acids in length. In some embodiments, the linker comprises the amino acid sequence SGGSSGGSSGSETPGTSESATPES (SEQ ID NO: 359). In some embodiments, the linker is 40 amino acids in length. In some embodiments, the linker comprises the amino acid sequence:

(SEQ ID NO: 360)
SGGSSGGSSGSETPGTSESATPESSGGSSGGSSGGSSGGS.

In some embodiments, the linker is 64 amino acids in length. In some embodiments, the linker comprises the amino acid sequence: SGGSSGGSSGSETPGTSESATPESSGGSSGGSSGGSSGGSSGSETPGTSESATPESSGGSSG GS (SEQ ID NO: 361). In some embodiments, the linker is 92 amino acids in length. In some embodiments the linker comprises the amino acid sequence:

(SEQ ID NO: 362)
PGSPAGSPTSTEEGTSESATPESGPGTSTEPSEGSAPGSPAGSPTSTEEG
TSTEPSEGSAPGTSTEPSEGSAPGTSESATPESGPGSEPATS.

In another embodiment, the base editor system comprises a component (protein) that interacts non-covalently with a deaminase (DNA deaminase), e.g., an adenosine, and transiently attracts the adenosine deaminase to the target nucleobase in a target polynucleotide sequence for specific editing, with minimal or reduced bystander or target-adjacent effects. Such a non-covalent system and method involving deaminase-interacting proteins serves to attract a DNA deaminase to a particular genomic target nucleobase and decouples the events of on-target and target-adjacent editing, thus enhancing the achievement of more precise single base substitution mutations. In an embodiment, the deaminase-interacting protein binds to the deaminase (e.g., adenosine deaminase) without blocking or interfering with the active (catalytic) site of the deaminase from engaging the target nucleobase (e.g., adenosine). Such as system, termed “MagnEdit,” involves interacting proteins tethered to a Cas9 and gRNA complex and can attract a co-expressed adenosine (either exogenous or endogenous) to edit a specific genomic target site, and is described in McCann, J. et al., 2020, “MagnEdit—interacting factors that recruit DNA-editing enzymes to single base targets,” Life-Science-Alliance, Vol. 3, No. 4 (e201900606), (doi 10.26508/Isa.201900606), the contents of which are incorporated by reference herein in their entirety. In an embodiment, the DNA deaminase is an adenosine deaminase variant (e.g., TadA*8) as described herein.

In another embodiment, a system called “Suntag,” involves non-covalently interacting components used for recruiting protein (e.g., adenosine deaminase) components, or multiple copies thereof, of base editors to polynucleotide target sites to achieve base editing at the site with reduced adjacent target editing, for example, as described in Tanenbaum, M. E. et al., “A protein tagging system for signal amplification in gene expression and fluorescence imaging,” Cell. 2014 Oct. 23; 159(3): 635-646. doi:10.1016/j.cell.2014.09.039; and in Huang, Y.-H. et al., 2017, “DNA epigenome editing using CRISPR-Cas SunTag-directed DNMT3A,” Genome Biol 18: 176. doi:10.1186/s13059-017-1306-z, the contents of each of which are incorporated by reference herein in their entirety. In an embodiment, the DNA deaminase is an adenosine deaminase variant (e.g., TadA*8) as described herein.

Nucleic Acid Programmable DNA Binding Proteins with Guide RNAs

Some aspects of this disclosure provide complexes comprising any of the fusion proteins provided herein, and a guide RNA bound to a nucleic acid programmable DNA binding protein (napDNAbp)domain (e.g., a Cas9 (e.g., a dCas9, a nuclease active Cas9, or a Cas9 nickase)) of the fusion protein. These complexes are also termed ribonucleoproteins (RNPs). In some embodiments, the guide nucleic acid (e.g., guide RNA) is from 15-100 nucleotides long and comprises a sequence of at least 10 contiguous nucleotides that is complementary to a target sequence. In some embodiments, the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. In some embodiments, the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence. In some embodiments, the target sequence is a DNA sequence. In some embodiments, the target sequence is an RNA sequence. In some embodiments, the target sequence is a sequence in the genome of a bacteria, yeast, fungi, insect, plant, or animal. In some embodiments, the target sequence is a sequence in the genome of a human. In some embodiments, the 3′ end of the target sequence is immediately adjacent to a canonical PAM sequence (NGG). In some embodiments, the 3′ end of the target sequence is immediately adjacent to a non-canonical PAM sequence (e.g., a sequence listed in Table 2 or 5′-NAA-3′). In some embodiments, the guide nucleic acid (e.g., guide RNA) is complementary to a sequence in a G6PC allele bearing GSD1a targetable mutations.

Some aspects of this disclosure provide methods of using the fusion proteins, or complexes provided herein. For example, some aspects of this disclosure provide methods comprising contacting a DNA molecule with any of the fusion proteins provided herein, and with at least one guide RNA, wherein the guide RNA is about 15-100 nucleotides long and comprises a sequence of at least 10 contiguous nucleotides that is complementary to a target sequence. In some embodiments, the 3′ end of the target sequence is immediately adjacent to an AGC, GAG, TTT, GTG, or CAA sequence. In some embodiments, the 3′ end of the target sequence is immediately adjacent to an NGA, NGCG, NGN, NNGRRT, NNNRRT, NGCG, NGCN, NGTN, NGTN, NGTN, or 5′ (TTTV) sequence. In some embodiments, the 3′ end of the target sequence is immediately adjacent to an e.g., TTN, DTTN, GTTN, ATTN, ATTC, DTTNT, WTTN, HATY, TTTN, TTTV, TTTC, TG, RTR, or YTN PAM site.

It will be understood that the numbering of the specific positions or residues in the respective sequences depends on the particular protein and numbering scheme used. Numbering might differ, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues.

It will be apparent to those of skill in the art that in order to target any of the fusion proteins disclosed herein, to a target site, e.g., a site comprising a mutation to be edited, it is typically necessary to co-express the fusion protein together with a guide RNA. As explained in more detail elsewhere herein, a guide RNA typically comprises a tracrRNA framework allowing for napDNAbp (e.g., Cas9) binding, and a guide sequence, which confers sequence specificity to the napDNAbp:nucleic acid editing enzyme/domain fusion protein. Alternatively, the guide RNA and tracrRNA may be provided separately, as two nucleic acid molecules. In some embodiments, the guide RNA comprises a structure, wherein the guide sequence comprises a sequence that is complementary to the target sequence. The guide sequence is typically 20 nucleotides long. The sequences of suitable guide RNAs for targeting napDNAbp:nucleic acid editing enzyme/domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins to specific target sequences are provided herein.

Distinct portions of sgRNA are predicted to form various features that interact with Cas9 (e.g., SpyCas9) and/or the DNA target. Six conserved modules have been identified within native crRNA:tracrRNA duplexes and single guide RNAs (sgRNAs) that direct Cas9 endonuclease activity (see Briner et al., Guide RNA Functional Modules Direct Cas9 Activity and Orthogonality Mol Cell. 2014 Oct. 23; 56(2):333-339). The six modules include the spacer responsible for DNA targeting, the upper stem, bulge, lower stem formed by the CRISPR repeat:tracrRNA duplex, the nexus, and hairpins from the 3′ end of the tracrRNA. The upper and lower stems interact with Cas9 mainly through sequence-independent interactions with the phosphate backbone. In some embodiments, the upper stem is dispensable. In some embodiments, the conserved uracil nucleotide sequence at the base of the lower stem is dispensable. The bulge participates in specific side-chain interactions with the Rec1 domain of Cas9. The nucleobase of U44 interacts with the side chains of Tyr 325 and His 328, while G43 interacts with Tyr 329. The nexus forms the core of the sgRNA:Cas9 interactions and lies at the intersection between the sgRNA and both Cas9 and the target DNA. The nucleobases of A51 and A52 interact with the side chain of Phe 1105; U56 interacts with Arg 457 and Asn 459; the nucleobase of U59 inserts into a hydrophobic pocket defined by side chains of Arg 74, Asn 77, Pro 475, Leu 455, Phe 446, and Ile 448; C60 interacts with Leu 455, Ala 456, and Asn 459, and C61 interacts with the side chain of Arg 70, which in turn interacts with C15. In some embodiments, one or more of these mutations are made in the bulge and/or the nexus of a sgRNA for a Cas9 (e.g., spyCas9) to optimize sgRNA:Cas9 interactions.

Moreover, the tracrRNA nexus and hairpins are critical for Cas9 pairing and can be swapped to cross orthogonality barriers separating disparate Cas9 proteins, which is instrumental for further harnessing of orthogonal Cas9 proteins. In some embodiments, the nexus and hairpins are swapped to target orthogonal Cas9 proteins. In some embodiments, a sgRNA is dispensed of the upper stem, hairpin 1, and/or the sequence flexibility of the lower stem to design a guide RNA that is more compact and conformationally stable. In some embodiments, the modules are modified to optimize multiplex editing using a single Cas9 with various chimeric guides or by concurrently using orthogonal systems with different combinations of chimeric sgRNAs. Details regarding guide functional modules and methods thereof are described, for example, in Briner et al., Guide RNA Functional Modules Direct Cas9 Activity and Orthogonality Mol Cell. 2014 Oct. 23; 56(2):333-339, the contents of which is incorporated by reference herein in its entirety. The domains of the base editor disclosed herein can be arranged in any order. Non-limiting examples of a base editor comprising a fusion protein comprising e.g., a polynucleotide-programmable nucleotide-binding domain (e.g., Cas9) and a deaminase domain (e.g., adenosine deaminase) can be arranged as follows:
NH₂-[nucleobase editing domain]-Linker1-[nucleobase editing domain]-COOH;
NH₂-[deaminase]-Linker1-[nucleobase editing domain]-COOH;
NH₂-[deaminase]-Linker1-[nucleobase editing domain]-Linker2-[UGI]-COOH;
NH₂-[deaminase]-Linker1-[nucleobase editing domain]-COOH;
NH₂-[adenosine deaminase]-Linker1-[nucleobase editing domain]-COOH;
NH₂-[nucleobase editing domain]-[deaminase]-COOH;
NH₂-[deaminase]-[nucleobase editing domain]-[inosine BER inhibitor]-COOH;
NH₂-[deaminase]-[inosine BER inhibitor]-[nucleobase editing domain]-COOH;
NH₂-[inosine BER inhibitor]-[deaminase]-[nucleobase editing domain]-COOH;
NH₂-[nucleobase editing domain]-[deaminase]-[inosine BER inhibitor]-COOH;
NH₂-[nucleobase editing domain]-[inosine BER inhibitor]-[deaminase]-COOH;
NH₂-[inosine BER inhibitor]-[nucleobase editing domain]-[deaminase]-COOH;
NH2-[nucleobase editing domain]-Linker1-[deaminase]-Linker2-[nucleobase editing domain]-COOH;
NH2-[nucleobase editing domain]-Linker1-[deaminase]-[nucleobase editing domain]-COOH;
NH2-[nucleobase editing domain]-[deaminase]-Linker2-[nucleobase editing domain]-COOH;
NH2-[nucleobase editing domain]-[deaminase]-[nucleobase editing domain]-COOH;
NH2-[nucleobase editing domain]-Linker1-[deaminase]-Linker2-[nucleobase editing domain]-[inosine BER inhibitor]-COOH;
NH2-[nucleobase editing domain]-Linker1-[deaminase]-[nucleobase editing domain]-[inosine BER inhibitor]-COOH;
NH2-[nucleobase editing domain]-[deaminase]-Linker2-[nucleobase editing domain]-[inosine BER inhibitor]-COOH;
NH2-[nucleobase editing domain]-[deaminase]-[nucleobase editing domain]-[inosine BER inhibitor]-COOH;
NH2-[inosine BER inhibitor]-[nucleobase editing domain]-Linker1-[deaminase]-Linker2-[nucleobase editing domain]-COOH;
NH2-[inosine BER inhibitor]-[nucleobase editing domain]-Linker1-[deaminase]-[nucleobase editing domain]-COOH;
NH2-[inosine BER inhibitor]-[nucleobase editing domain]-[deaminase]-Linker2-[nucleobase editing domain]-COOH; or
NH2-[inosine BER inhibitor]NH2-[nucleobase editing domain]-[deaminase]-[nucleobase editing domain]-COOH.

In some embodiments, the base editing fusion proteins provided herein need to be positioned at a precise location, for example, where a target base is placed within a defined region (e.g., a “deamination window”). In some embodiments, a target can be within a 4-base region. In some embodiments, such a defined target region can be approximately 15 bases upstream of the PAM. See Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference.

A defined target region can be a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a napDNAbp domain. In some embodiments, an NLS of the base editor is localized C-terminal to a napDNAbp domain.

Non-limiting examples of protein domains which can be included in the fusion protein include a deaminase domain (e.g., adenosine deaminase), a uracil glycosylase inhibitor (UGI) domain, epitope tags, reporter gene sequences, and/or protein domains having one or more of the following activities: methylase activity, demethylase activity, transcription activation activity, transcription repression activity, transcription release factor activity, gene silencing activity, chromatin modifying activity, epigenetic modifying activity, histone modification activity, RNA cleavage activity, and nucleic acid binding activity. Additional domains can be a heterologous functional domain. Such heterologous functional domains can confer a function activity, such as modification of a target polypeptide associated with target DNA (e.g., a histone, a DNA binding protein, etc.), leading to, for example, histone methylation, histone acetylation, histone ubiquitination, and the like. Other functions and/or activities conferred can include transposase activity, integrase activity, recombinase activity, ligase activity, ubiquitin ligase activity, deubiquitinating activity, adenylation activity, deadenylation activity, SUMOylation activity, deSUMOylation activity, or any combination of the above.

Other functions conferred can include methyltransferase activity, demethylase activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, remodeling activity, protease activity, oxidoreductase activity, transferase activity, hydrolase activity, lyase activity, isomerase activity, synthase activity, synthetase activity, and demyristoylation activity, or any combination thereof.

A domain may be detected or labeled with an epitope tag, a reporter protein, other binding domains. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). Additional protein sequences can include amino acid sequences that bind DNA molecules or bind other cellular molecules, including but not limited to maltose binding protein (MBP), S-tag, Lex A DNA binding domain (DBD) fusions, GAL4 DNA binding domain fusions, and herpes simplex virus (HSV) BP16 protein fusions.

### Methods of Using Fusion Proteins Comprising Adenosine Deaminase and a Cas9 Domain

Some aspects of this disclosure provide methods of using the fusion proteins, or complexes provided herein. For example, some aspects of this disclosure provide methods comprising contacting a DNA molecule with any of the fusion proteins provided herein, and with at least one guide RNA, wherein the guide RNA is about 15-100 nucleotides long and comprises a sequence of at least 10 contiguous nucleotides that is complementary to a target sequence. In some embodiments, the 3′ end of the target sequence is immediately adjacent to a canonical PAM sequence (NGG). In some embodiments, the 3′ end of the target sequence is not immediately adjacent to a canonical PAM sequence (NGG). In some embodiments, the 3′ end of the target sequence is immediately adjacent to an AGC, GAG, TTT, GTG, or CAA sequence. In some embodiments, the 3′ end of the target sequence is immediately adjacent to an NGA, NGCG, NGN, NNGRRT, NNNRRT, NGCG, NGCN, NGTN, NGTN, NGTN, or 5′ (TTTV) sequence.

### Base Editor Efficiency

In some embodiments, the nucleobase editing proteins provided herein can be validated for gene editing-based human therapeutics in vitro. It will be understood by the skilled artisan that the nucleobase editing proteins provided herein, e.g., the fusion proteins comprising a polynucleotide programmable nucleotide binding domain (e.g., Cas9) and a nucleobase editing domain (e.g., an adenosine deaminase domain) can be used to edit a nucleotide from A to G or C to T.

As most of the known genetic variations associated with human disease are point mutations, methods that can more efficiently and cleanly make precise point mutations are needed. Base editing systems as provided herein provide a new way to provide genome editing without generating double-strand DNA breaks, without requiring a donor DNA template, and without inducing an excess of stochastic insertions and deletions.

In some embodiments, the present disclosure provides base editors that efficiently generate an intended mutation in a polynucleotide sequence without generating a significant number of unintended mutations, such as unintended point mutations. In some embodiments, an intended mutation is a mutation that is generated by a specific base editor (e.g., adenosine base editor) bound to a guide polynucleotide (e.g., gRNA), specifically designed to generate the intended mutation. In some embodiments, the intended mutation is in a gene associated with a target antigen associated with a disease or disorder, e.g., a mutation in the G6PC gene associated with GSD1a. In some embodiments, the intended mutation is an adenine (A) to guanine (G) point mutation (e.g., SNP) in a gene associated with a target antigen associated with a disease or disorder, e.g., a mutation in the G6PC gene associated with GSD1a. In some embodiments, the intended mutation is an adenine (A) to guanine (G) point mutation within the coding region or non-coding region of a gene (e.g., regulatory region or element).

The base editors as described herein advantageously modify a specific nucleotide base encoding a protein without generating a significant proportion of indels. An “indel”, as used herein, refers to the insertion or deletion of a nucleotide base within a nucleic acid. Such insertions or deletions can lead to frame shift mutations within a coding region of a gene. In some embodiments, it is desirable to generate base editors that efficiently modify (e.g. mutate) a specific nucleotide within a nucleic acid, without generating a large number of insertions or deletions (i.e., indels) in the nucleic acid. In some embodiments, it is desirable to generate base editors that efficiently modify (e.g. mutate or methylate) a specific nucleotide within a nucleic acid, without generating a large number of insertions or deletions (i.e., indels) in the nucleic acid. In certain embodiments, any of the base editors provided herein can generate a greater proportion of intended modifications (e.g., methylations) versus indels. In certain embodiments, any of the base editors provided herein can generate a greater proportion of intended modifications (e.g., mutations) versus indels.

In some embodiments, the base editors provided herein are capable of generating a ratio of intended mutations to indels (i.e., intended point mutations:unintended point mutations) that is greater than 1:1. In some embodiments, the base editors provided herein are capable of generating a ratio of intended mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method.

In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein can limit the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%. The number of indels formed at a nucleic acid region may depend on the amount of time a nucleic acid (e.g., a nucleic acid within the genome of a cell) is exposed to a base editor. In some embodiments, a number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a nucleic acid (e.g., a nucleic acid within the genome of a cell) to a base editor.

Some aspects of the disclosure are based on the recognition that any of the base editors provided herein are capable of efficiently generating an intended mutation in a nucleic acid (e.g. a nucleic acid within a genome of a subject) without generating a considerable number of unintended mutations (e.g., spurious off-target editing or bystander editing). In some embodiments, an intended mutation is a mutation that is generated by a specific base editor bound to a gRNA, specifically designed to generate the intended mutation. In some embodiments, the intended mutation is a mutation that generates a stop codon, for example, a premature stop codon within the coding region of a gene. In some embodiments, the intended mutation is a mutation that eliminates a stop codon. In some embodiments, the intended mutation is a mutation that alters the splicing of a gene. In some embodiments, the intended mutation is a mutation that alters the regulatory sequence of a gene (e.g., a gene promotor or gene repressor).

In some embodiments, any of the base editors provided herein are capable of generating a ratio of intended mutations to unintended mutations (e.g., intended mutations:unintended mutations) that is greater than 1:1. In some embodiments, any of the base editors provided herein are capable of generating a ratio of intended mutations to unintended mutations that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least 1000:1, or more. It should be appreciated that the characteristics of the base editors described herein, may be applied to any of the fusion proteins, or methods of using the fusion proteins provided herein.

Base editing is often referred to as a “modification”, such as, a genetic modification, a gene modification and modification of the nucleic acid sequence and is clearly understandable based on the context that the modification is a base editing modification. A base editing modification is therefore a modification at the nucleotide base level, for example as a result of the deaminase activity discussed throughout the disclosure, which then results in a change in the gene sequence, and may affect the gene product. In essence therefore, the gene editing modification described herein may result in a modification of the gene, structurally and/or functionally, wherein the expression of the gene product may be modified, for example, the expression of the gene is knocked out; or conversely, enhanced, or, in some circumstances, the gene function or activity may be modified. Using the methods disclosed herein, a base editing efficiency may be determined as the knockdown efficiency of the gene in which the base editing is performed, wherein the base editing is intended to knockdown the expression of the gene. A knockdown level may be validated quantitatively by determining the expression level by any detection assay, such as assay for protein expression level, for example, by flow cytometry; assay for detecting RNA expression such as quantitative RT-PCR, northern blot analysis, or any other suitable assay such as pyrosequencing; and may be validated qualitatively by nucleotide sequencing reactions.

In some embodiments, any of base editor systems provided herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% indel formation in the target polynucleotide sequence.

In some embodiments, targeted modifications, e.g., single base editing, are used simultaneously to target at least 4, 5, 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 different endogenous sequences for base editing with different guide RNAs.

In some embodiments, targeted modifications, e.g. single base editing, are used to sequentially target at least 4, 5, 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 50, or more different endogenous gene sequences for base editing with different guide RNAs.

Some aspects of the disclosure are based on the recognition that any of the base editors provided herein are capable of efficiently generating an intended mutation, such as a point mutation, in a nucleic acid (e.g., a nucleic acid within a genome of a subject) without generating a significant number of unintended mutations, such as unintended point mutations (i.e., mutation of bystanders). In some embodiments, any of the base editors provided herein are capable of generating at least 0.01% of intended mutations (i.e., at least 0.01% base editing efficiency). In some embodiments, any of the base editors provided herein are capable of generating at least 0.01%, 1%, 2%, 3%, 4%, 5%, 10%, 15%, 20%, 25%, 30%, 40%, 45%, 50%, 60%, 70%, 80%, 90%, 95%, or 99% of intended mutations.

In some embodiments, any of base editor systems comprising one of the ABE base editor variants described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% indel formation in the target polynucleotide sequence. In some embodiments, any of base editor systems comprising one of the ABE base editor variants described herein result in less than 0.8% indel formation in the target polynucleotide sequence. In some embodiments, any of base editor systems comprising one of the ABE base editor variants described herein result in at most 0.8% indel formation in the target polynucleotide sequence. In some embodiments, any of base editor systems comprising one of the ABE base editor variants described herein result in less than 0.3% indel formation in the target polynucleotide sequence. In some embodiments, any of base editor systems comprising one of the ABE base editor variants described results in lower indel formation in the target polynucleotide sequence compared to a base editor system comprising one of ABE7 base editors. In some embodiments, any of base editor systems comprising one of the ABE base editor variants described herein results in lower indel formation in the target polynucleotide sequence compared to a base editor system comprising an ABE7.10.

In some embodiments, any of base editor systems comprising one of the ABE base editor variants described herein has reduction in indel frequency compared to a base editor system comprising one of the ABE7 base editors. In some embodiments, any of base editor systems comprising one of the ABE base editor variants described herein has at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% reduction in indel frequency compared to a base editor system comprising one of the ABE7 base editors. In some embodiments, a base editor system comprising one of the ABE base editor variants described herein has at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% reduction in indel frequency compared to a base editor system comprising an ABE7.10.

Provided herein are adenosine deaminase variants (e.g., TadA variants) that have increased efficiency and specificity. In particular, the adenosine deaminase variants described herein are more likely to edit a desired base within a polynucleotide, and are less likely to edit bases that are not intended to be altered (e.g., “bystanders”).

In some embodiments, any of the base editing system comprising one of the ABE base editor variants described herein has reduced bystander editing or mutations. In some embodiments, an unintended editing or mutation is a bystander mutation or bystander editing, for example, base editing of a target base (e.g., A or C) in an unintended or non-target position in a target window of a target nucleotide sequence. In some embodiments, any of the base editing system comprising one of the ABE base editor variants described herein has reduced bystander editing or mutations compared to a base editor system comprising an ABE7 base editor, e.g., ABE7.10. In some embodiments, any of the base editing system comprising one of the ABE base editor variants described herein has reduced bystander editing or mutations by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% compared to a base editor system comprising an ABE7 base editor, e.g., ABE7.10. In some embodiments, any of the base editing system comprising one of the ABE base editor variants described herein has reduced bystander editing or mutations by at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3.0 fold compared to a base editor system comprising an ABE7 base editor, e.g., ABE7.10.

In some embodiments, any of the base editing system comprising one of the ABE base editor variants described herein has reduced spurious editing. In some embodiments, an unintended editing or mutation is a spurious mutation or spurious editing, for example, non-specific editing or guide independent editing of a target base (e.g., A or C) in an unintended or non-target region of the genome. In some embodiments, any of the base editing system comprising one of the ABE base editor variants described herein has reduced spurious editing compared to a base editor system comprising an ABE7 base editor, e.g., ABE7.10. In some embodiments, any of the base editing system comprising one of the ABE base editor variants described herein has reduced spurious editing by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% compared to a base editor system comprising an ABE7 base editor, e.g., ABE7.10. In some embodiments, any of the base editing system comprising one of the ABE base editor variants described herein has reduced spurious editing by at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3.0 fold compared to a base editor system comprising an ABE7 base editor, e.g., ABE7.10.

In some embodiments, any of the ABE base editor variants described herein have at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% base editing efficiency. In some embodiments, the base editing efficiency may be measured by calculating the percentage of edited nucleobases in a population of cells. In some embodiments, any of the ABE base editor variants described herein have base editing efficiency of at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% as measured by edited nucleobases in a population of cells.

In some embodiments, any of the ABE base editor variants described herein has higher base editing efficiency compared to the ABE7 base editors. In some embodiments, any of the ABE base editor variants described herein have at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 450%, or at least 500% higher base editing efficiency compared to an ABE7 base editor, e.g., ABE7.10.

In some embodiments, any of the ABE base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2, at least 3.3 fold, at least 3.4 fold, at least 3.5 fold, at least 3.6 fold, at least 3.7 fold, at least 3.8 fold, at least 3.9 fold, at least 4.0 fold, at least 4.1 fold, at least 4.2 fold, at least 4.3 fold, at least 4.4 fold, at least 4.5 fold, at least 4.6 fold, at least 4.7 fold, at least 4.8 fold, at least 4.9 fold, or at least 5.0 fold higher base editing efficiency compared to an ABE7 base editor, e.g., ABE7.10.

In some embodiments, any of the ABE base editor variants described herein have at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% on-target base editing efficiency. In some embodiments, any of the ABE base editor variants described herein have on-target base editing efficiency of at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% as measured by edited target nucleobases in a population of cells.

In some embodiments, any of the ABE base editor variants described herein has higher on-target base editing efficiency compared to the ABE7 base editors. In some embodiments, any of the ABE base editor variants described herein have at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 450%, or at least 500% higher on-target base editing efficiency compared to an ABE7 base editor, e.g., ABE7.10.

In some embodiments, any of the ABE base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2 fold, at least 3.3 fold, at least 3.4 fold, at least 3.5 fold, at least 3.6 fold, at least 3.7 fold, at least 3.8 fold, at least 3.9 fold, at least 4.0 fold, at least 4.1 fold, at least 4.2 fold, at least 4.3 fold, at least 4.4 fold, at least 4.5 fold, at least 4.6 fold, at least 4.7 fold, at least 4.8 fold, at least 4.9 fold, or at least 5.0 fold higher on-target base editing efficiency compared to an ABE7 base editor, e.g., ABE7.10.

The ABE base editor variants described herein may be delivered to a host cell via a plasmid, a vector, a LNP complex, or an mRNA. In some embodiments, any of the ABE base editor variants described herein is delivered to a host cell as an mRNA. In some embodiments, an ABE base editor delivered via a nucleic acid based delivery system, e.g., an mRNA, has on-target editing efficiency of at least at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% as measured by edited nucleobases. In some embodiments, an ABE base editor delivered by an mRNA system has higher base editing efficiency compared to an ABE base editor delivered by a plasmid or vector system. In some embodiments, any of the ABE base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300% higher, at least 310%, at least 320%, at least 330%, at least 340%, at least 350%, at least 360%, at least 370%, at least 380%, at least 390%, at least 400%, at least 450%, or at least 500% on-target editing efficiency when delivered by an mRNA system compared to when delivered by a plasmid or vector system. In some embodiments, any of the ABE base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2 fold, at least 3.3 fold, at least 3.4 fold, at least 3.5 fold, at least 3.6 fold, at least 3.7 fold, at least 3.8 fold, at least 3.9 fold, at least 4.0 fold, at least 4.1 fold, at least 4.2 fold, at least 4.3 fold, at least 4.4 fold, at least 4.5 fold, at least 4.6 fold, at least 4.7 fold, at least 4.8 fold, at least 4.9 fold, or at least 5.0 fold higher on-target editing efficiency when delivered by an mRNA system compared to when delivered by a plasmid or vector system.

In some embodiments, any of base editor systems comprising one of the ABE base editor variants described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% off-target editing in the target polynucleotide sequence.

In some embodiments, any of the ABE base editor variants described herein has lower guided off-target editing efficiency when delivered by an mRNA system compared to when delivered by a plasmid or vector system. In some embodiments, any of the ABE base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% lower guided off-target editing efficiency when delivered by an mRNA system compared to when delivered by a plasmid or vector system. In some embodiments, any of the ABE base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3.0 fold lower guided off-target editing efficiency when delivered by an mRNA system compared to when delivered by a plasmid or vector system. In some embodiments, any of the ABE base editor variants described herein has at least about 2.2 fold decrease in guided off-target editing efficiency when delivered by an mRNA system compared to when delivered by a plasmid or vector system.

In some embodiments, any of the ABE base editor variants described herein has lower guide-independent off-target editing efficiency when delivered by an mRNA system compared to when delivered by a plasmid or vector system. In some embodiments, any of the ABE base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% lower guide-independent off-target editing efficiency when delivered by an mRNA system compared to when delivered by a plasmid or vector system. In some embodiments, any of the ABE base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 5.0 fold, at least 10.0 fold, at least 20.0 fold, at least 50.0 fold, at least 70.0 fold, at least 100.0 fold, at least 120.0 fold, at least 130.0 fold, or at least 150.0 fold lower guide-independent off-target editing efficiency when delivered by an mRNA system compared to when delivered by a plasmid or vector system. In some embodiments, ABE base editor variants described herein has 134.0 fold decrease in guide-independent off-target editing efficiency (e.g., spurious RNA deamination) when delivered by an mRNA system compared to when delivered by a plasmid or vector system. In some embodiments, ABE base editor variants described herein does not increase guide-independent mutation rates across the genome.

In some embodiments, a single gene delivery event (e.g., by transduction, transfection, electroporation or any other method) can be used to target base editing of 5 sequences within a cell's genome. In some embodiments, a single gene delivery event can be used to target base editing of 6 sequences within a cell's genome. In some embodiments, a single gene delivery event can be used to target base editing of 7 sequences within a cell's genome. In some embodiments, a single electroporation event can be used to target base editing of 8 sequences within a cell's genome. In some embodiments, a single gene delivery event can be used to target base editing of 9 sequences within a cell's genome. In some embodiments, a single gene delivery event can be used to target base editing of 10 sequences within a cell's genome. In some embodiments, a single gene delivery event can be used to target base editing of 20 sequences within a cell's genome. In some embodiments, a single gene delivery event can be used to target base editing of 30 sequences within a cell's genome. In some embodiments, a single gene delivery event can be used to target base editing of 40 sequences within a cell's genome. In some embodiments, a single gene delivery event can be used to target base editing of 50 sequences within a cell's genome.

In some embodiments, the method described herein, for example, the base editing methods has minimum to no off-target effects.

In some embodiments, the base editing method described herein results in at least 50% of a cell population that have been successfully edited (i.e., cells that have been successfully engineered). In some embodiments, the base editing method described herein results in at least 55% of a cell population that have been successfully edited. In some embodiments, the base editing method described herein results in at least 60% of a cell population that have been successfully edited. In some embodiments, the base editing method described herein results in at least 65% of a cell population that have been successfully edited. In some embodiments, the base editing method described herein results in at least 70% of a cell population that have been successfully edited. In some embodiments, the base editing method described herein results in at least 75% of a cell population that have been successfully edited. In some embodiments, the base editing method described herein results in at least 80% of a cell population that have been successfully edited. In some embodiments, the base editing method described herein results in at least 85% of a cell population that have been successfully edited. In some embodiments, the base editing method described herein results in at least 90% of a cell population that have been successfully edited. In some embodiments, the base editing method described herein results in at least 95% of a cell population that have been successfully edited. In some embodiments, the base editing method described herein results in about 91%, 92%, 93%, 94%, 95%, 96%, 97%, 98%, 99% or 100% of a cell population that have been successfully edited.

In some embodiments, the live cell recovery following a base editing intervention is greater than at least 60%, 70%, 80%, 90% of the starting cell population at the time of the base editing event. In some embodiments, the live cell recovery as described above is about 70%. In some embodiments, the live cell recovery as described above is about 75%. In some embodiments, the live cell recovery as described above is about 80%. In some embodiments, the live cell recovery as described above is about 85%. In some embodiments, the live cell recovery as described above is about 90%, or about 91%, 92%, 93%, 94% 95%, 96%, 97%, 98%, or 99%, or 100% of the cells in the population at the time of the base editing event.

In some embodiments the engineered cell population can be further expanded in vitro by about 2 fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold.

The number of intended mutations and indels can be determined using any suitable method, for example, as described in International PCT Application Nos. PCT/2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor.

The number of indels formed at a target nucleotide region can depend on the amount of time a nucleic acid (e.g., a nucleic acid within the genome of a cell) is exposed to a base editor. In some embodiments, the number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing the target nucleotide sequence (e.g., a nucleic acid within the genome of a cell) to a base editor. It should be appreciated that the characteristics of the base editors as described herein can be applied to any of the fusion proteins, or methods of using the fusion proteins provided herein.

Details of base editor efficiency are described in International PCT Application Nos. PCT/2017/045381 (WO 2018/027078) and PCT/US2016/058344 (WO 2017/070632), each of which is incorporated herein by reference for its entirety. Also see Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference. In some embodiments, editing of a plurality of nucleobase pairs in one or more genes using the methods provided herein results in formation of at least one intended mutation. In some embodiments, said formation of said at least one intended mutation results in the disruption the normal function of a gene. In some embodiments, said formation of said at least one intended mutation results decreases or eliminates the expression of a protein encoded by a gene. It should be appreciated that multiplex editing can be accomplished using any method or combination of methods provided herein.

### Multiplex Editing

In some embodiments, the base editor system provided herein is capable of multiplex editing of a plurality of nucleobase pairs in one or more genes. In some embodiments, the plurality of nucleobase pairs is located in the same gene. In some embodiments, the plurality of nucleobase pairs is located in one or more gene, wherein at least one gene is located in a different locus. In some embodiments, the multiplex editing can comprise one or more guide polynucleotides. In some embodiments, the multiplex editing can comprise one or more base editor systems. In some embodiments, the multiplex editing can comprise one or more base editor systems with a single guide polynucleotide. In some embodiments, the multiplex editing can comprise one or more base editor systems with a plurality of guide polynucleotides. In some embodiments, the multiplex editing can comprise one or more guide polynucleotides with a single base editor system. In some embodiments, the multiplex editing can comprise at least one guide polynucleotide that does not require a PAM sequence to target binding to a target polynucleotide sequence. In some embodiments, multiplex editing can comprise at least one guide polynucleotide that requires a PAM sequence to target binding to a target polynucleotide sequence. In some embodiments, the multiplex editing can comprise a mix of at least one guide polynucleotide that does not require a PAM sequence to target binding to a target polynucleotide sequence and at least one guide polynucleotide that require a PAM sequence to target binding to a target polynucleotide sequence. It should be appreciated that the characteristics of the multiplex editing using any of the base editors as described herein can be applied to any combination of methods using any base editor provided herein. It should also be appreciated that the multiplex editing using any of the base editors as described herein can comprise a sequential editing of a plurality of nucleobase pairs.

In some embodiments, the plurality of nucleobase pairs are in one more genes. In some embodiments, the plurality of nucleobase pairs is in the same gene. In some embodiments, at least one gene in the one more genes is located in a different locus.

In some embodiments, the editing is editing of the plurality of nucleobase pairs in at least one protein coding region. In some embodiments, the editing is editing of the plurality of nucleobase pairs in at least one protein non-coding region. In some embodiments, the editing is editing of the plurality of nucleobase pairs in at least one protein coding region and at least one protein non-coding region.

In some embodiments, the editing is in conjunction with one or more guide polynucleotides. In some embodiments, the base editor system can comprise one or more base editor system. In some embodiments, the base editor system can comprise one or more base editor systems in conjunction with a single guide polynucleotide. In some embodiments, the base editor system can comprise one or more base editor system in conjunction with a plurality of guide polynucleotides. In some embodiments, the editing is in conjunction with one or more guide polynucleotide with a single base editor system. In some embodiments, the editing is in conjunction with at least one guide polynucleotide that does not require a PAM sequence to target binding to a target polynucleotide sequence. In some embodiments, the editing is in conjunction with at least one guide polynucleotide that require a PAM sequence to target binding to a target polynucleotide sequence. In some embodiments, the editing is in conjunction with a mix of at least one guide polynucleotide that does not require a PAM sequence to target binding to a target polynucleotide sequence and at least one guide polynucleotide that require a PAM sequence to target binding to a target polynucleotide sequence. It should be appreciated that the characteristics of the multiplex editing using any of the base editors as described herein can be applied to any of combination of the methods of using any of the base editors provided herein. It should also be appreciated that the editing can comprise a sequential editing of a plurality of nucleobase pairs.

In some embodiments, the base editor system capable of multiplex editing of a plurality of nucleobase pairs in one or more genes comprises one of the ABE base editor variants described herein. In some embodiments, the base editor system capable of multiplex editing of a plurality of nucleobase pairs in one or more genes comprises one of ABE7 base editors. In some embodiments, the base editor system capable of multiplex editing comprising one of the ABE base editor variants described herein has higher multiplex editing efficiency compared the base editor system capable of multiplex editing comprising one of ABE7 base editors. In some embodiments, the base editor system capable of multiplex editing comprising one of the ABE base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300% higher, at least 310%, at least 320%, at least 330%, at least 340%, at least 350%, at least 360%, at least 370%, at least 380%, at least 390%, at least 400%, at least 450%, or at least 500% higher multiplex editing efficiency compared the base editor system capable of multiplex editing comprising one of ABE7 base editors. In some embodiments, the base editor system capable of multiplex editing comprising one of the ABE base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2 fold, at least 3.3 fold, at least 3.4 fold, at least 3.5 fold, at least 4.0 fold, at least 4.5 fold, at least 5.0 fold, at least 5.5 fold, or at least 6.0 fold higher multiplex editing efficiency compared the base editor system capable of multiplex editing comprising one of ABE7 base editors.

### Delivery System

Nucleic Acid-Based Delivery of Nucleobase Editors and gRNAs

Nucleic acids encoding an adenosine deaminase nucleobase editor according to the present disclosure can be administered to subjects or delivered into cells in vitro, ex vivo, or in vivo by art-known methods or as described herein. For example, adenosine deaminase nucleobase editors can be delivered by, e.g., vectors (e.g., viral or non-viral vectors), non-vector based methods (e.g., using naked DNA, DNA complexes, lipid nanoparticles), or a combination thereof.

Nucleic acids encoding adenosine deaminase nucleobase editors can be delivered directly to cells (e.g., hematopoietic cells or their progenitors, hematopoietic stem cells, and/or induced pluripotent stem cells) as naked DNA or RNA, for instance by means of transfection or electroporation, or can be conjugated to molecules (e.g., N-acetylgalactosamine) promoting uptake by the target cells. Nucleic acid vectors, such as the vectors described herein can also be used.

Nucleic acid vectors can comprise one or more sequences encoding a domain of a fusion protein described herein. A vector can also comprise a sequence encoding a signal peptide (e.g., for nuclear localization, nucleolar localization, or mitochondrial localization), associated with (e.g., inserted into or fused to) a sequence coding for a protein. As one example, a nucleic acid vectors can include a Cas9 coding sequence that includes one or more nuclear localization sequences (e.g., a nuclear localization sequence from SV40), and an adenosine deaminase variant (e.g., TadA variant).

The nucleic acid vector can also include any suitable number of regulatory/control elements, e.g., promoters, enhancers, introns, polyadenylation signals, Kozak consensus sequences, or internal ribosome entry sites (IRES). These elements are well known in the art.

Nucleic acid vectors according to this disclosure include recombinant viral vectors. Exemplary viral vectors are set forth herein. Other viral vectors known in the art can also be used. In addition, viral particles can be used to deliver base editing system components in nucleic acid and/or peptide form. For example, “empty” viral particles can be assembled to contain any suitable cargo. Viral vectors and viral particles can also be engineered to incorporate targeting ligands to alter target tissue specificity.

In addition to viral vectors, non-viral vectors can be used to deliver nucleic acids encoding genome editing systems according to the present disclosure. One important category of non-viral nucleic acid vectors are nanoparticles, which can be organic or inorganic. Nanoparticles are well known in the art. Any suitable nanoparticle design can be used to deliver genome editing system components or nucleic acids encoding such components. For instance, organic (e.g. lipid and/or polymer) nanoparticles can be suitable for use as delivery vehicles in certain embodiments of this disclosure.

### Nucleic Acid-Based Delivery of Base Editor Systems

Nucleic acid molecules encoding a base editor system according to the present disclosure can be administered to subjects or delivered into cells in vitro or in vivo by art-known methods or as described herein. For example, a base editor system comprising a deaminase (e.g., cytidine or adenine deaminase) can be delivered by vectors (e.g., viral or non-viral vectors), or by naked DNA, DNA complexes, lipid nanoparticles, or a combination of the aforementioned compositions.

Nanoparticles, which can be organic or inorganic, are useful for delivering a base editor system or component thereof. Nanoparticles are well known in the art and any suitable nanoparticle can be used to deliver a base editor system or component thereof, or a nucleic acid molecule encoding such components. In one example, organic (e.g. lipid and/or polymer) nanoparticles are suitable for use as delivery vehicles in certain embodiments of this disclosure.

Exemplary lipids for use in nanoparticle formulations, and/or gene transfer are shown in Table 15 (below).

TABLE 15
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)prophy1]N,N,N-trimethylammonium | DOTMA | Cationic |
| chloride | | |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 20c | Cationic |
| 2,3-Dioleyloxy-N-[2(sperminecarboxamido-ethyl]-N,N- | DOSPA | Cationic |
| dimethyl-1-propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyl- | CLIP-6 | Cationic |
| oxymethyloxy)ethyl ]trimethylammoniun bromide | EDMPC | Cationic |
| Ethyldimyristoylphosphatidylcholine | | |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho sphocholine | DSEPC | Cationic |
| N-Palmitoyl D-erythro-sphingosyl carbamoyl-spermine | CCS | Cationic |
| N-t-Butyl-NO-tetradecyl-3-tetradecylaminopropionamidine | diC14-amidine | Cationic |
| Octadecenolyoxy[ethyl-2-heptadeceny1-3 hydroxyethyl] | DOTIM | Cationic |
| imidazolinium chloride | | |
| N1 -Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoley1-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

Table 16 lists exemplary polymers for use in gene transfer and/or nanoparticle formulations.

TABLE 16
Polymers Used for Gene Transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified | PLL |
| Poly(N-vinylpyrrolidone) | PVP |
| Poly(propylenimine) | PPI |
| Poly(amidoamine) | PAMAM |
| Poly(amidoethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | PHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Table 17 summarizes delivery methods for a polynucleotide encoding a fusion protein described herein.

TABLE 17

| | | Delivery into | | | Type of |
|---|---|---|---|---|---|
| | | Non-Dividing | Duration of | Genome | Molecule |
| Delivery | Vector/Mode | Cells | Expression | Integration | Delivered |
| Physical | (e.g., | YES | Transient | NO | Nucleic Acids |
| | electroporation, | | | | and Proteins |
| | particle gun, | | | | |
| | Calcium | | | | |
| | Phosphate | | | | |
| | transfection | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno- | YES | Stable | NO | DNA |
| | Associated | YES | Very | NO | DNA |
| | Virus (AAV) | | Transient | | |
| | Vaccinia Virus | | | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | YES | Transient | NO | Nucleic Acids |
| | Mammalian | | | | |
| | Virus-like | | | | |
| | Particles | YES | Transient | NO | Nucleic Acids |
| | Biological | | | | |
| | liposomes: | | | | |
| | Erythrocyte | | | | |
| | Ghosts and | | | | |
| | Exosomes | | | | |

In another aspect, the delivery of genome editing system components or nucleic acids encoding such components, for example, a nucleic acid binding protein such as, for example, Cas9 or variants thereof, and a gRNA targeting a genomic nucleic acid sequence of interest, may be accomplished by delivering a ribonucleoprotein (RNP) to cells. The RNP comprises the nucleic acid binding protein, e.g., Cas9, in complex with the targeting gRNA. RNPs may be delivered to cells using known methods, such as electroporation, nucleofection, or cationic lipid-mediated methods, for example, as reported by Zuris, J. A. et al., 2015, Nat. Biotechnology, 33(1):73-80. RNPs are advantageous for use in CRISPR base editing systems, particularly for cells that are difficult to transfect, such as primary cells. In addition, RNPs can also alleviate difficulties that may occur with protein expression in cells, especially when eukaryotic promoters, e.g., CMV or EF1A, which may be used in CRISPR plasmids, are not well-expressed. Advantageously, the use of RNPs does not require the delivery of foreign DNA into cells. Moreover, because an RNP comprising a nucleic acid binding protein and gRNA complex is degraded over time, the use of RNPs has the potential to limit off-target effects. In a manner similar to that for plasmid based techniques, RNPs can be used to deliver binding protein (e.g., Cas9 variants) and to direct homology directed repair (HDR).

A promoter used to drive base editor coding nucleic acid molecule expression can include AAV ITR. This can be advantageous for eliminating the need for an additional promoter element, which can take up space in the vector. The additional space freed up can be used to drive the expression of additional elements, such as a guide nucleic acid or a selectable marker. ITR activity is relatively weak, so it can be used to reduce potential toxicity due to over expression of the chosen nuclease.

Any suitable promoter can be used to drive expression of the base editor and, where appropriate, the guide nucleic acid. For ubiquitous expression, promoters that can be used include CMV, CAG, CBh, PGK, SV40, Ferritin heavy or light chains, etc. For brain or other CNS cell expression, suitable promoters can include: SynapsinI for all neurons, CaMKIIalpha for excitatory neurons, GAD67 or GAD65 or VGAT for GABAergic neurons, etc. For liver cell expression, suitable promoters include the Albumin promoter. For lung cell expression, suitable promoters can include SP-B. For endothelial cells, suitable promoters can include ICAM. For hematopoietic cells suitable promoters can include IFNbeta or CD45. For Osteoblasts suitable promoters can include OG-2.

In some embodiments, a base editor of the present disclosure is of small enough size to allow separate promoters to drive expression of the base editor and a compatible guide nucleic acid within the same nucleic acid molecule. For instance, a vector or viral vector can comprise a first promoter operably linked to a nucleic acid encoding the base editor and a second promoter operably linked to the guide nucleic acid.

The promoter used to drive expression of a guide nucleic acid can include: Pol III promoters such as U6 or H1 Use of Pol II promoter and intronic cassettes to express gRNA Adeno Associated Virus (AAV).

In particular embodiments, a fusion protein as described herein is encoded by a polynucleotide present in a viral vector (e.g., adeno-associated virus (AAV), AAV3, AAV3b, AAV4, AAV5, AAV6, AAV7, AAV8, AAV9, AAVrh8, AAV10, and variants thereof), or a suitable capsid protein of any viral vector. Thus, in some aspects, the disclosure relates to the viral delivery of a fusion protein. Examples of viral vectors include retroviral vectors (e.g. Maloney murine leukemia virus, MML-V), adenoviral vectors (e.g. AD100), lentiviral vectors (HIV and FIV-based vectors), herpesvirus vectors (e.g. HSV-2).

### Viral Vectors

A base editor described herein can therefore be delivered with viral vectors. In some embodiments, a base editor disclosed herein can be encoded on a nucleic acid that is contained in a viral vector. In some embodiments, one or more components of the base editor system can be encoded on one or more viral vectors. For example, a base editor and guide nucleic acid can be encoded on a single viral vector. In other embodiments, the base editor and guide nucleic acid are encoded on different viral vectors. In either case, the base editor and guide nucleic acid can each be operably linked to a promoter and terminator. The combination of components encoded on a viral vector can be determined by the cargo size constraints of the chosen viral vector.

The use of RNA or DNA viral based systems for the delivery of a base editor takes advantage of highly evolved processes for targeting a virus to specific cells in culture or in the host and trafficking the viral payload to the nucleus or host cell genome. Viral vectors can be administered directly to cells in culture, patients (in vivo), or they can be used to treat cells in vitro, and the modified cells can optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

Viral vectors can include lentivirus (e.g., HIV and FIV-based vectors), Adenovirus (e.g., AD100), Retrovirus (e.g., Maloney murine leukemia virus, MML-V), herpesvirus vectors (e.g., HSV-2), and Adeno-associated viruses (AAVs), or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing, the expression of the base editor and optional guide nucleic acid can be driven by a cell-type specific promoter.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (See, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

Retroviral vectors, especially lentiviral vectors, can require polynucleotide sequences smaller than a given length for efficient integration into a target cell. For example, retroviral vectors of length greater than 9 kb can result in low viral titers compared with those of smaller size. In some aspects, a base editor of the present disclosure is of sufficient size so as to enable efficient packaging and delivery into a target cell via a retroviral vector. In some embodiments, a base editor is of a size so as to allow efficient packing and delivery even when expressed together with a guide nucleic acid and/or other components of a targetable nuclease system.

Packaging cells are typically used to form virus particles that are capable of infecting a host cell. Such cells include 293 cells, which package adenovirus, and psi.2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, Adeno-associated virus (“AAV”) vectors used in gene therapy typically only possess ITR sequences from the AAV genome which are required for packaging and integration into the host genome. Viral DNA can be packaged in a cell line, which contains a helper plasmid encoding the other AAV genes, namely rep and cap, but lacking ITR sequences. The cell line can also be infected with adenovirus as a helper. The helper virus can promote replication of the AAV vector and expression of AAV genes from the helper plasmid. The helper plasmid in some cases is not packaged in significant amounts due to a lack of ITR sequences. Contamination with adenovirus can be reduced by, e.g., heat treatment to which adenovirus is more sensitive than AAV.

In applications where transient expression is preferred, adenoviral based systems can be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors can also be used to transduce cells with target nucleic acids, e.g., in the in vitro production of nucleic acids and peptides, and for in vivo and ex vivo gene therapy procedures (See, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). The construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

AAV is a small, single-stranded DNA dependent virus belonging to the parvovirus family. The 4.7 kb wild-type (wt) AAV genome is made up of two genes that encode four replication proteins and three capsid proteins, respectively, and is flanked on either side by 145-bp inverted terminal repeats (ITRs). The virion is composed of three capsid proteins, Vp1, Vp2, and Vp3, produced in a 1:1:10 ratio from the same open reading frame but from differential splicing (Vp1) and alternative translational start sites (Vp2 and Vp3, respectively). Vp3 is the most abundant subunit in the virion and participates in receptor recognition at the cell surface defining the tropism of the virus. A phospholipase domain, which functions in viral infectivity, has been identified in the unique N terminus of Vp1.

Similar to wt AAV, recombinant AAV (rAAV) utilizes the cis-acting 145-bp ITRs to flank vector transgene cassettes, providing up to 4.5 kb for packaging of foreign DNA. Subsequent to infection, rAAV can express a fusion protein as described herein and persist without integration into the host genome by existing episomally in circular head-to-tail concatemers. Although there are numerous examples of rAAV success using this system, in vitro and in vivo, the limited packaging capacity has limited the use of AAV-mediated gene delivery when the length of the coding sequence of the gene is equal or greater in size than the wt AAV genome.

Viral vectors can be selected based on the application. For example, for in vivo or ex vivo gene delivery, AAV can be advantageous over other viral vectors. In some embodiments, AAV allows low toxicity, which can be due to the purification method not requiring ultra-centrifugation of cell particles that can activate the immune response. In some embodiments, AAV allows low probability of causing insertional mutagenesis because it does not integrate into the host genome. Adenoviruses are commonly used as vaccines because of the strong immunogenic response they induce. Packaging capacity of the viral vectors can limit the size of the base editor that can be packaged into the vector.

AAV has a packaging capacity of about 4.5 Kb or 4.75 Kb including two 145 base inverted terminal repeats (ITRs). This means disclosed base editor as well as a promoter and transcription terminator can fit into a single viral vector. Constructs larger than 4.5 or 4.75 Kb can lead to significantly reduced virus production. For example, SpCas9 is quite large, the gene itself is over 4.1 Kb, which makes it difficult for packing into AAV. Therefore, embodiments of the present disclosure include utilizing a disclosed base editor which is shorter in length than conventional base editors. In some examples, the base editors are less than 4 kb. Disclosed base editors can be less than 4.5 kb, 4.4 kb, 4.3 kb, 4.2 kb, 4.1 kb, 4 kb, 3.9 kb, 3.8 kb, 3.7 kb, 3.6 kb, 3.5 kb, 3.4 kb, 3.3 kb, 3.2 kb, 3.1 kb, 3 kb, 2.9 kb, 2.8 kb, 2.7 kb, 2.6 kb, 2.5 kb, 2 kb, or 1.5 kb. In some embodiments, the disclosed base editors are 4.5 kb or less in length.

An AAV can be AAV1, AAV2, AAV5 or any combination thereof. One can select the type of AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82: 5887-5911 (2008)).

In some embodiments, lentiviral vectors are used. Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

Lentiviruses can be prepared as follows. After cloning pCasES10 (which contains a lentiviral transfer plasmid backbone), HEK293FT at low passage (p=5) were seeded in a T-75 flask to 50% confluence the day before transfection in DMEM with 10% fetal bovine serum and without antibiotics. After 20 hours, media is changed to OptiMEM (serum-free) media and transfection was done 4 hours later. Cells are transfected with 10 μg of lentiviral transfer plasmid (pCasES10) and the following packaging plasmids: 5 μg of pMD2.G (VSV-g pseudotype), and 7.5 μg of psPAX2 (gag/pol/rev/tat). Transfection can be done in 4 mL OptiMEM with a cationic lipid delivery agent (50 μl Lipofectamine 2000 and 100 μl Plus reagent). After 6 hours, the media is changed to antibiotic-free DMEM with 10% fetal bovine serum. These methods use serum during cell culture, but serum-free methods are preferred.

Lentivirus can be purified as follows. Viral supernatants are harvested after 48 hours. Supernatants are first cleared of debris and filtered through a 0.45 μm low protein binding (PVDF) filter. They are then spun in an ultracentrifuge for 2 hours at 24,000 rpm. Viral pellets are resuspended in 50 μl of DMEM overnight at 4° C. They are then aliquoted and immediately frozen at −80° C.

In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors are contemplated.

Any RNA of the systems, for example a guide RNA or a base editor-encoding mRNA, can be delivered in the form of RNA. Base editor-encoding mRNA can be generated using in vitro transcription. For example, nuclease mRNA can be synthesized using a PCR cassette containing the following elements: T7 promoter, optional kozak sequence (GCCACC), nuclease sequence, and 3′ UTR such as a 3′ UTR from beta globin-polyA tail. The cassette can be used for transcription by T7 polymerase. Guide polynucleotides (e.g., gRNA) can also be transcribed using in vitro transcription from a cassette containing a T7 promoter, followed by the sequence “GG”, and guide polynucleotide sequence.

To enhance expression and reduce possible toxicity, the base editor-coding sequence and/or the guide nucleic acid can be modified to include one or more modified nucleoside e.g. using pseudo-uridine, 5-Methyl-cytosine, 2′-O-methyl-3′-phosphonoacetate, 2′-O-methyl (‘M’), 2′-O-methyl-3′-phosphorothioate (‘MS’) and 2′-O-methyl-3′-thiophosphonoacetate (‘MSP’), 5-methoxyuridine, phosphorothioate, and N1-Methylpseudouridine. Methods for using chemically modified mRNAs and guide RNAs are known in the art and described, for example, by Jiang et al., Chemical modifications of adenine base editor mRNA and guide RNA expand its application scope. Nat Commun 11, 1979 (2020). https://doi.org/10.1038/s41467-020-15892-8, Callum et al., N1-Methylpseudouridine substitution enhances the performance of synthetic mRNA switches in cells, Nucleic Acids Research, Volume 48, Issue 6, 6 Apr. 2020, Page e35, and Andries et al., Journal of Controlled Release, Volume 217, 10 Nov. 2015, Pages 337-344, each of which is incorporated herein by reference in its entirety.

The small packaging capacity of AAV vectors makes the delivery of a number of genes that exceed this size and/or the use of large physiological regulatory elements challenging. These challenges can be addressed, for example, by dividing the protein(s) to be delivered into two or more fragments, wherein the N-terminal fragment is fused to a split intein-N and the C-terminal fragment is fused to a split intein-C. These fragments are then packaged into two or more AAV vectors. As used herein, “intein” refers to a self-splicing protein intron (e.g., peptide) that ligates flanking N-terminal and C-terminal exteins (e.g., fragments to be joined). The use of certain inteins for joining heterologous protein fragments is described, for example, in Wood et al., J. Biol. Chem. 289(21); 14512-9 (2014). For example, when fused to separate protein fragments, the inteins IntN and IntC recognize each other, splice themselves out and simultaneously ligate the flanking N- and C-terminal exteins of the protein fragments to which they were fused, thereby reconstituting a full-length protein from the two protein fragments. Other suitable inteins will be apparent to a person of skill in the art.

A fragment of a fusion protein as described herein can vary in length. In some embodiments, a protein fragment ranges from 2 amino acids to about 1000 amino acids in length. In some embodiments, a protein fragment ranges from about 5 amino acids to about 500 amino acids in length. In some embodiments, a protein fragment ranges from about 20 amino acids to about 200 amino acids in length. In some embodiments, a protein fragment ranges from about 10 amino acids to about 100 amino acids in length. Suitable protein fragments of other lengths will be apparent to a person of skill in the art.

In one embodiment, dual AAV vectors are generated by splitting a large transgene expression cassette in two separate halves (5′ and 3′ ends, or head and tail), where each half of the cassette is packaged in a single AAV vector (of <5 kb). The re-assembly of the full-length transgene expression cassette is then achieved upon co-infection of the same cell by both dual AAV vectors followed by: (1) homologous recombination (HR) between 5′ and 3′ genomes (dual AAV overlapping vectors); (2) ITR-mediated tail-to-head concatemerization of 5′ and 3′ genomes (dual AAV trans-splicing vectors); or (3) a combination of these two mechanisms (dual AAV hybrid vectors). The use of dual AAV vectors in vivo results in the expression of full-length proteins. The use of the dual AAV vector platform represents an efficient and viable gene transfer strategy for transgenes of >4.7 kb in size.

### Inteins

Inteins (intervening protein) are auto-processing domains found in a variety of diverse organisms, which carry out a process known as protein splicing. Protein splicing is a multi-step biochemical reaction comprised of both the cleavage and formation of peptide bonds. While the endogenous substrates of protein splicing are proteins found in intein-containing organisms, inteins can also be used to chemically manipulate virtually any polypeptide backbone. Exemplary intein polypeptide sequences are provided in the Sequence Listing as SEQ ID NOs: 381-388.

In protein splicing, the intein excises itself out of a precursor polypeptide by cleaving two peptide bonds, thereby ligating the flanking extein (external protein) sequences via the formation of a new peptide bond. This rearrangement occurs post-translationally (or possibly co-translationally). Intein-mediated protein splicing occurs spontaneously, requiring only the folding of the intein domain.

About 5% of inteins are split inteins, which are transcribed and translated as two separate polypeptides, the N-intein and C-intein, each fused to one extein. Upon translation, the intein fragments spontaneously and non-covalently assemble into the canonical intein structure to carry out protein splicing in trans. The mechanism of protein splicing entails a series of acyl-transfer reactions that result in the cleavage of two peptide bonds at the intein-extein junctions and the formation of a new peptide bond between the N- and C-exteins. This process is initiated by activation of the peptide bond joining the N-extein and the N-terminus of the intein. Virtually all inteins have a cysteine or serine at their N-terminus that attacks the carbonyl carbon of the C-terminal N-extein residue. This N to O/S acyl-shift is facilitated by a conserved threonine and histidine (referred to as the TXXH motif), along with a commonly found aspartate, which results in the formation of a linear (thio)ester intermediate. Next, this intermediate is subject to trans-(thio)esterification by nucleophilic attack of the first C-extein residue (+1), which is a cysteine, serine, or threonine. The resulting branched (thio)ester intermediate is resolved through a unique transformation: cyclization of the highly conserved C-terminal asparagine of the intein. This process is facilitated by the histidine (found in a highly conserved HNF motif) and the penultimate histidine and may also involve the aspartate. This succinimide formation reaction excises the intein from the reactive complex and leaves behind the exteins attached through a non-peptidic linkage. This structure rapidly rearranges into a stable peptide bond in an intein-independent fashion.

In some embodiments, a portion or fragment of a nuclease (e.g., Cas9) is fused to an intein. The nuclease can be fused to the N-terminus or the C-terminus of the intein. In some embodiments, a portion or fragment of a fusion protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, an N-terminal fragment of a base editor (e.g., ABE, CBE) is fused to a split intein-N and a C-terminal fragment is fused to a split intein-C. These fragments are then packaged into two or more AAV vectors. In some embodiments, the N-terminus of an intein is fused to the C-terminus of a fusion protein and the C-terminus of the intein is fused to the N-terminus of an AAV capsid protein.

In one embodiment, inteins are utilized to join fragments or portions of an adenosine deaminase base editor protein that is grafted onto an AAV capsid protein. The use of certain inteins for joining heterologous protein fragments is described, for example, in Wood et al., J. Biol. Chem. 289(21); 14512-9 (2014). For example, when fused to separate protein fragments, the inteins IntN and IntC recognize each other, splice themselves out and simultaneously ligate the flanking N- and C-terminal exteins of the protein fragments to which they were fused, thereby reconstituting a full-length protein from the two protein fragments. Other suitable inteins will be apparent to a person of skill in the art.

In some embodiments, an ABE was split into N- and C-terminal fragments at Ala, Ser, Thr, or Cys residues within selected regions of SpCas9. These regions correspond to loop regions identified by Cas9 crystal structure analysis. The N-terminus of each fragment is fused to an intein-N and the C-terminus of each fragment is fused to an intein C at amino acid positions S303, T310, T313, S355, A456, S460, A463, T466, S469, T472, T474, C574, S577, A589, and S590, which are indicated in bold capital letters in the sequence below.

(SEQ ID NO: 197)

| | |
|---|---|
| 1 | mdkkysigld igtnsvgwav itdeykvpsk kfkvlgntdr |
| | hsikknliga llfdsgetae |
| 61 | atrlkrtarr rytrrknric ylqeifsnem akvddsffhr |
| | leesflveed kkherhpifg |
| 121 | nivdevayhe kyptiyhlrk klvdstdkad lrliylalah |
| | mikfrghfli egdlnpdnsd |
| 181 | vdklfiqlvq tynqlfeenp inasgvdaka ilsarlsksr |
| | rlenliaqlp gekknglfgn |
| 241 | lialslgltp nfksnfdlae daklqlskdt ydddldnlla |
| | qigdqyadlf laaknlsdai |
| 301 | llSdilrvnT eiTkaplsas mikrydehhq dltllkalvr |
| | qqlpekykei ffdqskngya |
| 361 | gyidggasqe efykfikpil ekmdgteell vklnredllr |
| | kqrtfdngsi phqihlgelh |
| 421 | ailrrqedfy pflkdnreki ekiltfripy yvgplArgnS |
| | rfAwmTrkSe eTiTpwnfee |
| 481 | vvdkgasaqs fiermtnfdk nlpnekvlpk hsllyeyftv |
| | yneltkvkyv tegmrkpafl |
| 541 | sgeqkkaivd llfktnrkvt vkqlkedyfk kieCfdSvei |
| | sgvedrfnAS lgtyhdllki |
| 601 | ikdkdfldne enedilediv ltltlfedre mieerlktya |
| | hlfddkvmkq lkrrrytgwg |
| 661 | rlsrklingi rdkqsgktil dflksdgfan rnfmqlihdd |
| | sltfkediqk aqvsgqgdsl |
| 721 | hehianlags paikkgilqt vkvvdelvkv mgrhkpeniv |
| | iemarenqtt qkgqknsrer |
| 781 | mkrieegike lgsqilkehp ventqlqnek lylyylangr |
| | dmyvdqeldi nrlsdydvdh |
| 841 | ivpqsflkdd sidnkvltrs dknrgksdnv pseevvkkmk |
| | nywrqllnak litqrkfdnl |
| 901 | tkaergglse ldkagfikrq lvetrqitkh vaqildsrmn |
| | tkydendkli revkvitlks |
| 961 | klvsdfrkdf qfykvreinn yhhahdayln avvgtalikk |
| | ypklesefvy gdykvydvrk |
| 1021 | miakseqeig katakyffys nimnffktei tlangeirkr |
| | plietngetg eivwdkgrdf |
| 1081 | atvrkvlsmp qvnivkktev qtggfskesi lpkrnsdkli |
| | arkkdwdpkk yggfdsptva |
| 1141 | ysvlvvakve kgkskklksv kellgitime rssfeknpid |
| | fleakgykev kkdliiklpk |
| 1201 | yslfelengr krmlasagel qkgnelalps kyvnflylas |
| | hyeklkgspe dneqkqlfve |
| 1261 | qhkhyldeii eqisefskrv iladanldkv lsaynkhrdk |
| | pireqaenii hlftltnlga |
| 1321 | paafkyfdtt idrkrytstk evldatlihq sitglyetri |
| | dlsqlggd. |

### Use of Nucleobase Editors

The correction of point mutations in disease-associated genes and alleles provides new strategies for gene correction with applications in therapeutics and basic research.

The present disclosure provides methods for the treatment of a subject diagnosed with a disease associated with or caused by a point mutation that can be corrected by a base editor system provided herein. For example, in some embodiments, a method is provided that comprises administering to a subject having such a disease, e.g., a disease caused by a genetic mutation, an effective amount of a nucleobase editor (e.g., an adenosine deaminase base editor) that corrects the point mutation in the disease associated gene. The present disclosure provides methods for the treatment of GSD1a that are associated with or caused by a point mutation that can be corrected by deaminase mediated gene editing. Suitable diseases that can be treated with the strategies and fusion proteins provided herein will be apparent to those of skill in the art based on the instant disclosure.

Provided herein are methods of using a base editor or base editor system for editing a nucleobase in a target nucleotide sequence associated with a disease or disorder (e.g., GSD1a). In some embodiments, the activity of the base editor (e.g., comprising an adenosine deaminase and a Cas9 domain) results in a correction of the point mutation. In some embodiments, the target DNA sequence comprises a G→A point mutation associated with a disease or disorder, and deamination of the mutant A base results in a sequence that is not associated with a disease or disorder. In some embodiments, the target DNA sequence comprises a T→C point mutation associated with a disease or disorder, and the deamination of the mutant C base results in a sequence that is not associated with a disease or disorder.

In some embodiments, the deaminases (e.g., adenosine deaminase) provided herein are capable of deaminating a deoxyadenosine residue of DNA. Other aspects of the disclosure provide fusion proteins that comprise a deaminase (e.g., an adenosine deaminase) and a domain (e.g., a Cas9) capable of binding to a specific nucleotide sequence. For example, adenosine can be converted to an inosine residue, which typically base pairs with a cytosine residue. Such fusion proteins are useful inter alia for targeted editing of nucleic acid sequences. Such fusion proteins can be used for targeted editing of DNA in vitro, e.g., for the generation of mutant cells or animals; for the introduction of targeted mutations, e.g., for the correction of genetic defects in cells ex vivo, e.g., in cells obtained from a subject that are subsequently re-introduced into the same or another subject; and for the introduction of targeted mutations in vivo, e.g., the correction of genetic defects or the introduction of deactivating mutations in disease-associated genes in a G to A, or a T to C to mutation can be treated using the nucleobase editors provided herein. The present disclosure provides fusion proteins, nucleic acids, vectors, compositions, methods, kits, systems, etc. that utilize the deaminases and nucleobase editors.

### Use of Nucleobase Editors to Target Nucleotides in the G6PC Gene

The suitability of nucleobase editors that target a nucleotide in the G6PC gene is evaluated as described herein. In one embodiment, a single cell of interest is transfected, transduced, or otherwise modified with a nucleic acid molecule or molecules encoding a nucleobase editor described herein together with a small amount of a vector encoding a reporter (e.g., GFP). These cells can be immortalized human cell lines, such as 293T, K562 or U20S. Alternatively, primary human cells may be used. Cells may also be obtained from a subject or individual, such as from tissue biopsy, surgery, blood, plasma, serum, or other biological fluid. Such cells may be relevant to the eventual cell target,

Delivery may be performed using a viral vector as further described below. In one embodiment, transfection may be performed using lipid transfection (such as Lipofectamine, Metafectamine, or Fugene) or by electroporation. Following transfection, expression of GFP can be determined either by fluorescence microscopy or by flow cytometry to confirm consistent and high levels of transfection. These preliminary transfections can comprise different nucleobase editors to determine which combinations of editors give the greatest activity.

The activity of the nucleobase editor is assessed as described herein, i.e., by sequencing the target gene to detect alterations in the target sequence. For Sanger sequencing, purified PCR amplicons are cloned into a plasmid backbone, transformed, miniprepped and sequenced with a single primer. Sequencing may also be performed using next generation sequencing techniques. When using next generation sequencing, amplicons may be 300-500 bp with the intended cut site placed asymmetrically. Following PCR, next generation sequencing adapters and barcodes (for example Illumina multiplex adapters and indexes) may be added to the ends of the amplicon, e.g., for use in high throughput sequencing (for example on an Illumina MiSeq).

The fusion proteins that induce the greatest levels of target specific alterations in initial tests can be selected for further evaluation.

In particular embodiments, the nucleobase editors are used to target polynucleotides of interest. In one embodiment, a nucleobase editor as described herein is delivered to cells (e.g., hepatocytes) in conjunction with a guide RNA that is used to target a nucleic acid sequence, e.g., a G6PC polynucleotide harboring GSD1a-associated mutations, thereby altering the target gene, i.e., G6PC.

In some embodiments, a base editor is targeted by a guide RNA to introduce one or more edits to the sequence of a gene of interest (e.g. G6PC). In some embodiments, the one or more alterations are introduced into the glucose-6-phosphatase (G6PC) gene. In some embodiments the one or more alterations is R83C. In some embodiments, the one or more alterations is Q347X. In some embodiments, the alteration is introduced into a representative Homo sapiens G6PC protein, found under NCBI Reference Sequence No. AAA16222.1. In some embodiments, the alteration is introduced into a representative Homo sapiens G6PC nucleic acid sequence, found under GenBank Reference Sequence No. U01120.1.

### Methods for Editing Nucleic Acids

Some aspects of the disclosure provide methods for editing a nucleic acid. In some embodiments, the method is a method for editing a nucleobase of a nucleic acid (e.g., a base pair of a double-stranded DNA sequence). In some embodiments, the method comprises the steps of: a) contacting a target region of a nucleic acid (e.g., a double-stranded DNA sequence) with a complex comprising a base editor (e.g., a Cas9 domain fused to an adenosine deaminase) and a guide nucleic acid (e.g., gRNA), wherein the target region comprises a targeted nucleobase pair, b) inducing strand separation of said target region, c) converting a first nucleobase of said target nucleobase pair in a single strand of the target region to a second nucleobase, and d) cutting no more than one strand of said target region, where a third nucleobase complementary to the first nucleobase base is replaced by a fourth nucleobase complementary to the second nucleobase. In some embodiments, the method results in less than 20% indel formation in the nucleic acid. It should be appreciated that in some embodiments, step b is omitted. In some embodiments, the method results in less than 19% 1, 8% 1, 6% 1, 4% 1, 2% 1, 0%, 8%, 6%, 4%, 2%, 1%, 0.5%, 0.2%, or less than 0.1% indel formation. In some embodiments, the method further comprises replacing the second nucleobase with a fifth nucleobase that is complementary to the fourth nucleobase, thereby generating an intended edited base pair (e.g., C·G to T·A). In some embodiments, at least 5% of the intended base pairs are edited. In some embodiments, at least 10%, 15%, 20%, 25%, 30%, 35%, 40%, 45%, or 50% of the intended base pairs are edited.

In some embodiments, the ratio of intended products to unintended products in the target nucleotide is at least 2:1, 5:1, 10:1, 20:1, 30:1, 40:1, 50:1, 60:1, 70:1, 80:1, 90:1, 100:1, or 200:1, or more. In some embodiments, the ratio of intended mutation to indel formation is greater than 1:1, 10:1, 50:1, 100:1, 500:1, or 1000:1, or more. In some embodiments, the cut single strand (nicked strand) is hybridized to the guide nucleic acid. In some embodiments, the cut single strand is opposite to the strand comprising the first nucleobase. In some embodiments, the base editor comprises a Cas9 domain. In some embodiments, the base editor protects or binds the non-edited strand. In some embodiments, the base editor comprises nickase activity. In some embodiments, the intended edited base pair is upstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edited base pair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site. In some embodiments, the method does not require a canonical (e.g., NGG) PAM site. In some embodiments, the nucleobase editor comprises a linker. In some embodiments, the linker is 1-25 amino acids in length. In some embodiments, the linker is 5-20 amino acids in length. In some embodiments, linker is 10, 11, 12, 13, 14, 15, 16, 17, 18, 19, or 20 amino acids in length. In one embodiment, the linker is 32 amino acids in length. In another embodiment, a “long linker” is at least about 60 amino acids in length. In other embodiments, the linker is between about 3-100 amino acids in length. In some embodiments, the target region comprises a target window, wherein the target window comprises the target nucleobase pair. In some embodiments, the target window comprises 1-10 nucleotides. In some embodiments, the target window is 1-9, 1-8, 1-7, 1-6, 1-5, 1-4, 1-3, 1-2, or 1 nucleotides in length. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edited base pair is within the target window. In some embodiments, the target window comprises the intended edited base pair. In some embodiments, the method is performed using any of the base editors provided herein. In some embodiments, a target window is a methylation window.

In some embodiments, the disclosure provides methods for editing a nucleotide. In some embodiments, the disclosure provides a method for editing a nucleobase pair of a double-stranded DNA sequence. In some embodiments, the method comprises a) contacting a target region of the double-stranded DNA sequence with a complex comprising a base editor and a guide nucleic acid (e.g., gRNA), where the target region comprises a target nucleobase pair, b) inducing strand separation of said target region, c) converting a first nucleobase of said target nucleobase pair in a single strand of the target region to a second nucleobase, d) cutting no more than one strand of said target region, wherein a third nucleobase complementary to the first nucleobase base is replaced by a fourth nucleobase complementary to the second nucleobase, and the second nucleobase is replaced with a fifth nucleobase that is complementary to the fourth nucleobase, thereby generating an intended edited base pair, wherein the efficiency of generating the intended edited base pair is at least 5%. It should be appreciated that in some embodiments, step b is omitted. In some embodiments, at least 5% of the intended base pairs are edited. In some embodiments, at least 10%, 15%, 20%, 25%, 30%, 35%, 40%, 45%, or 50% of the intended base pairs are edited. In some embodiments base editing by a method described herein may have a base conversion efficiency of at least 10% at any particular gene site. In some embodiments, base editing by a method described herein may have a base conversion efficiency of at least 20%, at least 25%, at least 30%, at least 35%, at least 40%, at least 45%, at least 50% at least 55% or at least 60%, at least 65%, at least 70%, at least 75%, at least 80%, at least 85%, at least 90%, or at least 95%, 96%, 97%, 98% or at least 99% at any particular gene site. In some embodiments base editing by a method described herein may have a base conversion efficiency of at least 70% at any particular gene site. In some embodiments base editing by a method described herein may have a base conversion efficiency of at least 80% at any particular gene site. In some embodiments base editing by a method described herein may have a base conversion efficiency of at least 90% at any particular gene site.

In some embodiments, the method causes less than 19%, 18%, 16%, 14%, 12%, 10%, 8%, 6%, 4%, 2%, 1%, 0.5%, 0.2%, or less than 0.1% indel formation. In some embodiments, the ratio of intended product to unintended products at the target nucleotide is at least 2:1, 5:1, 10:1, 20:1, 30:1, 40:1, 50:1, 60:1, 70:1, 80:1, 90:1, 100:1, or 200:1, or more. In some embodiments, the ratio of intended mutation to indel formation is greater than 1:1, 10:1, 50:1, 100:1, 500:1, or 1000:1, or more. In some embodiments, the cut single strand is hybridized to the guide nucleic acid. In some embodiments, the cut single strand is opposite to the strand comprising the first nucleobase. In some embodiments, the nucleobase editor comprises nickase activity. In some embodiments, the intended edited base pair is upstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edited base pair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site. In some embodiments, the method does not require a canonical (e.g., NGG) PAM site. In some embodiments, the nucleobase editor comprises a linker. In some embodiments, the linker is 1-25 amino acids in length. In some embodiments, the linker is 5-20 amino acids in length. In some embodiments, the linker is 10, 11, 12, 13, 14, 15, 16, 17, 18, 19, or 20 amino acids in length. e.g., In some embodiments, the target region comprises a target window, wherein the target window comprises the target nucleobase pair. In some embodiments, the target window comprises 1-10 nucleotides. In some embodiments, the target window is 1-9, 1-8, 1-7, 1-6, 1-5, 1-4, 1-3, 1-2, or 1 nucleotides in length. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edited base pair occurs within the target window. In some embodiments, the target window comprises the intended edited base pair. In some embodiments, the nucleobase editor is any one of the base editors provided herein.

### Expression of Fusion Proteins in a Host Cell

Fusion proteins of the disclosure comprising an adenosine deaminase variant may be expressed in virtually any host cell of interest, including but not limited to, bacteria, yeast, fungi, insects, plants, and animal cells, using routine methods known to the skilled artisan. For example, a DNA encoding an adenosine deaminase of the disclosure can be cloned by designing suitable primers for the upstream and downstream of CDS based on the cDNA sequence. The cloned DNA may be directly, or after digestion with a restriction enzyme when desired, or after addition of a suitable linker and/or a nuclear localization signal ligated with a DNA encoding one or more additional components of a base editing system. The base editing system is translated in a host cell to form a complex.

A DNA encoding a protein domain described herein can be obtained by chemically synthesizing the DNA, or by connecting synthesized partly overlapping oligoDNA short chains by utilizing the PCR method and the Gibson Assembly method to construct a DNA encoding the full length thereof. The advantage of constructing a full-length DNA by chemical synthesis or a combination of PCR method or Gibson Assembly method is that the codon to be used can be designed in CDS full-length according to the host into which the DNA is introduced. In the expression of a heterologous DNA, the protein expression level is expected to increase by converting the DNA sequence thereof to a codon highly frequently used in the host organism. As the data of codon use frequency in host to be used, for example, the genetic code use frequency database (kazusa.or.jp/codon/index.html) disclosed in the home page of Kazusa DNA Research Institute can be used, or documents showing the codon use frequency in each host may be referred to. By reference to the obtained data and the DNA sequence to be introduced, codons showing low use frequency in the host from among those used for the DNA sequence may be converted to a codon coding the same amino acid and showing high use frequency.

An expression vector containing a DNA encoding a nucleic acid sequence-recognizing module and/or a nucleic acid base converting enzyme can be produced, for example, by linking the DNA to the downstream of a promoter in a suitable expression vector. In some embodiments, animal cell expression plasmids (e.g., pA1-11, pXT1, pRc/CMV, pRc/RSV, pcDNAI/Neo), and animal virus vectors such as retrovirus, vaccinia virus, adenovirus, and the like are used. In other embodiments, Escherichia coli-derived plasmids (e.g., pBR322, pBR325, pUC12, pUC13); Bacillus subtilis-derived plasmids (e.g., pUB110, pTP5, pC194); yeast-derived plasmids (e.g., pSH19, pSH15); insect cell expression plasmids (e.g., pFast-Bac); bacteriophages such as lambda phage and the like; insect virus vectors such as baculovirus and the like (e.g., BmNPV, AcNPV); and the like are used.

In some embodiments, any promoter appropriate for gene expression in a given host can be used. In a conventional method using DSB, since the survival rate of the host cell sometimes decreases markedly due to the toxicity, it is desirable to increase the number of cells by the start of the induction by using an inductive promoter. However, since sufficient cell proliferation can also be afforded by expressing the nucleic acid-modifying enzyme complex of the present disclosure, a constitution promoter can also be used without limitation.

For example, without limitation, when the host is an animal cell, SRα promoter, SV40 promoter, LTR promoter, CMV (cytomegalovirus) promoter, RSV (Rous sarcoma virus) promoter, MoMuLV (Moloney mouse leukemia virus) LTR, HSV-TK (simple herpes virus thymidine kinase) promoter and the like are used. Of these, CMV promoter, SRα promoter and the like are suitable for use.

In addition to those mentioned above, an expression vector containing an enhancer, splicing signal, terminator, polyA addition signal, a selection marker such as drug resistance gene, auxotrophic complementary gene and the like, replication origin and the like, on demand can be used.

An RNA encoding a protein domain described herein can be prepared, for example, by transcription to mRNA in an in vitro transcription system known per se by using a vector encoding DNA encoding the above-mentioned nucleic acid sequence-recognizing module and/or a nucleic acid base converting enzyme as a template.

A fusion protein of the disclosure can be intracellularly expressed by introducing an expression vector containing a DNA encoding a nucleic acid sequence-recognizing module and/or a nucleic acid base converting enzyme into a host cell, and culturing the host cell.

As the animal cell, cell lines such as monkey COS-7 cell, monkey Vero cell, Chinese hamster ovary (CHO) cell, dhfr gene-deficient CHO cell, mouse L cell, mouse AtT-20 cell, mouse myeloma cell, rat GH3 cell, human FL cell and the like, pluripotent stem cells such as iPS cell, ES cell and the like of human and other mammals, and primary cultured cells prepared from various tissues are used. Furthermore, zebrafish embryo, Xenopus oocyte and the like can also be used.

All the above-mentioned host cells may be haploid (monoploid), or polyploid (e.g., diploid, triploid, tetraploid and the like). In the conventional mutation introduction methods, mutation is, in principle, introduced into only one homologous chromosome to produce a hetero gene type. Therefore, desired phenotype is not expressed unless dominant mutation occurs, and homozygousness inconveniently requires labor and time. In contrast, according to the present disclosure, since mutation can be introduced into any allele on the homologous chromosome in the genome, the desired phenotype can be expressed in a single generation even in the case of recessive mutation, which is extremely useful since the problem of the conventional method can be solved.

An expression vector can be introduced by a known method (e.g., lysozyme method, competent method, PEG method, CaCl₂) coprecipitation method, electroporation method, the microinjection method, the particle gun method, lipofection method, Agrobacterium method and the like) according to the kind of the host.

A vector can be introduced into an animal cell according to the methods described in, for example, Cell Engineering additional volume 8, New Cell Engineering Experiment Protocol, 263-267 (1995) (published by Shujunsha), and Virology, 52, 456 (1973). A cell comprising a vector can be cultured according to a known method according to the type of host.

As a medium for culturing an animal cell, for example, minimum essential medium (MEM) containing about 5 to about 20% of fetal bovine serum (Science, 122, 501 (1952)), Dulbecco's modified Eagle medium (DMEM) (Virology, 8, 396 (1959)), RPMI 1640 medium (The Journal of the American Medical Association, 199, 519 (1967)), 199 medium (Proceeding of the Society for the Biological Medicine, 73, 1 (1950)) and the like are used. The pH of the medium is preferably about 6 to about 8. The culture is generally maintained at about 30° C. to about 40° C. Where necessary, aeration and stirring may be performed.

When a higher eukaryotic cell, such as animal cell, is used as a host cell, a DNA encoding a base editing system of the present disclosure (e.g., comprising an adenosine deaminase variant) is introduced into a host cell under the regulation of an inducible promoter (e.g., metallothionein promoter (induced by heavy metal ion), heat shock protein promoter (induced by heat shock), Tet-ON/Tet-OFF system promoter (induced by addition or removal of tetracycline or a derivative thereof), steroid-responsive promoter (induced by steroid hormone or a derivative thereof) etc.), the induction substance is added to the medium (or removed from the medium) at an appropriate stage to induce expression of the nucleic acid-modifying enzyme complex, culture is performed for a given period to carry out a base editing and, introduction of a mutation into a target gene, transient expression of the base editing system can be realized.

Alternatively, the above-mentioned inductive promoter can also be utilized as a vector removal mechanism when higher eukaryotic cells, such animal cells and the like, are used as host cells. That is, a vector is mounted with a replication origin that functions in a host cell, and a nucleic acid encoding a protein necessary for replication (e.g., SV40 on and large T antigen, oriP and EBNA-1 etc. for animal cells) of the expression of the nucleic acid encoding the protein is regulated by the above-mentioned inducible promoter. As a result, while the vector is autonomously replicatable in the presence of an induction substance, when the induction substance is removed, autonomous replication is not available, and the vector naturally falls off along with cell division (autonomous replication is not possible by the addition of tetracycline and doxycycline in Tet-OFF system vector).

### Precise Correction of Pathogenic Mutations

In some embodiments, the intended mutation is a precise correction of a pathogenic mutation or a disease-causing mutation. The pathogenic mutation can be a pathogenic single nucleotide polymorphism (SNP) or be caused by a SNP. For example, the pathogenic mutation can be an amino acid change in a protein encoded by a gene. In another example, the pathogenic mutation can be a pathogenic SNP in a gene. The precise correction can be to revert the pathogenic mutation back to its wild-type state. In some embodiments, the pathogenic mutation is a G→A point mutation associated with a disease or disorder, and wherein the deamination of the mutant A base with an A-to-G base editor (ABE) results in a sequence that is not associated with a disease or disorder. In some embodiments, the pathogenic mutation is a C→T point mutation. The C→T point mutation can be corrected, for example, by targeting an A-to-G base editor (ABE) to the opposite strand and editing the complement A of the pathogenic T nucleobase. A base editor can be targeted to a pathogenic SNP, or to the complement of the pathogenic SNP. The nomenclature of the description of pathogenic or disease-causing mutations and other sequence variations are described in den Dunnen, J. T. and Antonarakis, S. E., “Mutation Nomenclature Extensions and Suggestions to Describe Complex Mutations: A Discussion.” Human Mutation 15:712 (2000), the entire contents of which is hereby incorporated by reference.

In a particular embodiment, the disease or disorder is Glycogen Storage Disease Type 1 (GSD1 or Von Gierke Disease). In some embodiments, the disease or disorder is GSD1a. In some embodiments, the pathogenic mutation is in the G6PC gene. In some embodiments, the pathogenic mutation is Q347X of the G6PC gene. In some embodiments, the pathogenic mutation is R83C of the G6PC gene.

### Pharmaceutical Compositions

In some aspects, a pharmaceutical composition is provided, which comprises any of the base editors, fusion proteins, or the fusion protein-guide polynucleotide complexes described herein.

The pharmaceutical compositions as described herein can be prepared in accordance with known techniques. See, e.g., Remington, The Science And Practice of Pharmacy (21st ed. 2005). In some embodiments, the pharmaceutical composition comprises a pharmaceutically acceptable carrier. Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Pharmaceutically acceptable carriers can include agents that can stabilize, optimize or otherwise alter the form, consistency, viscosity, pH, pharmacokinetics, solubility of the formulation. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

Some nonlimiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

Pharmaceutical compositions can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions can also contain one or more osmotic modulating agents, i.e., a compound that modulates the osmotic properties (e.g., tonicity, osmolality, and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

In some embodiments, the pharmaceutical composition is formulated for delivery to a subject, e.g., for gene editing. Suitable routes of administrating the pharmaceutical composition described herein include, without limitation: topical, subcutaneous, suboccipital, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site. In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump can be used (see, e.g., Langer, 1990, Science 249: 1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228: 190; During et al., 1989, Ann. Neurol. 25:351; Howard et al., 1989, J. Neurosurg. 71: 105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic use as solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration can be a liquid, e.g., sterile saline, lactated Ringer's or Hank's solution. In addition, the pharmaceutical composition can be in solid forms and re-dissolved or suspended immediately prior to use. Lyophilized forms are also contemplated. The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6: 1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein can be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound as described herein in lyophilized form and (b) a second container containing a pharmaceutically acceptable diluent (e.g., sterile used for reconstitution or dilution of the lyophilized compound described herein. Optionally associated with such container(s) can be a notice in the form prescribed by a governmental agency regulating the manufacture, use or sale of pharmaceuticals or biological products, which notice reflects approval by the agency of manufacture, use, or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers can be formed from a variety of materials such as glass or plastic. In some embodiments, the container holds a composition that is effective for treating a disease described herein and can have a sterile access port. For example, the container can be an intravenous solution bag or a vial having a stopper pierceable by a hypodermic injection needle. The active agent in the composition is a compound as described and provided herein. In some embodiments, the label on or associated with the container indicates that the composition is used for treating the disease of choice. The article of manufacture can further comprise a second container comprising a pharmaceutically-acceptable buffer, such as phosphate-buffered saline, Ringer's solution, or dextrose solution. It can further include other materials desirable from a commercial and user standpoint, including other buffers, diluents, filters, needles, syringes, and package inserts with instructions for use.

In some embodiments, any of the fusion proteins, gRNAs, and/or complexes described herein are provided as part of a pharmaceutical composition. In some embodiments, the pharmaceutical composition comprises any of the fusion proteins provided herein. In some embodiments, the pharmaceutical composition comprises any of the complexes provided herein. In some embodiments, the pharmaceutical composition comprises a ribonucleoprotein complex comprising an RNA-guided nuclease (e.g., Cas9) that forms a complex with a gRNA and a cationic lipid. In some embodiments pharmaceutical composition comprises a gRNA, a nucleic acid programmable DNA binding protein, a cationic lipid, and a pharmaceutically acceptable excipient. Pharmaceutical compositions can optionally comprise one or more additional therapeutically active substances.

In some embodiments, compositions provided herein are administered to a subject, for example, to a human subject, in order to effect a targeted genomic modification within the subject. In some embodiments, cells are obtained from the subject and contacted with any of the pharmaceutical compositions provided herein. In some embodiments, cells removed from a subject and contacted ex vivo with a pharmaceutical composition are re-introduced into the subject, optionally after the desired genomic modification has been effected or detected in the cells. Methods of delivering pharmaceutical compositions comprising nucleases are known, and are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties. Although the descriptions of pharmaceutical compositions provided herein are principally directed to pharmaceutical compositions which are suitable for administration to humans, it will be understood by the skilled artisan that such compositions are generally suitable for administration to animals or organisms of all sorts, for example, for veterinary use.

Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, domesticated animals, pets, and commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein can be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit. Pharmaceutical formulations can additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated in its entirety herein by reference) discloses various excipients used in formulating pharmaceutical compositions and known techniques for the preparation thereof. See also PCT application PCT/US2010/055131 (Publication number WO2011/053982 A8, filed Nov. 2, 2010), incorporated in its entirety herein by reference, for additional suitable methods, reagents, excipients and solvents for producing pharmaceutical compositions comprising a nuclease.

Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

The compositions, as described above, can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well-known to the medical practitioner. For therapeutic applications, it is that amount sufficient to achieve a medically desirable result.

In some embodiments, compositions in accordance with the present disclosure can be used for treatment of any of a variety of diseases, disorders, and/or conditions.

### Methods of Treating Glycogen Storage Disease Type 1a (GSD1a)

Provided also are methods of treating Glycogen Storage Disease Type 1a (GSD1a) and/or the genetic mutations in G6PC that cause GSD1a that comprise administering to a subject (e.g., a mammal, such as a human) a therapeutically effective amount of a pharmaceutical composition that comprises a polynucleotide encoding a base editor system (e.g., Adenosine Deaminase Base Editor (ABE) and gRNA) described herein. In some embodiments, the base editor is a fusion protein that comprises a polynucleotide programmable DNA binding domain and an adenosine deaminase domain. A cell of the subject is transduced with the base editor and one or more guide polynucleotides that target the base editor to effect an A·T to G·C alteration of a nucleic acid sequence containing mutations in the G6PC gene.

The methods herein include administering to the subject (including a subject identified as being in need of such treatment, or a subject suspected of being at risk of disease and in need of such treatment) an effective amount of a composition described herein. Identifying a subject in need of such treatment can be in the judgment of a subject or a health care professional and can be subjective (e.g., opinion) or objective (e.g., measurable by a test or diagnostic method).

The therapeutic methods, in general, comprise administration of a therapeutically effective amount of a pharmaceutical composition comprising, for example, a vector encoding a base editor and a gRNA that targets the G6PC gene of a subject (e.g., a human patient) in need thereof. Such treatment will be suitably administered to a subject, particularly a human subject, suffering from, having, susceptible to, or at risk for GSD1a. The compositions herein may be also used in the treatment of any other disorders in which GSD1a may be implicated.

In one embodiment, a method of monitoring treatment progress is provided. The method includes the step of determining a level of diagnostic marker (Marker) (e.g., SNP associated with GSD1a) or diagnostic measurement (e.g., screen, assay) in a subject suffering from or susceptible to a disorder or symptoms thereof associated with GSD1a in which the subject has been administered a therapeutic amount of a composition herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment or therapy as described herein; this pre-treatment level of Marker can then be compared to the level of Marker in the subject after the treatment or therapy commences, to determine the efficacy of the treatment or therapy.

In some embodiments, cells are obtained from the subject and contacted with a pharmaceutical composition as provided herein. In some embodiments, cells removed from a subject and contacted ex vivo with a pharmaceutical composition are re-introduced into the subject, optionally after the desired genomic modification has been affected or detected in the cells.

Methods of delivering pharmaceutical compositions comprising nucleases are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties. Although the descriptions of pharmaceutical compositions provided herein are principally directed to pharmaceutical compositions which are suitable for administration to humans, it will be understood by the skilled artisan that such compositions are generally suitable for administration to animals or organisms of all sorts, for example, for veterinary use.

### Kits

Various aspects of this disclosure provide kits comprising a base editor system. In one embodiment, the kit comprises a nucleic acid construct comprising a nucleotide sequence encoding a nucleobase editor fusion protein. The fusion protein comprises a deaminase (e.g., adenine deaminase) and a nucleic acid programmable DNA binding protein (napDNAbp). In some embodiments, the kit comprises at least one guide RNA capable of targeting a nucleic acid molecule of interest, e.g., G6PC GSD1a associated mutations. In some embodiments, the kit comprises a nucleic acid construct comprising a nucleotide sequence encoding at least one guide RNA.

The kit provides, in some embodiments, instructions for using the kit to edit one or more G6PC GSD1a associated mutations. The instructions will generally include information about the use of the kit for editing nucleic acid molecules. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit can comprise instructions in the form of a label or separate insert (package insert) for suitable operational parameters. In yet another embodiment, the kit can comprise one or more containers with appropriate positive and negative controls or control samples, to be used as standard(s) for detection, calibration, or normalization. The kit can further comprise a second container comprising a pharmaceutically-acceptable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use. In certain embodiments, the kit is useful for the treatment of a subject having Glycogen Storage Disease Type 1a (GSD1a).

The practice of the embodiments described and provided herein employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides described and provided herein, and, as such, may be considered in making and practicing the disclosed and described embodiments. Particularly useful techniques for particular embodiments will be discussed in the sections that follow.

The following examples are put forth so as to provide those of ordinary skill in the art with a complete disclosure and description of how to make and use the assay, screening, and therapeutic methods described herein, and are not intended to limit the scope of any of the embodiments and/or the disclosure described herein.

### EXAMPLES

### Example 1: Precise Correction In Vivo in Heterozygous Transgenic Glycogen Storage Disease Type 1a (GSD1a) R83C Mice

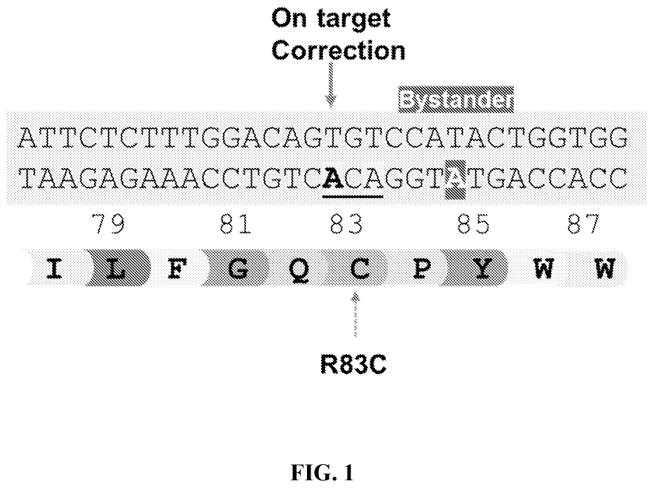

Glycogen storage disease type 1a (GSD1a) is caused by a mutation in the glucose-6-phosphatase (G6PC) gene, which affects about 80% of patients with GSD1a. The R83C mutation affects about 900 US patients annually diagnosed with Glycogen storage disease type 1a (GSD1a). This mutation is a single base substitution that introduces a cysteine at position 83 (R83C) of the G6PC protein. A precise correction of R83C will likely restore expression of G6PC and normalize glucose metabolism. A representative G6PC nucleotide target sequence (ATTCTCTTTGGACAGTGTCCATACTGGTGG (SEQ ID NO 399) having complementary sequence TAAGAGAAACCTGTCACAGGTATGACCACC (SEQ ID NO: 400): and corresponding amino acid sequence (ILFGQCPYWW (SEQ ID NO: 401)) indicating on target and bystander site “a” nucleobases for correction of the R83C mutation are shown in FIG. 1. A precise correction at this site would yield the following conversion: TGT>CGT or TGT>CGC (Cysteine>Arginine).

The G6PC gRNA sequence hybridizes to the complement of the G6PC target sequence shown below:

(SEQ ID NO: 395)
CAGTATGGACACTGTCCAAAGAGAAT

The NNGRRT PAM sequence (i.e., Staphylococcus aureus Cas9 (saCas9)) is underlined above.

The gRNA sequence is as follows: CAGUAUGGACACUGUCCAAA (SEQ ID NO: 370).

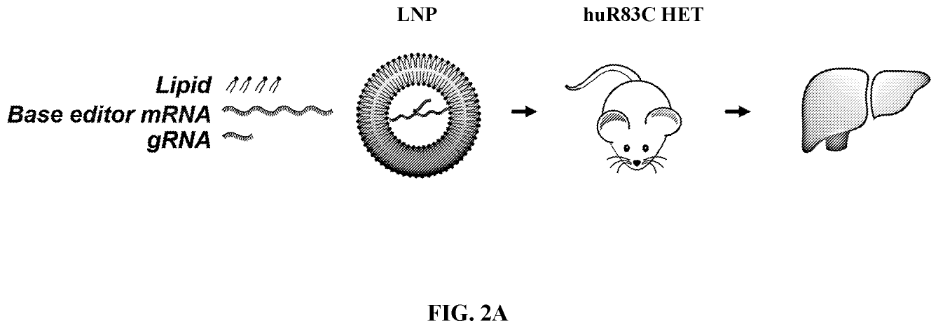

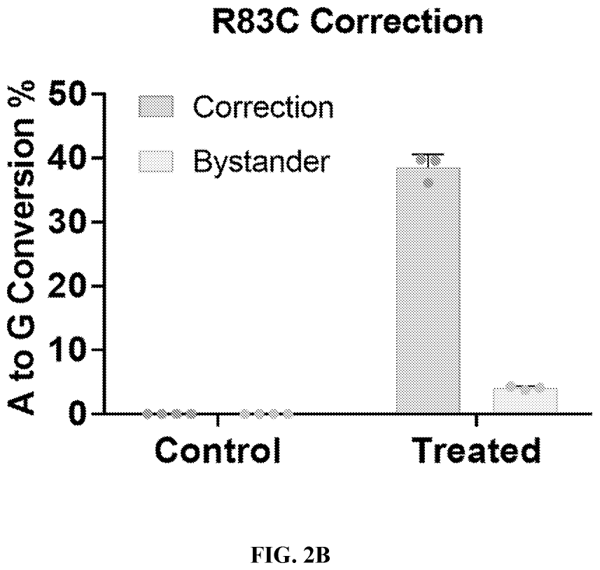

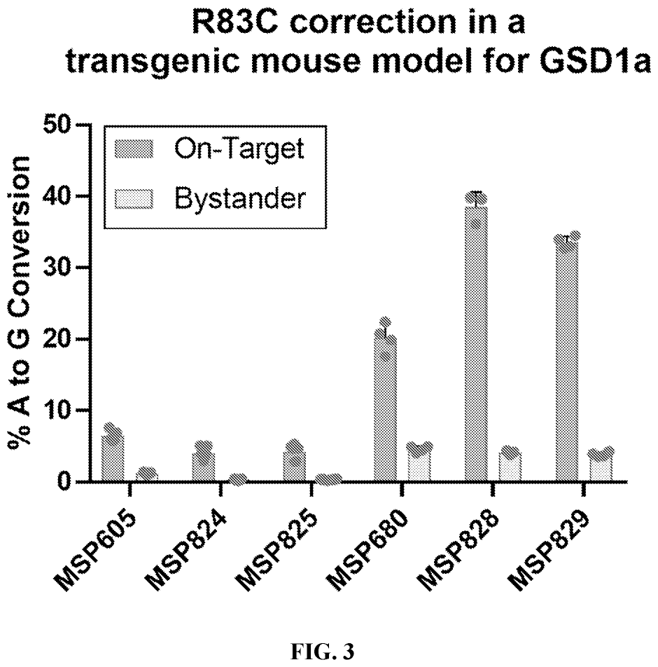

The base-editing efficiency of adenosine deaminase base editors (ABE) using TadA variants MSP605, MSP824, MSP825, MSP680, MSP828, and MSP829 (see Table 18) and saCas9n was evaluated in vivo using a transgenic mouse model heterozygous for huG6PC, harboring the R83C mutation for Glycogen storage disease type 1a (GSD1a) (FIGS. 2B and 3). The use of saCas9 for efficient in vivo genome editing and exemplification of an saCas9 sgRNA scaffold are described in A. Ran et al. (2015, Nature, Vol. 520, pages 186-191). The engineering of Cas9 variants, e.g., SaCas9, with relaxed PAM recognition specificities is described by B. P. Kleinstiver et al., 2014, Nature Biotechnol., 33(12):1293-1299.

TABLE 18
Adenosine Deaminase Base Editor Variants

| TadA | |
|---|---|
| Variant | mRNA base-editor variant |
| MSP605 | dimeric TadA-ABE7.10 (Y147T + Q154S + V82G)-saCas9n |
| MSP824 | dimeric TadA-ABE7.10 (Y147D + Q154S + V82G + F149Y + |
| | D167N)-saCas9n |
| MSP825 | dimeric TadA-ABE7.10 (Y147D + Q154S + V82G + L36H + |
| | N157K + F149Y + D167N)-saCas9n |
| MSP680 | monomeric TadA-ABE7.10 (Y147T + Q154S + V82G + |
| | I76Y)-saCas9n |
| MSP828 | monomeric TadA-ABE7.10 (Y147D + Q154S + V82G + |
| | I76Y + F149Y + D167N)-saCas9n |
| MSP829 | monomeric TadA-ABE7.10 (Y147D + Q154S + V82G + |
| | I76Y + L36H + N157K + F149Y + D167N)-saCas9n |

FIG. 2A depicts the in vivo workflow used to introduce the base editors into the transgenic mice. Lipid nanoparticles (LNP) carrying base editor mRNA and gRNA were dosed via intravenous (IV) injection into the transgenic mice at a dose of 1 mg/kg. Next-generation sequencing data from whole-liver extracts revealed significant correction for R83C (FIGS. 2B and 3). TadA variant MSP828 demonstrated about 40% precise correction of the R83C mutation, with low bystander editing. This level of mutation correction is expected to restore glucose homeostasis.

The level of in vivo correction for Glycogen storage disease type 1a (GSD1a) by base-editing was greater than that achieved by HDR-base (homology directed repair) methodologies and was achieved without insertion or double-stranded breaks. These results demonstrate base-editing technology as an effective approach for mutation correction for Glycogen storage disease type 1a (GSD1a) and its therapeutic potential.

### Example 2: In Vivo Base Editing Correction of Metabolic Defects in GSD1a R83C Mice GSD1a Overview

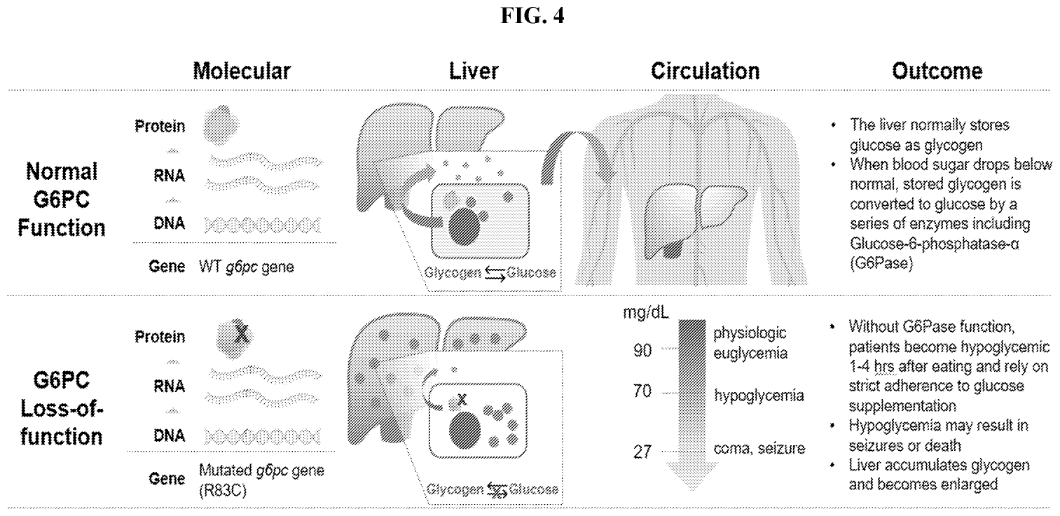

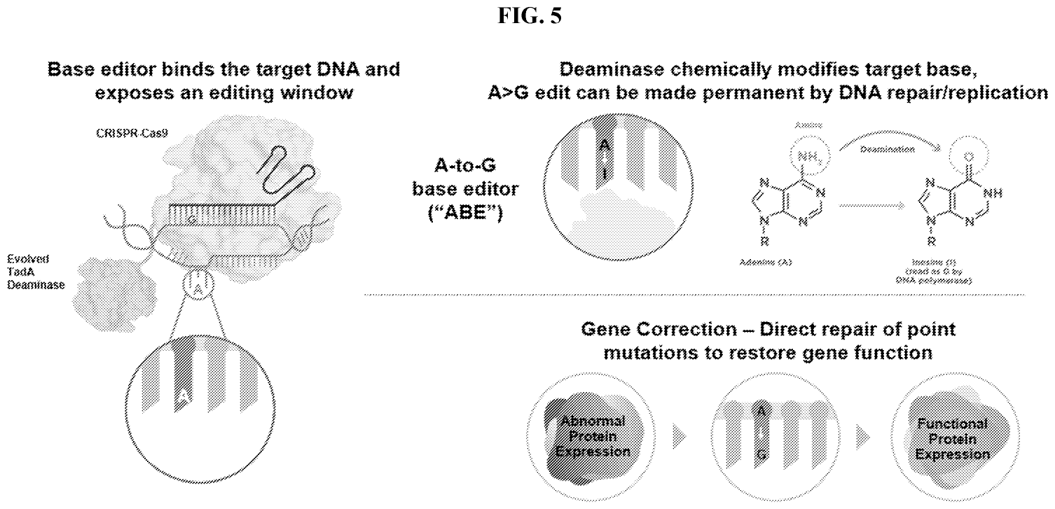

As depicted schematically in FIG. 4, (GSD1a) is an autosomal recessive disorder caused by mutations in the G6PC gene. The most prevalent pathogenic mutation identified in Caucasian GSD1a patients is R83C, located in the active site of the enzyme and associated with inactivation of G6Pase. A loss of G6Pase function can result in life-threatening hypoglycemia, seizures and even death. To mitigate hypoglycemia, patients must maintain strict and frequent adherence to glucose supplementation through day and night, by way of a slow glucose release formula. One missed or delayed dose can result in emergency hypoglycemia. Among many complications, enlarged liver, accumulation of uric acid, lactate, and lipids are common in GSD1a patients.

### Utility of the Described Base Editors for Generating Permanent and Predictable Single

### Nucleotide Substitutions

The R83C mutation introduces a single G>A conversion in the g6pc gene. Adenine base editors (ABEs) as described herein effect the programmable conversion of A to G in genomic DNA, thus supporting their utility to correct this mutation. As shown schematically in FIG. 5, the adenine base editor is a fusion protein containing an evolved TadA deaminase connected to CRISPR-Cas enzyme. The base editor binds to target DNA that is complementary to the guide-RNA (superimposed on the CRISPR-Cas9 enzyme) and exposes a stretch of single-stranded DNA. The deaminase converts the target adenine into inosine, and the Cas enzyme nicks the opposite strand, which is then repaired, completing the base pair conversion. Thus, the direct repair of a point mutation has the potential for restoration of gene function.

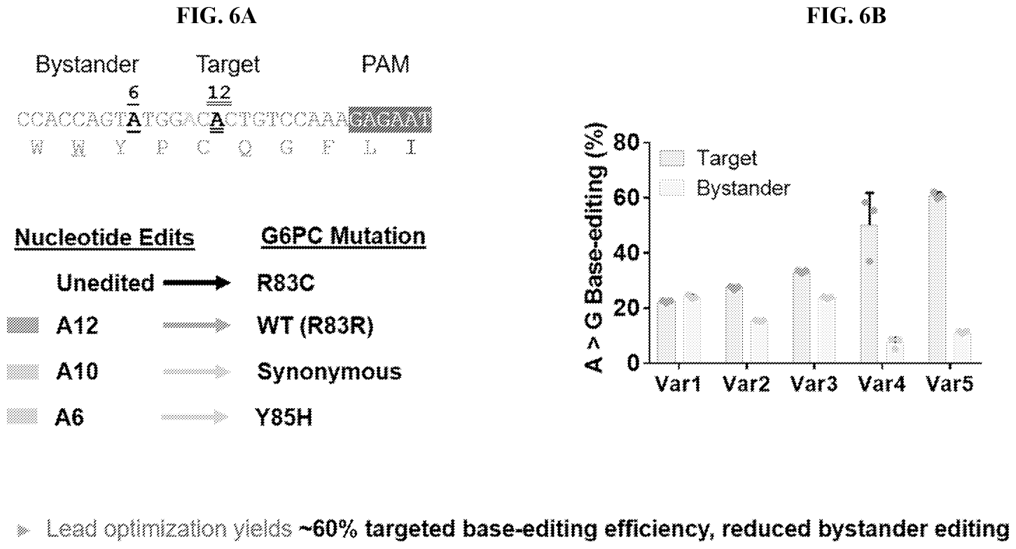

In this Example, base-editors for A>G conversion in the g6pc gene were optimized for correction of R83C. Shown in FIG. 6A is the target DNA sequence (CCACCAGTATGGACACTGTCCAAAGAGAAT (SEQ ID NO: 402)) and underlying amino acid translation for the GSD1a R83C mutation (WWYPCQGFLI; SEQ ID NO: 403). The target nucleobase to be edited is represented by double underlining, at position 12. The editing window also includes a possible bystander, shown represented by single underlining at position 6. An edit that may result in a synonymous conversion is shown at position 10.

For screening, a HEK293 cell line that expressed the G6PC transgene harboring the R83C mutation was generated and was transfected with base-editor mRNA and gRNA. Allele frequencies were assessed by high-throughput targeted amplicon Next-Generation Sequencing. Variants 1-5 represent a combination of gRNA and base-editor RNA, engineered for optimized target correction. Variant 5 yielded approximately 60% targeted base-editing efficiency for R83C correction and limited bystander editing (FIG. 6B).

### Mouse In Vivo Disease Model and Demonstration of In Vivo Correction of the R83C Single Nucleotide Mutation

### In Vivo Correction of R83C Base Editing

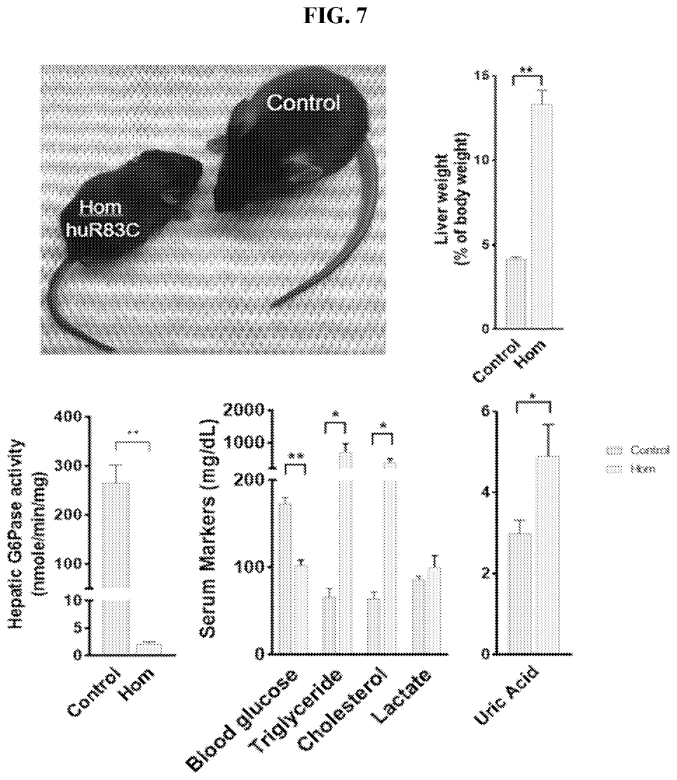

To validate base-editing efficiency for R83C correction in vivo, a novel GSD1a mouse that expresses the human G6PC-R83C transgene in place of mouse G6pc was generated. It was confirmed that mice homozygous for huR83C exhibited postnatal lethality and rarely survived to weaning (21 days). On glucose supplementation therapy, the animals survived to at least 3 weeks of age and revealed characteristic pathological signatures of GSD1a, such as reduced body weight, enlarged livers, significant G6Pase inhibition, and abnormal serum metabolites compared to littermate controls (FIG. 7). This phenotype is consistent with published and clinical reports in humans.

For the in vivo experiments, LNP-mediated delivery was tested in transgenic mice that were heterozygous for huR83C due to neonatal lethality of homozygous mice. The schematic in FIG. 2A depicts in vivo workflow, with lipid nanoparticle, or LNP, co-formulations of base-editor mRNA and gRNA dosed via IV injection. Given neonatal lethality of the homozygous mice, LNP-dosing was administered via the temporal vein shortly post birth, and activity was compared with that in adult mice. Next Generation Sequencing (NGS) analysis of whole liver extracts revealed approximately 40% base-editing efficiency in adults and up to ˜60% efficiency in newborns, with a broader range in efficiencies (FIG. 8A). Bystander editing remained low in adults and newborns. (FIG. 8A).

Newborn mice homozygous for huR83C were treated with lipid nanoparticles (LNP) containing guide RNA and mRNA encoding ABE. It was found that the treated mice survived and grew normally to 3 weeks of age, without hypoglycemia-induced seizures, in the absence of glucose therapy. The treated homozygous huR83C mice displayed editing efficiencies up to ˜60% in total liver extracts, consistent with littermate controls that were heterozygous for huR83C (FIG. 8B). It was thus demonstrated that LNP-mediated R83C correction was associated with the survival of the homozygous huR83C mice.

### Reversal of GSD-1a Pathology Via Base-Editing for Correction of R83C In Vivo

At 3 weeks, it was validated and confirmed that the treated homozygous huR83C mice displayed proper metabolic function, with restoration of near-normal serum metabolites, including glucose, triglycerides, cholesterol, lactate, and uric acid, as demonstrated by the darker-color bars in FIG. 9A, compared to controls. Moreover, the results of biochemical assays of G6PC activity (as assessed biochemically and via lead-phosphate staining) in LNP-treated homozygous huR83C mice were consistent with those of litter-mate controls. (FIG. 9A).

Hepatomegaly is another clinical presentation of GSD1a and is primarily caused by excess glycogen and lipid deposition in the liver. To evaluate the extent of hepatomegaly in homozygous huG6PC-R83C mice post base-editing, liver sections were collected from 3 wk old newborn mice and immune-histochemical analysis were conducted via hematoxylin and eosin (H&E) and Oil red O staining (FIG. 9B). Significant lipid deposition (heavy H&E staining) and enlarged hepatocytes was visualized in liver sections from homozygous mice exhibiting negligible G6Pase activity (FIG. 9B, center panels, H&E), consistent with GSD-1a. In the case of base-edited homozygous huG6PC-R83C mice showing restored G6PC activity (“HOM huR83C”, right panels, FIG. 9B), lipid deposition was significantly reduced and consistent with controls (left panel), (FIG. 9B, Lipid), and restoration of hepatocyte size was apparent. Accordingly, the immuno-histochemical analyses revealed normal hepatocyte size and lipid deposition in LNP-treated mice. (FIG. 9B). Taken together, the data demonstrate the ability of base-editing to correct the R83C mutation and to reverse the metabolic defects and pathology associated with GSD1a. In addition, these data lend further support of the functional restoration and positive clinical outcomes via base-editing for GsD-1a.

As described in this Example, novel adenine base editors and guide RNA that achieved precise correction of R83C in vitro and in vivo were generated and validated. LNP-mediated delivery of ABE and gRNA yielded significant base-editing efficiency, namely, up to ˜60% base editing efficiency, with restoration of hepatic G6Pase activity and metabolic function consistent with controls.

### Single LNP Dose Administration Maintains Euglycemia During a 24 Hour Fasting Challenge Via Base Editing

A hallmark symptom of GSD-1a pathology is fasting hypoglycemia, with a precipitous decline in blood glucose levels within minutes. A full proof-of-concept study was conducted in GSD-1a transgenic mice, homozygous for huG6PC-R83C, to test whether the animals could sustain a 24 hour (hr) fast after base-editing treatment as described herein. In this study, 100% animal survival was achieved post-24 hr fasting period in LNP-treated (1.5 mpk) GSD-1a animals and in healthy controls. In addition, normal fasting glucose levels were measured in control mice and in treated mice pre- and post-24 hr fasting, which maintained levels above hypoglycemic therapeutic threshold (>60 mg/dL), (FIG. 10).

Kaplan-Meier Survival Estimates for Homozygous huG6PC-R83C Mice

Kaplan-Meier survival curves were generated to estimate the survival of newborn transgenic mice homozygous for huG6PC-R83C, either post base-editing via ABE mRNA (ABE-treated) or untreated (Untreated), (FIG. 12) Newborn mice were genotyped via PCR analysis of genomic tail DNA using the following primers, a universal forward primer (5′-ACCTACTGATGATGCACCTTTGATCAATAGAT-3′), (SEQ ID NO: 424), a mouse specific reverse primer (5′-CATCACCCCTCGGGATGGTTCTT-3′), (SEQ ID NO: 425), a human specific reverse primer 1 (5′-CAGCCCAGAATCCCAACCACAAAAT-3′), (SEQ ID NO: 426), and a human specific reverse primer 2 (5′-AGACCAGCTCGACTTGGGATGG-3′), (SEQ ID NO: 427). Survival was noted for transgenic mice homozygous for huG6PC-R83C. Untreated mice were either still-born (n=6) or died at 8 hours (n=6) and 24 hours (n=1). Administration of 15% glucose injections extended survival of the animals to 32 hours (n=5), 48 hours (n=2), and 56 hours (n=2). All ABE-treated mice homozygous for huG6PC-R83C survived to the termination of study at 3 weeks.

G6PC Target Sequences for Use with Base Editors to Correct the R83C Mutation

In addition to the G6PC target sequence and guide RNA described in Example 1, alternative G6PC target sequences that can be used in conjunction with the base editors to effect base editing to correct the R83C mutation as described herein include those shown in Table 19. As shown, the target sequences include the types of PAMs and base editors, such as IBEs as described herein, suitable for use. In the protospacer sequences in Table 19, the position of the targeted “A” nucleotide (i.e., A8-A15) is shown in bold/underline. G6PC gRNA sequences hybridize to the complement of the G6PC target sequence shown in Table 19. The PAM sequences (e.g., SpCas9) are underlined in Table 19.

Inlaid base editors (IBEs) noted in Table 19 refer to structures of Cas9 and TadA having an architecture in which the deaminase domains are internal to (embedded inside) a CRISPR-Cas protein, e.g., Cas9. The IBE architecture allows for a greater breadth of potential base editing targets compared with other base editors and is not limited by the requirement of a suitably positioned Cas9 protospacer adjacent motif sequence. Such IBEs exhibited shifted editing windows and exhibited greater editing efficiency, thus allowing for the editing of targets outside the canonical editing window with reduced DNA and RNA off-target editing frequency. Accordingly, IBEs expand the breadth of potential base editing targets by extending the range of editing windows that can be created for any given CRISPR-Cas protein used to target the DNA. Through the insertion of the deaminase into a CRISPR protein at different strategic positions, the active site of the deaminase can be repositioned, making IBEs capable of editing outside the traditional editing window. IBE architectures are described hereinabove and in S. Haihua Chu et al., The CRISPR Journal, Vol. 4, No. 2; published online 20 Apr. 2021 (DOI: 10.1089/crispr.2020.0144).

TABLE 19
Protospacer + PAM sequences (5′ to 3′) for
correcting the R83C mutation, where the PAM
sequence is underlined

CCACCAGTATGGACACTGTC CAAA (SEQ ID NO: 416) with
spCas9-NRRH A15 can use IBE architecture
CACCAGTATGGACACTGTCC AAAG (SEQ ID NO: 417) with
spCas9-NRRH A14 can use IBE architecture
ACCAGTATGGACACTGTCCA AAGA (SEQ ID NO: 418) with
spCas9-NRRH A13 can use IBE architecture
CCAGTATGGACACTGTCCAA AGAG (SEQ ID NO: 419) with
spCas9-NRRH A12 can use IBE architecture
CAGTATGGACACTGTCCAAA GAGA (SEQ ID NO: 420) with
spCas9-NRRH A11 can use IBE architecture
AGTATGGACACTGTCCAAAG AGA (SEQ ID NO: 421) with
spCas9-NGA A10 can use IBE architecture
GTATGGACACTGTCCAAAGA GAAT (SEQ ID NO: 422) with
spCas9-NRRH A9 can use IBE architecture
TATGGACACTGTCCAAAGAG AATC (SEQ ID NO: 423) with
spCas9-NRTH A8 can use IBE architecture

The gRNA sequences which hybridize to the complement of the G6PC target sequence in Table 19 are as follows (5′ to 3′): CCACCAGUAUGGACACUGUC (SEQ ID NO: 371); CACCAGUAUGGACACUGUCC (SEQ ID NO: 372); ACCAGUAUGGACACUGUCCA (SEQ ID NO: 373); CCAGUAUGGACACUGUCCAA (SEQ ID NO: 374); CAGUAUGGACACUGUCCAAA (SEQ ID NO: 370); AGUAUGGACACUGUCCAAAG (SEQ ID NO: 375); GUAUGGACACUGUCCAAAGA (SEQ ID NO: 376); and UAUGGACACUGUCCAAAGAG (SEQ ID NO: 377).

A protospacer and PAM sequence for use in the products, compositions and methods described herein is, (5′ to 3′), CAGTATGGACACTGTCCAAAGAGAAT (SEQ ID NO: 395), in which the PAM sequence, GAGAAT, is underlined. The gRNA sequence, as presented supra, which hybridizes to the complement of the target sequence is CAGUAUGGACACUGUCCAAA (3′ PAM sequence GAGAAT as shown in the sequence above) (SEQ ID NO: 370).

The gRNA sequence used in the methods described herein comprises or consists of:

(SEQ ID NO: 409)
CACCAGUAUGGACACUGUCCAAAGUUUUAGUACUCUGUAAUGAAAAUUAC
AGAAUCUACUAAAACAAGGCAAAAUGCCGUGUUUAUCUCGUCAACUUGUU
GGCGAGAUUUU
or
(SEQ ID NO: 410)
CCACCAGUAUGGACACUGUCCAAAGUUUUAGUACUCUGUAAUGAAAAUUA
CAGAAUCUACUAAAACAAGGCAAAAUGCCGUGUUUAUCUCGUCAACUUGU
UGGCGAGAUUUU.

In some embodiments, the gRNA sequence used in the methods described herein comprises one or more modified nucleosides. Two exemplary sequences are provided below:

sgRNA_096: 23 nt protospacer
(SEQ ID NO: 409)
mCsmAsmCsCAGUAUGGACACUGUCCAAAGUUUUAGUACUCUGUAAUGAA
AAUUACAGAAUCUACUAAAACAAGGCAAAAUGCCGUGUUUAUCUCGUCAA
CUUGUUGGCGAGAmUsmUsmUsU
sgRNA_097: 34 nt protospacer
(SEQ ID NO: 410)
mCsmCsmAsCCAGUAUGGACACUGUCCAAAGUUUUAGUACUCUGUAAUGA
AAAUUACAGAAUCUACUAAAACAAGGCAAAAUGCCGUGUUUAUCUCGUCA
ACUUGUUGGCGAGAmUsmUsmUsU.

### Example 3: Optimization Via New ABE Variants for R83C Correction

To diversify and expand a selection of saABE variants for R83C correction, the adenosine deaminase domain (referred to as a TadA domain) was engineered via directed evolution to include a combination of mutations. Such mutations were based, at least in part, on novel molecular engineering efforts. Table 20 provides a list of novel SaABE variants, 823-829, and shows mutations present in the TadA domain that were tested alongside two other TadA variants, MSP605 and MSP680, both in vitro and in vivo. Combinations of mutations, including Y147D, F149Y, and D167N, in the TadA deaminase yielded an increase in on-target editing and reduction in bystander editing (FIG. 3). By way of further example, other engineered saABE variants that demonstrated effective on-target editing and reduced bystanding editing include at least one or more of the following mutations: L36H; I76Y; V82G; Q154S; and N157K, e.g., an saABE variant containing at least a combination of I76Y; V82G; Q154S.

TABLE 20
Novel saABE variants for use in correcting a GSD-Ia-R83C mutation

| | | TadA Amino Acid Number | | | | | | | |
|---|---|---|---|---|---|---|---|---|---|
| Variant | | 36 | 76 | 82 | 147 | 149 | 154 | 157 | 167 |
| TadA-7.10 | | L | I | V | Y | F | Q | N | D |
| 605 | dimer | | | G | T | | S | | |
| 680 | mono | | Y | G | T | | S | | |
| 823 | dimer | H | | G | T | | S | K | |
| 824 | dimer | | | G | D | Y | S | | N |
| 825 | dimer | H | | G | D | Y | S | K | N |
| 827 | mono | H | Y | G | T | | S | K | |
| 828 | mono | | Y | G | D | Y | S | | N |
| 829 | mono | H | Y | G | D | Y | S | K | N |

### Example 4: Base Editor Sequences

Polynucleotide (mRNA, DNA) sequences and corresponding amino acid sequence of a representative base editor as described herein are presented below.

MSP828 Base Editor mRNA Sequence

The MSP828 base-editor mRNA open reading frame (ORF) is shown by underlining in the below mRNA sequence.

(SEQ ID NO: 396)
AGGAAAUAAGAGAGAAAAGAAGAGUAAGAAGAAAUAUAAGAGCCACCAUGAGCGAGGUCGAG
UUCUCUCACGAAUAUUGGAUGAGACACGCUCUCACCCUGGCUAAGAGAGCCAGGGACGAAAG
AGAGGUGCCAGUUGGCGCUGUCCUGGUGUUGAACAAUCGCGUCAUCGGAGAAGGAUGGAAUC
GCGCCAUUGGCCUGCACGAUCCAACCGCACAUGCCGAAAUUAUGGCUCUGCGGCAAGGCGGC
CUCGUGAUGCAAAAUUACAGACUGUACGAUGCUACCCUCUACGGCACCUUCGAGCCCUGUGU
CAUGUGUGCUGGGGCAAUGAUUCACUCCCGGAUUGGCCGCGUGGUGUUUGGAGUGCGGAAUG
CCAAGACUGGCGCCGCUGGAUCUCUGAUGGACGUCCUGCACUAUCCUGGGAUGAACCACCGG
GUCGAGAUCACAGAGGGAAUUCUGGCUGACGAGUGCGCAGCCCUGCUGUGCGACUUCUAUAG
AAUGCCCAGAUCGGUGUUCAACGCCCAGAAAAAAGCUCAGAGCAGUACCAAUUCCGGCGGAA
GCAGCGGAGGAUCUUCUGGAAGCGAAACCCCAGGCACCAGCGAGUCUGCCACACCAGAAUCA
UCUGGCGGUAGCUCCGGCGGCUCCAAGAGAAAUUACAUCCUGGGCCUCGCCAUCGGCAUCAC
CUCUGUCGGCUACGGCAUCAUCGACUACGAGACACGGGAUGUGAUUGAUGCCGGCGUGCGGC
UGUUCAAAGAGGCCAACGUCGAGAACAACGAGGGCCGCAGAUCUAAGAGGGGAGCCAGACGG
CUGAAGAGAAGGCGGAGACACAGAAUCCAGAGAGUGAAGAAGCUGCUGUUCGACUACAACCU
GCUGACCGACCACAGCGAGCUGAGCGGCAUCAACCCUUAUGAGGCCAGAGUGAAGGGCCUGA
GCCAGAAGCUGAGCGAGGAAGAGUUUAGCGCCGCACUGCUGCACCUGGCCAAGAGAAGAGGC
GUGCACAACGUGAACGAAGUGGAAGAGGACACCGGCAACGAGCUGUCCACCAAAGAGCAGAU
CAGCAGAAACAGCAAGGCCCUGGAAGAGAAAUACGUGGCCGAACUGCAGCUGGAACGGCUGA
AAAAGGAUGGCGAAGUGCGGGGCAGCAUCAACCGGUUCAAGACCAGCGACUACGUGAAAGAA
GCCAAACAGCUGCUGAAGGUGCAGAAGGCCUACCACCAGCUGGACCAGAGCUUCAUCGACAC
CUACAUCGACCUGCUGGAAACCCGGCGGACCUACUAUGAAGGACCUGGCGAGGGAAGCCCCU
UCGGCUGGAAGGACAUCAAAGAAUGGUACGAGAUGCUGAUGGGCCACUGCACCUACUUUCCC
GAGGAACUGCGGAGCGUGAAGUACGCCUACAACGCCGACCUGUACAACGCCCUGAACGACCU
GAACAACCUCGUGAUCACCCGGGACGAGAACGAGAAGCUGGAAUAUUACGAGAAGUUCCAGA
UCAUCGAGAACGUGUUCAAGCAGAAGAAGAAGCCCACACUGAAGCAGAUCGCCAAAGAGAUC
CUGGUCAACGAGGAAGAUAUCAAGGGCUACAGAGUGACCAGCACCGGCAAGCCCGAGUUCAC
CAACCUGAAGGUGUACCACGACAUCAAGGAUAUCACAGCCCGGAAAGAGAUUAUUGAGAACG
CCGAGCUGCUGGACCAAAUCGCCAAGAUCCUGACCAUCUACCAGUCCUCCGAGGACAUCCAA
GAGGAACUGACCAAUCUGAACUCCGAGCUGACCCAAGAAGAGAUCGAGCAGAUCUCUAAUCU
GAAGGGGUACACAGGCACCCACAACCUGAGCCUGAAGGCCAUCAACCUGAUCCUGGACGAGC
UGUGGCACACCAACGACAACCAGAUUGCCAUCUUCAACCGGCUGAAGCUGGUGCCCAAGAAG
GUGGACCUCAGCCAGCAGAAAGAAAUCCCCACCACACUGGUGGACGACUUCAUUCUGAGCCC
CGUGGUCAAGAGAAGCUUCAUCCAGAGCAUCAAAGUGAUCAACGCCAUCAUCAAGAAGUACG
GGCUGCCCAACGAUAUCAUCAUCGAGCUGGCCCGCGAGAAGAACUCCAAGGACGCUCAGAAA
AUGAUCAACGAGAUGCAGAAGCGGAACCGGCAGACCAACGAGCGGAUCGAGGAAAUCAUCCG
GACCACCGGCAAAGAGAACGCCAAGUACCUGAUCGAGAAGAUCAAGCUGCACGACAUGCAAG
AGGGCAAGUGUCUGUACAGCCUGGAAGCCAUUCCUCUGGAAGAUCUGCUGAACAAUCCCUUC
AACUACGAGGUGGACCACAUCAUCCCCAGAAGCGUGUCCUUCGACAACAGCUUCAACAACAA
GGUGCUCGUGAAGCAAGAGGAAAACUCCAAGAAGGGCAACAGAACCCCAUUCCAGUACCUGA
GCAGCUCCGACAGCAAGAUCAGCUACGAAACCUUCAAGAAGCACAUCCUGAAUCUGGCCAAA
GGCAAGGGCCGCAUCAGCAAGACCAAGAAAGAAUACCUGCUCGAGGAACGGGACAUCAACAG
AUUCAGCGUGCAGAAAGACUUCAUCAAUCGGAACCUGGUGGACACCAGAUACGCCACCAGAG
GCCUGAUGAAUCUGCUGAGAAGCUACUUCCGCGUGAACAAUCUGGACGUGAAAGUCAAGUCC
AUCAACGGCGGCUUCACCAGCUUUCUGCGGAGAAAGUGGAAGUUCAAGAAAGAGCGGAACAA
GGGCUAUAAGCACCACGCCGAGGACGCCCUGAUCAUUGCCAACGCCGAUUUCAUCUUCAAAG
AGUGGAAGAAACUGGACAAGGCCAAAAAAGUGAUGGAAAACCAGAUGUUCGAGGAAAAGCAG
GCCGAGAGCAUGCCCGAGAUCGAAACCGAGCAAGAGUACAAAGAGAUUUUCAUCACGCCCCA
CCAGAUCAAGCACAUUAAGGACUUCAAGGACUACAAGUACAGCCACCGCGUGGACAAGAAGC
CUAAUAGAGAGCUGAUUAACGACACCCUGUACAGCACCCGGAAGGACGACAAGGGCAAUACC
CUGAUCGUCAACAACCUGAACGGCCUGUACGACAAGGACAACGACAAGCUCAAGAAGCUGAU
CAACAAGAGCCCCGAGAAACUGCUGAUGUACCACCACGAUCCUCAGACCUACCAGAAACUGA
AGCUCAUCAUGGAACAGUACGGCGACGAGAAGAAUCCCCUGUACAAGUACUACGAGGAAACC
GGGAACUACCUGACCAAGUACUCCAAAAAGGACAAUGGGCCCGUGAUCAAGAAGAUUAAGUA
UUACGGCAACAAGCUGAAUGCCCACCUGGACAUCACCGACGACUACCCCAACUCCAGAAACA
AGGUGGUCAAGCUGUCCCUGAAGCCUUACAGAUUCGACGUGUACCUGGACAACGGCGUGUAC
AAGUUCGUGACCGUGAAGAACCUGGAUGUGAUCAAAAAAGAAAACUACUACGAAGUGAACAG
CAAGUGCUAUGAGGAAGCCAAGAAACUCAAGAAAAUCAGCAACCAGGCCGAGUUUAUCGCCU
CCUUCUACAACAACGAUCUGAUCAAGAUCAACGGGGAGCUGUAUAGAGUGAUUGGGGUCAAC
AAUGACCUGCUGAACCGGAUCGAAGUCAACAUGAUCGACAUCACCUACCGCGAGUACCUCGA
GAACAUGAACGACAAGAGGCCUCCACGGAUCAUUAAGACAAUCGCCAGCAAGACGCAGAGCA
UUAAGAAGUACAGCACUGACAUUCUGGGCAACCUGUACGAAGUCAAGAGCAAAAAGCACCCG
CAGAUUAUCAAGAAAGGCGAGGGCGCCGACAAGAGAACAGCCGAUGGUUCCGAGUUCGAAAG
CCCCAAGAAGAAGAGGAAAGUCUAGUUAAUUAAGCUGCCUUCUGCGGGGCUUGCCUUCUGGC
CAUGCCCUUCUUCUCUCCCUUGCACCUGUACCUCUUGGUCUUUGAAUAAAGCCUGAGUAGGA
AGAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAA
MSP828 base editor DNA sequence
(SEQ ID NO: 397)
ATGAGCGAGGTCGAGTTCTCTCACGAATATTGGATGAGACACGCTCTCACCCTGGCTAAGAG
AGCCAGGGACGAAAGAGAGGTGCCAGTTGGCGCTGTCCTGGTGTTGAACAATCGCGTCATCG
GAGAAGGATGGAATCGCGCCATTGGCCTGCACGATCCAACCGCACATGCCGAAATTATGGCT
CTGCGGCAAGGCGGCCTCGTGATGCAAAATTACAGACTGTACGATGCTACCCTCTACGGCAC
CTTCGAGCCCTGTGTCATGTGTGCTGGGGCAATGATTCACTCCCGGATTGGCCGCGTGGTGT
TTGGAGTGCGGAATGCCAAGACTGGCGCCGCTGGATCTCTGATGGACGTCCTGCACTATCCT
GGGATGAACCACCGGGTCGAGATCACAGAGGGAATTCTGGCTGACGAGTGCGCaGCCCTGCT
GTGCgacTTCTaTAGAATGCCCAGAtcGGTGTTCAACGCCCAGAAAAAAGCTCAGAGCAGtA
CCaATTCCGGCGGAAGCAGCGGAGGATCTTCTGGAAGCGAAACCCCAGGCACCAGCGAGTCT
GCCACACCAGAATCATCTGGCGGTAGCTCCGGCGGCTCCAAGAGAAATTACATCCTGGGCCT
CGCCATCGGCATCACCTCTGTCGGCTACGGCATCATCGACTACGAGACACGGGATGTGATTG
ATGCCGGCGTGCGGCTGTTCAAAGAGGCCAACGTCGAGAACAACGAGGGCCGCAGATCTAAG
AGGGGAGCCAGACGGCTGAAGAGAAGGCGGAGACACAGAATCCAGAGAGTGAAGAAGCTGCT
GTTCGACTACAACCTGCTGACCGACCACAGCGAGCTGAGCGGCATCAACCCTTATGAGGCCA
GAGTGAAGGGCCTGAGCCAGAAGCTGAGCGAGGAAGAGTTTAGCGCCGCACTGCTGCACCTG
GCCAAGAGAAGAGGCGTGCACAACGTGAACGAAGTGGAAGAGGACACCGGCAACGAGCTGTC
CACCAAAGAGCAGATCAGCAGAAACAGCAAGGCCCTGGAAGAGAAATACGTGGCCGAACTGC
AGCTGGAACGGCTGAAAAAGGATGGCGAAGTGCGGGGCAGCATCAACCGGTTCAAGACCAGC
GACTACGTGAAAGAAGCCAAACAGCTGCTGAAGGTGCAGAAGGCCTACCACCAGCTGGACCA
GAGCTTCATCGACACCTACATCGACCTGCTGGAAACCCGGCGGACCTACTATGAAGGACCTG
GCGAGGGAAGCCCCTTCGGCTGGAAGGACATCAAAGAATGGTACGAGATGCTGATGGGCCAC
TGCACCTACTTTCCCGAGGAACTGCGGAGCGTGAAGTACGCCTACAACGCCGACCTGTACAA
CGCCCTGAACGACCTGAACAACCTCGTGATCACCCGGGACGAGAACGAGAAGCTGGAATATT
ACGAGAAGTTCCAGATCATCGAGAACGTGTTCAAGCAGAAGAAGAAGCCCACACTGAAGCAG
ATCGCCAAAGAGATCCTGGTCAACGAGGAAGATATCAAGGGCTACAGAGTGACCAGCACCGG
CAAGCCCGAGTTCACCAACCTGAAGGTGTACCACGACATCAAGGATATCACAGCCCGGAAAG
AGATTATTGAGAACGCCGAGCTGCTGGACCAAATCGCCAAGATCCTGACCATCTACCAGTCC
TCCGAGGACATCCAAGAGGAACTGACCAATCTGAACTCCGAGCTGACCCAAGAAGAGATCGA
GCAGATCTCTAATCTGAAGGGGTACACAGGCACCCACAACCTGAGCCTGAAGGCCATCAACC
TGATCCTGGACGAGCTGTGGCACACCAACGACAACCAGATTGCCATCTTCAACCGGCTGAAG
CTGGTGCCCAAGAAGGTGGACCTCAGCCAGCAGAAAGAAATCCCCACCACACTGGTGGACGA
CTTCATTCTGAGCCCCGTGGTCAAGAGAAGCTTCATCCAGAGCATCAAAGTGATCAACGCCA
TCATCAAGAAGTACGGGCTGCCCAACGATATCATCATCGAGCTGGCCCGCGAGAAGAACTCC
AAGGACGCTCAGAAAATGATCAACGAGATGCAGAAGCGGAACCGGCAGACCAACGAGCGGAT
CGAGGAAATCATCCGGACCACCGGCAAAGAGAACGCCAAGTACCTGATCGAGAAGATCAAGC
TGCACGACATGCAAGAGGGCAAGTGTCTGTACAGCCTGGAAGCCATTCCTCTGGAAGATCTG
CTGAACAATCCCTTCAACTACGAGGTGGACCACATCATCCCCAGAAGCGTGTCCTTCGACAA
CAGCTTCAACAACAAGGTGCTCGTGAAGCAAGAGGAAAACTCCAAGAAGGGCAACAGAACCC
CATTCCAGTACCTGAGCAGCTCCGACAGCAAGATCAGCTACGAAACCTTCAAGAAGCACATC
CTGAATCTGGCCAAAGGCAAGGGCCGCATCAGCAAGACCAAGAAAGAATACCTGCTCGAGGA
ACGGGACATCAACAGATTCAGCGTGCAGAAAGACTTCATCAATCGGAACCTGGTGGACACCA
GATACGCCACCAGAGGCCTGATGAATCTGCTGAGAAGCTACTTCCGCGTGAACAATCTGGAC
GTGAAAGTCAAGTCCATCAACGGCGGCTTCACCAGCTTTCTGCGGAGAAAGTGGAAGTTCAA
GAAAGAGCGGAACAAGGGCTATAAGCACCACGCCGAGGACGCCCTGATCATTGCCAACGCCG
ATTTCATCTTCAAAGAGTGGAAGAAACTGGACAAGGCCAAAAAAGTGATGGAAAACCAGATG
TTCGAGGAAAAGCAGGCCGAGAGCATGCCCGAGATCGAAACCGAGCAAGAGTACAAAGAGAT
TTTCATCACGCCCCACCAGATCAAGCACATTAAGGACTTCAAGGACTACAAGTACAGCCACC
GCGTGGACAAGAAGCCTAATAGAGAGCTGATTAACGACACCCTGTACAGCACCCGGAAGGAC
GACAAGGGCAATACCCTGATCGTCAACAACCTGAACGGCCTGTACGACAAGGACAACGACAA
GCTCAAGAAGCTGATCAACAAGAGCCCCGAGAAACTGCTGATGTACCACCACGATCCTCAGA
CCTACCAGAAACTGAAGCTCATCATGGAACAGTACGGCGACGAGAAGAATCCCCTGTACAAG
TACTACGAGGAAACCGGGAACTACCTGACCAAGTACTCCAAAAAGGACAATGGGCCCGTGAT
CAAGAAGATTAAGTATTACGGCAACAAGCTGAATGCCCACCTGGACATCACCGACGACTACC
CCAACTCCAGAAACAAGGTGGTCAAGCTGTCCCTGAAGCCTTACAGATTCGACGTGTACCTG
GACAACGGCGTGTACAAGTTCGTGACCGTGAAGAACCTGGATGTGATCAAAAAAGAAAACTA
CTACGAAGTGAACAGCAAGTGCTATGAGGAAGCCAAGAAACTCAAGAAAATCAGCAACCAGG
CCGAGTTTATCGCCTCCTTCTACAACAACGATCTGATCAAGATCAACGGGGAGCTGTATAGA
GTGATTGGGGTCAACAATGACCTGCTGAACCGGATCGAAGTCAACATGATCGACATCACCTA
CCGCGAGTACCTCGAGAACATGAACGACAAGAGGCCTCCACGGATCATTAAGACAATCGCCA
GCAAGACGCAGAGCATTAAGAAGTACAGCACTGACATTCTGGGCAACCTGTACGAAGTCAAG
AGCAAAAAGCACCCGCAGATTATCAAGAAAGGCgagGGCGCCGACAAGAGAACAgccgatgg
ttccgagttcgaaagccccaagaagaagaggaaagtctaG

Translated protein (amino acid) sequence of the MSP828 base editor

(SEQ ID NO: 398)
MSEVEFSHEYWMRHALTLAKRARDEREVPVGAVLVLNNRVIGEGWNRAIGLHDPTAHAEIMA
LRQGGLVMQNYRLYDATLYGTFEPCVMCAGAMIHSRIGRVVFGVRNAKTGAAGSLMDVLHYP
GMNHRVEITEGILADECAALLCDFYRMPRSVFNAQKKAQSSTNSGGSSGGSSGSETPGTSES
ATPESSGGSSGGSKRNYILGLAIGITSVGYGIIDYETRDVIDAGVRLFKEANVENNEGRRSK
RGARRLKRRRRHRIQRVKKLLFDYNLLTDHSELSGINPYEARVKGLSQKLSEEEFSAALLHL
AKRRGVHNVNEVEEDTGNELSTKEQISRNSKALEEKYVAELQLERLKKDGEVRGSINRFKTS
DYVKEAKQLLKVQKAYHOLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPTLKQ
IAKEILVNEEDIKGYRVTSTGKPEFTNLKVYHDIKDITARKEIIENAELLDQIAKILTIYOS
SEDIQEELTNLNSELTQEEIEQISNLKGYTGTHNLSLKAINLILDELWHTNDNQIAIFNRLK
LVPKKVDLSQQKEIPTTLVDDFILSPVVKRSFIQSIKVINAIIKKYGLPNDIIIELAREKNS
KDAQKMINEMQKRNRQTNERIEEIIRTTGKENAKYLIEKIKLHDMQEGKCLYSLEAIPLEDL
LNNPFNYEVDHIIPRSVSFDNSFNNKVLVKQEENSKKGNRTPFQYLSSSDSKISYETFKKHI
LNLAKGKGRISKTKKEYLLEERDINRFSVOKDFINRNLVDTRYATRGLMNLLRSYFRVNNLD
VKVKSINGGFTSFLRRKWKFKKERNKGYKHHAEDALIIANADFIFKEWKKLDKAKKVMENQM
FEEKQAESMPEIETEQEYKEIFITPHQIKHIKDFKDYKYSHRVDKKPNRELINDTLYSTRKD
DKGNTLIVNNLNGLYDKDNDKLKKLINKSPEKLLMYHHDPQTYQKLKLIMEQYGDEKNPLYK
YYEETGNYLTKYSKKDNGPVIKKIKYYGNKLNAHLDITDDYPNSRNKVVKLSLKPYRFDVYL
DNGVYKFVTVKNLDVIKKENYYEVNSKCYEEAKKLKKISNQAEFIASFYNNDLIKINGELYR
VIGVNNDLLNRIEVNMIDITYREYLENMNDKRPPRIIKTIASKTQSIKKYSTDILGNLYEVK
SKKHPQ [symbol] V

In the above MSP828 protein (amino acid) sequence, the TadA 7.10 domain is indicated by underlining; the linker sequence is indicated by bold font; the SaCas9 sequence is indicated by double underlining; and the nuclear localization signal (bpNLS) is indicated by dotted line underlining.

### Example 5: Heavily Modified SaCas9 gRNA Sequences

This example provides representative saCas9 guide RNA sequences designed to include numerous sequence modifications (heavily modified SaCas9 gRNA sequences or “heavy mods”).

Chemical modifications to RNAs, e.g., gRNAs, sgRNAs, provide protection to the RNA molecule and allow for efficient genomic and base editing. Chemically protected gRNAs can enhance stability and editing efficiency in vivo and ex vivo. Non-limiting examples of suitable chemical modifications include 2′-O-methyl (2′-OMe), phosphorothioate (PS), 2′-O-methyl thioPACE (MSP), 2′-O-methyl-PACE (MP), 2′-fluoro RNA (2′-F-RNA), and constrained ethyl (S-cEt), which may be employed in the synthesis of sgRNAs. In a particular embodiment, the chemical modifications are 2′-O-methyl (2′-OMe) modifications. The modified guide RNAs may improve saCas9 efficacy and also specificity. The effect of an individual modification varies based on the position and combination of chemical modifications used as well as the inter- and intramolecular interactions with other modified nucleotides. By way of example, S-cEt has been used to improve oligonucleotide intramolecular folding.

Heavy mod designs currently do not exist for Cas orthologs such as saCas9, Cas12, etc., e.g., those which are not SpCas9. Such SpCas9 heavy mods can increase base editing ˜2 fold in vivo. For such modifications, mN=2′-OMe; Ns=phosphorothioate (PS), where “N” represents the term nucleotide, as would be understood by one having skill in the art. In some cases, a nucleotide (N) may contain two modifications, for example, both a 2′-OMe and a PS modification. Representative heavy mods gRNAs (heavy mod series 1 gRNAs for R83C (saCas9), as shown in (1) below) were utilized in experiments in which adult transgenic mice heterozygous for huG6PC-R83C were dosed at a sub-saturating dose of 1 mpk of 1:1 ratio of gRNA:editor mRNA (FIG. 11). The heavy mods as described here provide a new universal approach to increase in vivo base editing that is independent of sequence.

Provided below are examples of heavy mod gRNA sequences and end mod gRNA sequences. In particular, shown below are sequences in connection with the following: (1) heavy mods series 1; (2) heavy mods series 2; (3) protospacer extension; (4) direct repeat/anti-repeat lengths/sequence context, and the “base” sequences (end mods); and (5) gRNA sequences (end mod sgRNA sequences) designed for correcting the huG6PC-R83C mutation as described and exemplified supra. As will be appreciated by those skilled in the art, Cas9 evolved to use a trans-activating RNA (tracrRNA) that base pairs via an anti-repeat sequence to the repeat sequence of individual crRNAs. A more commonly used single gRNA constitutes a fusion of the tracrRNA and crRNA through a short RNA loop between the repeat-anti-repeat sequences in the Upper Stein (G. Mullally et al., 2020, Nucleic Acids Research, 48(12):6811-6823; M. Jinek et al., 2012, Science, 337:816-821). The direct repeat regions contain sequences for processing pre-crRNA into mature crRNA and tracrRNA binding. The “direct repeat region” combined with the tracrRNA forms the scaffold portion of a gRNA and the “spacer region” forms the target sequence.

In each of the following sequences, mN=2′OMe; Ns=phosphorothioate, where “N” designates any nucleotide or nucleobase. In the below sequences, “s” indicates that the preceding nucleotide possesses a 3′ phosphorothioate, and the “m” indicates the following nucleotide is a 2′ OMe. A nucleotide with a phosphorothioate and 2′ OMe is denoted as “mNs;” when there are two modifications next to each other, the notation is “mNsmNs.”

1. Heavy mods series 1
gRNA578: 50% modified with 2′OMe
(SEQ ID NO: 404)
mCsmAsmGsmUAUmGmGmACAmCUGUCCAAAmGUUUUmAmGmUACUCmUGmUmAmAmUGmAA
AmAmUmUmACmAGAAUCUACmUmAAAACAAGGCAAmAAUGmCCmGUGUmUmUmAmUmCmUmC
mGmUmCmAmAmCmUmUmGmUmUmGmGmCmGmAmGmAmUsmUsmUsmU
gRNA579: 50% modified, 5′ extended with 3 Us (uridines)
(SEQ ID NO: 405)
mUsmUsmUsCAGmUAUmGmGmACAmCUGUCCAAAmGUUUUmAmGmUACUCmUGmUmAmAmUG
mAAAmAmUmUmACmAGAAUCUACmUmAAAACAAGGCAAmAAUGmCCmGUGUmUmUmAmUmCm
UmCmGmUmCmAmAmCmUmUmGmUmUmGmGmCmGmAmGmAmUsmUsmUsmU
gRNA580: 66% modified
(SEQ ID NO: 404)
mCsmAsGsmUAUmGmGmAmCAmCmUGUCCAAmAmGUmUUmUmAmGmUACUmCmUmGmUmAmA
mUGmAmAmAmAmUmUmACmAmGAAmUCUACmUmAmAAACAAGmGCAAmAAUGmCmCmGUGmU
mUmUmAmUmCmUmCmGmUmCmAmAmCmUmUmGmUmUmGmGmCmGmAmGmAmUsmUsmUsmU
gRNA581: 69% modified
(SEQ ID NO: 404)
mCsmAsGsmUmAUmGmGmAmCAmCUGUCCAAmAmGUUUmUAmGmUACUmCmUmGmUmAmAmU
mGmAmAmAmAmUmUmAmCmAmGmAAmUCUACUmAmAAACAAmGmGmCmAmAmAAUmGmCmCG
UGmUmUmUmAmUmCmUmCmGmUmCmAmAmCmUmUmGmUmUmGmGmCmGmAmGmAmUsmUsmU
smU
gRNA582: 84% modified
(SEQ ID NO: 404)
mCsmAsGsmUmAUmGmGmAmCAmCmUGUCmCmAAmAmGmUmUmUmUmAmGmUAmCmUmCmUm
GmUmAmAmUmGmAmAmAmAmUmUmAmCmAmGmAmAmUCUACmUmAmAmAAmCAmAmGmGmCm
AmAmAAUmGmCmCmGmUGmUmUmUmAmUmCmUmCmGmUmCmAmAmCmUmUmGmUmUmGmGmC
mGmAmGmAmUsmUsmUsmU
2. Heavy mods series 2
gRNA964: 108 nt (longer DR/anti-DR); 24% modified; hairpin (DR/anti-DR) mods only
(SEQ ID NO: 404)
mCsmAsmGsUAUGGACACUGUCCAAAGUUUUAGUACmUmCmUmGmUmAmAmUmGmAmAmAmA
mUmUmAmCmAmGmAAUCUACUAAAACAAGGCAAAAUGCCGUGUUUAUCUCGUCAACUUGUUG
GCGAGAUsmUsmUsmU
gRNA965: 108 nt (longer DR/anti-DR); 48% modified; hairpin (DR/anti-DR) mods + 3′
mods
(SEQ ID NO: 404)
mCsmAsmGsUAUGGACACUGUCCAAAGUUUUAGUACmUmCmUmGmUmAmAmUmGmAmAmAmA
mUmUmAmCmAmGmAAUCUACUAAAACAAGGCAAAAUGCCGUGUmUmUmAmUmCmUmCmGmUm
CmAmAmCmUmUmGmUmUmGmGmCmGmAmGmAmUsmUsmUsmU
gRNA966: 108 nt (longer DR/anti-DR); 53% modified; hairpin (DR/anti-DR) mods + 3′
mods + protospacer mods
(SEQ ID NO: 404)
mCsmAsmGsmUAUmGmGmACAmCUGUCCAAAmGUUUUAGUACmUmCmUmGmUmAmAmUmGmA
mAmAmAmUmUmAmCmAmGmAAUCUACUAAAACAAGGCAAAAUGCCmGUGUmUmUAmUmCmUm
CmGmUmCmAmAmCmUmUmGmUmUmGmGmCmGmAmGmAUsmUsmUsmU
gRNA967: 100 nt (shorter DR/anti-DR); 43% modified; hairpin (DR/anti-DR) mods + 3′
mods
(SEQ ID NO: 406)
mCsmAsmGsUAUGGACACUGUCCAAAGUUUUAGUACmUmCmUmGGmAmAmAmCmAmGmAAUC
UACUAAAACAAGGCAAAAUGCCGUGUmUmUmAmUmCmUmCmGmUmCmAmAmCmUmUmGmUmU
mGmGmCmGmAmGmAmUsmUsmUsmU
gRNA968: 100 nt (shorter DR/anti-DR); 49% modified; hairpin (DR/anti-DR) mods + 3′
mods + protospacer mods
(SEQ ID NO: 406)
mCsmAsmGsmUAUmGmGmACAmCUGUCCAAAmGUUUUAGUACmUmCmUmGmGmAmAmAmCmA
mGmAAUCUACUAAAACAAGGCAAAAUGCCmGUGUmUmUAmUmCmUmCmGmUmCmAmAmCmUm
UmGmUmUmGmGmCmGmAmGmAUsmUsmUsmU
3. Variable length protospacers and sgRNAs
sgRNA_094: 21 nt protospacer
(SEQ ID NO: 407)
mCsmCsmAsGUAUGGACACUGUCCAAAGUUUUAGUACUCUGUAAUGAAAAUUACAGAAUCUA
CUAAAACAAGGCAAAAUGCCGUGUUUAUCUCGUCAACUUGUUGGCGAGAmUsmUsmUsU
sgRNA_095: 22 nt protospacer
(SEQ ID NO: 408)
mAsmCsmCsAGUAUGGACACUGUCCAAAGUUUUAGUACUCUGUAAUGAAAAUUACAGAAUCU
ACUAAAACAAGGCAAAAUGCCGUGUUUAUCUCGUCAACUUGUUGGCGAGAmUsmUsmUsU
sgRNA_096: 23 nt protospacer
(SEQ ID NO: 409)
mCsmAsmCsCAGUAUGGACACUGUCCAAAGUUUUAGUACUCUGUAAUGAAAAUUACAGAAUC
UACUAAAACAAGGCAAAAUGCCGUGUUUAUCUCGUCAACUUGUUGGCGAGAmUsmUsmUsU
sgRNA_097: 34 nt protospacer
(SEQ ID NO: 410)
mCsmCsmASCCAGUAUGGACACUGUCCAAAGUUUUAGUACUCUGUAAUGAAAAUUACAGAAU
CUACUAAAACAAGGCAAAAUGCCGUGUUUAUCUCGUCAACUUGUUGGCGAGAmUsmUsmUsU
4. Modified direct repeat / anti-direct repeat sequences and gRNAs
gRNA868: R83C sgRNA_029 16 bp DR; 104 nt
(SEQ ID NO: 411)
mCsmAsmGsUAUGGACACUGUCCAAAGUUUUAGUACUCUGUAGAAAUACAGAAUCUACUAAA
ACAAGGCAAAAUGCCGUGUUUAUCUCGUCAACUUGUUGGCGAGAUsmUsmUsmU
gRNA869: R83C sgRNA_029 16 bp CG DR; 104 nt
(SEQ ID NO: 412)
mCsmAsmGsUAUGGACACUGUCCAAAGUUUUAGUACUCUGCGGAAACGCAGAAUCUACUAAA
ACAAGGCAAAAUGCCGUGUUUAUCUCGUCAACUUGUUGGCGAGAUsmUsmUsmU
gRNA870: R83C sgRNA_029 14 bp DR ran et al; 100 nt
(SEQ ID NO: 406)
mCsmAsmGsUAUGGACACUGUCCAAAGUUUUAGUACUCUGGAAACAGAAUCUACUAAAACAA
GGCAAAAUGCCGUGUUUAUCUCGUCAACUUGUUGGCGAGAUsmUsmUsmU
gRNA871: R83C sgRNA_029 12 bp DR; 96 nt
(SEQ ID NO: 413)
mCsmAsmGsUAUGGACACUGUCCAAAGUUUUAGUACUCGAAAGAAUCUACUAAAACAAGGCA
AAAUGCCGUGUUUAUCUCGUCAACUUGUUGGCGAGAUsmUsmUsmU
gRNA872: R83C sgRNA_029 12 bp CC DR; 96 nt
(SEQ ID NO: 414)
mCsmAsmGsUAUGGACACUGUCCAAAGUUUUAGUACCCGAAAGCAUCUACUAAAACAAGGCA
AAAUGCCGUGUUUAUCUCGUCAACUUGUUGGCGAGAUsmUsmUsmU
5. End mod sgRNA sequences designed for use in correcting the huG6PC R83C mutation
sgRNA_029
(SEQ ID NO: 415)
mCsmAsmGsUAUGGACACUGUCCAAAGUUUUAGUACUCUGUAAUGAAAAUUACAGAAUCUAC
UAAAACAAGGCAAAAUGCCGUGUUUAUCUCGUCAACUUGUUGGCGAGAUsmUsmUsmU
sgRNA_093
(SEQ ID NO: 415)
mCsmAsmGsUAUGGACACUGUCCAAAGUUUUAGUACUCUGUAAUGAAAAUUACAGAAUCUAC
UAAAACAAGGCAAAAUGCCGUGUUUAUCUCGUCAACUUGUUGGCGAGAmUsmUsmUsU

### Example 6: Materials and Methods

Materials and methods utilized in the examples and experiments therein as described supra are set forth below.

### Animal Care

All animal studies were conducted under Taconic's Excluded Flora health standard. To sustain survival of huG6PC-R83C mice, a glucose therapy consisting of daily administered subcutaneous injections of 100-150 ul of 15% glucose per mouse. Glucose injections were not administered to mice post LNP treatment with base-editor mRNA and gRNA.

### In Vivo LNP-Dosing Work-Flow

To correct the p.R83C mutation in the huG6PC-R83C homozygous mice, LNP co-formulations of base-editor mRNA and gRNA were administered at a 1.5 mpk (milligram per kilogram) dose via the temporal vein of mice at age P1, shortly post birth. Glucose therapy was not administered to LNP-treated mice. LNP-treated mice continued to be cared for alongside littermate controls by the respective birth mother until weaning (21 days), at which point they were phenotyped. For all studies, age matched wild-type and heterozygous huG6PC-R83C littermates were used as controls. At day 21, genomic DNA harvested from livers, growth characteristics, and serum and liver markers were analyzed.

### Lipid Nanoparticle (LNP) Formulations

The base editor (mRNA encoding the base editor) and guide RNA were co-encapsulated at a 1:1 weight ratio in a lipid nanoparticle. The LNPs were generated by rapidly mixing an aqueous solution of the RNA at a pH of 3.0 with an ethanol solution containing four lipid components: an ionizable lipid, DSPC, cholesterol, and a lipid-anchored PEG. The two solutions were mixed using the benchtop microfluidics device from Precision Nanosystems. Post mixing, the formulations were dialyzed overnight at 4° C. against 1× TBS (Sigma-Aldrich, catalog #94158). They were subsequently concentrated down using 100K MWCO Amicon Ultra centrifugation tubes (Millipore Sigma, catalog #UFC910096), and filtered with 0.2 micron filters (Pall corporation, Catalog #4602). Total RNA concentration of the was determined using Quant-iT Ribogreen (ThermoFisher Scientific, catalog #R11491); particle size was determined by using the Malvern Panalytical Zetasizer.

### Next Generation Sequencing (NGS)

Next generation sequencing (NGS) was used to determine the frequency of base-edited alleles in genomic DNA from whole liver extracts of LNP-treated animals. Following LNP-treatment, mice were euthanized, and the entire liver was removed and snap frozen in liquid nitrogen. Frozen mouse livers were ground to a powder form using Geno/Grinder 2010 (Ops Diagnostics, Lebanon, NJ, USA), and genomic DNA was isolated from the liver powder using Quick Extract lysis buffer according to manufacture's specifications. Genomic DNA was directly used in subsequent PCR amplification steps to produce a ˜170-nucleotide fragment harboring huG6PC exon 2 using the primer pair: Forward primer, GGGCATTAAACTCCTTTGGG (SEQ ID NO: 393) and reverse primer, AGTCTCACAGGTTACAGGGA (SEQ ID NO: 394). NGS adapters were added, and the resulting amplicons were sequenced using an Illumina MiSeq instrument according to the manufacturer's instructions.

### Serum Metabolites

To measure serum metabolites, blood was collected from R83C humanized transgenic mice. Serum was then separated and extracted from whole blood, which was subsequently used for metabolite assays. For a relevant and comprehensive post-study assessment, serum glucose, serum cholesterol, and serum triglycerides were all analyzed. Serum glucose and serum cholesterol were measured using ThermoFisher Scientific (Waltham, MA, USA) Infinity Glucose Liquid Stable Reagent (Cat #: TR15421) and Infinity Cholesterol Liquid Stable Reagent (Cat #: TR13421), respectively. Serum triglycerides were measured using the Serum Triglyceride Quantification Kit (Cat #: MAK266) from Sigma-Aldrich (St. Louis, MO, USA). Uric acid was measured using the Uric Acid Liquid Stable Reagent per manufacturer specifications (Thermo Fisher Scientific (Waltham, MA, USA). Serum lactate was analyzed using the EnzyFluo L-Lactate Assay Kit from BioAssay Systems (Hayward, CA, USA).

Fasting blood glucose analysis of mice involved blood sampling via the tail vein pre- and post-24 hours after food deprivation. Blood glucose levels were measured using the HemoCue Glucose 201 System (HemoCue America, CA, USA).

### Glucose-6-Phosphatase-Alpha Activity Assay

Liver microsome isolation and microsomal phosphohydrolase assays were performed as described by Lei, K.-J., et al., 1996, Nature Genetics, 13(2):203-9. Assay methodology in Arnaotova et al. (2021, Mol. Therapy., Vol. 29, No 4) is described as follows: “Glucose-6-phosphatase dependent substrate transport in the glycogen storage disease type-1a mouse. Nat. Genet. 13, 203-209). In phosphoydrolase assays, reaction mixtures (50 uL) containing 50 mM sodium cacodylate buffer (pH 6.5), 2 mM EDTA, 10 mM Glucose-6-phosphate (G6P), and appropriate amounts of microsomal preparations were incubated at 30° C. for 10 minutes. Disrupted microsomal membranes were prepared by incubating intact membranes in 0.2% deoxycholate for 20 minutes at 4° C. Non-specific phosphatase activity was estimated by pre-incubating disrupted microsomal preparations at pH 5 for 10 minutes at 37° C. to inactivate the acid-labile G6Pase-alpha. One unit of G6Pase-alpha activity represents one nmol G6P hydrolysis per minute per mg microsomal protein. The lower level of quantitation for the microsomal G6Pase-alpha assay is 2 units.”

Enzyme histochemical analysis of G6Pase-alpha was performed as described in Lee, Y. M., Jun, H. S. Pan, C.-J. Lin, S. R., Wilson, L. H., Mansfield, B. C., and Chou, J. Y. (2012). Prevention of hepatocyellular adenoma and correction of metabolic abnormalities in murine glycogen storage disease type Ia by gene therapy. Hepatology 56, 1719-1729. As described in Arnaotova et al., (2021, Mol. Therapy., Vol. 29, No 4), 10 um-thick liver tissue sections were incubated for 10 min at room temperature in a solution containing 40 mM Tris-maleate (pH 6.5), 10 mM G6P, 300 mM sucrose, and 3.6 mM lead nitrate. After rinsing, liver sections were incubated for 2 min at room temperature in 0.09% ammonium sulfide solution, and the trapped lead phosphate was visualized following conversion to the brown-colored lead sulfide.

### Immunohistochemistry

Immunohistochemical procedures were performed as described in Arnaotova et al., 2021, Mol. Therapy., Vol. 29, No 4. In brief, H&E staining was performed on liver sections preserved in 10% neutral buffered formalin, and Oil Red O staining was performed on cryopreserved optimal cutting temperature compound (OCT) embedded liver sections following standard procedures. The stained sections were visualized using the Imager A2m microscope with Axiocam 506 camera and Zen 2.6 software (Carl Zeiss, White Plains, NY, USA).

### Other Embodiments

From the foregoing description, it will be apparent that variations and modifications may be made to the embodiments as described herein to be adopted to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All publications, patents, and patent applications mentioned in this specification are herein incorporated by reference to the same extent as if each individual publication, patent, or patent application was specifically and individually indicated to be incorporated by reference. Absent any indication otherwise, publications, patents, and patent applications mentioned in this specification are incorporated herein by reference in their entireties.

1. An adenosine deaminase variant comprising a glycine (G) at amino acid position 82, a threonine (T) or an aspartic acid (D) at amino acid position 147, a serine (S) at amino acid position 154, and one or more of a histidine (H) at amino acid position 36, a tyrosine at amino acid position 76, a tyrosine at amino acid position 149, a lysine (K) at amino acid position 157, and an asparagine (N) at amino acid position 167 of the following amino acid sequence, wherein the adenosine deaminase has at least about 85% identity to said amino acid sequence: MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 1), or corresponding alterations in another adenosine deaminase.

2. An adenosine deaminase variant comprising any of the following combinations of alterations
a) I76Y+V82G+Y147T+Q154S;
b) L36H+V82G+Y147T+Q154S+N157K;
c) V82G+Y147D+F149Y+Q154S+D167N;
d) L36H+V82G+Y147D+F149Y+Q154S+N157K+D167N;
e) L36H+I76Y+V82G+Y147T+Q154S+N157K;
f) I76Y+V82G+Y147D+F149Y+Q154S+D167N;
g) Y147D+F149Y+D167N;
h) L36H; I76Y; V82G; Q154S; and N157K;
i) I76Y; V82G; Q154S; or
j) L36H+I76Y+V82G+Y147D+F149Y+Q154S+N157K+D167N with reference to SEQ ID NO: 1: MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 1), or corresponding combinations of alterations in another adenosine deaminase.

3. The adenosine deaminase variant of claim 1, comprising the following combination of alterations I76Y+V82G+Y147D+F149Y+Q154S+D167N of SEQ ID NO: 1, or corresponding alterations in another adenosine deaminase.

4. The adenosine deaminase variant of claim 1, wherein the adenosine deaminase has at least about 90% identity to SEQ ID NO: 1.

5-6. (canceled)

7. A fusion protein or complex comprising a polynucleotide programmable DNA binding domain and at least one adenosine deaminase variant domain, wherein the adenosine deaminase variant domain comprises a glycine (G) at amino acid position 82, a threonine (T) or an aspartic acid (D) at amino acid position 147, a serine (S) at amino acid position 154, and one or more of a histidine (H) at amino acid position 36, a tyrosine at amino acid position 76, a tyrosine at amino acid position 149, a lysine (K) at amino acid position 157, and an asparagine (N) at amino acid position 167 of the following amino acid sequence, wherein the adenosine deaminase has at least about 85% identity to said amino acid sequence MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 1), or corresponding alterations in another adenosine deaminase.

8-10. (canceled)

11. The fusion protein or complex of claim 1 comprising a polynucleotide programmable DNA binding domain and at least one adenosine deaminase variant domain, wherein the adenosine deaminase variant domain comprises any of the following combinations of alterations
a) I76Y+V82G+Y147T+Q154S;
b) L36H+V82G+Y147T+Q154S+N157K;
c) V82G+Y147D+F149Y+Q154S+D167N;
d) L36H+V82G+Y147D+F149Y+Q154S+N157K+D167N;
e) L36H+I76Y+V82G+Y147T+Q154S+N157K;
f) I76Y+V82G+Y147D+F149Y+Q154S+D167N;
g) Y147D+F149Y+D167N;
h) L36H; I76Y; V82G; Q154S; and N157K;
i) I76Y; V82G; Q154S; or
j) L36H+I76Y+V82G+Y147D+F149Y+Q154S+N157K+D167N
with reference to SEQ ID NO: 1: MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIIHSRIGRVVFGVRNAKTGAAGSLMDVLHYPGMNH RVEITEGILADECAALLCYFFRMPRQVFNAQKKAQSSTD (SEQ ID NO: 1), or corresponding combinations of alterations in another adenosine deaminase.

12-14. (canceled)

15. The fusion protein or complex of claim 7, wherein the fusion protein comprises a TadA*7.10 adenosine deaminase domain and an adenosine deaminase variant domain; or wherein the polynucleotide programmable DNA binding domain is a Cas9 domain.

16-18. (canceled)

19. The fusion protein or complex of claim 7, wherein the polynucleotide programmable DNA binding domain comprises a modified SaCas9 having an altered protospacer-adjacent motif (PAM) specificity;
wherein the SaCas9 has protospacer-adjacent motif (PAM) specificity for the nucleic acid sequence 5′-NNGRRT-3′ or 5′-GAGAAT-3′, and wherein the SaCas9 is a nuclease active SaCas9, a nuclease inactive SaCas9 (SaCas9d), or a SaCas9 nickase (SaCas9n);
wherein the linker comprises the amino acid sequence: SGGSSGGSSGSETPGTSESATPES (SEQ ID NO: 359); and
further comprises one or more nuclear localization signals.

20-30. (canceled)

31. A base editor system comprising the fusion protein or complex of claim 7, and one or more guide polynucleotides.

32-41. (canceled)

42. The base editor system of claim 31, wherein the guide polynucleotide comprises or consists essentially of one of the following sequences:
(SEQ ID NO: 409)
mCsmAsmCsCAGUAUGGACACUGUCCAAAGUUUUAGUACUCUGUAAUGAA
AAUUACAGAAUCUACUAAAACAAGGCAAAAUGCCGUGUUUAUCUCGUCAA
CUUGUUGGCGAGAmUsmUsmUsU
or
(SEQ ID NO: 410)
mCsmCsmASCCAGUAUGGACACUGUCCAAAGUUUUAGUACUCUGUAAUGA
AAAUUACAGAAUCUACUAAAACAAGGCAAAAUGCCGUGUUUAUCUCGUCA
ACUUGUUGGCGAGAmUsmUsmUSU,
wherein “m” denotes a 2′-O-methyl and “s” denotes a phosphorothioate; or
wherein the guide polynucleotide comprises a nucleic acid sequence, in 5′ to 3′ orientation, selected from CCACCAGUAUGGACACUGUC (SEQ ID NO: 371); CACCAGUAUGGACACUGUCC (SEQ ID NO: 372); ACCAGUAUGGACACUGUCCA (SEQ ID NO: 373); CCAGUAUGGACACUGUCCAA (SEQ ID NO: 374); CAGUAUGGACACUGUCCAAA (SEQ ID NO: 370); AGUAUGGACACUGUCCAAAG (SEQ ID NO: 375); GUAUGGACACUGUCCAAAGA (SEQ ID NO: 376); or UAUGGACACUGUCCAAAGAG (SEQ ID NO: 377).

43-44. (canceled)

45. A polynucleotide encoding the adenosine deaminase variant of claim 1.

46-48. (canceled)

49. A cell comprising the polynucleotide of claim 45.

50. A cell comprising the adenosine deaminase variant of claim 1.

51-56. (canceled)

57. A method of treating a genetic disease in a subject in need thereof, the method comprising administering to a cell of the subject the base editor system of claim 31 or a polynucleotide encoding the base editor system.

58. A method of treating a genetic disease in a subject in need thereof, the method comprising administering to the subject the cell of claim 49.

59. The method of claim 57, wherein after treatment the cell expresses a G6PC polypeptide capable of catalyzing the hydrolysis of D-glucose 6-phosphate to D-glucose and orthophosphate.

60. (canceled)

61. A method for correcting a single nucleotide polymorphism (SNP) in a polynucleotide, the method comprising:
contacting a target nucleotide sequence, at least a portion of which is located in the polynucleotide or its reverse complement, with the base editor system of claim 31 and editing the SNP by deaminating the SNP or its complement nucleobase upon targeting of the base editor to the target nucleotide sequence, wherein deaminating the SNP or its complement nucleobase corrects the SNP.

62. The method of claim 61, wherein the SNP is associated with Glycogen Storage Disease Type 1a (GSD1a).

63. (canceled)

64. A method of editing a glucose-6-phosphatase (G6PC) polynucleotide comprising a single nucleotide polymorphism (SNP) associated with Glycogen Storage Disease Type 1a (GSD1a), the method comprising contacting the G6PC polynucleotide with a fusion protein or complex of claim 7 in a complex with one or more guide polynucleotides, wherein one or more of the guide polynucleotides targets the base editor to effect an A·T to G·C alteration of the SNP associated with GSD1a.

65. (canceled)

66. The method of claim 57, wherein the SNP changes a glutamine (Q) to a non-glutamine (X) amino acid or changes an arginine (R) to a non-arginine (X) in a G6PC polypeptide; wherein the SNP results in expression of an G6PC polypeptide having a non-glutamine (X) amino acid at position 347 or a non-arginine (X) amino acid at position 83; wherein the base editor correction replaces the non-glutamine amino acid (X) at position 347 with a glutamine or the non-arginine amino acid (X) at position 83 with an arginine; wherein the SNP results in expression of a G6PC polypeptide that prematurely terminates at amino acid position 347 or at a cysteine at position 83; wherein the SNP encodes one or more of Q347X and/or R83C.

67-72. (canceled)

73. The method of claim 64, wherein the guide polynucleotide comprises a nucleic acid sequence, from 5′-3′, selected from the group consisting of CAGUAUGGACACUGUCCAAA (SEQ ID NO: 370); CCACCAGUAUGGACACUGUC (SEQ ID NO: 371); CACCAGUAUGGACACUGUCC (SEQ ID NO: 372); ACCAGUAUGGACACUGUCCA (SEQ ID NO: 373); CCAGUAUGGACACUGUCCAA (SEQ ID NO: 374); AGUAUGGACACUGUCCAAAG (SEQ ID NO: 375); GUAUGGACACUGUCCAAAGA (SEQ ID NO: 376); and UAUGGACACUGUCCAAAGAG (SEQ ID NO: 377).

74. (canceled)

75. A vector comprising the polynucleotide of claim 45.

76-77. (canceled)

78. A composition comprising the fusion protein or complex of claim 7.

79-81. (canceled)

82. A composition comprising the polynucleotide of claim 45.

83. (canceled)

84. A composition comprising the cell of claim 49.

85-87. (canceled)

88. A kit comprising the fusion protein or complex of claim 7.

89-92. (canceled)

93. A modified guide RNA (gRNA) comprising modified nucleotides, wherein the guide comprises from 5′ to 3′ a polynucleotide sequence selected from the group consisting of:
(SEQ ID NO: 404)
CAGUAUGGACACUGUCCAAAGUUUUAGUACUCUGUAAUGAAAAUUACAGA
AUCUACUAAAACAAGGCAAAAUGCCGUGUUUAUCUCGUCAACUUGUUGGC
GAGAUUUU;
(SEQ ID NO: 405)
UUUCAGUAUGGACACUGUCCAAAGUUUUAGUACUCUGUAAUGAAAAUUAC
AGAAUCUACUAAAACAAGGCAAAAUGCCGUGUUUAUCUCGUCAACUUGUU
GGCGAGAUUUU;
(SEQ ID NO: 406)
CAGUAUGGACACUGUCCAAAGUUUUAGUACUCUGGAAACAGAAUCUACUA
AAACAAGGCAAAAUGCCGUGUUUAUCUCGUCAACUUGUUGGCGAGAUUU
U;
(SEQ ID NO: 407)
CCAGUAUGGACACUGUCCAAAGUUUUAGUACUCUGUAAUGAAAAUUACAG
AAUCUACUAAAACAAGGCAAAAUGCCGUGUUUAUCUCGUCAACUUGUUGG
CGAGAUUUU;
(SEQ ID NO: 408)
ACCAGUAUGGACACUGUCCAAAGUUUUAGUACUCUGUAAUGAAAAUUACA
GAAUCUACUAAAACAAGGCAAAAUGCCGUGUUUAUCUCGUCAACUUGUUG
GCGAGAUUUU;
(SEQ ID NO: 409)
CACCAGUAUGGACACUGUCCAAAGUUUUAGUACUCUGUAAUGAAAAUUAC
AGAAUCUACUAAAACAAGGCAAAAUGCCGUGUUUAUCUCGUCAACUUGUU
GGCGAGAUUUU;
(SEQ ID NO: 410)
CCACCAGUAUGGACACUGUCCAAAGUUUUAGUACUCUGUAAUGAAAAUUA
CAGAAUCUACUAAAACAAGGCAAAAUGCCGUGUUUAUCUCGUCAACUUGU
UGGCGAGAUUUU;
(SEQ ID NO: 411)
CAGUAUGGACACUGUCCAAAGUUUUAGUACUCUGUAGAAAUACAGAAUCU
ACUAAAACAAGGCAAAAUGCCGUGUUUAUCUCGUCAACUUGUUGGCGAGA
UUUU;
(SEQ ID NO: 412)
CAGUAUGGACACUGUCCAAAGUUUUAGUACUCUGCGGAAACGCAGAAUCU
ACUAAAACAAGGCAAAAUGCCGUGUUUAUCUCGUCAACUUGUUGGCGAGA
UUUU;
(SEQ ID NO: 413)
CAGUAUGGACACUGUCCAAAGUUUUAGUACUCGAAAGAAUCUACUAAAAC
AAGGCAAAAUGCCGUGUUUAUCUCGUCAACUUGUUGGCGAGAUUUU;
(SEQ ID NO: 414)
CAGUAUGGACACUGUCCAAAGUUUUAGUACCCGAAAGCAUCUACUAAAAC
AAGGCAAAAUGCCGUGUUUAUCUCGUCAACUUGUUGGCGAGAUUUU;
and
(SEQ ID NO: 415)
CAGUAUGGACACUGUCCAAAGUUUUAGUACUCUGUAAUGAAAAUUACAGA
AUCUACUAAAACAAGGCAAAAUGCCGUGUUUAUCUCGUCAACUUGUUGGC
GAGAUUUU.

94-112. (canceled)

113. A modified guide RNA (gRNA) comprising a nucleic acid sequence, from 5′ to 3′, selected from
(SEQ ID NO: 404)
mCsmAsmGsmUAUmGmGmACAmCUGUCCAAAmGUUUUmAmGmUACUCmUG
mUmAmAmUGmAAAmAmUmUmACmAGAAUCUACmUmAAAACAAGGCAAmAAUGm
CCmGUGUmUmUmAmUmCmUmCmGmUmCmAmAmCmUmUmGmUmUmGmGmCmGm
AmGmAmUsmUsmUsmU;
(SEQ ID NO: 405)
mUsmUsmUsCAGmUAUmGmGmACAmCUGUCCAAAmGUUUUmAmGmUACUC
mUGmUmAmAmUGmAAAmAmUmUmACmAGAAUCUACmUmAAAACAAGGCAAmAA
UGmCCmGUGUmUmUmAmUmCmUmCmGmUmCmAmAmCmUmUmGmUmUmGmGmC
mGmAmGmAmUsmUsmUsmU;
(SEQ ID NO: 404)
mCsmAsGsmUAUmGmGmAmCAmCmUGUCCAAmAmGUmUUmUmAmGmUACU
mCmUmGmUmAmAmUGmAmAmAmAmUmUmACmAmGAAmUCUACmUmAmAAACA
AGmGCAAmAAUGmCmCmGUGmUmUmUmAmUmCmUmCmGmUmCmAmAmCmUmU
mGmUmUmGmGmCmGmAmGmAmUsmUsmUsmU;
(SEQ ID NO: 404)
mCsmAsGsmUmAUmGmGmAmCAmCUGUCCAAmAmGUUUmUAmGmUACUmC
mUmGmUmAmAmUmGmAmAmAmAmUmUmAmCmAmGmAAmUCUACUmAmAAACA
AmGmGmCmAmAmAAUmGmCmCGUGmUmUmUmAmUmCmUmCmGmUmCmAmAmC
mUmUmGmUmUmGmGmCmGmAmGmAmUsmUsmUsmU;
or
(SEQ ID NO: 404)
mCsmAsGsmUmAUmGmGmAmCAmCmUGUCmCmAAmAmGmUmUmUmUmAmG
mUAmCmUmCmUmGmUmAmAmUmGmAmAmAmAmUmUmAmCmAmGmAmAmUCUA
CmUmAmAmAAmCAmAmGmGmCmAmAmAAUmGmCmCmGmUGmUmUmUmAmUmC
mUmCmGmUmCmAmAmCmUmUmGmUmUmGmGmCmGmAmGmAmUsmUsmUsmU,
wherein “m” denotes a 2′-O-methyl and “s” denotes a phosphorothioate; or
selected from
(SEQ ID NO: 404)
mCsmAsmGsUAUGGACACUGUCCAAAGUUUUAGUACmUmCmUmGmUmAmAmUmG
mAmAmAmAmUmUmAmCmAmGmAAUCUACUAAAACAAGGCAAAAUGCCGUGUUU
AUCUCGUCAACUUGUUGGCGAGAUsmUsmUsmU;
(SEQ ID NO: 404)
mCsmAsmGsUAUGGACACUGUCCAAAGUUUUAGUACmUmCmUmGmUmAmA
mUmGmAmAmAmAmUmUmAmCmAmGmAAUCUACUAAAACAAGGCAAAAUGCCGU
GUmUmUmAmUmCmUmCmGmUmCmAmAmCmUmUmGmUmUmGmGmCmGmAmGmA
mUsmUsmUsmU;
(SEQ ID NO: 404)
mCsmAsmGsmUAUmGmGmACAmCUGUCCAAAmGUUUUAGUACmUmCmUmG
mUmAmAmUmGmAmAmAmAmUmUmAmCmAmGmAAUCUACUAAAACAAGGCAAAA
UGCCmGUGUmUmUAmUmCmUmCmGmUmCmAmAmCmUmUmGmUmUmGmGmCmG
mAmGmAUsmUsmUsmU;
(SEQ ID NO: 406)
mCsmAsmGsUAUGGACACUGUCCAAAGUUUUAGUACmUmCmUmGGmAmAm
AmCmAmGmAAUCUACUAAAACAAGGCAAAAUGCCGUGUmUmUmAmUmCmUmCm
GmUmCmAmAmCmUmUmGmUmUmGmGmCmGmAmGmAmUsmUsmUsmU;
(SEQ ID NO: 406)
mCsmAsmGsmUAUmGmGmACAmCUGUCCAAAmGUUUUAGUACmUmCmUmG
mGmAmAmAmCmAmGmAAUCUACUAAAACAAGGCAAAAUGCCmGUGUmUmUAmU
mCmUmCmGmUmCmAmAmCmUmUmGmUmUmGmGmCmGmAmGmAUsmUsmUsmU,
wherein “m” denotes a 2′-O-methyl and “s” denotes a phosphorothioate;
selected from
(SEQ ID NO: 407)
mCsmCsmAsGUAUGGACACUGUCCAAAGUUUUAGUACUCUGUAAUGAAAAU
UACAGAAUCUACUAAAACAAGGCAAAAUGCCGUGUUUAUCUCGUCAACUUGUUG
GCGAGAmUsmUsmUsU;
(SEQ ID NO: 408)
mAsmCsmCsAGUAUGGACACUGUCCAAAGUUUUAGUACUCUGUAAUGAAAA
UUACAGAAUCUACUAAAACAAGGCAAAAUGCCGUGUUUAUCUCGUCAACUUGUU
GGCGAGAmUsmUsmUsU;
(SEQ ID NO: 409)
mCsmAsmCsCAGUAUGGACACUGUCCAAAGUUUUAGUACUCUGUAAUGAAA
AUUACAGAAUCUACUAAAACAAGGCAAAAUGCCGUGUUUAUCUCGUCAACUUGU
UGGCGAGAmUsmUsmUsU;
or
(SEQ ID NO: 410)
mCsmCsmAsCCAGUAUGGACACUGUCCAAAGUUUUAGUACUCUGUAAUGAA
AAUUACAGAAUCUACUAAAACAAGGCAAAAUGCCGUGUUUAUCUCGUCAACUUG
UUGGCGAGAmUsmUsmUsU, wherein “m” denotes a 2′-O-methyl and “s”
denotes a phosphorothioate; selected from
(SEQ ID NO: 411)
mCsmAsmGsUAUGGACACUGUCCAAAGUUUUAGUACUCUGUAGAAAUACAG
AAUCUACUAAAACAAGGCAAAAUGCCGUGUUUAUCUCGUCAACUUGUUGGCGAG
AUsmUsmUsmU;
(SEQ ID NO: 412)
mCsmAsmGsUAUGGACACUGUCCAAAGUUUUAGUACUCUGCG GAAA
CGCAGAAUCUACUAAAACAAGGCAAAAUGCCGUGUUUAUCUCGUCAACUUGUUG
GCGAGAUsmUsmUsmU;
(SEQ ID NO: 406)
mCsmAsmGsUAUGGACACUGUCCAAAGUUUUAGUACUCUGGAAACAGAAUC
UACUAAAACAAGGCAAAAUGCCGUGUUUAUCUCGUCAACUUGUUGGCGAGAUsmU
smUsmU;
(SEQ ID NO: 413)
mCsmAsmGsUAUGGACACUGUCCAAAGUUUUAGUACUCGAAAGAAUCUACU
AAAACAAGGCAAAAUGCCGUGUUUAUCUCGUCAACUUGUUGGCGAGAUsmUsmUs
mU;
(SEQ ID NO: 414)
mCsmAsmGsUAUGGACACUGUCCAAAGUUUUAGUACCCGAAAGCAUCUACU
AAAACAAGGCAAAAUGCCGUGUUUAUCUCGUCAACUUGUUGGCGAGAUsmUsmUs
mU[[]], wherein “m” denotes a 2′-O-methyl and “s” denotes a
phosphorothioate; or selected from
(SEQ ID NO: 415)
mCsmAsmGsUAUGGACACUGUCCAAAGUUUUAGUACUCUGUAAUGAAAAUU
ACAGAAUCUACUAAAACAAGGCAAAAUGCCGUGUUUAUCUCGUCAACUUGUUGG
CGAGAUsmUsmUsmU;
or
(SEQ ID NO: 415)
mCsmAsmGsUAUGGACACUGUCCAAAGUUUUAGUACUCUGUAAUGAAAAUU
ACAGAAUCUACUAAAACAAGGCAAAAUGCCGUGUUUAUCUCGUCAACUUGUUGG
CGAGAmUsmUsmUsU, wherein “m” denotes a 2′-O-methyl and “s”
denotes a phosphorothioate.
wherein “m” denotes a 2′-O-methyl and “s” denotes a phosphorothioate.

114-117. (canceled)

118. A formulation comprising a lipid nanoparticle comprising an mRNA expressing a base editor and a gRNA, wherein the base editor comprises a Cas9 domain and at least one adenosine deaminase variant comprising V82G, Y147T/D, Q154S, and one or more of L36H, I76Y, F149Y, N157K, and D167N with reference to SEQ ID NO: 1: MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 1), or corresponding alterations in another adenosine deaminase; and the gRNA comprises
(SEQ ID NO: 370)
CAGUAUGGACACUGUCCAAA.

119. A formulation comprising a lipid nanoparticle comprising an mRNA expressing a base editor, wherein the base editor comprises a Cas9 domain and at least one adenosine deaminase variant comprising V82G, Y147T/D, Q154S, and one or more of L36H, I76Y, F149Y, N157K, and D167N with reference to SEQ ID NO: 1: MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 1), or corresponding alterations in another adenosine deaminase; and a gRNA comprising
(SEQ ID NO: 370)
CAGUAUGGACACUGUCCAAA.

120-124. (canceled)
